SEQUENCE LISTING (169 sequences)

SEQ ID NO: 1 - DNA, 1954 bp, Lactuca sativa
  source 1..1954: mol_type = unassigned DNA
acaatatctgcacgctcaaaataagacaatggcatctcttgcacaatcaccaaccaccaccaccaccaccggtggacggtgcttctcctcctcctccacgtactcttcttccttctctttcaaatcatctcaagttcccatagcacgaatcacgaaccatcgccatgcagtttcatgcaaaggcgccctagatgatgatgaccatcaccatgaaaactcaggcaaatttgataggagaaacgtcttattaggtctcggaggtctttacggcgccgccgccacttttgggtcaaactcattggcgtatgcagctccgattatggcaccggacctcacaaaatgtggtccggctgacttaccccaaggggctgtacctacaaactgttgccctccatacaccacaaagattcacgatttcaaacttccaccaccgtcaaccaccttccgagtccgtccggcagctcatttggctaataaagattacatagccaagttcaataaagccatcgagctcatgaaagctctcccagatgacgatcctcgtagtttcaagcaacaagctgctgttcattgtgcgtattgcgatggggcatacgatcaagtcggtttccctgatctcgagcttcaagtccatggctcatggttgttcttacctttccaccgctattacttatacttcttcgagaaaatttgtggcaaattaatcgatgatccaaatttcgcaatccctttttggaactgggatgcacctgatggcatgaagatccctgatatttacacgaataagaaatctccgttgtacgatgctcttcgtgatgcgaagcatcaaccaccgtctctgattgatcttgactacaatggtgacgatgaaaatcttagccgatcgaaacaaacctccacaaatctcacaattatgtacagacaaatggtgtctagttccaagactgctagtcttttcatgggtagtccttatcgtgcaggtgatgaggctagccctggctctggctcgctcgagagcataccacatggcccggttcatatctggaccggagataggaaccagcaaaatggtgaagacatgggtaacttttattctgcagccagagaccctattttctatgcacatcatgcgaatatcgacagaatgtggtcagtttggaaaactctaggaggaagaaggaatgattttacagataaagactggcttgattcttcgttcttgttctacgatgagaacgctgaaatggttcgagtcaaggtgagggattgtctcgactccaagaagcttgggtacgtttatcaggatgtagagataccatggctaaaaagcaaacccgaaccacgtctgaaaagggctttgagcaagatcaagaagctcgctgtagctcgagccgatgaacacataccctttgcaaaagatgtttttccggcgagtcttgataaggtgataaaagtgctggttccaaggccgaagaaatcaaggagcaagaaacagaaagaggatgaagaagaaattttggtgatagaaggaattgaactgaagagagatgagtttgcgaagtttgatgtgtttgtgaacgatgaagatgacgggatgagggccacggctgataagacggagttcgccggaagttttgttaatgtccctcataagcataagcatgggaagaatgtgaagacaagattgaggttaggaataagtgagcttttggaggatttgggagctgaagatgatgacaacgtgttggtgacattggtgccgaaaaacaaaggtggtgaagtttccattaaagggattaaaatcgagcatgaggattgataaaaataacttttcattttcttgaaaaataaaaaactatgattttgagttgtttcggtaaatatgttgtcgcttggttaatgtatcatcaataaaaataaatttcgaaatcaaagttggatttgaaccccacaa

SEQ ID NO: 2 - protein, 596 aa, Lactuca sativa
MASLAQSPTTTTTTGGRCFSSSSTYSSSFSFKSSQVPIARITNHRHAVSCKGALDDDDHHHENSGKFDRRNVLLGLGGLYGAAATFGSNSLAYAAPIMAPDLTKCGPADLPQGAVPTNCCPPYTTKIHDFKLPPPSTTFRVRPAAHLANKDYIAKFNKAIELMKALPDDDPRSFKQQAAVHCAYCDGAYDQVGFPDLELQVHGSWLFLPFHRYYLYFFEKICGKLIDDPNFAIPFWNWDAPDGMKIPDIYTNKKSPLYDALRDAKHQPPSLIDLDYNGDDENLSRSKQTSTNLTIMYRQMVSSSKTASLFMGSPYRAGDEASPGSGSLESIPHGPVHIWTGDRNQQNGEDMGNFYSAARDPIFYAHHANIDRMWSVWKTLGGRRNDFTDKDWLDSSFLFYDENAEMVRVKVRDCLDSKKLGYVYQDVEIPWLKSKPEPRLKRALSKIKKLAVARADEHIPFAKDVFPASLDKVIKVLVPRPKKSRSKKQKEDEEEILVIEGIELKRDEFAKFDVFVNDEDDGMRATADKTEFAGSFVNVPHKHKHGKNVKTRLRLGISELLEDLGAEDDDNVLVTLVPKNKGGEVSIKGIKIEHED

SEQ ID NO: 3 - DNA, 2001 bp, Lactuca sativa
  source 1..2001: mol_type = unassigned DNA
ttatataaatatagatcacttcacataatctcatgcatcgtgctcaaaattatgacatcctttgcatcatcaccgaccaaaaccctaacaggcacggcctccaggagtgaaaggaggatctcttcctcatccaactactcttcttccttctcttttaagtcatctcaagttcccatagccagaatctccaaacatcgccatgcagtttcatgcaaaaccctagatgacgatcaccaccaccatgcaaactccggcaaacttgataggagaaacatcctgttaggcctcggtggtctttatggtactgccgccacttttgggtctaattcaccagccattgcagctccgatcatggcacccgacctctcaaaatgtggtccggccgacttgcccgaaggtgctgtatccacagactgttgccctccatacaccacaaagattctcgatttcaaacttccaccaccgtcaaacaccttccgagtccgtccggcagctcatttggctaatgaagattacataggcaagttcaataaagccatcgagctcatgaaagctctcccagatgacgatcctcgtagctttaagcagcaagcaaatgttcattgtgcctattgcgatggcgcgtatgaccaagtcggttttccagatctggagcttcaagtacataactcatggctgttcttccctttccatcgctattacatgtacttcttcgagaaaatttgtggcaagttaattgatgacccaaatttcgcaattccattttggaactgggatgcaccagatgggatgaaaatccctgatatttacacaaataagaaatcttcgttgtatgatcctcttcgcgatgtggaccatcaaccaccgtctttgattgatcttgacttcaatggtgtcgacgaaaatcttagcccctctgaacaaacgtccaaaaatctcacagttatgtatagacaaatggtgtctagttccaagacttctactcttttcatgggtagtccttatcgtgcaggcgatgatgctagccctggtagtggttcgatcgagaacacaccacataacccagttcatatctgggccggtgagtggaagcataataatggcaaaaacatgggcaaactttattctgcagccagggaccctcttttctatgcacatcatgggaatattgatagaatgtggtcagtttggaaaacactaggtggaagaaggaaggactttactgataaagattggcttgattcttcgttcttgttttacgatgagaacgctgagttgaatcgagtcaaggtgagggattgtctcgacaccaagaatcttggctacgtttatcaagatgtagagataccatggctaaaaagcaaacctgttccacgtcggacaaagcccaagcagaagcccaaaaacaaaaacaacaagcaagctgtggctcgagccgacgaatacataccatttgcaaaagatgtttttccggcgagtcttaatgaggtcatcaaagtgctggttccacggcccaagatatcaaggagtaagaaacagaaagaagaagaagaagagattttggtgatcgaaggaattgaagtgaagatagatgagtttgtgaagtttgatgtgtttgtcaatgatgaagatgacgggatgagggccaccgcagataagacggagtttgccggaagttttgtgaatgtccctcatactcataagcatgggaagaatttgaagacgagattgaggctagggataagtgagcttttggaggatttgaatgctgaagatgatgaaaatgtgttggtgacattggtgcccaaaactaggggtagtggaatttccattgcagagatcaaaatcgagcatgaagaatgatcaaaatacgttcactttctctaaaataaaaataaaggaacgtttatgatggaggaatatattatgtttttcgatatgtattaccatataaaaataattttcgcaatcaaataaaggaacgtttatgatggaggaata

SEQ ID NO: 4 - protein, 603 aa, Lactuca sativa
MTSFASSPTKTLTGTASRSERRISSSSNYSSSFSFKSSQVPIARISKHRHAVSCKTLDDDHHHHANSGKLDRRNILLGLGGLYGTAATFGSNSPAIAAPIMAPDLSKCGPADLPEGAVSTDCCPPYTTKILDFKLPPPSNTFRVRPAAHLANEDYIGKFNKAIELMKALPDDDPRSFKQQANVHCAYCDGAYDQVGFPDLELQVHNSWLFFPFHRYYMYFFEKICGKLIDDPNFAIPFWNWDAPDGMKIPDIYTNKKSSLYDPLRDVDHQPPSLIDLDFNGVDENLSPSEQTSKNLTVMYRQMVSSSKTSTLFMGSPYRAGDDASPGSGSIENTPHNPVHIWAGEWKHNNGKNMGKLYSAARDPLFYAHHGNIDRMWSVWKTLGGRRKDFTDKDWLDSSFLFYDENAELNRVKVRDCLDTKNLGYVYQDVEIPWLKSKPVPRRTKPKQKPKNKNNKQAVARADEYIPFAKDVFPASLNEVIKVLVPRPKISRSKKQKEEEEEILVIEGIEVKIDEFVKFDVFVNDEDDGMRATADKTEFAGSFVNVPHTHKHGKNLKTRLRLGISELLEDLNAEDDENVLVTLVPKTRGSGISIAEIKIEHEE

SEQ ID NO: 5 - DNA, 1967 bp, Lactuca sativa
  source 1..1967: mol_type = unassigned DNA
ctcttcacatatttcatgcacgctcaaactacaagactatggcatccttttcaccatcacaagccacctcttacacgagtggaaggaggttctcttcctcatcgacctactcttcttccttctcttttaagtcatctcaagttcccatagccagaatctccaaacatcgccatgcagtttcatgcaaaaccctagatgatgatcaccaccaccatgcaaattccggcaaacttgataggagaaacgtccttttaggccttggaggtctttatggtactgccgccaactttgggtctaattcactggcctttgcagatccgatcatgggacccgacctcagtaaatgtggtccggctgagttaccccaaggggctatacctacaaattgttgtcctccattcaccacaaagattatcgatttcaaacttccaccacagtcaaaccccctccgtgttcgaccagctgcacatttggttgataaagactacatagacaaattcagtaaagctatcgaactcatgaaagctctcccagatgacgatcctcgtagtttcaagcaacaagctaatgttcactgtgcctattgtgatgccgcatatgtccaactcggttatccagatgtggagcttcaagtacataactcatggctgttcttccctttccatcgttgttacctatacttctttgagaaaatttgtggcaaattaattgatgacccaacttttgcaattccattttggaactgggatgcgccagttgggatgaaaatccctgatatttacacagataagaattcttcgttatacgatactcttcgtgatgcgaaacatcaaccaccgactgtggttgatcttgactacaatggtttcgacaacaatcttagcccctctgaacaaacgtccacaaatctcacgattatgtatagacaaatggtgtctaatgccaagactgctagtcttttcatgggtagtccttatcgtgcaggtgatgaccctagccctggtgctggctcgctcgagagcgtgccacataacccggttcatatctggaccggggataggaaccagccaaatggtgaagacatgggtaacttttattctgcaggcaaagaccctattttctttgcacatcatgggaatctcgatagattgtggtcagtttggaaaacactaggtggaagaaggaaggattttactgataatgattggcttgattcttcgttcttgttgtacgatgagaacgctgagttgaatcgagtcaaggtgagggattgtgtcgactccaagaatatgaattatgtttatcaagatgttgagttaccatggctagaaagcaaacctgttccacgactgcaaaaggcttccagaaacatcaagaagcatgcccatgaacacataccctttgcaaaagatgtttttccggcgagtcttgataaggtgatcaaagtgcgggttccaaggcttaagaaatcaaggaccaagaaacagaaagaggaggaagaagagattttggttattgaagggattgaagtgaagagagatgagtttgtgaagtttgatgtgttggtgaacgatgatgatgatgggacccaggccacagcagctaaaacggagttcgccggaagttttgcgagtgtccctcatatgcataagcatgggaagaattggaagacgaaattgaggatagggataactgaccttttggaggatttgaaggatgaagaagatcacaatgtgttggtgacattggtgcccaaaactagtggtggtgatatttccattggagggatcaaaatcgagcatgaagaatgttaaacagacgttctcactttctataaataaaataaaagaaagtctatgatctattaagatattatgtttctctatatgtattaccatataaaagtaattatcacaataaagttatatatgattcgaacttgtgaatgttaattgcaagtcattgaa

SEQ ID NO: 6 - protein, 591 aa, Lactuca sativa
MASFSPSQATSYTSGRRFSSSSTYSSSFSFKSSQVPIARISKHRHAVSCKTLDDDHHHHANSGKLDRRNVLLGLGGLYGTAANFGSNSLAFADPIMGPDLSKCGPAELPQGAIPTNCCPPFTTKIIDFKLPPQSNPLRVRPAAHLVDKDYIDKFSKAIELMKALPDDDPRSFKQQANVHCAYCDAAYVQLGYPDVELQVHNSWLFFPFHRCYLYFFEKICGKLIDDPTFAIPFWNWDAPVGMKIPDIYTDKNSSLYDTLRDAKHQPPTVVDLDYNGFDNNLSPSEQTSTNLTIMYRQMVSNAKTASLFMGSPYRAGDDPSPGAGSLESVPHNPVHIWTGDRNQPNGEDMGNFYSAGKDPIFFAHHGNLDRLWSVWKTLGGRRKDFTDNDWLDSSFLLYDENAELNRVKVRDCVDSKNMNYVYQDVELPWLESKPVPRLQKASRNIKKHAHEHIPFAKDVFPASLDKVIKVRVPRLKKSRTKKQKEEEEEILVIEGIEVKRDEFVKFDVLVNDDDDGTQATAAKTEFAGSFASVPHMHKHGKNWKTKLRIGITDLLEDLKDEEDHNVLVTLVPKTSGGDISIGGIKIEHEEC

SEQ ID NO: 7 - DNA, 2045 bp, Lactuca sativa
  source 1..2045: mol_type = unassigned DNA
accacaccacctatagatgatggcttccctcagcttgtccactcttcccacctccacccccacaaaaaagcctttattttccaaaacctcctcccacgtgaagcaatcccatcgcttcaaagtctcatgcaactccgccgctaacaacaatgagaaaacagtcaaaaactctgaaaccccgaagctcatactacccaaaacaccacttgaaatgcagaatgttgaccggagaaacctgctcctggggcttggaggtctctacggcgctgccaacttgacatccatcccatcagcctttggcactcccatcgctgctccggacaatatttcagattgtgttactgcgtcgtcaaacctccagaacgccaatgacgctgtaaggggtttagcttgttgccctccagtactctcaacagataaaccaaaagattacgtcttgcctaccaacccagtccttcgtgttcgaccagctgcacagagagctactgacgagtacatcgtaaagtacaaagcagcgattcaagccatgaagaatctccccgacgagcatccacacagttggaagcaacaagctaagatccactgcgcttattgcaacggtggttacaatcaagaacagagtggtttcccggacatacaactccagattcacaacacatggctcttctttcctttccaccgatggtacctctacttctacgagaggattttggggaagttgattaatgatccaactttcgctttaccatactggaactgggataaccctaccggaatggtgctccctgccatgttcgaaaccgacggcaaaaggaaccctatctttgacccttacaggaatgccacacacctcccaccagctatctttgaagtgggatataatgggacagacagtggcgccacttgtatagaccagataagcgctaatctgtctttgatgtacaagcaaatgatcaccaacgctcctgatacaacaacgttcttcggtggagaatttgttgctggggatgaccctcttaacaaagagtttaacgttgctgggtccatagaggctggggttcacactgcggcgcatagatgggtgggtgatcctaggatggccaacagcgaggacatggggaacttctactccgcagggtatgatcctctcttttacgtccaccatgccaacgtcgaccggatgtggaaaatctggaaagatttgggaatcaagggacacactgaaccgacgtccaccgactggctagatgcttcatacgtgttttatgatgagaacgaagagcttgtacgtgtctataaccgagacagtgtaaacatgactgcaatgggatacgactatgaaaggtccgaaatcccgtggctccatagtcgatcggttccacataccaagggggccaatgttgcagctaaactggtcggaatcgtgaagaaggtggaagacgttacattcccgttgaagttaaatgagacagtgaaggttcttgtgaaaaggcctactaagaagaggaacaagaagaacaagcaggaagcgaatgagatgttgttcttgaataaaatcaagttcgatggcgaggagtttgtcaagtttgacgtgtttgtcaatgacgttgacgatggagtggagactaccgcagctgagagtgagtttgctggtagtttctcacaattgccccatggccataaacatggcaccaagatgtcaatgacgagtggggcggcgtttgggcttacggagctgttggaggacattgaagctgaagatgatgactctattttggtgactttggtgcccaagatagggtgtgatgatgtgactgtcggtgagattaagattaagttggttcccattgtctgaagttcattgatgtaacatcgttttcatttgcgtttgtatgcatgggtaaaacagttttctgtgtttggtcatacgaggatgtttgtggttctcgtaatctaataatgaccattttgtcaagtttgttgtcatgcttgattgtaactcctatgtttggatatcaataaacattatcgagtactattttagt

SEQ ID NO: 8 - protein, 612 aa, Lactuca sativa
MMASLSLSTLPTSTPTKKPLFSKTSSHVKQSHRFKVSCNSAANNNEKTVKNSETPKLILPKTPLEMQNVDRRNLLLGLGGLYGAANLTSIPSAFGTPIAAPDNISDCVTASSNLQNANDAVRGLACCPPVLSTDKPKDYVLPTNPVLRVRPAAQRATDEYIVKYKAAIQAMKNLPDEHPHSWKQQAKIHCAYCNGGYNQEQSGFPDIQLQIHNTWLFFPFHRWYLYFYERILGKLINDPTFALPYWNWDNPTGMVLPAMFETDGKRNPIFDPYRNATHLPPAIFEVGYNGTDSGATCIDQISANLSLMYKQMITNAPDTTTFFGGEFVAGDDPLNKEFNVAGSIEAGVHTAAHRWVGDPRMANSEDMGNFYSAGYDPLFYVHHANVDRMWKIWKDLGIKGHTEPTSTDWLDASYVFYDENEELVRVYNRDSVNMTAMGYDYERSEIPWLHSRSVPHTKGANVAAKLVGIVKKVEDVTFPLKLNETVKVLVKRPTKKRNKKNKQEANEMLFLNKIKFDGEEFVKFDVFVNDVDDGVETTAAESEFAGSFSQLPHGHKHGTKMSMTSGAAFGLTELLEDIEAEDDDSILVTLVPKIGCDDVTVGEIKIKLVPIV

SEQ ID NO: 9 - DNA, 2107 bp, Lactuca sativa
  source 1..2107: mol_type = unassigned DNA
cttatagcaccacccatagatgatggcttctctcgccttgtctagtcttcccacctccaccacaaccaaaaaacccttattttccaaaacatcctcgcatgttaagccattccatcgcttcaaagtttcatgcaatgcacccgctgataacaatgacaaaaccgtcaataattctgataccccaaagctcatactacccaaaacaccacttgaaacgcagaacgtagacaggagaaacttgcttctgggactcggaggtctctacggcgctgccaacttgacgaccattccgtcagcctttggcattcccatcgctgctccagacaatatttcagactgtgttgctgcgacttcaaacctaaggaacagcaaagacgctataaggggactagcgtgttgtcctccggtgctttcaacaaacaaaccaatggattacgtccttccttcaaaccctgtgattcgtgttcgaccagctgcacagaaagccactgccgattacattgctaagtatcaacaagcaattcaagccatgaaggatctccccgaggaccacccacatagctggaagcaacaaggcaagattcactgtgcttattgcaacggtggttacaatcaagaacaaagtggttacccgaatttacaacttcagattcacaactcatggctcttctttcctttccaccggtggtacctctatttctacgagaagatattggggaagttgattaatgatccaactttcgctctaccttactggaactgggataaccctactggaatggttattcctgccatgttcgaacagaacagcaaaactaactctctgtttgaccctttaagggatgcgaaacacctcccaccttctatctttgatgttgaatatgctggtgcagacactggtgccacttgtatagaccagatagccattaatctgtcttcaatgtacagacagatggtcaccaactccactgatacaaaacgattcttcggtggcgaatttgtagctggaaatgaccctcttgcgagcgagttcaacgtagctgggaccgtagaagctggggttcacactgcggctcaccgctgggtgggtaattctaggatggccaacagcgaagacatggggaacttctactctcgcaggatatgatcctctcttttacgtccaccatgcgaatgtcgacaggatgtggcaaatctggaaagatattgacaagaagacacacaaggatccgacctctggcgactggctaaatgcatcatacgtgttttacgatgagaatgaaaatcttgtacgtgtctacaaccgagactgtgtagacattaatcggatgggatatgactacgaaaggtcagcaatcccatggatccgtagtcggccgactgcacatgcgaagggggcgaacgttgctgctaagtctgctggaatcgtgcagaaggtggaggatatcgtattcccgctgaagttaaacaagatagtgaaggttctagtgaagaggccagctacaaacaggaccaaggaggaaaaggagaaagcaaatgagctgttgttcgtgaatggaatcacgtttgatgctgagcggtttctaaagattgacgtgtttgtcaacgacgtcgacgatggaattcagaccaccgctgctgatagtgagtttgctggtagtttcgcacagttgccacataaccatggcgacaagatgtttatgaggagtggggcagcgttcgggatcacggagctcttggaagacattgaagctgaaggtgatgactctgttgttgtgacattggtgccgagaacagggtgtgatgaagtaactattggcgagatcaagattcagctggttcccattgtttaaagtctattgaagtaatgcattttcaattgtcattagtatgcatgggtacgtaaatctgttcgctgtctggttatcgaggatttttgatgttctcgtaaccaaataataaggattgtcattccatgtttggaatcgtgtaaccgcaggcatgcatatgtttgattgttatttttacttgaagcacttctgttttagtaatctctgttttcctgttttacaaaaggtaaagaattcgctgtatgtgctttgcataa

SEQ ID NO: 10 - protein, 375 aa, Lactuca sativa
MMASLALSSLPTSTTTKKPLFSKTSSHVKPFHRFKVSCNAPADNNDKTVNNSDTPKLILPKTPLETQNVDRRNLLLGLGGLYGAANLTTIPSAFGIPIAAPDNISDCVAATSNLRNSKDAIRGLACCPPVLSTNKPMDYVLPSNPVIRVRPAAQKATADYIAKYQQAIQAMKDLPEDHPHSWKQQGKIHCAYCNGGYNQEQSGYPNLQLQIHNSWLFFPFHRWYLYFYEKILGKLINDPTFALPYWNWDNPTGMVIPAMFEQNSKTNSLFDPLRDAKHLPPSIFDVEYAGADTGATCIDQIAINLSSMYRQMVTNSTDTKRFFGGEFVAGNDPLASEFNVAGTVEAGVHTAAHRWVGNSRMANSEDMGNFYSRRI

SEQ ID NO: 11 - DNA, 2101 bp, Lactuca sativa
  source 1..2101: mol_type = unassigned DNA
cgatcaacgttagcatgcacaccaccaaaagctcatggcttctcttcccacaccaacagttacagctgccggagccaccactaaaacctactcttcttctttcaccaccacttcccctgtcatctcttcttggccgttgttctcgaagaaatgtgcccttaataagccactaaaacacaaaatctcttgcaatgctggttcttctgagaactccttgaacaaccttgatcgccggaatgttcttctcggtctcggtggtcttgccggagctgtgaacttgacgtctgttccgtctgtcggagctgcgccaatatccgccccggatatttccaaatgtgggactaaccctctttcagggtttagacctggggagagcactcccaccggcggcgactgttgcccgcctgactccccccagatcatggacttcaagttccctaagaatgaggcgttcagggtgagacccgcagcccatttgctcagccctaagtacattgctaaattcaacgaagcgatcaaacgcatgaaggaacttcccgaaaccgatcctcgaaactttctgcaacaagcacacattcactgtgcttactgcaatggcgcttacactcaatcttcaagtggatttcccgatattgaaatccagattcataactcatggctgttcttccccttccaccgttggtatctctacttttacgagagaatcctggggagcttgatcgatgatcccactttcgctttgccattctggaactgggacacccctgccggaatgacaattccgaaatactttaacgatcccaaaaacgcagtttttgatcccaaaagaaaccaaggtcacttgcaaggagtcgtcgatctgggttacaatgggaaagattcagacactactgatatcgaaaaggtgaagaacaatctcgcgataatgtatcgtcaaatggtgacaaacgccaccgaccccacagctttcttcggtggtgagtatcgtgccggaatcgaacccattagcggtggtggatcagtcgaacaaagcccacacacacctgttcaccggtgggtcggtgacccaagagaacttaacggtgaaaacctcggtaacttctactccgccggtcgtgacacgctcttttactgtcaccattccaacgtcgatcgaatgtggtcgttgtggaagatgcagggaggcaaacacaaggacatcaccgatcccgattggctcaacacctctttcgtgttttacgacgaaaacaagaatcttgttcgagtgtatgttaaggattgtttgtacacaaaccagctagggtacgactaccagagagtcgacgtaccatggctaaaaagcaagccagtcccacgtgcacccaggtctggagttgcgaggaaatccatcggaaaagtaaaacaggcgaaggaagtttccttcccggtgaaactcgacaagaccgtgaaggttttggtggcaagaccgaagaaatcaagaagcaagaaggagaaggaggaccaagaggagcttttgattgttcagggtatcacttatgatagcgagaagtacgtgaagtttgatgtgtatgtgaacgacgaggacgacgatgctagtgcaccagatcagactgagttcgccggaagtttcgcacagttgccacacaaacacaagggtaagacgatgagcaagaccaacttccgcgccggactgacggagctgctggaggatctggaggccgacgacgacgacaatgttttggtgacgattgtcccaaggtctggatccgaagacatcaccattgataacatcaagatcatctacgcttgatttcaagatttgacgactctcagttgggatcattagttaaatatgtttgattaatgatcttgctattcccttaattattattattattatcagcgtagttcgacccttgattagtggtgagggttgatggttgtcaccggaatgtttgggtttgagtccgagttcatgtgattatgggtcggatttaaaataaaatctgagtcgagtctgtggtcatgtcgtaatttggggtttcacttttgaccaaatcaatgttaattataatttaaataaattattattagttctcca

SEQ ID NO: 12 - protein, 591 aa, Lactuca sativa
MASLPTPTVTAAGATTKTYSSSFTTTSPVISSWPLFSKKCALNKPLKHKISCNAGSSENSLNNLDRRNVLLGLGGLAGAVNLTSVPSVGAAPISAPDISKCGTNPLSGFRPGESTPTGGDCCPPDSPQIMDFKFPKNEAFRVRPAAHLLSPKYIAKFNEAIKRMKELPETDPRNFLQQAHIHCAYCNGAYTQSSSGFPDIEIQIHNSWLFFPFHRWYLYFYERILGSLIDDPTFALPFWNWDTPAGMTIPKYFNDPKNAVFDPKRNQGHLQGVVDLGYNGKDSDTTDIEKVKNNLAIMYRQMVTNATDPTAFFGGEYRAGIEPISGGGSVEQSPHTPVHRWVGDPRELNGENLGNFYSAGRDTLFYCHHSNVDRMWSLWKMQGGKHKDITDPDWLNTSFVFYDENKNLVRVYVKDCLYTNQLGYDYQRVDVPWLKSKPVPRAPRSGVARKSIGKVKQAKEVSFPVKLDKTVKVLVARPKKSRSKKEKEDQEELLIVQGITYDSEKYVKFDVYVNDEDDDASAPDQTEFAGSFAQLPHKHKGKTMSKTNFRAGLTELLEDLEADDDDNVLVTIVPRSGSEDITIDNIKIIYA

SEQ ID NO: 13 - DNA, 1982 bp, Lactuca sativa
  source 1..1982: mol_type = unassigned DNA
gtttttgtatgttttctttccaatcttttgcaaccttcacttccatcaccaccagaacactaccaaattccacctcggatcgccgctacaacagttaccccaaacaaatccaccaccttcaaatctcatgcaacgtcgcaccagatgacaaagagaagttagtcgtagtcccagaaacccaaaaacttattctgccaaaatcatctctcgacacacttaatgtagatcggaggaacatgctcctcgggcttggcggcctttacaccaccgtcaacttcacctcccctgcagcatttgctgcacctatcacgacgccaaacttctccacatgcgtgacctcaaatttaggtttccaggacccaaataaggccgtcagaagcagagcatgttgtccaccggcgccagcgacgtcaacagcccccaaagactttgtgttccctaaagaccaagtgatccggattagaccagcggcacatagaaccaccaccgagtacgttgctaagtacaaagcagcaatccaagcgatgagagatctcccagatgaacacccacacagtttcgttgcacaagcaaaaattcattgcgcttactgcaacggtggttacactcaaatcgcgagtggttttccagataaagaactccagattcacaactcatggctcttctttcctttccatcgttggtacttgtatttctacgagagaatcctcgggaagttaatcgatgatccaactttcgctttaccttactggaactgggaccatcccaacggaatgacgtttcccgcatttttggaggacgattctgccttcgacgcttaccgtaatcgaaagcacttaccaccagcacttgttgacctcaactacagtggctcagatagacacgctacttgtattcgacagataactagcaatatgacattaatgtataagcaaatgatcagcaacgccggtgacacgacaagcttctttggtagcgaatatcgggctggcaacgacgcgtatagaaatggtgacccatctgtcgggtcgatagaggctggttgtcacactgcggtgcatagatggatgggtgacccaggaatgccgaacaacgaggacatggggaacttctactctgcggggtatgaccctgcgttctacatccaccatgccaatgtcgaccggatgtggaaactatggaaggatatgggcatcaaaggacactctgaacctacacatctggattggcgtaacgcatcgtacgtgttttatgatgaaaacgaacagcttgttcgtgtctacaacaaagattgcgtcagtttggaaaagctaaaatacgattatgaatactccccacccctctggaaaataagccgatccagtatacgtcgtacccttcccgaacccattccatataacatgaaatctgctgaaacggttaaacaactgccagacgtgaagttccccttgaagctagacaagataacgaaggtagtagtgaagaggccagccaaaagcagaagtcaagaagacaaggaaaaagcaaatgagctgttgttgattaaaggaatcaagtttaatagcgacaagttcatcaagtttgatgtgtttgtgaatggacaagatgatgtcagcgaaagttttgaagaagagagtgagtttgcaggtagtttcgcgcagttgccacataaccatggtgacgacatgttaatgaagagtggcataaggtttgggttaacggagcttttggaggaaatggaggcggaggatgatgagtttattttggtgactttggtgccaaaggtgtggtttgaggaagtgaccattgacgaaatcaaggtggagttggttcctattatctgattcataatctaaaattgatgagcatcgggtacgtaattaacgtagcgtatacgtaccaacgctgtaaatacaaaaaagtattattattcaactaaagtaccaaagagatactcaatttagttgtagtaacggagaagg

SEQ ID NO: 14 - protein, 611 aa, Lactuca sativa
MFSFQSFATFTSITTRTLPNSTSDRRYNSYPKQIHHLQISCNVAPDDKEKLVVVPETQKLILPKSSLDTLNVDRRNMLLGLGGLYTTVNFTSPAAFAAPITTPNFSTCVTSNLGFQDPNKAVRSRACCPPAPATSTAPKDFVFPKDQVIRIRPAAHRTTTEYVAKYKAAIQAMRDLPDEHPHSFVAQAKIHCAYCNGGYTQIASGFPDKELQIHNSWLFFPFHRWYLYFYERILGKLIDDPTFALPYWNWDHPNGMTFPAFLEDDSAFDAYRNRKHLPPALVDLNYSGSDRHATCIRQITSNMTLMYKQMISNAGDTTSFFGSEYRAGNDAYRNGDPSVGSIEAGCHTAVHRWMGDPGMPNNEDMGNFYSAGYDPAFYIHHANVDRMWKLWKDMGIKGHSEPTHLDWRNASYVFYDENEQLVRVYNKDCVSLEKLKYDYEYSPPLWKISRSSIRRTLPEPIPYNMKSAETVKQLPDVKFPLKLDKITKVVVKRPAKSRSQEDKEKANELLLIKGIKFNSDKFIKFDVFVNGQDDVSESFEEESEFAGSFAQLPHNHGDDMLMKSGIRFGLTELLEEMEAEDDEFILVTLVPKVWFEEVTIDEIKVELVPII

SEQ ID NO: 15 - DNA, 1920 bp, Lactuca sativa
  source 1..1920: mol_type = unassigned DNA
atggcttcactgagcttcactttagctatggccaccaccccctcttcttccccattcttttccaaaccagcgaaccaacgtcagttgataaagacacatgctaagcaaacccaccgcttccaaatgtcatgcaatgttccatcagacgaccatgaaaaaccaatcatcaatacccctcaacatcaaaagctcatactaccaaaaacatcactcgacatgcagaacgtagacagaaggaatttgctcctggggcttggtgggctctacagcgccgtcaacttgacgggtctcccatccgcttttgccgatcctatcacgactccttcttttaatccaaattgcagggacgccggaacgggcttcgatgtcaaaaaaggccttcttagaactactgcatgttgccctccggagtccaagaagggtcccgagaaacaattcgaattccctaaacatgacgaaatacgcatcagatatcccatacactgtgccccggaaggatacatgaataaatttaaggaggcgatgaggctaatgagggctctcccagatgacgaccctcgcagtttcaagaaccaagccaaaattcattgcgcctactgcaatggcagctacactcaaatggctacaggttcccaacaagaactcctgattcacttcaactggctgttttttcccttccatcgatggtacctttatttcttcgagaggatactcggagaactgattggtgatccaacattcgggttaccatactggagctgggacgagcgtgagggaatgaaaattccacctacgttccgagaagggggagagtctaaccctttatatgatatctaccggaataacattcgcaactatgaagctattgtcgatcttgacttcaatggtaaagatcgcgaagatacgactgacgactatcagataaaaatcaatcagcatgctatgtatcgccagatgatgagaaatgccttcgatacaaaaagcttctttggtggtaagtatgtcgctggtaatacacccattgatgccaaagactcttcagttgcatccatagaggccggttgtcataccgcgattcacagatgggtgcgtgaccctggaagtccgaatggtgaagacatgggtaatttctactctgccgggtatgatcctttgttctatgtccaccattccaatgtcgacaggatgtgggcactttggaaagaaatgggggaaagcaaccgcgaccccatacacccagactggttaaacgcatcatatgtgttttacgacgagaaacaaaatcctgttcgtgtctacaataaacaatgcgtggatatggaaaagctcaaatacaaataccatggtccagaaatccccagctgggtcaattctcggccgaaaccaaagtgcagcgcttcggaaagatcccaaatcgatatcacgtcagccacaaaagatgtgaagaaccgaaccctcaccaacgtagatacgtttgtgttagtgaggcctgaaactgctagaacaaggaccgtggatgaatcagaaatagaggtcttgacgttgaacaacattagtttcaacggtaacaaagccgtcaagtttgacgtgcttgttaacgcttgtaacattgacacaaacaagttcaccccggctgatagcgagtatgcgggttcttttgcaacagttccacataaccatgacatgaaaattagtactacgttcaggtttcccttaagagagctgttgaaagatattggagctgagggaaatacagcaattcaagtcaccattgtgacgcaagagaaagaaaccgagaatatcagcattggcgagatcaagatcgaggattactctttagccgagatctcgaaggcgtcacttcccactgggctacagggtgccggagctaatgtcggcgtcgacgatctaacagaatag

SEQ ID NO: 16 - protein, 639 aa, Lactuca sativa
MASLSFTLAMATTPSSSPFFSKPANQRQLIKTHAKQTHRFQMSCNVPSDDHEKPIINTPQHQKLILPKTSLDMQNVDRRNLLLGLGGLYSAVNLTGLPSAFADPITTPSFNPNCRDAGTGFDVKKGLLRTTACCPPESKKGPEKQFEFPKHDEIRIRYPIHCAPEGYMNKFKEAMRLMRALPDDDPRSFKNQAKIHCAYCNGSYTQMATGSQQELLIHFNWLFFPFHRWYLYFFERILGELIGDPTFGLPYWSWDEREGMKIPPTFREGGESNPLYDIYRNNIRNYEAIVDLDFNGKDREDTTDDYQIKINQHAMYRQMMRNAFDTKSFFGGKYVAGNTPIDAKDSSVASIEAGCHTAIHRWVRDPGSPNGEDMGNFYSAGYDPLFYVHHSNVDRMWALWKEMGESNRDPIHPDWLNASYVFYDEKQNPVRVYNKQCVDMEKLKYKYHGPEIPSWVNSRPKPKCSASERSQIDITSATKDVKNRTLTNVDTFVLVRPETARTRTVDESEIEVLTLNNISFNGNKAVKFDVLVNACNIDTNKFTPADSEYAGSFATVPHNHDMKISTTFRFPLRELLKDIGAEGNTAIQVTIVTQEKETENISIGEIKIEDYSLAEISKASLPTGLQGAGANVGVDDLTE

SEQ ID NO: 17 - DNA, 2222 bp, Lactuca sativa
  source 1..2222: mol_type = unassigned DNA
atggcttcttttagcttttacactcttcctacttccacctccaccatcaagaatcccttattttctaaaagctcctcccatgtgaagcactcacatcgcttcaggtcttcatgcaaagccgctgctgatagcaatgacaaatatgtcgaaaatcctgataccccaaaactcatactaccaaaatcaccctcgcttgatacgcagaacgttgacaggagaaacttgctcctgggactcggaggtctctacagcgctgccaacttcaccagcattccgtcagcctttggcgttcccatcgaagcaccagacattaatatttcaaagtgtgttactgccaccgtaaggggagtctcagcagaggctataaggggattaacttgttgccctccggtgtttgactcatcggccaaaccagcgccgtacgaatttccggataaccaggtaattcgtatgcgaccagcggcacagagagtcagtgcagactacaaaaaagactttcgaaaggcagttgagataatgaagggatataacgacaatgacccacacagttggacgcaacaagctaaagttcattgtgcgtactgcaacggcgcttacactcaagtaaaaagtggtttggagttcgagaagtatataatccaagttcacaactcatggctcttctttccattccaccgttggtacctctatttcctcgagaagataatgggaaaggcgctcggggatgacactttcgctctaccatactggaactgggaccaccctaccggaatgacgattcctgccatgtacgaagacaaattaaaaaatccggatggcaacgttgatacccctgaaaacactagattcaactctctctttgatcctttaaggaatacatcacacatcgcaccagctctaattgattttcagtattatcctcagaaacaagaagtttataattgtgcagaccagatagagattaatctgtctataatgtacaatcagatgatcgccaacgcccttgatacaaaatcgttctttggtggcgaacttgtggctggtgaaaaccccaatgaaaacaaaaaggctgggtccatagaggatggggttcacacgattgcccaccaatgggtgggtaacaatagattgaagaacggagaagacatgggaaacttctactccgcaggctatgaccctctgttttacggccaccatgcgaacgttgaccggatgtggaaaatctggaaaggtatgaacaggagacaccatgcaccatcctcgaccgactggctagatgcatcctacgtattttatgatgagaatagaaaacttgtacgtgtctacaaccgtgactgtgtagacactagaacgatggggtatgattatgagaggtccgagatcccatggatccgaaatcgacctaatccacatcccaagggaggcaaagataaaggaaatgctcgcaaacccgacaaagcgacggtgaaggatctcagtttcccagtgaggttaaaccagacattggaggttcgagtgatgagacctgcgaaaaggactacggaggacaaggagcgcaccgagatcgcgattgagaagttggtccttcaaggcgtacgatatgattgtgagcgctttgtcaagttcgatgtgataatgaacgaccctgataatggagtcgatgtcaccccagttgacactgagtttcttggttatttctcacggttgccccatggcatggtcgctgaaaacagaatgaaagagattagtgggatatcatttgccatcaaagaccgcttgaaaatcctaaaagttgaaaatgatgattctattgttgtgaaaattgtgcccagagcagggtgcgaggatgtaactattcagaacatcgaggttgtgatggatcccgtagacaatattgtacctttagcggagagcctggttgtgcaagatcggaacagcgatgaacttactttggagggcccgactgcgctggattcgaattcggacgactctggctcggagtgataaattaaatcgtgtcgttgtgtatcttgtatatgtgtgatcactttataagtttaagtttgtattcgaatcgccaatgtggtgatatttgcattttgccattattaataataatccaatgctcttgtagaagaaattgctctgagtgtctagagttttgtctgaatgggtacgtacggttacttttttgtacgtcaataatgcacttatttgactta

SEQ ID NO: 18 - protein, 667 aa, Lactuca sativa
MASFSFYTLPTSTSTIKNPLFSKSSSHVKHSHRFRSSCKAAADSNDKYVENPDTPKLILPKSPSLDTQNVDRRNLLLGLGGLYSAANFTSIPSAFGVPIEAPDINISKCVTATVRGVSAEAIRGLTCCPPVFDSSAKPAPYEFPDNQVIRMRPAAQRVSADYKKDFRKAVEIMKGYNDNDPHSWTQQAKVHCAYCNGAYTQVKSGLEFEKYIIQVHNSWLFFPFHRWYLYFLEKIMGKALGDDTFALPYWNWDHPTGMTIPAMYEDKLKNPDGNVDTPENTRFNSLFDPLRNTSHIAPALIDFQYYPQKQEVYNCADQIEINLSIMYNQMIANALDTKSFFGGELVAGENPNENKKAGSIEDGVHTIAHQWVGNNRLKNGEDMGNFYSAGYDPLFYGHHANVDRMWKIWKGMNRRHHAPSSTDWLDASYVFYDENRKLVRVYNRDCVDTRTMGYDYERSEIPWIRNRPNPHPKGGKDKGNARKPDKATVKDLSFPVRLNQTLEVRVMRPAKRTTEDKERTEIAIEKLVLQGVRYDCERFVKFDVIMNDPDNGVDVTPVDTEFLGYFSRLPHGMVAENRMKEISGISFAIKDRLKILKVENDDSIVVKIVPRAGCEDVTIQNIEVVMDPVDNIVPLAESLVVQDRNSDELTLEGPTALDSNSDDSGSE

SEQ ID NO: 19 - DNA, 632 bp, Lactuca sativa
  source 1..632: mol_type = unassigned DNA
ttgaattgtttattataaagatttagcagaaatgaaaagctctttgcattgatcacaaggttctttaacttctttttagcaaacccaacatggccggaataccaactacacctgccacttttccgatgagtctagacaaaccaacgacggtgatggtggcgaggccagcaaagaaggaaagagagaaggaggaagaagaagtattggtgatagaagggatagaaatcaatagaaatgagtttgtgaagtttgatgtgttcattaacgatgaggatgaggagacagcggctggtggtggggctgagaaagctgagtgtgccggtagctttgtgaacgtgccacataagcacaggaacggacatagtggtgatggtgggaaggtgaaaaagacacagttgagaattggaataagtgagttattggaggatttgggtgtcgaagaagatgacgaagatgtggtggtgaaattggtgccaaggtgtgaaaatgttcatgtcacaattggtggtattaagattgagaatgagtgattaattaattaattaatgagttcttatcttagttattttcttttgttatcgagtggtttatgatcccttataataaatgttatacttaatgttgtcaagatc

SEQ ID NO: 20 - protein, 146 aa, Lactuca sativa
MAGIPTTPATFPMSLDKPTTVMVARPAKKEREKEEEEVLVIEGIEINRNEFVKFDVFINDEDEETAAGGGAEKAECAGSFVNVPHKHRNGHSGDGGKVKKTQLRIGISELLEDLGVEEDDEDVVVKLVPRCENVHVTIGGIKIENE

SEQ ID NO: 21 - DNA, 2118 bp, Lactuca sativa
  source 1..2118: mol_type = unassigned DNA
tttcccacaatccgcgtctatataatggagctcagcagaacggatttaacacctacgacaatgatggcttcatatatcttttccaccgttccctcagccaccgaagtcaccaccaacaacttctcccattcctcgatattttccaaaacttccactcaccgattcaaatatactcatgaaaaccaaacccatcgtttcaaagtctcatgcaacaaaacctcagacgacaaatatgatacactggaaacatcacttgacaagaagaatgtagaccgaagaaacttgcttctggggctcggtggaactctctacggtgccgccaacttgaccttcctcccgtccgccttctcggtgcccattgctgctcccaatgtttcagactgtgctattgccagtaaaggtatacacaacatcaaagatgctgtaaggggggtagcttgttgcccaccagtactgacactaaattccccaaaaaattacgtcttcccgaaggagaccgcagttcgtatccgtccggcagcacaaagagcctctgacgattacattgacaagtataaagcagcaattaaggccatgagggatctcccagatgaccacccacacagtttcaagcaacaagccaagatccattgcgcttattgcaatggctcttacactcaaaaagagagtggcaaggaatatgaacacctcacactccagattcataactcgtggctcttctttcctttccaccggtggtacctctatttctacgaaaggatattgggaaagttgattgacgacccaactttcgcgataccatactggaattgggacaaccccaccggaatgataatccctgacttgtttgaaaaacccatccaagtaagggaacgcaaagaaaaccccgtctttgacgcttacagggatgccagacacctcccaccagctcttgttgatatcgattataacggtgaagaccgtggcgtttcatgtatagaccaaataactattaatttgtctgcaatgtataagcagatgatcagtaatgctagtgacccaacaagcttcttcggtggcagatacgtcgctgggatggaccacgatgacaaaaatagtcatggaaatccatcggttgggtccatagaagctggttgtcacacagcggtgcaccgatgggtggctgatcctcggatgccgaacaatgaagacatgggaaacttctactccgcagggtatgaccctatcttttatgcccaccacgccaatgttgaccggatgtggaaaatctggaaagagttgggtatcaggggacaccgtgaaccaaccgacaaggactggctagatgcatcatacgtgttttacgatgagaatgaagaacttgtacgtgtctataaccgagactgtgtggacttgaataagcttaactacgactatgaaacatcccgtatcccgtgggccaggaatcggccgatcccacgtgctaagaatcctcaaatggcagcgaggtcagcccgaatggggaggagttttcatgacgtgcaatttccggtgaagttagacgggatagtaaaggtgctagtgaagagaccttatgtaaacaggactaaggaggagaaggagaaagcaaatgagatattgatgttgaatgggatttgttttgatagcgagaagtttgtaaagtttgatgtgtatgtggatgacaaggacgatgaaccagaaaccactgcggctgatagcgagtttgcaggtagctttgcgcagttgcctcaccatcaatcaggcgagaagatgttcatgacaagtgccgcgagattcgggttaacagagttgttggaggacattgaagctgaagatgatgaatctattatggtgactttggttcctaggacagggtccgatgatatcacaatttcagagatcaagattgagctggtccccatcgtttgaacttcaataatgtaatcgcattttcatgtccatgtaatatgttcgttttctgtgttgtgtttaattaggagaatttgctgagttctcttaacctcaaaaaagggcaataagttggtagcgttaatgtgttatcactcgtgcatgtgtatttcaattaaagttgatcaactaataaaaaattttttgttg

SEQ ID NO: 22 - protein, 634 aa, Lactuca sativa
MELSRTDLTPTTMMASYIFSTVPSATEVTTNNFSHSSIFSKTSTHRFKYTHENQTHRFKVSCNKTSDDKYDTLETSLDKKNVDRRNLLLGLGGTLYGAANLTFLPSAFSVPIAAPNVSDCAIASKGIHNIKDAVRGVACCPPVLTLNSPKNYVFPKETAVRIRPAAQRASDDYIDKYKAAIKAMRDLPDDHPHSFKQQAKIHCAYCNGSYTQKESGKEYEHLTLQIHNSWLFFPFHRWYLYFYERILGKLIDDPTFAIPYWNWDNPTGMIIPDLFEKPIQVRERKENPVFDAYRDARHLPPALVDIDYNGEDRGVSCIDQITINLSAMYKQMISNASDPTSFFGGRYVAGMDHDDKNSHGNPSVGSIEAGCHTAVHRWVADPRMPNNEDMGNFYSAGYDPIFYAHHANVDRMWKIWKELGIRGHREPTDKDWLDASYVFYDENEELVRVYNRDCVDLNKLNYDYETSRIPWARNRPIPRAKNPQMAARSARMGRSFHDVQFPVKLDGIVKVLVKRPYVNRTKEEKEKANEILMLNGICFDSEKFVKFDVYVDDKDDEPETTAADSEFAGSFAQLPHHQSGEKMFMTSAARFGLTELLEDIEAEDDESIMVTLVPRTGSDDITISEIKIELVPIV

SEQ ID NO: 23 - DNA, 1036 bp, Lactuca sativa
  source 1..1036: mol_type = unassigned DNA
ttttttggcaatataaatagtgaacgctagttctcgtaattcgggtaggtgtatgatggcttcttttaacttgtacaccgttcccgccgccactacggccaccaccaacataatcaaaactaactactaccaacttaagactcaagcaaagcaaacccatcgcttgaaagcgtcatgcaacgcaatcccggataaaaacaatgataaagcacttgaaacttcacttcaaatgataaacattgaccggagaaacataatactccgcctcggtggtctctttgtggccagcaacatgacctcggttcctttggcctacgctaatgcaattgcagctcggtctaatcactcggtttgtgctgcttcgcctttaggcatacagaatcttggaacccccgtaaaggaacccatggagaatagggaagatgtagacacgaataagctcggatacgaatacaaatggtccgaaatcccatggggaaggagtcaaccgactgaatatggcaaggattcaaaatttgtagataagtctataggaatagagaagaaggtgggggaggtggaatttccagtgaagttaaacaagacagttaaggtactcgtgaagaggccggctgttaataggaccaaggaggacaaacagaaagcgaatgagattttgttggtaaatggagtgagattcgatggtgagaagtacgtcaagttcgatgtctttgtcaacgacatagacaatggaaccgagaccaccccggctgacagtgagtttgctggtagtttcgcacagcttccgcatggcaaaaccgacaggatgatgatgatgagcggagttaggtttgggttaacggagcttttggaggacataaaagctgaagatgatgaatatgttttggtgaaattggtgcctaggactgggtgtgatgacgttacagtttccgagatcaagattgaactggagggaataatgtcaatggacaagaagtcaaagtttgttgaatctgaatatgagaatgtgattgattacttgtacatatgtttatag

SEQ ID NO: 24 - protein, 327 aa, Lactuca sativa
MMASFNLYTVPAATTATTNIIKTNYYQLKTQAKQTHRLKASCNAIPDKNNDKALETSLQMINIDRRNIILRLGGLFVASNMTSVPLAYANAIAARSNHSVCAASPLGIQNLGTPVKEPMENREDVDTNKLGYEYKWSEIPWGRSQPTEYGKDSKFVDKSIGIEKKVGEVEFPVKLNKTVKVLVKRPAVNRTKEDKQKANEILLVNGVRFDGEKYVKFDVFVNDIDNGTETTPADSEFAGSFAQLPHGKTDRMMMMSGVRFGLTELLEDIKAEDDEYVLVKLVPRTGCDDVTVSEIKIELEGIMSMDKKSKFVESEYENVIDYLYICL

SEQ ID NO: 25 - DNA, 2032 bp, Lactuca sativa
  source 1..2032: mol_type = unassigned DNA
gttcgaagaagtgagtggagtaacccaaaacaaaataacaatggcttctttccaacttgttaaccccttcgccagcaccacaagaaaactgccagattccacctccagccgtcgtctcaagactcaccctcaaaaaaaccaccgcttcaaagtctcatgcaacgtcgcacaagatggcaatgagaagctactcttagtcccagatagcaaaaaccttatactacctaaaccatcactcgacacgcttaatgtagatcggaggaacttgctactgggactcggtggcctttacagcaccgtcaacttcacctctcttccggcggccattgccgcccctatcaccacgcctgacatctccacatgcatcccgtcagagcaaggcttcaacgtgcaggactccgtaagaagcaaccaatgttgtccgccgatgatgaccacaaccccgaaagactttgtgttcccaaaagacaaaacaattcgggttagaccagcggcacatagagccacccccgagtacatagcaaagtacaaagcagcaatccaagcgatgaaagatctcccagatgaccacccacatagtttcgttcaacaagctaaaattcattgcgcttactgcaacggtggttatactcaagtcgcaagtggttatgctgataagcaactccagattcacaactcatggctcttctttcctttccatcgctggtacttgtatttctacgagagaatcctcgggaagttaattgatgatcccactttcgctttaccttactggaactgggacaatcccgccggaatgtcatttccagctttttttgaaaccgacggcaagagaaaccctgtctttgacgcgttccgcaacgtcaaccatgtatcaccagaaacagttgtcgatctcgactacaatggctcagatagtggcgctccttgtcttcaacagataagcaccaatcttgctgcaatgtataagcagatgatcagcaacgctactgacccgttaagtttctttggtggcgagtttcgggctggagatgacccttttggaaatagtgacccatctgtcggatcaatagaggctggttgtcacactgcgatgcacagatggacgggaaatccgagaatgccaaacaacgaggacatggggaatttctactctgcggggtacgaccctgcgttctacgtccaccatgcgaatgttgaccgtatgtggaaagtatggaaggatttaggtatcaaaggacacactgaacctacggaccctgattggcttaatgcatcatatgtgttttatgatgaaaacgaagagcttgtacgtgtttacaacaaagattgtgtccaaactgaaaacttaaaatatgatttcgaattatccccactcccttggctcaagaaccgaccggttgcacataccaaaccagagaccaccacgaaacctgttgaaaaggttaaagtgccggacgtgaagttccccattaagctagacaagatacagaaggtccttgtgaagcggccagcgaaaaaccgaagccaatcagaaaaagaaaaagcgactgagcagttgttgatcaaaggaatcaagtttaatgtctccaagttcgtcaagtttgatgtgtttgttaatgaccaagatgatgttcccacaagctctgcatccgagagcgagtttgcaggtagtttcgcacagttgcctcatcaccatggtggccacaaaaagttaatgacaagtgcagcaaggttcggtttaacagagcttttggaggacatcggagccgaggatgatgagtatattttggtgacattggtgccaaaggtaggggccgaagatctcaccgttgatgaaatcaaagtggagttggttcctattgtttaaacaaccatgtaatcttatctacacaataaggtatatatttaaataattcgtaggccttgcattccattgcatggttgcgacttttatgtacgaatattaataacttcattgtgtcctttttctacgagtaaatggagtgaactcaacatcttttgcttgttc

SEQ ID NO: 26 - protein, 609 aa, Lactuca sativa
MASFQLVNPFASTTRKLPDSTSSRRLKTHPQKNHRFKVSCNVAQDGNEKLLLVPDSKNLILPKPSLDTLNVDRRNLLLGLGGLYSTVNFTSLPAAIAAPITTPDISTCIPSEQGFNVQDSVRSNQCCPPMMTTTPKDFVFPKDKTIRVRPAAHRATPEYIAKYKAAIQAMKDLPDDHPHSFVQQAKIHCAYCNGGYTQVASGYADKQLQIHNSWLFFPFHRWYLYFYERILGKLIDDPTFALPYWNWDNPAGMSFPAFFETDGKRNPVFDAFRNVNHVSPETVVDLDYNGSDSGAPCLQQISTNLAAMYKQMISNATDPLSFFGGEFRAGDDPFGNSDPSVGSIEAGCHTAMHRWTGNPRMPNNEDMGNFYSAGYDPAFYVHHANVDRMWKVWKDLGIKGHTEPTDPDWLNASYVFYDENEELVRVYNKDCVQTENLKYDFELSPLPWLKNRPVAHTKPETTTKPVEKVKVPDVKFPIKLDKIQKVLVKRPAKNRSQSEKEKATEQLLIKGIKFNVSKFVKFDVFVNDQDDVPTSSASESEFAGSFAQLPHHHGGHKKLMTSAARFGLTELLEDIGAEDDEYILVTLVPKVGAEDLTVDEIKVELVPIV

SEQ ID NO: 27 - DNA, 2060 bp, Lactuca sativa
  source 1..2060: mol_type = unassigned DNA
aaaggagtatgaagcacaccaaaccaacatggcttctttgagctttactttagccactcccaccacctcttcctcgccgttcttttcacaaacaaccaccaaacaacgacggttgatgaagacacatgggaagcaaacccatcgcttccaagtctcatgcaacgtctcatcaaataaccatgaaaaaccactccccaaaaaccctcaaccacaaaaacttatactaccacaaacatcactcgacttgcagaacgtcgacagaaggaatttgctcctgggtctcggcggagtctacagcaccgccaccttgtccggtctgccaccggcctttgcagaagctatcaaggctccgttcaatcagcctgatcgaccatgcaaagatgccgtatccggcttcgacattaataaaaagctacttagacctattgactgttgccctctgtccaaaaatggcccggagagtcatttcaagttccctgataaatcaagcaaaactcgcatcagatatccactacacaaacttccagtcgggtatctcgataaatatatggatgcgattcagaaaatgaaggatctcccagatagcgacccacgcagtttcaataaccaagctaaagttcattgcgcttactgcaatggcagttacactcaaaacggtcaagaactccagattcacaactcctggctcttctttcccttccatcggtggtacctttatttctacgagaggatactgggagatctcattggtgattcgacattcgggttaccctactggaactgggacaaccccgaaggaatgacaattccacacttcttcgtagagaaacaatgtaacaactataagttcgaaaacggagaaaaccctctatatgataagtatcgggacgaaagtcaccttcggtatgaattggtcgatcttgactactcagggagaaaccgcgacctgtgttacgatcagaaagaaatcaatctggctactatgaataggcagatgatgcgcaacgcctttgatgcaacaagcttcttcggtggcaaatatgtagccggtgatgaaccgattccccgaggagataatgtagttggatccgtggaggctggttgtcatacggctgttcacagatgggttgggaaccctgatccaaaagggaataaagaggacatgggcaacttctactctgcgggatatgatcctttgttctacgtccaccattctaatgtggaccgaatgtggactctttggaagcaaatgggaggcaaagaaccgacagatactgactgggaaaacgcgtcatacgtgttttacgatgagaaacaaaatcccgtacgtgtctacaacaaacaatcggtggatttgagcaaccttaaatacgaataccacagctcagccactccatggacggatagaccaccaagatcacgctgcaacagacccggttacccgaagaggaacaacacaaaggacttcccaaatcagaaggacccgccagaagctttgacattaaccgatagtactgtgaggcttcgagtaaagaggcctcctgcttctaaaaacaggaacgctgagcaaaagaaaagtgaaaaagagatcttgtgcttgattggaatcagtttcgattgtaccgaagctgcaaaatttgacgtgtttgtgaatgattgtgacgaagaacagatcaccccgtgtgatagtgagaatgtgggttctttcgcggctgttccacatgctaaaggcatggcaatgggttgcaagtctgggatgaggttttcgttaacagagttgttggaggaaacaaaagctgagggggatgagtctattcgggtgacaattgtgccgaggacgacacccggcaagaaagtcaaagtcaccattgatgctatcgagatccggttgattccggttcttgaaaaataatcaaactgatcaaatgcgttgatgatggattccacaatcacgaaatgaataagttggagtctgtgtagttcgataaacgtacgttgtgatttctaatgcatgttttatttgaatagaaatttgttttaaacaaaagtctcttcatttc

SEQ ID NO: 28 - protein, 627 aa, Lactuca sativa
MASLSFTLATPTTSSSPFFSQTTTKQRRLMKTHGKQTHRFQVSCNVSSNNHEKPLPKNPQPQKLILPQTSLDLQNVDRRNLLLGLGGVYSTATLSGLPPAFAEAIKAPFNQPDRPCKDAVSGFDINKKLLRPIDCCPLSKNGPESHFKFPDKSSKTRIRYPLHKLPVGYLDKYMDAIQKMKDLPDSDPRSFNNQAKVHCAYCNGSYTQNGQELQIHNSWLFFPFHRWYLYFYERILGDLIGDSTFGLPYWNWDNPEGMTIPHFFVEKQCNNYKFENGENPLYDKYRDESHLRYELVDLDYSGRNRDLCYDQKEINLATMNRQMMRNAFDATSFFGGKYVAGDEPIPRGDNVVGSVEAGCHTAVHRWVGNPDPKGNKEDMGNFYSAGYDPLFYVHHSNVDRMWTLWKQMGGKEPTDTDWENASYVFYDEKQNPVRVYNKQSVDLSNLKYEYHSSATPWTDRPPRSRCNRPGYPKRNNTKDFPNQKDPPEALTLTDSTVRLRVKRPPASKNRNAEQKKSEKEILCLIGISFDCTEAAKFDVFVNDCDEEQITPCDSENVGSFAAVPHAKGMAMGCKSGMRFSLTELLEETKAEGDESIRVTIVPRTTPGKKVKVTIDAIEIRLIPVLEK

SEQ ID NO: 29 - DNA, 4086 bp, synthetic construct
  misc_feature 1..4086: note = synthetic construct
  source 1..4086: mol_type = other DNA
atgggtttggactctactgcgcctaagaaaaagagaaaagtcgggattcacggcgtgccagcggccatgactcagttcgagggtttcactaacctgtaccaagtgtccaagaccctgaggttcgagctgatcccgcaaggaaagaccctgaagcacatccaggaacagggcttcatcgaagaggataaggcccgcaacgaccactacaaggaactgaagcccattattgatcggatctacaagacctacgccgaccagtgcttgcagctggtgcagctggactgggaaaatctgtccgccgcgattgattcctaccggaaggagaaaaccgaagagactcgcaacgctctcattgaggaacaggccacctaccggaacgccattcacgactactttattggccgcactgacaacctcaccgatgcaatcaacaagcgccacgccgagatctacaagggcctgttcaaggcggaactgtttaacgggaaggtcctgaagcaactgggaactgtgaccaccaccgagcatgagaacgccctgctccgctccttcgacaagttcaccacctacttctcgggattctacgagaatcgcaaaaacgtgttcagcgcggaagatatctcaaccgccatcccccaccggattgtgcaggacaacttccctaagttcaaggaaaactgccatatcttcacgcgcctgatcactgctgtgccgagtctgagagagcacttcgagaacgtgaagaaggctatcggcatcttcgtgtccacctcgattgaggaagtgttctccttcccgttctacaatcagctcctgactcaaacccagattgacctgtacaaccagcttctgggggggatttcccgggaagcgggaactgagaagatcaagggactcaacgaagtgctgaacctggcaatccagaagaacgacgaaaccgcgcacatcatcgcaagcctccctcaccgcttcattcctctgttcaagcaaattctttccgaccgcaacaccctgtcgttcatcctggaagaattcaagagcgacgaagaagtcattcagagcttctgcaagtacaagactctgctgaggaacgaaaacgtgctggaaaccgccgaggccctgttcaacgaactgaactcaatcgacctgacgcacattttcatttcccataagaagctggaaactatctcctccgccctctgtgaccactgggacaccctgagaaatgcgttgtatgagcgccggatctccgagttgactgggaagattactaagtccgcgaaggaaaaagtgcagcgctccctgaaacacgaagatatcaaccttcaggagatcatctcagccgccggaaaggaactgtcagaggccttcaagcaaaagacttcagagatcctgtcgcacgcccacgccgctttggaccagcccctgcccaccaccctgaagaagcaggaagaaaaggaaatcctgaagtctcagctcgactcactgcttgggctgtaccatctcctcgattggttcgccgtcgacgagtccaacgaagtcgacccggaattctcggcccggctgaccggtatcaagcttgagatggagccaagcctctccttttacaacaaggcccggaactacgccaccaaaaagccttactcagtggaaaagttcaagcttaactttcaaatgccgaccctggccagcggctgggacgtgaacaaggagaagaacaacggcgccatcctgtttgtgaagaacggactgtattaccttggaattatgcccaaacagaagggtcgctacaaggcactgtccttcgagccgaccgaaaagacttcggaaggttttgacaagatgtactacgattacttcccggacgcggctaagatgatccccaagtgcagcactcagctgaaggccgtgaccgcacactttcaaacccataccaccccgattcttctgagcaacaactttatcgagccactggagattaccaaggaaatctacgacctgaacaaccccgaaaaggaacctaaaaagtttcagaccgcctacgccaagaaaactggcgaccagaagggatacagagaagccctctgcaagtggattgacttcacccgggatttcctgtccaagtacactaagaccacttccattgacctctcgtcgctgcggccgtcctcgcaatacaaggacctgggggagtactacgccgagctcaacccgctgctctaccacataagcttccagcggattgccgagaaagaaatcatggacgccgtcgaaaccggaaagctgtacctcttccaaatctataacaaggacttcgcgaagggtcaccatggaaagccaaacctccacaccctctattggaccggactcttctcgccggaaaacctggccaagacatccatcaagttgaatgggcaggccgaactcttctaccgccccaagtctcggatgaagcgaatggcccaccggctgggagaaaagatgctcaacaagaagctaaaggaccaaaagaccccaatccctgacaccctgtaccaggaactgtacgattacgtgaaccacaggcttagccacgacttatccgacgaagcccgggcgctgctgccgaacgtcatcaccaaggaagtgtcgcacgagatcatcaaggaccgccggtttacctccgacaaattcttcttccacgtgcccattaccctgaactaccaggccgccaactcacccggattcatcaacgaccggatcctgcagtatatcgcgaaggagaaggatcttcacgtgatcggaattgaccggggggagaggaacctgatctacgtgtccgtgattgacacttgtgggaacatcgtagagcagaagtccttcaacatcgtgaacggctacgactaccagatcaagctcaagcaacaggagggcgcacggcagatcgcaagaaaggaatggaaggaaatcggaaaaatcaaggaaatcaaagagggatacctgagcctcgtcatccacgagatcagcaagatggtcattaagtacaatgcgatcatcgccatggaggacttgtcctacggattcaagaaaggacggttcaaagtggagagacaagtgtatcagaagttcgagactatgctcatcaacaagctgaactacctggtgttcaaggatatcagcattacggaaaacggcggactcctcaagggataccagctgacttacattcccgataagctgaagaatgtcggtcatcagtgcggctgtattttctacgtgccggcagcctacacctccaagatcgaccctactaccggtttcgtgaacatttttaagttcaaagatctcaccgtggacgcaaagcgcgaattcattaagaagttcgactcaatccgctacgacagcgagaagaacctgttctgcttcactttcgactacaacaacttcattacccaaaacaccgtcatgtccaagtccagctggagcgtgtacacctatggagtgcggatcaagcggcggtttgtgaacggccggttctcgaatgagtccgacacaattgatatcaccaaagatatggaaaagacactggagatgactgatatcaactggagggatggccacgatttgagacaggacattattgactacgagatagtccagcatatctttgagattttcagactgaccgtgcagatgcgcaattccctgtcggaactggaagatcgggactacgatagactgattagccccgtgctgaacgaaaacaacatcttctacgattccgccaaagctggagatgcgctgccaaaagacgctgacgctaacggcgcctactgcatcgcgctgaagggcctctacgaaatcaagcaaatcaccgagaactggaaggaggacggaaagttctcccgcgacaagctgaagatctcaaacaaggactggtttgacttcatccagaacaagcggtacctgaagcgccctgctgctaccaaaaaggccggccaggccaagaagaaaaagggctcgtacccctacgatgtgccggattacgcctacccttacgatgtccccgactacgcttacccgtacgacgtgcctgactacgcctaa

SEQ ID NO: 30 - protein, 1361 aa, synthetic construct
  REGION 1..1361: note = synthetic protein
MGLDSTAPKKKRKVGIHGVPAAMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKKGSYPYDVPDYAYPYDVPDYAYPYDVPDYA

SEQ ID NO: 31 - DNA, 450 bp, Arabidopsis thaliana
  source 1..450: mol_type = unassigned DNA
aaaagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgatt

SEQ ID NO: 32 - DNA, 649 bp, Cauliflower mosaic virus
  source 1..649: mol_type = unassigned DNA
ggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgcgacaagctgactctagcagatcctccaga

SEQ ID NO: 33 - DNA, 514 bp, Cassava vein mosaic virus
  source 1..514: mol_type = unassigned DNA
cagaaggtaattatccaagatgtagcatcaagaatccaatgtttacgggaaaaactatggaagtattatgtgaactcagcaagaagcagatcaatatgcggcacatattcaacctatgttcaaaaatgaagaatgtacagatacaagatcctatactgccagaatacgaagaagaatacatagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttg

SEQ ID NO: 34 - DNA, 1217 bp, Arabidopsis thaliana
  source 1..1217: mol_type = unassigned DNA
gaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacag

SEQ ID NO: 35 - DNA, 23 bp, synthetic construct
  misc_feature 1..23: note = guideRNA
  source 1..23: mol_type = other DNA
atgggtagtccttatcgtgcagg

SEQ ID NO: 36 - DNA, 23 bp, synthetic construct
  misc_feature 1..23: note = guideRNA
  source 1..23: mol_type = other DNA
cgtccaccatgcgaatgtcgaca

SEQ ID NO: 37 - DNA, 23 bp, synthetic construct
  misc_feature 1..23: note = guideRNA
  source 1..23: mol_type = other DNA
ttcggtggtgagtatcgtgccgg

SEQ ID NO: 38 - DNA, 23 bp, synthetic construct
  misc_feature 1..23: note = guideRNA
  source 1..23: mol_type = other DNA
cgatgagtctagacaaaccaacg

SEQ ID NO: 39 - DNA, 23 bp, synthetic construct
  misc_feature 1..23: note = guideRNA
  source 1..23: mol_type = other DNA
tttggtggcgagtttcgggctgg

SEQ ID NO: 40 - DNA, 23 bp, synthetic construct
  misc_feature 1..23: note = guideRNA
  source 1..23: mol_type = other DNA
atgcaacaagcttcttcggtggc

SEQ ID NO: 41 - DNA, 29 bp, synthetic construct
  misc_feature 1..29: note = oligonucleotide
  source 1..29: mol_type = other DNA
gttatgtaactaactttttcacattatgc

SEQ ID NO: 42 - DNA, 24 bp, synthetic construct
  misc_feature 1..24: note = oligonucleotide
  source 1..24: mol_type = other DNA
atgtgggaagaaatatcaaatatg

SEQ ID NO: 43 - DNA, 20 bp, synthetic construct
  misc_feature 1..20: note = oligonucleotide
  source 1..20: mol_type = other DNA
atagggtctctggctgcaga

SEQ ID NO: 44 - DNA, 23 bp, synthetic construct
  misc_feature 1..23: note = oligonucleotide
  source 1..23: mol_type = other DNA
cgatcgaaacaaacctccacaaa

SEQ ID NO: 45 - DNA, 20 bp, synthetic construct
  misc_feature 1..20: note = oligonucleotide
  source 1..20: mol_type = other DNA
ggaccatcaaccaccgtctt

SEQ ID NO: 46 - DNA, 23 bp, synthetic construct
  misc_feature 1..23: note = oligonucleotide
  source 1..23: mol_type = other DNA
aactgggttatgtggtgtgttct

SEQ ID NO: 47 - DNA, 20 bp, synthetic construct
  misc_feature 1..20: note = oligonucleotide
  source 1..20: mol_type = other DNA
gcgaaacatcaaccaccgac

SEQ ID NO: 48 - DNA, 23 bp, synthetic construct
  misc_feature 1..23: note = oligonucleotide
  source 1..23: mol_type = other DNA
ggtccagatatgaaccgggttat

SEQ ID NO: 49 - DNA, 23 bp, synthetic construct
  misc_feature 1..23: note = oligonucleotide
  source 1..23: mol_type = other DNA
ctgggtgggtaattctaggatgg

SEQ ID NO: 50 - DNA, 23 bp, synthetic construct
  misc_feature 1..23: note = oligonucleotide
  source 1..23: mol_type = other DNA
ctcggttgtagacacgtacaaga

SEQ ID NO: 51 - DNA, 20 bp, synthetic construct
  misc_feature 1..20: note = oligonucleotide
  source 1..20: mol_type = other DNA
aggtcacttgcaaggagtcg

SEQ ID NO: 52 - DNA, 23 bp, synthetic construct
  misc_feature 1..23: note = oligonucleotide
  source 1..23: mol_type = other DNA
ttaagttctcttgggtcaccgac

SEQ ID NO: 53 - DNA, 23 bp, synthetic construct
  misc_feature 1..23: note = oligonucleotide
  source 1..23: mol_type = other DNA
ataccaactacacctgccacttt

SEQ ID NO: 54 - DNA, 23 bp, synthetic construct
  misc_feature 1..23: note = oligonucleotide
  source 1..23: mol_type = other DNA
tatgtccgttcctgtgcttatgt

SEQ ID NO: 55 - DNA, 20 bp, synthetic construct
  misc_feature 1..20: note = oligonucleotide
  source 1..20: mol_type = other DNA
tccgcaacgtcaaccatgta

SEQ ID NO: 56 - DNA, 23 bp, synthetic construct
  misc_feature 1..23: note = oligonucleotide
  source 1..23: mol_type = other DNA
cctctattgatccgacagatggg

SEQ ID NO: 57 - DNA, 23 bp, synthetic construct
  misc_feature 1..23: note = oligonucleotide
  source 1..23: mol_type = other DNA
acgaaagtcaccttcggtatgaa

SEQ ID NO: 58 - DNA, 23 bp, synthetic construct
  misc_feature 1..23: note = oligonucleotide
  source 1..23: mol_type = other DNA
atctgtgaacagccgtatgacaa

SEQ ID NO: 59 - DNA, 21 bp, synthetic construct
  misc_feature 1..21: note = oligonucleotide
  source 1..21: mol_type = other DNA
tctcaagttcccatagcacga

SEQ ID NO: 60 - DNA, 21 bp, synthetic construct
  misc_feature 1..21: note = oligonucleotide
  source 1..21: mol_type = other DNA
tgcatacgccaatgagtttga

SEQ ID NO: 61 - DNA, 20 bp, synthetic construct
  misc_feature 1..20: note = oligonucleotide
  source 1..20: mol_type = other DNA
agcacgaatcacgaaccatc

SEQ ID NO: 62 - DNA, 20 bp, synthetic construct
  misc_feature 1..20: note = oligonucleotide
  source 1..20: mol_type = other DNA
tgaggtccggtgccataatc

SEQ ID NO: 63 - DNA, 20 bp, synthetic construct
  misc_feature 1..20: note = oligonucleotide
  source 1..20: mol_type = other DNA
ttcatatctgggccggtgag

SEQ ID NO: 64 - DNA, 22 bp, synthetic construct
  misc_feature 1..22: note = oligonucleotide
  source 1..22: mol_type = other DNA
acaagaacgaagaatcaagcca

SEQ ID NO: 65 - DNA, 20 bp, synthetic construct
  misc_feature 1..20: note = oligonucleotide
  source 1..20: mol_type = other DNA
ccctctgaacaaacgtccaa

SEQ ID NO: 66 - DNA, 21 bp, synthetic construct
  misc_feature 1..21: note = oligonucleotide
  source 1..21: mol_type = other DNA
tgaactgggttatgtggtgtg

SEQ ID NO: 67 - DNA, 21 bp, synthetic construct
  misc_feature 1..21: note = oligonucleotide
  source 1..21: mol_type = other DNA
ggccttggaggtctttatggt

SEQ ID NO: 68 - DNA, 20 bp, synthetic construct
  misc_feature 1..20: note = oligonucleotide
  source 1..20: mol_type = other DNA
agccggaccacatttactga

SEQ ID NO: 69 - DNA, 21 bp, synthetic construct
  misc_feature 1..21: note = oligonucleotide
  source 1..21: mol_type = other DNA
ccatcacaagccacctcttac

SEQ ID NO: 70 - DNA, 23 bp, synthetic construct
  misc_feature 1..23: note = oligonucleotide
  source 1..23: mol_type = other DNA
acgtttctcctatcaagtttgcc

SEQ ID NO: 71 - DNA, 21 bp, synthetic construct
  misc_feature 1..21: note = oligonucleotide
  source 1..21: mol_type = other DNA
aaatcccgtggctccatagtc

SEQ ID NO: 72 - DNA, 21 bp, synthetic construct
  misc_feature 1..21: note = oligonucleotide
  source 1..21: mol_type = other DNA
ttcgcttcctgcttgttcttc

SEQ ID NO: 73 - DNA, 20 bp, synthetic construct
  misc_feature 1..20: note = oligonucleotide
  source 1..20: mol_type = other DNA
tttacgtccaccatgccaac

SEQ ID NO: 74 - DNA, 21 bp, synthetic construct
  misc_feature 1..21: note = oligonucleotide
  source 1..21: mol_type = other DNA
agtcgtatcccattgcagtca

SEQ ID NO: 75 - DNA, 21 bp, synthetic construct
  misc_feature 1..21: note = oligonucleotide
  source 1..21: mol_type = other DNA
tgtacgtgtctacaaccgaga

SEQ ID NO: 76 - DNA, 21 bp, synthetic construct
  misc_feature 1..21: note = oligonucleotide
  source 1..21: mol_type = other DNA
cgattccagcagacttagcag

SEQ ID NO: 77 - DNA, 21 bp, synthetic construct
  misc_feature 1..21: note = oligonucleotide
  source 1..21: mol_type = other DNA
gcaaatgagctgttgttcgtg

SEQ ID NO: 78 - DNA, 21 bp, synthetic construct
  misc_feature 1..21: note = oligonucleotide
  source 1..21: mol_type = other DNA
tatgtggcaactgtgcgaaac

SEQ ID NO: 79 - DNA, 21 bp, synthetic construct
  misc_feature 1..21: note = oligonucleotide
  source 1..21: mol_type = other DNA
cagttgccacacaaacacaag

SEQ ID NO: 80 - DNA, 21 bp, synthetic construct
  misc_feature 1..21: note = oligonucleotide
  source 1..21: mol_type = other DNA
gatccagaccttgggacaatc

SEQ ID NO: 81 - DNA, 21 bp, synthetic construct
  misc_feature 1..21: note = oligonucleotide
  source 1..21: mol_type = other DNA
attagcggtggtggatcagtc

SEQ ID NO: 82 - DNA, 20 bp, synthetic construct
  misc_feature 1..20: note = oligonucleotide
  source 1..20: mol_type = other DNA
gtttgcctccctgcatcttc

SEQ ID NO: 83 - DNA, 21 bp, synthetic construct
  misc_feature 1..21: note = oligonucleotide
  source 1..21: mol_type = other DNA
tatcacgacgccaaacttctc

SEQ ID NO: 84 - DNA, 21 bp, synthetic construct
  misc_feature 1..21: note = oligonucleotide
  source 1..21: mol_type = other DNA
ctatgtgccgctggtctaatc

SEQ ID NO: 85 - DNA, 21 bp, synthetic construct
  misc_feature 1..21: note = oligonucleotide
  source 1..21: mol_type = other DNA
ataaggccgtcagaagcagag

SEQ ID NO: 86 - DNA, 21 bp, synthetic construct
  misc_feature 1..21: note = oligonucleotide
  source 1..21: mol_type = other DNA
aacgaaactgtgtgggtgttc

SEQ ID NO: 87 - DNA, 21 bp, synthetic construct
  misc_feature 1..21: note = oligonucleotide
  source 1..21: mol_type = other DNA
ggcctgaaactgctagaacaa

SEQ ID NO: 88 - DNA, 20 bp, synthetic construct
  misc_feature 1..20: note = oligonucleotide
  source 1..20: mol_type = other DNA
aacccgcatactcgctatca

SEQ ID NO: 89 - DNA, 21 bp, synthetic construct
  misc_feature 1..21: note = oligonucleotide
  source 1..21: mol_type = other DNA
gcgaagatacgactgacgact

SEQ ID NO: 90 - DNA, 21 bp, synthetic construct
  misc_feature 1..21: note = oligonucleotide
  source 1..21: mol_type = other DNA
ccagcgacatacttaccacca

SEQ ID NO: 91 - DNA, 21 bp, synthetic construct
  misc_feature 1..21: note = oligonucleotide
  source 1..21: mol_type = other DNA
acttctactccgcaggctatg

SEQ ID NO: 92 - DNA, 20 bp, synthetic construct
  misc_feature 1..20: note = oligonucleotide
  source 1..20: mol_type = other DNA
gtgcatggtgtctcctgttc

SEQ ID NO: 93 - DNA, 21 bp, synthetic construct
  misc_feature 1..21: note = oligonucleotide
  source 1..21: mol_type = other DNA
taacgacaatgacccacacag

SEQ ID NO: 94 - DNA, 21 bp, synthetic construct
  misc_feature 1..21: note = oligonucleotide
  source 1..21: mol_type = other DNA
gtggaatggaaagaagagcca

SEQ ID NO: 95 - DNA, 21 bp, synthetic construct
  misc_feature 1..21: note = oligonucleotide
  source 1..21: mol_type = other DNA
acataagcacaggaacggaca

SEQ ID NO: 96 - DNA, 21 bp, synthetic construct
  misc_feature 1..21: note = oligonucleotide
  source 1..21: mol_type = other DNA
caccaccacatcttcgtcatc

SEQ ID NO: 97 - DNA, 21 bp, synthetic construct
  misc_feature 1..21: note = oligonucleotide
  source 1..21: mol_type = other DNA
atggccggaataccaactaca

SEQ ID NO: 98 - DNA, 19 bp, synthetic construct
  misc_feature 1..19: note = oligonucleotide
  source 1..19: mol_type = other DNA
ccgctgtctcctcatcctc

SEQ ID NO: 99 - DNA, 22 bp, synthetic construct
  misc_feature 1..22: note = oligonucleotide
  source 1..22: mol_type = other DNA
tgtatgtggatgacaaggacga

SEQ ID NO: 100 - DNA, 21 bp, synthetic construct
  misc_feature 1..21: note = oligonucleotide
  source 1..21: mol_type = other DNA
cttctcgcctgattgatggtg

SEQ ID NO: 101 - DNA, 21 bp, synthetic construct
  misc_feature 1..21: note = oligonucleotide
  source 1..21: mol_type = other DNA
ggatgccgaacaatgaagaca

SEQ ID NO: 102 - DNA, 22 bp, synthetic construct
  misc_feature 1..22: note = oligonucleotide
  source 1..22: mol_type = other DNA
cctgatacccaactctttccag

SEQ ID NO: 103 - DNA, 21 bp, synthetic construct
  misc_feature 1..21: note = oligonucleotide
  source 1..21: mol_type = other DNA
aggaccaaggaggacaaacag

SEQ ID NO: 104 - DNA, 21 bp, synthetic construct
  misc_feature 1..21: note = oligonucleotide
  source 1..21: mol_type = other DNA
tgcgaaactaccagcaaactc

SEQ ID NO: 105 - DNA, 20 bp, synthetic construct
  misc_feature 1..20: note = oligonucleotide
  source 1..20: mol_type = other DNA
gacaatggaaccgagaccac

SEQ ID NO: 106 - DNA, 21 bp, synthetic construct
  misc_feature 1..21: note = oligonucleotide
  source 1..21: mol_type = other DNA
ccaaacctaactccgctcatc

SEQ ID NO: 107 - DNA, 22 bp, synthetic construct
  misc_feature 1..22: note = oligonucleotide
  source 1..22: mol_type = other DNA
acgtcaaccatgtatcaccaga

SEQ ID NO: 108 - DNA, 21 bp, synthetic construct
  misc_feature 1..21: note = oligonucleotide
  source 1..21: mol_type = other DNA
cttaacgggtcagtagcgttg

SEQ ID NO: 109 - DNA, 22 bp, synthetic construct
  misc_feature 1..22: note = oligonucleotide
  source 1..22: mol_type = other DNA
gcaactccagattcacaactca

SEQ ID NO: 110 - DNA, 21 bp, synthetic construct
  misc_feature 1..21: note = oligonucleotide
  source 1..21: mol_type = other DNA
gcgtcaaagacagggtttctc

SEQ ID NO: 111 - DNA, 21 bp, synthetic construct
  misc_feature 1..21: note = oligonucleotide
  source 1..21: mol_type = other DNA
cctattgactgttgccctctg

SEQ ID NO: 112 - DNA, 21 bp, synthetic construct
  misc_feature 1..21: note = oligonucleotide
  source 1..21: mol_type = other DNA
atcgagatacccgactggaag

SEQ ID NO: 113 - DNA, 20 bp, synthetic construct
  misc_feature 1..20: note = oligonucleotide
  source 1..20: mol_type = other DNA
ggaatttgctcctgggtctc

SEQ ID NO: 114 - DNA, 21 bp, synthetic construct
  misc_feature 1..21: note = oligonucleotide
  source 1..21: mol_type = other DNA
atacggcatctttgcatggtc

SEQ ID NO: 115 - DNA, 19 bp, synthetic construct
  misc_feature 1..19: note = oligonucleotide
  source 1..19: mol_type = other DNA
aaatgctgacgtggcctga

SEQ ID NO: 116 - DNA, 23 bp, synthetic construct
  misc_feature 1..23: note = oligonucleotide
  source 1..23: mol_type = other DNA
ctttctcatccagtcctgaacac

SEQ ID NO: 117 - DNA, 23 bp, synthetic construct
  misc_feature 1..23: note = oligonucleotide
  source 1..23: mol_type = other DNA
cagctgaggaatggatttcaaga

SEQ ID NO: 118 - DNA, 21 bp, synthetic construct
  misc_feature 1..21: note = oligonucleotide
  source 1..21: mol_type = other DNA
gggtcgacatggttcacaatc

SEQ ID NO: 119 - DNA, 19 bp, synthetic construct
  misc_feature 1..19: note = guideRNA
  source 1..19: mol_type = other DNA
tgggtagtccttatcgtgc

SEQ ID NO: 120 - DNA, 19 bp, synthetic construct
  misc_feature 1..19: note = guideRNA
  source 1..19: mol_type = other DNA
tggggaacttctactccgc

SEQ ID NO: 121 - DNA, 19 bp, synthetic construct
  misc_feature 1..19: note = guideRNA
  source 1..19: mol_type = other DNA
tcggtggtgagtatcgtgc

SEQ ID NO: 122 - DNA, 19 bp, synthetic construct
  misc_feature 1..19: note = guideRNA
  source 1..19: mol_type = other DNA
aaaccaacgacggtgatgg

SEQ ID NO: 123 - DNA, 19 bp, synthetic construct
  misc_feature 1..19: note = guideRNA
  source 1..19: mol_type = other DNA
ttggtggcgagtttcgggc

SEQ ID NO: 124 - DNA, 19 bp, synthetic construct
  misc_feature 1..19: note = guideRNA
  source 1..19: mol_type = other DNA
tcggtggcaaatatgtagc

SEQ ID NO: 125 - DNA, 18 bp, synthetic construct
  misc_feature 1..18: note = oligonucleotide
  source 1..18: mol_type = other DNA
taaggaaccaagttcggc

SEQ ID NO: 126 - DNA, 18 bp, synthetic construct
  misc_feature 1..18: note = oligonucleotide
  source 1..18: mol_type = other DNA
acaccaagccttccacag

SEQ ID NO: 127 - DNA, 22 bp, synthetic construct
  misc_feature 1..22: note = oligonucleotide
  source 1..22: mol_type = other DNA
gtctggaggatctgctagagtc

SEQ ID NO: 128 - DNA, 19 bp, synthetic construct
  misc_feature 1..19: note = oligonucleotide
  source 1..19: mol_type = other DNA
cgtcttcaaagcaagtgga

SEQ ID NO: 129 - DNA, 20 bp, synthetic construct
  misc_feature 1..20: note = oligonucleotide
  source 1..20: mol_type = other DNA
tggcaggatatattgtggtg

SEQ ID NO: 130 - DNA, 23 bp, synthetic construct
  misc_feature 1..23: note = oligonucleotide
  source 1..23: mol_type = other DNA
gtactcttgccgactacaacatc

SEQ ID NO: 131 - DNA, 22 bp, synthetic construct
  misc_feature 1..22: note = oligonucleotide
  source 1..22: mol_type = other DNA
gtctggaggatctgctagagtc

SEQ ID NO: 132 - DNA, 21 bp, synthetic construct
  misc_feature 1..21: note = oligonucleotide
  source 1..21: mol_type = other DNA
gtttacccgccaatatatcct

SEQ ID NO: 133 - DNA, 26 bp, synthetic construct
  misc_feature 1..26: note = oligonucleotide
  source 1..26: mol_type = other DNA
agggcgaattcgacccagctttcttg

SEQ ID NO: 134 - DNA, 21 bp, synthetic construct
  misc_feature 1..21: note = oligonucleotide
  source 1..21: mol_type = other DNA
gtttacccgccaatatatcct

SEQ ID NO: 135 - DNA, 23 bp, synthetic construct
  misc_feature 1..23: note = oligonucleotide
  source 1..23: mol_type = other DNA
atgtctctgtttggaaatgtttc

SEQ ID NO: 136 - DNA, 23 bp, synthetic construct
  misc_feature 1..23: note = oligonucleotide
  source 1..23: mol_type = other DNA
tcccatcactcaatagaaagttc

SEQ ID NO: 137 - DNA, 24 bp, synthetic construct
  misc_feature 1..24: note = oligonucleotide
  source 1..24: mol_type = other DNA
aatgacaaactcatcagggaagtg

SEQ ID NO: 138 - DNA, 20 bp, synthetic construct
  misc_feature 1..20: note = oligonucleotide
  source 1..20: mol_type = other DNA
ggcagctcatttggctaata

SEQ ID NO: 139 - DNA, 20 bp, synthetic construct
  misc_feature 1..20: note = oligonucleotide
  source 1..20: mol_type = other DNA
cgaagaatcaagccagtctt

SEQ ID NO: 140 - DNA, 19 bp, synthetic construct
  misc_feature 1..19: note = oligonucleotide
  source 1..19: mol_type = other DNA
gcagctcatttggctaatg

SEQ ID NO: 141 - DNA, 20 bp, synthetic construct
  misc_feature 1..20: note = oligonucleotide
  source 1..20: mol_type = other DNA
cgaagaatcaagccaatctt

SEQ ID NO: 142 - DNA, 22 bp, synthetic construct
  misc_feature 1..22: note = oligonucleotide
  source 1..22: mol_type = other DNA
gattcaagccatgaagaatctc

SEQ ID NO: 143 - DNA, 23 bp, synthetic construct
  misc_feature 1..23: note = oligonucleotide
  source 1..23: mol_type = other DNA
tgtgtcccttgattcccaaatct

SEQ ID NO: 144 - DNA, 18 bp, synthetic construct
  misc_feature 1..18: note = oligonucleotide
  source 1..18: mol_type = other DNA
agccactgccgattacat

SEQ ID NO: 145 - DNA, 21 bp, synthetic construct
  misc_feature 1..21: note = oligonucleotide
  source 1..21: mol_type = other DNA
cgtatgatgcatttagccagt

SEQ ID NO: 146 - DNA, 20 bp, synthetic construct
  misc_feature 1..20: note = oligonucleotide
  source 1..20: mol_type = other DNA
ctaagaatgaggcgttcagg

SEQ ID NO: 147 - DNA, 20 bp, synthetic construct
  misc_feature 1..20: note = oligonucleotide
  source 1..20: mol_type = other DNA
gtttgcctccctgcatcttc

SEQ ID NO: 148 - DNA, 18 bp, synthetic construct
  misc_feature 1..18: note = oligonucleotide
  source 1..18: mol_type = other DNA
attgcgcttactgcaatg

SEQ ID NO: 149 - DNA, 20 bp, synthetic construct
  misc_feature 1..20: note = oligonucleotide
  source 1..20: mol_type = other DNA
tctgtcggttctttgcctcc

SEQ ID NO: 150 - DNA, 630 bp, synthetic construct
  misc_feature 1..630: note = consensus sequence
  source 1..630: mol_type = other DNA
cctcgtagtttcaagcaacaagctgctgttcattgtgcgtattgcgatggggcatacgatcaagtcggtttccctgatctcgagcttcaagtccatggctcatggttgttcttacctttccaccgctattacttatacttcttcgagaaaatttgtggcaaattaatcgatgatccaaatttcgcaatccctttttggaactgggatgcacctgatggcatgaagatccctgatatttacacgaataagaaatctccgttgtacgatgctcttcgtgatgcgaagcatcaaccaccgtctctgattgatcttgactacaatggtgacgatgaaaatcttagccgatcgaaacaaacctccacaaatctcacaattatgtacagacaaatggtgtctagttccaagactgctagtcttttcatgggtagtccttatcgtgcaggtgatgaggctagccctggctctggctcgctcgagagcataccacatggcccggttcatatctggaccggagataggaaccagcaaaatggtgaagacatgggtaacttttattctgcagccagagaccctattttctatgcacatcatgcgaatatcgacagaatgtggtcagtttggaaaactcta

SEQ ID NO: 151 - DNA, 630 bp, synthetic construct
  misc_feature 1..630: note = consensus sequence
  source 1..630: mol_type = other DNA
cctcgtagctttaagcagcaagcaaatgttcattgtgcctattgcgatggcgcgtatgaccaagtcggttttccagatctggagcttcaagtacataactcatggctgttcttccctttccatcgctattacatgtacttcttcgagaaaatttgtggcaagttaattgatgacccaaatttcgcaattccattttggaactgggatgcaccagatgggatgaaaatccctgatatttacacaaataagaaatcttcgttgtatgatcctcttcgcgatgtggaccatcaaccaccgtctttgattgatcttgacttcaatggtgtcgacgaaaatcttagcccctctgaacaaacgtccaaaaatctcacagttatgtatagacaaatggtgtctagttccaagacttctactcttttcatgggtagtccttatcgtgcaggcgatgatgctagccctggtagtggttcgatcgagaacacaccacataacccagttcatatctgggccggtgagtggaagcataataatggcaaaaacatgggcaaactttattctgcagccagggaccctcttttctatgcacatcatgggaatattgatagaatgtggtcagtttggaaaacacta

SEQ ID NO: 152 - DNA, 655 bp, synthetic construct
  misc_feature 1..655: note = consensus sequence
  source 1..655: mol_type = other DNA
tccacacagttggaagcaacaagctaagatccactgcgcttattgcaacggtggttacaatcaagaacagagtggtttcccggacatacaactccagattcacaacacatggctcttctttcctttccaccgatggtacctctacttctacgagaggattttggggaagttgattaatgatccaactttcgctttaccatactggaactgggataaccctaccggaatggtgctccctgccatgttcgaaaccgacggcaaaaggaaccctatctttgacccttacaggaatgccacacacctcccaccagctatctttgaagtgggatataatgggacagacagtggcgccacttgtatagaccagataagcgctaatctgtctttgatgtacaagcaaatgatcaccaacgctcctgatacaacaacgttcttcggtggagaatttgttgctggggatgaccctcttaacaaagagtttaacgttgctgggtccatagaggctggggttcacactgcggcgcatagatgggtgggtgatcctaggatggccaacagcgaggacatggggaacttctactccgcagggtatgatcctctcttttacgtccaccatgccaacgtcgaccggatgtggaaaatctggaaagatttg

SEQ ID NO: 153 - DNA, 655 bp, synthetic construct
  misc_feature 1..655: note = consensus sequence
  source 1..655: mol_type = other DNA
cccacatagctggaagcaacaaggcaagattcactgtgcttattgcaacggtggttacaatcaagaacaaagtggttacccgaatttacaacttcagattcacaactcatggctcttctttcctttccaccggtggtacctctatttctacgagaagatattggggaagttgattaatgatccaactttcgctctaccttactggaactgggataaccctactggaatggttattcctgccatgttcgaacagaacagcaaaactaactctctgtttgaccctttaagggatgcgaaacacctcccaccttctatctttgatgttgaatatgctggtgcagacactggtgccacttgtatagaccagatagccattaatctgtcttcaatgtacagacagatggtcaccaactccactgatacaaaacgattcttcggtggcgaatttgtagctggaaatgaccctcttgcgagcgagttcaacgtagctgggaccgtagaagctggggttcacactgcggctcaccgctgggtgggtaattctaggatggccaacagcgaagacatggggaacttctactccgcaggatatgatcctctcttttacgtccaccatgcgaatgtcgacaggatgtggcaaatctggaaagatatt

SEQ ID NO: 154 - DNA, 634 bp, synthetic construct
  misc_feature 1..634: note = consensus sequence
  source 1..634: mol_type = other DNA
tcctcgaaactttctgcaacaagcacacattcactgtgcttactgcaatggcgcttacactcaatcttcaagtggatttcccgatattgaaatccagattcataactcatggctgttcttccccttccaccgttggtatctctacttttacgagagaatcctggggagcttgatcgatgatcccactttcgctttgccattctggaactgggacacccctgccggaatgacaattccgaaatactttaacgatcccaaaaacgcagtttttgatcccaaaagaaaccaaggtcacttgcaaggagtcgtcgatctgggttacaatgggaaagattcagacactactgatatcgaaaaggtgaagaacaatctcgcgataatgtatcgtcaaatggtgacaaacgccaccgaccccacagctttcttcggtggtgagtatcgtgccggaatcgaacccattagcggtggtggatcagtcgaacaaagcccacacacacctgttcaccggtgggtcggtgacccaagagaacttaacggtgaaaacctcggtaacttctactccgccggtcgtgacacgctcttttactgtcaccattccaacgtcgatcgaatgtggtcgttgtggaagatgcag

SEQ ID NO: 155 - DNA, 664 bp, synthetic construct
  misc_feature 1..664: note = consensus sequence
  source 1..664: mol_type = other DNA
cccacgcagtttcaataaccaagctaaagttcattgcgcttactgcaatggcagttacactcaaaacggtcaagaactccagattcacaactcctggctcttctttcccttccatcggtggtacctttatttctacgagaggatactgggagatctcattggtgattcgacattcgggttaccctactggaactgggacaaccccgaaggaatgacaattccacacttcttcgtagagaaacaatgtaacaactataagttcgaaaacggagaaaaccctctatatgataagtatcgggacgaaagtcaccttcggtatgaattggtcgatcttgactactcagggagaaaccgcgacctgtgttacgatcagaaagaaatcaatctggctactatgaataggcagatgatgcgcaacgcctttgatgcaacaagcttcttcggtggcaaatatgtagccggtgatgaaccgattccccgaggagataatgtagttggatccgtggaggctggttgtcatacggctgttcacagatgggttgggaaccctgatccaaaagggaataaagaggacatgggcaacttctactctgcgggatatgatcctttgttctacgtccaccattctaatgtggaccgaatgtggactctttggaagcaaatg

SEQ ID NO: 156 - DNA, 56 bp, Lactuca sativa
  source 1..56: mol_type = unassigned DNA
agttccaagacttctactcttttcatgggtagtccttatcgtgcaggcgatgatgc

SEQ ID NO: 157 - DNA, 58 bp, Lactuca sativa
  source 1..58: mol_type = unassigned DNA
agttccaagacttctactcttttcatgggtagtccttatcgtgcaggcgatgatgcta

SEQ ID NO: 158 - DNA, 57 bp, Lactuca sativa
  source 1..57: mol_type = unassigned DNA
agttccaagacttctactcttttcatgggtagtccttatcggcaggcgatgatgcta

SEQ ID NO: 159 - DNA, 58 bp, Lactuca sativa; identical to SEQ ID NO: 157
  source 1..58: mol_type = unassigned DNA

SEQ ID NO: 160 - DNA, 54 bp, Lactuca sativa
  source 1..54: mol_type = unassigned DNA
agttccaagacttctactcttttcatgggtagtccttagcaggcgatgatgcta

SEQ ID NO: 161 - DNA, 74 bp, Lactuca sativa
  source 1..74: mol_type = unassigned DNA
gtgacaaacgccaccgaccccacagctttcttcggtggtgagtatcgtgccggaatcgaacccattagcggtgg

SEQ ID NO: 162 - DNA, 74 bp, Lactuca sativa
  source 1..74: mol_type = unassigned DNA
gtgtctaatgccaagactgctagtcttttcatgggtagtccttatcgtgcaggtgatgaccctagccctggtgc

SEQ ID NO: 163 - DNA, 74 bp, Lactuca sativa; identical to SEQ ID NO: 161
  source 1..74: mol_type = unassigned DNA

SEQ ID NO: 164 - DNA, 74 bp, Lactuca sativa; identical to SEQ ID NO: 161
  source 1..74: mol_type = unassigned DNA

SEQ ID NO: 165 - DNA, 111 bp, Lactuca sativa
  source 1..111: mol_type = unassigned DNA
gtgacaaacgccaccgaccccacagctttcttcggtggtgacaaacgccaccgaccccacagctttcttcggtggtaagtatcgtgccggaatcgaacccattagcggtgg

SEQ ID NO: 166 - DNA, 111 bp, Lactuca sativa
  source 1..111: mol_type = unassigned DNA
gtgacaaacgccaccgaccccacagctttcttcggtggtgacaaacgccaccgaccccacagctttcttcggttgtgagtatcgtgccggaatcgaacccattagcggtgg

SEQ ID NO: 167 - DNA, 111 bp, Lactuca sativa
  source 1..111: mol_type = unassigned DNA
gtgacaaacgccaccgaccccacagctttcttcggtggtgacaaacgccaccgaccccacagctttcttcggtggttagtatcgtgccggaatcgaacccattagcggtgg

SEQ ID NO: 168 - DNA, 74 bp, Lactuca sativa; identical to SEQ ID NO: 161
  source 1..74: mol_type = unassigned DNA

SEQ ID NO: 169 - DNA, 74 bp, Lactuca sativa; identical to SEQ ID NO: 161
  source 1..74: mol_type = unassigned DNA

(19) United States
(12) Patent Application Publication
(10) Pub. No.: US 2025/0129376 A1
(43) Pub. Date: Apr. 24, 2025

(54) LETTUCE WITH INCREASED SHELF LIFE

(71) Applicant: GREENVENUS, LLC, Davis, CA (US)

(72) Inventors: Sekhar BODDUPALLI, Germantown, MD (US); Arianne TREMBLAY, Germantown, MD (US); Eric AASEN, Germantown, MD (US); Jyoti R. ROUT, Woodland, CA (US); Zhongsen LI, Germantown, MD (US)

(21) Appl. No.: 18/990,497

(22) Filed: Dec. 20, 2024

Related U.S. Application Data
(62) Division of application No. 17/771,687, filed on Apr. 25, 2022, now Pat. No. 12,203,086.
(60) Provisional application No. 62/925,853, filed on Oct. 25, 2019.

(51) Int. Cl. C12N 15/82; C12N 9/02; C12N 9/22; C12N 15/11

(52) U.S. Cl. CPC C12N 15/8243; C12N 9/0061; C12N 9/22; C12N 15/11; C12N 15/8213; C12Y 110/03002; C12N 2310/20

(57) ABSTRACT

The present application is directed to lettuce plants comprising a mutation in each of at least two different PPO genes, where said mutations reduce the activity of PPO protein compared to a wild type lettuce plant. The present application is also directed to methods for making such lettuce plants.

This application is a division of U.S. patent application Ser. No. 17/771,687, filed Apr. 25, 2022, which is a national stage application under 35 U.S.C. § 371 of International Application No. PCT/US2020/054082, filed Oct. 2, 2020, which claims the priority benefit of U.S. Provisional Patent Application Ser. No. 62/925,853, filed Oct. 25, 2019, which are hereby incorporated by reference in their entirety.

### SEQUENCE LISTING

The present application contains a Sequence Listing which has been submitted electronically in XML format and is hereby incorporated by reference in its entirety. Said XML copy, created on Dec. 17, 2024, is named 257432_001006_ST26.xml, and is 242,584 bytes in size.

### FIELD

The present application is directed to lettuce plants with increased shelf life. The lettuce plants have combinations of polyphenol oxidase (“PPO”) gene mutations to reduce browning, reduce tip burn, create longer shelf life, and improve nutrition as compared to non- mutated varieties.

### BACKGROUND

Lettuce is one of the most commonly consumed “ready to eat” leafy vegetables in the United States and world. The United States alone produces over 8 billion pounds of lettuce worth $1.9B (USDA-ERS: Vegetable and Pulses Data-2017). At least 90% of that total is consumed domestically. Cut salads, however, are a highly perishable commodity and significant loss occurs due to cut-surface browning during storage and distribution. Several practices such as optimized growing conditions (reduced irrigation/nitrogen and early-harvesting) and post-harvest practices (application of antioxidant chemicals, low oxygen-packing, and storage at low temperature) are utilized to reduce browning; however, they are not completely effective in addition to adding significantly to the cost of production and storage. Key enzymes in plants responsible for the browning are polyphenol oxidase (“PPO”), phenylalanine ammonia lyase (“PAL”), and peroxidases. PPO is among the major contributors in wound-induced browning in most fruits and vegetables. Breeding strategies targeting PAL and PPO pathways have been attempted with limited success.

The lettuce genome contains at least 19 putative PPO genes, and the expression level of PPO genes in lettuce varies significantly. It is unknown which genes or combination of genes, when mutated or when gene function is suppressed are necessary and sufficient to achieve non-browning lettuce.

There is a need in the art to determine the PPO gene(s), when mutated or suppressed, that is/are necessary and sufficient to achieve a variety of non-browning lettuce that will have longer shelf life to help reduce waste of cultivated lettuce for consumption.

### SUMMARY

One aspect of the present application is directed to a lettuce plant comprising a mutation in each of at least two different PPO genes, where said mutations reduce the activity of PPO protein compared to a wild type lettuce plant.

Another aspect of the present application is directed to a method of making a lettuce plant with reduced PPO activity. This method involves introducing a mutation into each of at least two different PPO genes of a lettuce plant, where said mutations reduce the activity of PPO protein compared to a wild type lettuce plant.

A further aspect of the present application is directed to a method of editing PPO genes of lettuce plant. This method involves introducing into a lettuce plant a polynucleotide construct comprising a first nucleic acid sequence encoding a gene editing nuclease, a promoter that is functional in plants operably linked to said first nucleic acid sequence, a second nucleic acid sequence encoding a plurality of gRNAs in a polycistronic arrangement targeting at least two PPO genes of choice to edit, and a second promoter that is functional in plants, operably linked to said second nucleic acid sequence.

Another aspect of the present application is directed to a polynucleotide construct for editing polyphenol oxidase genes of a plant comprising a nucleic acid sequence encoding a plurality of gRNAs targeting at least two PPO genes operably linked to a promoter.

### BRIEF DESCRIPTION OF THE DRAWINGS

FIG. 1 is a schematic illustration showing the ID536 expression vector for polycistronic expression of gRNAs for editing PPO genes in lettuce.

FIGS. 2A-B show examples of detected edits made by the SynNuc1 nuclease in the PPO-B gene (FIG. 2A) (SEQ ID NOs: 156-160) and the PPO-G gene (FIG. 2B) (SEQ ID NOs:161-169) by deep sequencing.

FIGS. 3A-B are a schematic illustration showing the ID123 expression vector for polycistronic expression of gRNAs for editing PPO genes in lettuce in FIG. 3A and the ID124 expression vector for polycistronic expression of gRNAs for editing PPO genes in lettuce in FIG. 3B.

FIG. 4 is a graph showing the expression of PPO types using two sets of primers (shown by black and grey bars) for each PPO gene, and these were used to monitor expression levels in leaves. The PDS gene was used as an expression level control.

FIG. 5 shows an alignment of 11 Romaine lettuce PPO genes. Except for PPO-O, all PPO-gRNAs were designed to target the metal-binding domain of each respective PPO. Boxes represent exons. The gRNAs (A, D, G, O, R, and S) are shown at left of the respective genes and the positions of gRNAs in the genes are shown with arrowheads. The gRNA-A targeted 3 PPO genes (A, B, and C) and gRNA-D targeted 4 PPO genes (D, E, N, and P).

FIG. 6 is a table showing individual events corresponding to ID123 and ID124 constructs listed as a line ID. “Het” and “Homo” represent one or both copies of PPO genes mutated, respectively. “WT” represents wild type PPO. Arrows represent events that were advanced to T1.

FIG. 7 shows PPO enzymatic activity of T1-siblings 8-8-4-24, 8-8-4-73, 8-8-4-94, 8-14-13-4, 8-14-13-25, 8-14-13-40, and two wild type genotypes. PPO enzymatic activity was analyzed in twelve week old growth-chamber grown lettuce leaves.

FIG. 8 shows the progression of events to fixed genotypes from T₀ to T₂.

FIGS. 9A-C show schematic illustrations of the ID123 construct (FIG. 9A) and the ID124 construct (FIG. 9B). NPTII is the plant selectable marker conferring kanamycin resistance in the transformed plants. The gene editing nuclease expression is driven by the CaMV 35S promoter. Positions of PCR primers used to detect the boundaries of the ID123 and ID124 constructs are indicated in FIGS. 9A-B. FIG. 9C shows images of agarose gels showing the result of PCR screening of T1 plants with the indicated primers sets. Plant 8-14-13-44 is a control. A178/A181 amplifies the endogenous PDF gene and is a PCR positive control.

FIG. 10 is a table showing the results of sequencing of edited PPO genes in 10 PPO KO varieties at the T₂ and T₃ generation.

FIG. 11 provides photographic images of freshly harvested T3 plants 14-2-24-5 and 14-2-96-13 compared to unmodified isogenic (wild type) control lettuce. Isogenic controls showed tip burn in 80% of the plants while only 40% of 14-2-96-13 plants showed tip burn. 14-2-24-5 showed no tip burn at all.

FIG. 12 provides photographic images of T3 plants 14-2-24-5 and 14-2-96-13 compared to unmodified Isogenic control lettuce after 7 days of cold storage and light. A significant percentage of 14-2-24-5 and 14-2-96-13 plants appeared healthy and saleable, while only 53% of the control lettuce appeared attractive.

FIG. 13 provides photographic images showing the scale and representative leaves for rating appearance of lettuce leaves.

FIG. 14 is a graph assessing browning by overall quality of indicated plant lines at harvest (day 0), and days 3, 7, and 10 post-harvest stored in cold/light conditions. Underlined lines showed better qualities over time than control. Boxed lines showed optimal results.

FIG. 15 provides photographic images showing a comparison of the indicated plant lines at 9 days post-harvest in poor conditions (70° F./dark) (top panel) and in optimal storage conditions (cold/light) (bottom panel).

FIG. 16 provides photographic images showing a comparison of the indicated plant lines at 14 days post-harvest in poor conditions (70° F./dark) (top panel) and in optimal storage conditions (cold/light) (bottom panel).

FIG. 17 shows a pair of graphs assessing browning by overall quality (left) and midvein quality (right) for the indicated plant lines at days 14, 16, 19, 22, and 26 post-harvest.

FIGS. 18A-B are photographic images showing 14-2-24-5 leaves (FIG. 18A) and control Isogenic leaves (FIG. 18B) after 26 days in cold and light storage.

FIG. 19 provides a graphical representation of the locations of PPO genes on the 9 lettuce chromosomes.

FIGS. 20A-B show the shelf life assessment of lines of lettuce as compared to control over days 12, 20, and 28 post-harvest. FIG. 20A shows the average shelf life score (with standard deviation) for 6 lines and 1 control lettuce; * indicates statistical significance from control at Day 20; ‡indicates statistical significance from control at Day 28; FIG. 20B shows the number of projected days for which each line and control would have an acceptable shelf life score (i.e., <13 days) and the number of days added to shelf life as compared to control.

FIG. 21 is a graph showing the browning rate as a percentage per day under cold/light conditions for 6 lines and 1 control lettuce; gray lines represent the standard deviation of 4 replicates of control and 1% is the maximum value.

FIG. 22 is a graph showing % PPO activity in lettuce lines with various PPO gene mutation combinations compared to wild type isogenic controls.

FIG. 23 is a group of photographic images and a rating scale used to score browning in cut lettuce pieces after storage.

FIGS. 24A-B are graphs showing the results of a shelf-life study of PPO mutant line GVR-110 (14-2-96-13) (“GV110”) compared to the control line, Isogenic. FIG. 24A shows the average discoloration of cut lettuce at 1, 7, 10, 14, 20, and 27 days after processing with a score of 5 being severe discoloration and a score of 1 being no discoloration. GVR-110 had significantly less discoloration at days 14, 20, and 27 after processing (P≤0.024). FIG. 24B shows the average overall visual quality score at 1, 7, 10, 14, 20, and 27 days after processing with a score of 9 being excellent quality and a score of 1 being unusable. GVR-110 maintained a high average visual quality score throughout the study and had significantly higher quality scores compared to isogenic controls at day 20 (P≤0.016) and day 27 (P≤0.0003) after processing.

FIG. 25 is a graph showing the total polyphenolics levels in various PPO mutant lines compared to control lettuce lines.

FIG. 26 is a graph showing the average polyphenolic levels over time (at harvest, and 7, 14, and 21 days after harvest (“DAH”)) of PPO mutant lines 24-5, 76-14, and 9-11 compared to a wild type line.

FIG. 27 is a graph showing CO₂ production due to fermentation of cut lettuce during a shelf life study at day 0, 6, 10, 14, 17, and 21 after processing. PPO mutant lines GVR-102 (“Seed Type A”), GVR-107 (“Seed Type B”), GVR-108 (“Seed Type C”), and GVR-110 (“Seed Type D”) were evaluated for CO₂ production along with isogenic control (“Plant Control”), and a control supplied by the seed supplier (“S Control”).

FIG. 28 is a graph showing the organoleptic total score of the same lines shown in FIG. 27 over the course of a shelf life study at day 0, 6, 10, 14, 17, and 21 after processing. PPO mutant lines GVR-102 (“Seed Type A”), GVR-107 (“Seed Type B”), GVR-108 (“Seed Type C”), and GVR-110 (“Seed Type D”) were evaluated for organoleptic parameters along with isogenic control (“Plant Control”), and a control supplied by the seed supplier (“S Control”).

FIG. 29 is a table and graph showing the nutritional composition of PPO mutant lines GVR-102 (“Seed Type A”), GVR-108 (“Seed Type C”), GVR-110 (“Seed Type D”), and an isogenic control (“Control”) at 30 days after processing.

FIGS. 30A-B are graphs showing the height (FIG. 30A) and whole plant area (FIG. 30B) measurements of hydroponically grown PPO mutant lines GVR-102 (“73-14”), GVR-107 (“24-21”), GVR-108 (“24-5”), and GVR-110 (“96-13”) compared to controls Green Romaine (isogenic) (“GR”) and Red Romaine variety (“RR”).

FIGS. 31A-B are a chart and graph showing the results of a shelf life study of the hydroponically grown PPO mutant lines of FIGS. 30A-B.

FIGS. 32A-F are illustrations of sequence identities of PPO genes compared to other lettuce varieties in their tyrosinase domains. FIGS. 32A-F show that the lettuce varieties are identical in their PPO-A, B, D, E, G, and S tyrosinase gene sequences, respectively.

FIG. 33 is a graph showing a lack of any significant differences in disease resistance in PPO edited lettuce lines compared to their isogenic controls.

FIG. 34 is a graph showing that lettuce plants of the present application with multiple PPO edits performed equivalent to market standard.

### DETAILED DESCRIPTION

In this disclosure, a number of terms and abbreviations are used. The following definitions are provided and should be helpful in understanding the scope and practice of the present invention.

The term “mutation” means a human-induced change in the genetic sequence compared to a wild type sequence. The mutation may be, without limitation, from one or more nucleotide insertions, one or more nucleotide substitutions, one or more nucleotide deletions, or any combination thereof. In one embodiment, mutations are those that cause the gene to not be expressed or not be properly expressed, or those that inactivate the protein, such as from the introduction of a frameshift to the coding region leading to a premature stop codon. A mutation that leads to a change in the expression of the gene such that the function of the encoded protein is eliminated or substantially decreased, such as a premature stop codon, is referred to herein as a “knockout” mutation.

The term “isolated” for the purposes of the present application designates a biological material (e.g., nucleic acid or protein) that has been removed from its original environment (the environment in which it is naturally present). For example, a polynucleotide present in the natural state in a plant or an animal is not isolated. The same polynucleotide is “isolated” if it is separated from the adjacent nucleic acids in which it is naturally present. The term “purified” does not require the material to be present in a form exhibiting absolute purity, exclusive of the presence of other compounds. Rather, it is a relative definition. A polynucleotide is in the “purified” state after purification of the starting material or of the natural material by at least one order of magnitude, preferably 2 or 3 and preferably 4 or 5 orders of magnitude.

A “nucleic acid”, “nucleotide”, or “polynucleotide” is a polymeric compound comprised of covalently linked subunits called nucleotides. Nucleic acids include polyribonucleic acid (“RNA”) and polydeoxyribonucleic acid (“DNA”), both of which may be single-stranded or double-stranded. DNA includes but is not limited to, cDNA, genomic DNA, plasmid DNA, synthetic DNA, and semi-synthetic DNA. DNA may be linear, circular, or supercoiled.

A “nucleic acid molecule” refers to the phosphate ester polymeric form of ribonucleosides (adenosine, guanosine, uridine, or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single-stranded form, or a double-stranded helix. Double stranded DNA-DNA, DNA-RNA, and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

The term “fragment” when referring to a polynucleotide will be understood to mean a nucleotide sequence of reduced length relative to the reference nucleic acid and comprising, over the common portion, a nucleotide sequence identical to the reference nucleic acid. Such a nucleic acid fragment according to the present application may be, where appropriate, included in a larger polynucleotide of which it is a constituent. Such fragments comprise, or alternatively consist of, oligonucleotides ranging in length from at least 8, 10, 12, 15, 18, 20 to 25, 30, 40, 50, 70, 80, 100, 200, 500, 1000, 1500, or any number or range therein, consecutive nucleotides of a nucleic acid according to the present application.

As used herein, an “isolated nucleic acid fragment” is a polymer of RNA or DNA that is single- or double-stranded, optionally containing synthetic, non-natural, or altered nucleotide bases. An isolated nucleic acid fragment in the form of a polymer of DNA may be comprised of one or more segments of cDNA, genomic DNA, or synthetic DNA.

A “gene” refers to an assembly of nucleotides that encode a polypeptide, and includes cDNA and genomic DNA nucleic acids. “Gene” also refers to a nucleic acid fragment that expresses a specific protein or polypeptide, optionally including regulatory sequences preceding (5′ noncoding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Heterologous” DNA refers to DNA not naturally located in the cell, or in a chromosomal site of the cell. Preferably, the heterologous DNA includes a gene or polynucleotides foreign to the cell.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the disclosure herein, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase or transcription factors.

A coding sequence is “under the control” of transcriptional and translational control sequences in a cell when RNA polymerase transcribes the coding sequence into mRNA, which is then RNA spliced (if the coding sequence contains introns) and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

As used herein a “protein” is a polypeptide that performs a structural or functional role in a living cell.

An “isolated polypeptide” or “isolated protein” or “isolated peptide” is a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be present, for example, due to incomplete purification, addition of stabilizers, or compounding into a pharmaceutically acceptable preparation.

As used herein “reference sequence” means a nucleic acid or amino acid used as a comparator for another nucleic acid or amino acid, respectively, when determining sequence identity.

As used herein “percent identity” or “% identical” refers to the exactness of a match between a reference sequence and a sequence being compared to it when optimally aligned. For example, sequence alignments and percent identity calculations may be determined using a variety of comparison methods designed to detect homologous sequences including, but not limited to, the Multalin program (Corpet, “Multiple Sequence Alignment with Hierarchical Clustering,” Nucleic Acids Res. 16:10881-90 (1988), which is hereby incorporated by reference in its entirety) or the Megalign® program of the LASERGENE® bioinformatics computing suite (DNASTAR® Inc., Madison, Wis.). Sequences may also be aligned using algorithms known in the art including, but not limited to, CLUSTAL V algorithm or the BLASTN or BLAST 2 sequence programs.

As used herein, “operatively linked” DNA segments, means one polynucleotide sequence is joined to another so that the polynucleotides are in association for transcriptional and/or translation control and can be expressed in a suitable host cell.

The term “about” typically encompasses a range up to 10% of a stated value.

The term “control” plant or “wild type” plant refer to a plant that does not contain any of the mutations described in the present application.

### Lettuce Plants with PPO Gene Mutations

One aspect of the present application is directed to a lettuce plant comprising a mutation in each of at least two different PPO genes, where said mutations reduce the activity of PPO protein compared to a wild type lettuce plant.

Mutations occurring in the PPO genes of the lettuce plant of the present application may be present in any of a lettuce plant's PPO genes. In one embodiment, the lettuce plant comprises a mutation in each of at least two different PPO genes. In certain embodiments, the at least two different PPO genes are selected from the group consisting of PPO-A, PPO-B, PPO-C, PPO-D, PPO-E, PPO-G, PPO-J, PPO-M, PPO-N, PPO-O, PPO-P, PPO-Q, PPO-R, and PPO-S. In some embodiments, the at least two different PPO genes are selected from the group consisting of PPO-A, PPO-B, PPO-C, PPO-D, PPO-E, PPO-G, PPO-O, PPO-P, PPO-R, and PPO-S. Non-limiting examples of gene and protein sequences of PPO genes in lettuce are provided as follows.

PPO-A gene sequence (SEQ ID NO: 1):
ACAATATCTG CACGCTCAAA ATAAGACAAT GGCATCTCTT GCACAATCAC CAACCACCAC
CACCACCACC GGTGGACGGT GCTTCTCCTC CTCCTCCACG TACTCTTCTT CCTTCTCTTT
CAAATCATCT CAAGTTCCCA TAGCACGAAT CACGAACCAT CGCCATGCAG TTTCATGCAA
AGGCGCCCTA GATGATGATG ACCATCACCA TGAAAACTCA GGCAAATTTG ATAGGAGAAA
CGTCTTATTA GGTCTCGGAG GTCTTTACGG CGCCGCCGCC ACTTTTGGGT CAAACTCATT
GGCGTATGCA GCTCCGATTA TGGCACCGGA CCTCACAAAA TGTGGTCCGG CTGACTTACC
CCAAGGGGCT GTACCTACAA ACTGTTGCCC TCCATACACC ACAAAGATTC ACGATTTCAA
ACTTCCACCA CCGTCAACCA CCTTCCGAGT CCGTCCGGCA GCTCATTTGG CTAATAAAGA
TTACATAGCC AAGTTCAATA AAGCCATCGA GCTCATGAAA GGTCTCGGAG ATGACGATCC
TCGTAGTTTC AAGCAACAAG CTGCTGTTCA TTGTGCGTAT TGCGATGGGG CATACGATCA
AGTCGGTTTC CCTGATCTCG AGCTTCAAGT CCATGGCTCA TGGTTGTTCT TACCTTTCCA
CCGCTATTAC TTATACTTCT TCGAGAAAAT TTGTGGCAAA TTAATCGATG ATCCAAATTT
CGCAATCCCT TTTTGGAACT GGGATGCACC TGATGGCATG AAGATCCCTG ATATTTACAC
GAATAAGAAA TCTCCGTTGT ACGATGCTCT TCGTGATGCG AAGCATCAAC CACCGTCTCT
GATTGATCTT GACTACAATG GTGACGATGA AAATCTTAGC CGATCGAAAC AAACCTCCAC
AAATCTCACA ATTATGTACA GACAAATGGT GTCTAGTTCC AAGACTGCTA GTCTTTTCAT
GGGTAGTCCT TATCGTGCAG GTGATGAGGC TAGCCCTGGC TCTGGCTCGC TCGAGAGCAT
ACCACATGGC CCGGTTCATA TCTGGACCGG AGATAGGAAC CAGCAAAATG GTGAAGACAT
GGGTAACTTT TATTCTGCAG CCAGAGACCC TATTTTCTAT GCACATCATG CGAATATCGA
CAGAATGTGG TCAGTTTGGA AAACTCTAGG AGGAAGAAGG AATGATTTTA CAGATAAAGA
CTGGCTTGAT TCTTCGTTCT TGTTCTACGA TGAGAACGCT GAAATGGTTC GAGTCAAGGT
GAGGGATTGT CTCGACTCCA AGAAGCTTGG GTACGTTTAT CAGGATGTAG AGATACCATG
GCTAAAAAGC AAACCCGAAC CACGTCTGAA AAGGGCTTTG AGCAAGATCA AGAAGCTCGC
TGTAGCTCGA GCCGATGAAC ACATACCCTT TGCAAAAGAT GTTTTTCCGG CGAGTCTTGA
TAAGGTGATA AAAGTGCTGG TTCCAAGGCC GAAGAAATCA AGGAGCAAGA AACAGAAAGA
GGATGAAGAA GAAATTTTGG TGATAGAAGG AATTGAACTG AAGAGAGATG AGTTTGCGAA
GTTTGATGTG TTTGTGAACG ATGAAGATGA CGGGATGAGG GCCACGGCTG ATAAGACGGA
GTTCGCCGGA AGTTTTGTTA ATGTCCCTCA TAAGCATAAG CATGGGAAGA ATGTGAAGAC
AAGATTGAGG TTAGGAATAA GTGAGCTTTT GGAGGATTTG GGAGCTGAAG ATGATGACAA
CGTGTTGGTG ACATTGGTGC CGAAAAACAA AGGTGGTGAA GTTTCCATTA AAGGGATTAA
AATCGAGCAT GAGGATTGAT AAAAATAACT TTTCATTTTC TTGAAAAATA AAAAACTATG
ATTTTGAGTT GTTTCGGTAA ATATGTTGTC GCTTGGTTAA TGTATCATCA ATAAAAATAA
ATTTCGAAAT CAAAGTTGGA TTTGAACCCC ACAA
PPO-A protein sequence (SEQ ID NO: 2):
MASLAQSPTT TTTTGGRCFS SSSTYSSSFS FKSSQVPIAR ITNHRHAVSC KGALDDDDHH
HENSGKFDRR NVLLGLGGLY GAAATFGSNS LAYAAPIMAP DLTKCGPADL PQGAVPTNCC
PPYTTKIHDF KLPPPSTTFR VRPAAHLANK DYIAKFNKAI ELMKALPDDD PRSFKQQAAV
HCAYCDGAYD QVGFPDLELQ VHGSWLFLPF HRYYLYFFEK ICGKLIDDPN FAIPFWNWDA
PDGMKIPDIY TNKKSPLYDA LRDAKHQPPS LIDLDYNGDD ENLSRSKQTS TNLTIMYRQM
VSSSKTASLF MGSPYRAGDE ASPGSGSLES IPHGPVHIWT GDRNQQNGED MGNFYSAARD
PIFYAHHANI DRMWSVWKTL GGRRNDFTDK DWLDSSFLFY DENAEMVRVK VRDCLDSKKL
GYVYQDVEIP WLKSKPEPRL KRALSKIKKL AVARADEHIP FAKDVFPASL DKVIKVLVPR
PKKSRSKKQK EDEEEILVIE GIELKRDEFA KFDVFVNDED DGMRATADKT EFAGSFVNVP
HKHKHGKNVK TRLRLGISEL LEDLGAEDDD NVLVTLVPKN KGGEVSIKGI KEIHED
PPO-B gene sequence (SEQ ID NO: 3):
TTATATAAAT ATAGATCACT TCACATAATC TCATGCATCG TGCTCAAAAT TATGACATCC
TTTGCATCAT CACCGACCAA AACCCTAACA GGCACGGCCT CCAGGAGTGA AAGGAGGATC
TCTTCCTCAT CCAACTACTC TTCTTCCTTC TCTTTTAAGT CATCTCAAGT TCCCATAGCC
AGAATCTCCA AACATCGCCA TGCAGTTTCA TGCAAAACCC TAGATGACGA TCACCACCAC
CATGCAAACT CCGGCAAACT TGATAGGAGA AACATCCTGT TAGGCCTCGG TGGTCTTTAT
GGTACTGCCG CCACTTTTGG GTCTAATTCA CCAGCCATTG CAGCTCCGAT CATGGCACCC
GACCTCTCAA AATGTGGTCC GGCCGACTTG CCCGAAGGTG CTGTATCCAC AGACTGTTGC
CCTCCATACA CCACAAAGAT TCTCGATTTC AAACTTCCAC CACCGTCAAA CACCTTCCGA
GTCCGTCCGG CAGCTCATTT GGCTAATGAA GATTACATAG GCAAGTTCAA TAAAGCCATC
GAGCTCATGA AAGCTCTCCC AGATGACGAT CCTCGTAGCT TTAAGCAGCA AGCAAATGTT
CATTGTGCCT ATTGCGATGG CGCGTATGAC CAAGTCGGTT TTCCAGATCT GGAGCTTCAA
GTACATAACT CATGGCTGTT CTTCCCTTTC CATCGCTATT ACATGTACTT CTTCGAGAAA
ATTTGTGGCA AGTTAATTGA TGACCCAAAT TTCGCAATTC CATTTTGGAA CTGGGATGCA
CCAGATGGGA TGAAAATCCC TGATATTTAC ACAAATAAGA AATCTTCGTT GTATGATCCT
CTTCGCGATG TGGACCATCA ACCACCGTCT TTGATTGATC TTGACTTCAA TGGTGTCGAC
GAAAATCTTA GCCCCTCTGA ACAAACGTCC AAAAATCTCA CAGTTATGTA TAGACAAATG
GTGTCTAGTT CCAAGACTTC TACTCTTTTC ATGGGTAGTC CTTATCGTGC AGGCGATGAT
GCTAGCCCTG GTAGTGGTTC GATCGAGAAC ACACCACATA ACCCAGTTCA TATCTGGGCC
GGTGAGTGGA AGCATAATAA TGGCAAAAAC ATGGGCAAAC TTTATTCTGC AGCCAGGGAC
CCTCTTTTCT ATGCACATCA TGGGAATATT GATAGAATGT GGTCAGTTTG GAAAACACTA
GGTGGAAGAA GGAAGGACTT TACTGATAAA GATTGGCTTG ATTCTTCGTT CTTGTTTTAC
GATGAGAACG CTGAGTTGAA TCGAGTCAAG GTGAGGGATT GTCTCGACAC CAAGAATCTT
GGCTACGTTT ATCAAGATGT AGAGATACCA TGGCTAAAAA GCAAACCTGT TCCACGTCGG
ACAAAGCCCA AGGAGAAGCC CAAAAACAAA AACAACAAGC AAGCTGTGGC TCGAGCCGAC
GAATACATAC CATTTGCAAA AGATGTTTTT CCGGCGAGTC TTAATGAGGT CATCAAAGTG
CTGGTTCCAC GGCCCAAGAT ATCAAGGAGT AAGAAACAGA AAGAAGAAGA AGAAGAGATT
TTGGTGATCG AAGGAATTGA AGTGAAGATA GATGAGTTTG TGAAGTTTGA TGTGTTTGTC
AATGATGAAG ATGACGGGAT GAGGGCCACC GCAGATAAGA CGGAGTTTGC CGGAAGTTTT
GTGAATGTCC CTCATACTCA TAAGCATGGG AAGAATTTGA AGACGAGATT GAGGCTAGGG
ATAAGTGAGC TTTTGGAGGA TTTGAATGCT GAAGATGATG AAAATGTGTT GGTGACATTG
GTGCCCAAAA CTAGGGGTAG TGGAATTTCC ATTGCAGAGA TCAAAATCGA GCATGAAGAA
TGATCAAAAT ACGTTCACTT TCTCTAAAAT AAAAATAAAG GAACGTTTAT GATGGAGGAA
TATATTATGT TTTTCGATAT GTATTACCAT ATAAAAATAA TTTTCGCAAT CAAATAAAGG
AACGTTTATG ATGGAGGAAT A
PPO-B protein sequence (SEQ ID NO: 4):
MTSFASSPTK TLTGTASRSE RRISSSSNYS SSFSFKSSQV PIARISKHRH AVSCKTLDDD
HHHHANSGKL DRRNILLGLG GLYGTAATFG SNSPAIAAPI MAPDLSKCGP ADLPEGAVST
DCCPPYTTKI LDFKLPPPSN TFRVRPAAHL ANEDYIGKFN KAIELMKALP DDDPRSFKQQ
ANVHCAYCDG AYDQVGFPDL ELQVHNSWLF FPFHRYYMYF FEKICGKLID DPNFAIPFWN
WDAPDGMKIP DIYTNKKSSL YDPLRDVDHQ PPSLIDLDFN GVDENLSPSE QTSKNLTVMY
RQMVSSSKTS TLFMGSPYRA GDDASPGSGS IENTPHNPVH IWAGEWKHNN GKNMGKLYSA
ARDPLFYAHH GNIDRMWSVW KTLGGRRKDF TDKDWLDSSF LFYDENAELN RVKVRDCLDT
KNLGYVYQDV EIPWLKSKPV PRRTKPKQKP KNKNNKQAVA RADEYIPFAK DVFPASLNEV
IKVLVPRPKI SRSKKQKEEE EEILVIEGIE VKIDEFVKFD VFVNDEDDGM RATADKTEFA
GSFVNVPHTH KHGKNLKTRL RLGISELLED LNAEDDENVL VTLVPKTRGS GISIAEIKIE
HEE
PPO-C gene sequence (SEQ ID NO: 5):
CTCTTCACAT ATTTCATGCA CGCTCAAACT ACAAGACTAT GGCATCCTTT TCACCATCAC
AAGCCACCTC TTACACGAGT GGAAGGAGGT TCTCTTCCTC ATCGACCTAC TCTTCTTCCT
TCTCTTTTAA GTCATCTCAA GTTCCCATAG CCAGAATCTC CAAACATCGC CATGCAGTTT
CATGCAAAAC CCTAGATGAT GATCACCACC ACCATGCAAA TTCCGGCAAA CTTGATAGGA
GAAACGTCCT TTTAGGCCTT GGAGGTCTTT ATGGTACTGC CGCCAACTTT GGGTCTAATT
CACTGGCCTT TGCAGATCCG ATCATGGGAC CCGACCTCAG TAAATGTGGT CCGGCTGAGT
TACCCCAAGG GGCTATACCT ACAAATTGTT GTCCTCCATT CACCACAAAG ATTATCGATT
TCAAACTTCC ACCACAGTCA AACCCCCTCC GTGTTCGACC AGCTGCACAT TTGGTTGATA
AAGACTACAT AGACAAATTC AGTAAAGCTA TCGAACTCAT GAAAGCTCTC CCAGATGACG
ATCCTCGTAG TTTCAAGCAA CAAGCTAATG TTCACTGTGC CTATTGTGAT GCCGCATATG
TCCAACTCGG TTATCCAGAT GTGGAGCTTC AAGTACATAA CTCATGGCTG TTCTTCCCTT
TCCATCGTTG TTACCTATAC TTCTTTGAGA AAATTTGTGG CAAATTAATT GATGACCCAA
CTTTTGCAAT TCCATTTTGG AACTGGGATG CGCCAGTTGG GATGAAAATC CCTGATATTT
ACACAGATAA GAATTCTTCG TTATACGATA CTCTTCGTGA TGCGAAACAT CAACCACCGA
CTGTGGTTGA TCTTGACTAC AATGGTTTCG ACAACAATCT TAGCCCCTCT GAACAAACGT
CCACAAATCT CACGATTATG TATAGACAAA TGGTGTCTAA TGCCAAGACT GCTAGTCTTT
TCATGGGTAG TCCTTATCGT GCAGGTGATG ACCCTAGCCC TGGTGCTGGC TCGCTCGAGA
GCGTGCCACA TAACCCGGTT CATATCTGGA CCGGGGATAG GAACCAGCCA AATGGTGAAG
ACATGGGTAA CTTTTATTCT GCAGGCAAAG ACCCTATTTT CTTTGCACAT CATGGGAATC
TCGATAGATT GTGGTCAGTT TGGAAAACAC TAGGTGGAAG AAGGAAGGAT TTTACTGATA
ATGATTGGCT TGATTCTTCG TTCTTGTTGT ACGATGAGAA CGCTGAGTTG AATCGAGTCA
AGGTGAGGGA TTGTGTCGAC TCCAAGAATA TGAATTATGT TTATCAAGAT GTTGAGTTAC
CATGGCTAGA AAGCAAACCT GTTCCACGAC TGCAAAAGGC TTCCAGAAAC ATCAAGAAGC
ATGCCCATGA ACACATACCC TTTGCAAAAG ATGTTTTTCC GGCGAGTCTT GATAAGGTGA
TCAAAGTGCG GGTTCCAAGG CTTAAGAAAT CAAGGACCAA GAAACAGAAA GAGGAGGAAG
AAGAGATTTT GGTTATTGAA GGGATTGAAG TGAAGAGAGA TGAGTTTGTG AAGTTTGATG
TGTTGGTGAA CGATGATGAT GATGGGACCC AGGCCACAGC AGCTAAAACG GAGTTCGCCG
GAAGTTTTGC GAGTGTCCCT CATATGCATA AGCATGGGAA GAATTGGAAG ACGAAATTGA
GGATAGGGAT AACTGACCTT TTGGAGGATT TGAAGGATGA AGAAGATCAC AATGTGTTGG
TGACATTGGT GCCCAAAACT AGTGGTGGTG ATATTTCCAT TGGAGGGATC AAAATCGAGC
ATGAAGAATG TTAAACAGAC GTTCTCACTT TCTATAAATA AAATAAAAGA AAGTCTATGA
TCTATTAAGA TATTATGTTT CTCTATATGT ATTACCATAT AAAAGTAATT ATCACAATAA
AGTTATATAT GATTCGAACT TGTGAATGTT AATTGCAAGT CATTGAA
PPO-C protein sequence (SEQ ID NO: 6):
MASFSPSQAT SYTSGRRFSS SSTYSSSFSF KSSQVPIARI SKHRHAVSCK TLDDDHHHHA
NSGKLDRRNV LLGLGGLYGT AANFGSNSLA FADPIMGPDL SKCGPAELPQ GAIPTNCCPP
FTTKIIDFKL PPQSNPLRVR PAAHLVDKDY IDKFSKAIEL MKALPDDDPR SFKQQANVHC
AYCDAAYVQL GYPDVELQVH NSWLFFPFHR CYLYFFEKIC GKLIDDPTFA IPFWNWDAPV
GMKIPDIYTD KNSSLYDTLR DAKHQPPTVV DLDYNGFDNN LSPSEQTSTN LTIMYRQMVS
NAKTASLFMG SPYRAGDDPS PGAGSLESVP HNPVHIWTGD RNQPNGEDMG NFYSAGKDPI
FFAHHGNLDR LWSVWKTLGG RRKDFTDNDW LDSSFLLYDE NAELNRVKVR DCVDSKNMNY
VYQDVELPWL ESKPVPRLQK ASRNIKKHAH EHIPFAKDVF PASLDKVIKV RVPRLKKSRT
KKQKEEEEEI LVIEGIEVKR DEFVKFDVLV NDDDDGTQAT AAKTEFAGSF ASVPHMHKHG
KNWKTKLRIG ITDLLEDLKD EEDHNVLVTL VPKTSGGDIS IGGIKIEHEE C
PPO-D gene sequence (SEQ ID NO: 7):
ACCACACCAC CTATAGATGA TGGCTTCCCT CAGCTTGTCC ACTCTTCCCA CCTCCACCCC
CACAAAAAAG CCTTTATTTT CCAAAACCTC CTCCCACGTG AAGCAATCCC ATCGCTTCAA
AGTCTCATGC AACTCCGCCG CTAACAACAA TGAGAAAACA GTCAAAAACT CTGAAACCCC
GAAGCTCATA CTACCCAAAA CACCACTTGA AATGCAGAAT GTTGACCGGA GAAACCTGCT
CCTGGGGCTT GGAGGTCTCT ACGGCGCTGC CAACTTGACA TCCATCCCAT CAGCCTTTGG
CACTCCCATC GCTGCTCCGG ACAATATTTC AGATTGTGTT ACTGCGTCGT CAAACCTCCA
GAACGCCAAT GACGCTGTAA GGGGTTTAGC TTGTTGCCCT CCAGTACTCT CAACAGATAA
ACCAAAAGAT TACGTCTTGC CTACCAACCC AGTCCTTCGT GTTCGACCAG CTGCACAGAG
AGCTACTGAC GAGTACATCG TAAAGTACAA AGCAGCGATT CAAGCCATGA AGAATCTCCC
CGACGAGCAT CCACACAGTT GGAAGCAACA AGCTAAGATC CACTGCGCTT ATTGCAACGG
TGGTTACAAT CAAGAACAGA GTGGTTTCCC GGACATACAA CTCCAGATTC ACAACACATG
GCTCTTCTTT CCTTTCCACC GATGGTACCT CTACTTCTAC GAGAGGATTT TGGGGAAGTT
GATTAATGAT CCAACTTTCG CTTTACCATA CTGGAACTGG GATAACCCTA CCGGAATGGT
GCTCCCTGCC ATGTTCGAAA CCGACGGCAA AAGGAACCCT ATCTTTGACC CTTACAGGAA
TGCCACACAC CTCCCACCAG CTATCTTTGA AGTGGGATAT AATGGGACAG ACAGTGGCGC
CACTTGTATA GACCAGATAA GCGCTAATCT GTCTTTGATG TACAAGCAAA TGATCACCAA
CGCTCCTGAT ACAACAACGT TCTTCGGTGG AGAATTTGTT GCTGGGGATG ACCCTCTTAA
CAAAGAGTTT AACGTTGCTG GGTCCATAGA GGCTGGGGTT CACACTGCGG CGCATAGATG
GGTGGGTGAT CCTAGGATGG CCAACAGCGA GGACATGGGG AACTTCTACT CCGCAGGGTA
TGATCCTCTC TTTTACGTCC ACCATGCCAA CGTCGACCGG ATGTGGAAAA TCTGGAAAGA
TTTGGGAATC AAGGGACACA CTGAACCGAC GTCCACCGAC TGGCTAGATG CTTCATACGT
GTTTTATGAT GAGAACGAAG AGCTTGTACG TGTCTATAAC CGAGACAGTG TAAACATGAC
TGCAATGGGA TACGACTATG AAAGGTCCGA AATCCCGTGG CTCCATAGTC GATCGGTTCC
ACATACCAAG GGGGCCAATG TTGCAGCTAA ACTGGTCGGA ATCGTGAAGA AGGTGGAAGA
CGTTACATTC CCGTTGAAGT TAAATGAGAC AGTGAAGGTT CTTGTGAAAA GGCCTACTAA
GAAGAGGAAC AAGAAGAACA AGCAGGAAGC GAATGAGATG TTGTTCTTGA ATAAAATCAA
GTTCGATGGC GAGGAGTTTG TCAAGTTTGA CGTGTTTGTC AATGACGTTG ACGATGGAGT
GGAGACTACC GCAGCTGAGA GTGAGTTTGC TGGTAGTTTC TCACAATTGC CCCATGGCCA
TAAACATGGC ACCAAGATGT CAATGACGAG TGGGGCGGCG TTTGGGCTTA CGGAGCTGTT
GGAGGACATT GAAGCTGAAG ATGATGACTC TATTTTGGTG ACTTTGGTGC CCAAGATAGG
GTGTGATGAT GTGACTGTCG GTGAGATTAA GATTAAGTTG GTTCCCATTG TCTGAAGTTC
ATTGATGTAA CATCGTTTTC ATTTGCGTTT GTATGCATGG GTAAAACAGT TTTCTGTGTT
TGGTCATACG AGGATGTTTG TGGTTCTCGT AATCTAATAA TGACCATTTT GTCAAGTTTG
TTGTCATGCT TGATTGTAAC TCCTATGTTT GGATATCAAT AAACATTATC GAGTACTATT
TTAGT
PPO-D protein sequence (SEQ ID NO: 8):
MMASLSLSTL PTSTPTKKPL FSKTSSHVKQ SHRFKVSCNS AANNNEKTVK NSETPKLILP
KTPLEMQNVD RRNLLLGLGG LYGAANLTSI PSAFGTPIAA PDNISDCVTA SSNLQNANDA
VRGLACCPPV LSTDKPKDYV LPTNPVLRVR PAAQRATDEY IVKYKAAIQA MKNLPDEHPH
SWKQQAKIHC AYCNGGYNQE QSGFPDIQLQ IHNTWLFFPF HRWYLYFYER ILGKLINDPT
FALPYWNWDN PTGMVLPAME ETDGKRNPIF DPYRNATHLP PAIFEVGYNG TDSGATCTDQ
ISANLSLMYK QMITNAPDTT TFFGGEFVAG DDPLNKEFNV AGSIEAGVHT AAHRWVGDPR
MANSEDMGNF YSAGYDPLFY VHHANVDRMW KIWKDLGIKG HTEPTSTDWL DASYVFYDEN
EELVRVYNRD SVNMTAMGYD YERSEIPWLH SRSVPHTKGA NVAAKLVGIV KKVEDVTFPL
KLNETVKVLV KRPTKKRNKK NKQEANEMLF LNKIKFDGEE FVKFDVFVND VDDGVETTAA
ESEFAGSFSQ LPHGHKHGTK MSMTSGAAFG LTELLEDIEA EDDDSILVTL VPKIGCDDVT
VGEIKIKLVP TV
PPO-E gene sequence (SEQ ID NO: 9):
CTTATAGCAC CACCCATAGA TGATGGCTTC TCTCGCCTTG TCTAGTCTTC CCACCTCCAC
CACAACCAAA AAACCCTTAT TTTCCAAAAC ATCCTCGCAT GTTAAGCCAT TCCATCGCTT
CAAAGTTTCA TGCAATGCAC CCGCTGATAA CAATGACAAA ACCGTCAATA ATTCTGATAC
CCCAAAGCTC ATACTACCCA AAACACCACT TGAAACGCAG AACGTAGACA GGAGAAACTT
GCTTCTGGGA CTCGGAGGTC TCTACGGCGC TGCCAACTTG ACGACCATTC CGTCAGCCTT
TGGCATTCCC ATCGCTGCTC CAGACAATAT TTCAGACTGT GTTGCTGCGA CTTCAAACCT
AAGGAACAGC AAAGACGCTA TAAGGGGACT AGCGTGTTGT CCTCCGGTGC TTTCAACAAA
CAAACCAATG GATTACGTCC TTCCTTCAAA CCCTGTGATT CGTGTTCGAC CAGCTGCACA
GAAAGCCACT GCCGATTACA TTGCTAAGTA TCAACAAGCA ATTCAAGCCA TGAAGGATCT
CCCCGAGGAC CACCCACATA GCTGGAAGCA ACAAGGCAAG ATTCACTGTG CTTATTGCAA
CGGTGGTTAC AATCAAGAAC AAAGTGGTTA CCCGAATTTA CAACTTCAGA TTCACAACTC
ATGGCTCTTC TTTCCTTTCC ACCGGTGGTA CCTCTATTTC TACGAGAAGA TATTGGGGAA
GTTGATTAAT GATCCAACTT TCGCTCTACC TTACTGGAAC TGGGATAACC CTACTGGAAT
GGTTATTCCT GCCATGTTCG AACAGAACAG CAAAACTAAC TCTCTGTTTG ACCCTTTAAG
GGATGCGAAA CACCTCCCAC CTTCTATCTT TGATGTTGAA TATGCTGGTG CAGACACTGG
TGCCACTTGT ATAGACCAGA TAGCCATTAA TCTGTCTTCA ATGTACAGAC AGATGGTCAC
CAACTCCACT GATACAAAAC GATTCTTCGG TGGCGAATTT GTAGCTGGAA ATGACCCTCT
TGCGAGCGAG TTCAACGTAG CTGGGACCGT AGAAGCTGGG GTTCACACTG CGGCTCACCG
CTGGGTGGGT AATTCTAGGA TGGCCAACAG CGAAGACATG GGGAACTTCT ACTCTCGCAG
GATATGATCC TCTCTTTTAC GTCCACCATG CGAATGTCGA CAGGATGTGG CAAATCTGGA
AAGATATTGA CAAGAAGACA CACAAGGATC CGACCTCTGG CGACTGGCTA AATGCATCAT
ACGTGTTTTA CGATGAGAAT GAAAATCTTG TACGTGTCTA CAACCGAGAC TGTGTAGACA
TTAATCGGAT GGGATATGAC TACGAAAGGT CAGCAATCCC ATGGATCCGT AGTCGGCCGA
CTGCACATGC GAAGGGGGCG AACGTTGCTG CTAAGTCTGC TGGAATCGTG CAGAAGGTGG
AGGATATCGT ATTCCCGCTG AAGTTAAACA AGATAGTGAA GGTTCTAGTG AAGAGGCCAG
CTACAAACAG GACCAAGGAG GAAAAGGAGA AAGCAAATGA GCTGTTGTTC GTGAATGGAA
TCACGTTTGA TGCTGAGCGG TTTCTAAAGA TTGACGTGTT TGTCAACGAC GTCGACGATG
GAATTCAGAC CACCGCTGCT GATAGTGAGT TTGCTGGTAG TTTCGCACAG TTGCCACATA
ACCATGGCGA CAAGATGTTT ATGAGGAGTG GGGCAGCGTT CGGGATCACG GAGCTCTTGG
AAGACATTGA AGCTGAAGGT GATGACTCTG TTGTTGTGAC ATTGGTGCCG AGAACAGGGT
GTGATGAAGT AACTATTGGC GAGATCAAGA TTCAGCTGGT TCCCATTGTT TAAAGTCTAT
TGAAGTAATG CATTTTCAAT TGTCATTAGT ATGCATGGGT ACGTAAATCT GTTCGCTGTC
TGGTTATCGA GGATTTTTGA TGTTCTCGTA ACCAAATAAT AAGGATTGTC ATTCCATGTT
TGGAATCGTG TAACCGCAGG CATGCATATG TTTGATTGTT ATTTTTAGTT GAAGCACTTC
TGTTTTAGTA ATCTCTGTTT TCCTGTTTTA CAAAAGGTAA AGAATTCGCT GTATGTGCTT
TGCATAA
PPO-E protein sequence (SEQ ID NO: 10):
MMASLALSSL PTSTTTKKPL FSKTSSHVKP FHRFKVSCNA PADNNDKTVN NDSTPKLILP
KTPLETQNVD RRNLLLGLGG LYGAANLTTI PSAFGIPIAA PDNISDCVAA TSNLRNSKDA
IRGLACCPPV LSTNKPMDYV LPSNPVIRVR PAAQKATADY IAKYQQAIQA MKDLPEDHPH
SWKQQGKIHC AYCNGGYNQE QSGYPNLQLQ IHNSWLFFPF HRWYLYFYEK ILGKLINDPT
FALPYWNWDN PTGMVIPAMF EQNSKTNSLF DPLRDAKHLP PSIFDVEYAG ADTGATCIDQ
IAINLSSMYR QMVTNSTDTK RFFGGEFVAG NDPLASEFNV AGTVEAGVHT AAHRWVGNSR
MANSEDMGNF YSRRI
PPO-G gene sequence (SEQ ID NO: 11):
CGATCAACGT TAGCATGCAC ACCACCAAAA GCTCATGGCT TCTCTTCCCA CACCAACAGT
TACAGCTGCC GGAGCCACCA CTAAAACCTA CTCTTCTTCT TTCACCACCA CTTCCCCTGT
CATCTCTTCT TGGCCGTTGT TCTCGAAGAA ATGTGCCCTT AATAAGCCAC TAAAACACAA
AATCTCTTGC AATGCTGGTT CTTCTGAGAA CTCCTTGAAC AACCTTGATC GCCGGAATGT
TCTTCTCGGT CTCGGTGGTC TTGCCGGAGC TGTGAACTTG ACGTCTGTTC CGTCTGTCGG
AGCTGCGCCA ATATCCGCCC CGGATATTTC CAAATGTGGG ACTAACCCTC TTTCAGGGTT
TAGACCTGGG GAGAGCACTC CCACCGGCGG CGACTGTTGC CCGCCTGACT CCCCCCAGAT
CATGGACTTC AAGTTCCCTA AGAATGAGGC GTTCAGGGTG AGACCCGCAG CCCATTTGCT
CAGCCCTAAG TACATTGCTA AATTCAACGA AGCGATCAAA CGCATGAAGG AACTTCCCGA
AACCGATCCT CGAAACTTTC TGCAACAAGC ACACATTCAC TGTGCTTACT GCAATGGCGC
TTACACTCAA TCTTCAAGTG GATTTCCCGA TATTGAAATC CAGATTCATA ACTCATGGCT
GTTCTTCCCC TTCCACCGTT GGTATCTCTA CTTTTACGAG AGAATCCTGG GGAGCTTGAT
CGATGATCCC ACTTTCGCTT TGCCATTCTG GAACTGGGAC ACCCCTGCCG GAATGACAAT
TCCGAAATAC TTTAACGATC CCAAAAACGC AGTTTTTGAT CCCAAAAGAA ACCAAGGTCA
CTTGCAAGGA GTCGTCGATC TGGGTTACAA TGGGAAAGAT TGAGACACTA CTGATATCGA
AAAGGTGAAG AACAATCTCG CGATAATGTA TCGTCAAATG GTGACAAACG CCACCGACCC
CACAGCTTTC TTCGGTGGTG AGTATCGTGC CGGAATCGAA CCCATTAGCG GTGGTGGATC
AGTCGAACAA AGCCCACACA CACCTGTTCA CCGGTGGGTC GGTGACCCAA GAGAACTTAA
CGGTGAAAAC CTCGGTAACT TCTACTCCGC CGGTCGTGAC ACGCTCTTTT ACTGTCACCA
TTCCAACGTC GATCGAATGT GGTCGTTGTG GAAGATGCAG GGAGGCAAAC ACAAGGACAT
CACCGATCCC GATTGGCTCA ACACCTCTTT CGTGTTTTAC GACGAAAACA GAAATCTTGT
TCGAGTGTAT GTTAAGGATT GTTTGTACAC AAACCAGCTA GGGTACGACT ACCAGAGAGT
CGACGTACCA TGGCTAAAAA GCAAGCCAGT CCCACGTGCA CCCAGGTCTG GAGTTGCGAG
GAAATCCATC GGAAAAGTAA AACAGGGGAA GGAAGTTTCC TTCCCGGTGA AACTCGACAA
GACCGTGAAG GTTTTGGTGG CAAGACCGAA GAAATCAAGA AGCAAGAAGG AGAAGGAGGA
CCAAGAGGAG CTTTTGATTG TTCAGGGTAT CACTTATGAT AGCGAGAAGT ACGTGAAGTT
TGATGTGTAT GTGAACGACG AGGACGACGA TGCTAGTGCA CCAGATCAGA CTGAGTTCGC
CGGAAGTTTC GCACAGTTGC CACACAAACA CAAGGGTAAG ACGATGAGCA AGACCAACTT
CCGCGCCGGA CTGACGGAGC TGCTGGAGGA TCTGGAGGCC GACGACGACG ACAATGTTTT
GGTGACGATT GTCCCAAGGT CTGGATCCGA AGACATCACC ATTGATAACA TCAAGATCAT
CTACGCTTGA TTTCAAGATT TGACGACTCT CAGTTGGGAT CATTAGTTAA ATATGTTTGA
TTAATGATCT TGCTATTCCC TTAATTATTA TTATTATTAT CAGGGTAGTT CGACCCTTGA
TTAGTGGTGA GGGTTGATGG TTGTCACCGG AATGTTTGGG TTTGAGTCCG AGTTCATGTG
ATTATGGGTC GGATTTAAAA TAAAATCTGA GTCGAGTCTG TGGTCATGTC GTAATTTGGG
GTTTCACTTT TGACCAAATC AATGTTAATT ATAATTTAAA TAAATTATTA TTAGTTCTCC
A
PPO-G protein sequence (SEQ ID NO: 12):
MASLPTPTVT AAGATTKTYS SSFTTTSPVI SSWPLFSKKC ALNKPLKHKI ACNAGSSENS
LNNLDRRNVL LGLGGLAGAV NLTSVPSVGA APISAPDISK CGTNPLSGFR PGESTPTGGD
CCPPDSPQIM DFKFPKNEAF RVRPAAHLLS PKYIAKFNEA IKRMKELPET DPRNFLQQAH
IHCAYCNGAY TQSSSGFPDI EIQIHNSWLF FPFHRWYLYF YERILGSLID DPTFALPFWN
WDTPAGMTIP KYFNDPKNAV FDPKRNQGHL QGVVDLGYNG KDSDTTDIEK VKNNLAIMYR
QMVTNATDPT AFFGGEYRAG IEPISGGGSV EQSPHTPVHR WVGDPRELNG ENLGNFYSAG
RDTLFYCHHS NVDRMWSLWK MQGGKHKDIT DPDWLNTSFV FYDENKNLVR VYVKDCLYTN
QLGYDYQRVD VPWLKSKPVP RAPRSGVARK SIGKVKQAKE VSFPVKLDKT VKVLVARPKK
SRSKKEKEDQ EELLIVQGIT YDSEKYVKFD VYVNDEDDDA SAPDQTEFAG SFAQLPHKHK
GKTMSKTNFR AGLTELLEDL EADDDDNVLV TIVPRSGSED ITIDNIKIIY A
PPO-J gene sequence (SEQ ID NO: 13):
GTTTTTGTAT GTTTTCTTTC CAATCTTTTG CAACCTTCAC TTCCATCACC ACCAGAACAC
TACCAAATTC CACCTCGGAT CGCCGCTACA ACAGTTACCC CAAACAAATC CACCACCTTC
AAATCTCATG CAACGTCGCA CCAGATGACA AAGAGAAGTT AGTCGTAGTC CCAGAAACCC
AAAAACTTAT TCTGCCAAAA TCATCTCTCG ACACACTTAA TGTAGATCGG AGGAACATGC
TCCTCGGGCT TGGGGGCCTT TACACCACCG TCAACTTCAC CTCCCCTGCA GCATTTGCTG
CACCTATCAC GACGCCAAAC TTCTCCACAT GCGTGACCTC AAATTTAGGT TTCCAGGACC
CAAATAAGGC CGTCAGAAGC AGAGCATGTT GTCCACCGGC GCCAGCGACG TCAACAGCCC
CCAAAGACTT TGTGTTCCCT AAAGACCAAG TGATCCGGAT TAGACCAGGG GCACATAGAA
CCACCACCGA GTACGTTGCT AAGTACAAAG CAGCAATCCA AGCGATGAGA GATCTCCCAG
ATGAACACCC ACACAGTTTC GTTGCACAAG CAAAAATTCA TTGCGCTTAC TGCAACGGTG
GTTACACTCA AATCGCGAGT GGTTTTCCAG ATAAAGAACT CCAGATTCAC AACTCATGGC
TCTTCTTTCC TTTCCATCGT TGGTACTTGT ATTTCTACGA GAGAATCCTC GGGAAGTTAA
TCGATGATCC AACTTTCGCT TTACCTTACT GGAACTGGGA CCATCCCAAC GGAATGACGT
TTCCCGCATT TTTGGAGGAC GATTCTGCCT TCGACGCTTA CCGTAATCGA AAGCACTTAC
CACCAGCACT TGTTGACCTC AACTACAGTG GCTCAGATAG ACACGCTACT TGTATTCGAC
AGATAACTAG CAATATGACA TTAATGTATA AGCAAATGAT CAGCAACGCC GGTGACACGA
CAAGCTTCTT TGGTAGCGAA TATCGGGCTG GCAACGACGC GTATAGAAAT GGTGACCCAT
CTGTCGGGTC GATAGAGGCT GGTTGTCACA CTGCGGTGCA TAGATGGATG GGTGACCCAG
GAATGCCGAA CAACGAGGAC ATGGGGAACT TCTACTCTGC GGGGTATGAC CCTGCGTTCT
ACATCCACCA TGCCAATGTC GACCGGATGT GGAAACTATG GAAGGATATG GGCATCAAAG
GACACTCTGA ACCTACACAT CTGGATTGGC GTAACGCATC GTACGTGTTT TATGATGAAA
ACGAACAGCT TGTTCGTGTC TACAACAAAG ATTGCGTCAG TTTGGAAAAG CTAAAATACG
ATTATGAATA CTCCCCACCC CTCTGGAAAA TAAGCCGATC CAGTATACGT CGTACCCTTC
CCGAACCCAT TCCATATAAC ATGAAATCTG CTGAAACGGT TAAACAACTG CCAGACGTGA
AGTTCCCCTT GAAGCTAGAC AAGATAACGA AGGTAGTAGT GAAGAGGCCA GCCAAAAGCA
GAAGTCAAGA AGACAAGGAA AAAGCAAATG AGCTGTTGTT GATTAAAGGA ATCAAGTTTA
ATAGCGACAA GTTCATCAAG TTTGATGTGT TTGTGAATGG ACAAGATGAT GTCAGCGAAA
GTTTTGAAGA AGAGAGTGAG TTTGCAGGTA GTTTCGCGCA GTTGCCACAT AACCATGGTG
ACGACATGTT AATGAAGAGT GGCATAAGGT TTGGGTTAAC GGAGCTTTTG GAGGAAATGG
AGGCGGAGGA TGATGAGTTT ATTTTGGTGA CTTTGGTGCC AAAGGTGTGG TTTGAGGAAG
TGACCATTGA CGAAATCAAG GTGGAGTTGG TTCCTATTAT CTGATTCATA ATCTAAAATT
GATGAGCATC GGGTACGTAA TTAACGTAGC GTATACGTAC CAACGCTGTA AATACAAAAA
AGTATTATTA TTCAACTAAA GTACCAAAGA GATACTCAAT TTAGTTGTAG TAACGGAGAA
GG
PPO-J protein sequence (SEQ ID NO: 14):
MFSFQSFATF TSITTRTLPN STSDRRYNSY PKQIHHLQIS CVNAPDDKEK LVVVPETQKL
ILPKSSLDTL NVDRRNMLLG LGGLYTTVNF TSPAAGAAPI TTPNFSTCVT SNLGFQDPNK
AVRSRACCPP APATSTAPKD FVFPKDQVIR IRPAAHRTTT EYVAKYKAAI QAMRDLPDEH
PHSFVAQAKI HCAYCNGGYT QIASGFPDKE LQIHNSWLFF PFHRWYLYFY ERILGKLIDD
PTFALPYWNW DHPNGMTFPA FLEDDSAFDA YRNRKHLPPA LVDLNYSGSD RHATCIRQIT
SNMTLMYKQM ISNAGDTTSF FGSEYRAGND AYRNGDPSVG SIEAGCHTAV HRWMGDPGMP
NNEDMGNFYS AGYDPAFYTH HANVDRMWKL WKDMGIKGHS EPTHLDWRNA SYVFYDENEQ
LVRVYNKDCV SLEKLKYDYE YSPPLWKISR SSIRRTLPEP IPYNMKSAET VKQLPDVKFP
LKLDKITKVV VKRPAKSRSQ EDKEKANELL LIKGIKFNSD KFIKFDVFVN GQDDVSESFE
EESEFAGSFA QLPHNHGDDM LMKSGIRFGL TELLEEMEAE DDEFILVTLV PKVWFEEVTI
DEIKVELVPI I
PPO-M gene sequence (SEQ ID NO: 15):
ATGGCTTCAC TGAGCTTCAC TTTAGCTATG GCCACCACCC CCTCTTCTTC CCCATTCTTT
TCCAAACCAG CGAACCAACG TCAGTTGATA AAGACACATG CTAAGCAAAC CCACCGCTTC
CAAATGTCAT GCAATGTTCC ATCAGACGAC CATGAAAAAC CAATCATCAA TACCCCTCAA
CATCAAAAGC TCATACTACC AAAAACATCA CTCGACATGC AGAACGTAGA CAGAAGGAAT
TTGCTCCTGG GGCTTGGTGG GCTCTACAGC GCCGTCAACT TGACGGGTCT CCCATCCGCT
TTTGCCGATC CTATCAGGAC TCCTTCTTTT AATCCAAATT GCAGGGACGC CGGAACGGGC
TTCGATGTCA AAAAAGGCCT TCTTAGAACT ACTGCATGTT GCCCTCCGGA GTCCAAGAAG
GGTCCCGAGA AACAATTCGA ATTCCCTAAA CATGACGAAA TACGCATCAG ATATCCCATA
CACTGTGCCC CGGAAGGATA CATGAATAAA TTTAAGGAGG CGATGAGGCT AATGAGGGCT
CTCCCAGATG ACGACCCTCG CAGTTTCAAG AACCAAGCCA AAATTCATTG CGCCTACTGC
AATGGCAGCT ACACTCAAAT GGCTACAGGT TCCCAACAAG AACTCCTGAT TCACTTCAAC
TGGCTGTTTT TTCCCTTCCA TCGATGGTAC CTTTATTTCT TCGAGAGGAT ACTCGGAGAA
CTGATTGGTG ATCCAACATT CGGGTTACCA TACTGGAGCT GGGACGAGCG TGAGGGAATG
AAAATTCCAC CTACGTTCCG AGAAGGGGGA GAGTCTAACC CTTTATATGA TATCTACCGG
AATAACATTC GCAACTATGA AGCTATTGTC GATCTTGACT TCAATGGTAA AGATCGCGAA
GATACGACTG AGGACTATCA GATAAAAATC AATCAGCATG CTATGTATCG CCAGATGATG
AGAAATGCCT TCGATACAAA AAGCTTCTTT GGTGGTAAGT ATGTCGCTGG TAATACACCC
ATTGATGCCA AAGACTCTTC AGTTGCATCC ATAGAGGCCG GTTGTCATAC CGCGATTCAC
AGATGGGTGC GTGACCCTGG AAGTCCGAAT GGTGAAGACA TGGGTAATTT CTACTCTGCC
GGGTATGATC CTTTGTTCTA TGTCCACCAT TCCAATGTCG ACAGGATGTG GGCACTTTGG
AAAGAAATGG GGGAAAGCAA CCGCGACCCC ATACACCCAG ACTGGTTAAA CGCATCATAT
GTGTTTTACG ACGAGAAACA AAATCCTGTT CGTGTCTACA ATAAACAATG CGTGGATATG
GAAAAGCTCA AATACAAATA CCATGGTCCA GAAATCCCCA GCTGGGTCAA TTCTCGGCCG
AAACCAAAGT GCAGCGCTTC GGAAAGATCC CAAATCGATA TCACGTCAGC CACAAAAGAT
GTGAAGAACC GAACCCTCAC CAACGTAGAT ACGTTTGTGT TAGTGAGGCC TGAAACTGCT
AGAACAAGGA CCGTGGATGA ATCAGAAATA GAGGTCTTGA CGTTGAACAA CATTAGTTTC
AACGGTAACA AAGCCGTCAA GTTTGACGTG CTTGTTAACG CTTGTAACAT TGACACAAAC
AAGTTCACCC CGGCTGATAG CGAGTATGCG GGTTCTTTTG CAACAGTTCC ACATAACCAT
GACATGAAAA TTAGTACTAC GTTCAGGTTT CCCTTAAGAG AGCTGTTGAA AGATATTGGA
GCTGAGGGAA ATACAGCAAT TCAAGTCACC ATTGTGACGC AAGAGAAAGA AACCGAGAAT
ATCAGCATTG GCGAGATCAA GATCGAGGAT TACTCTTTAG CCGAGATCTC GAAGGCGTCA
CTTCCCACTG GGCTACAGGG TGCCGGAGCT AATGTCGGCG TCGACGATCT AACAGAATAG
PPO-M protein sequence (SEQ ID NO: 16):
MASLSFTLAM ATTPSSSPFF SKPANQRQLI KTHAKQTHRF QMSCNVPSDD HEKPIINTPQ
HQKLILPKTS LDMQNVDRRN LLLGLGGLYS AVNLTGLPSA FADPITTPSF NPNCRDAGTG
FDVKKGLLRT TACCPPESKK GPEKQFEFPK HDEIRIRYPI HCAPEGYMNK FKEAMRLMRA
LPDDDPRSFK NQAKIHCAYC NGSYTQMATG SQQELLIHFN WLFFPFHRWY LYFFERILGE
LIGDPTFGLP YWSWDEREGM KIPPTFREGG ESNPLYDIYR NNIRNYEAIV DLDFNGKDRE
DTTDDYQIKI NQHAMYRQMM RNAFDTKSFF GGKYVAGNTP IDAKDSSVAS IEAGCHTAIH
RWVRDPGSPN GEDMGNFYSA GYDPLFYVHH SNVDRMWALW KEMGESNRDP IHPDWLNASY
VFYDEKQNPV RVYNKQCVDM EKLKYKYHGP EIPSWVNSRP KPKCSASERS QIDITSATKD
VKNRTLTNVD TFVLVRPETA RTRTVDESEI EVLTLNNISF NGNKAVKFDV LVNACNIDTN
KFTPADSEYA GSFATVPHNH DMKISTTFRF PLRELLKDIG AEGNTAIQVT IVTQEKETEN
ISIGEIKIED YSLAEISKAS LPTGLQGAGA NVGVDDLTE
PPO-N gene sequence (SEQ ID NO: 17):
ATGGCTTCTT TTAGCTTTTA CACTCTTCCT ACTTCCACCT CCACCATCAA GAATCCCTTA
TTTTCTAAAA GCTCCTCCCA TGTGAAGCAC TCACATCGCT TCAGGTCTTC ATGCAAAGCC
GCTGCTGATA GCAATGACAA ATATGTCGAA AATCCTGATA CCCCAAAACT CATACTACCA
AAATCACCCT CGCTTGATAC GCAGAACGTT GACAGGAGAA ACTTGCTCCT GGGACTCGGA
GGTCTCTACA GCGCTGCCAA CTTCACCAGC ATTCCGTCAG CCTTTGGCGT TCCCATCGAA
GCACGAGACA TTAATATTTC AAAGTGTGTT ACTGCCACCG TAAGGGGAGT CTCAGCAGAG
GCTATAAGGG GATTAACTTG TTGCCCTCCG GTGTTTGACT CATCGGCCAA ACCAGCGCCG
TACGAATTTC CGGATAACCA GGTAATTCGT ATGCGACCAG CGGCACAGAG AGTCAGTGCA
GACTACAAAA AAGACTTTCG AAAGGCAGTT GAGATAATGA AGGGATATAA CGACAATGAC
CCACACAGTT GGACGCAACA AGCTAAAGTT CATTGTGCGT ACTGCAACGG CGCTTACACT
CAAGTAAAAA GTGGTTTGGA GTTCGAGAAG TATATAATCC AAGTTCACAA CTCATGGCTC
TTCTTTCCAT TCCACCGTTG GTACCTCTAT TTCCTCGAGA AGATAATGGG AAAGGCGCTC
GGGGATGACA CTTTCGCTCT ACCATACTGG AACTGGGACC ACCCTACCGG AATGACGATT
CCTGCCATGT ACGAAGACAA ATTAAAAAAT CCGGATGGCA ACGTTGATAC CCCTGAAAAC
ACTAGATTCA ACTCTCTCTT TGATCCTTTA AGGAATACAT CACACATCGC ACCAGCTCTA
ATTGATTTTC AGTATTATCC TCAGAAACAA GAAGTTTATA ATTGTGCAGA CCAGATAGAG
ATTAATCTGT CTATAATGTA CAATCAGATG ATCGCCAACG CGCTTGATAC AAAATCGTTC
TTTGGTGGCG AACTTGTGGC TGGTGAAAAC CCCAATGAAA ACAAAAAGGC TGGGTCCATA
GAGGATGGGG TTCACACGAT TGCCCACCAA TGGGTGGGTA ACAATAGATT GAAGAACGGA
GAAGACATGG GAAACTTCTA CTCCGCAGGC TATGACCCTC TGTTTTACGG CCACCATGCG
AACGTTGACC GGATGTGGAA AATCTGGAAA GGTATGAACA GGAGACACCA TGCACCATCC
TCGACCGACT GGCTAGATGC ATCCTACGTA TTTTATGATG AGAATAGAAA ACTTGTACGT
GTCTACAACC GTGACTGTGT AGACACTAGA ACGATGGGGT ATGATTATGA GAGGTCCGAG
ATCCCATGGA TCCGAAATCG ACCTAATCCA CATCCCAAGG GAGGCAAAGA TAAAGGAAAT
GCTCGCAAAC CCGACAAAGC GACGGTGAAG GATCTCAGTT TCCCAGTGAG GTTAAACCAG
ACATTGGAGG TTCGAGTGAT GAGACCTGCG AAAAGGACTA CGGAGGACAA GGAGCGCACC
GAGATCGCGA TTGAGAAGTT GGTCCTTCAA GGCGTACGAT ATGATTGTGA GCGCTTTGTC
AAGTTCGATG TGATAATGAA CGACCCTGAT AATGGAGTCG ATGTCACCCC AGTTGACACT
GAGTTTCTTG GTTATTTCTC ACGGTTGCCC CATGGCATGG TCGCTGAAAA CAGAATGAAA
GAGATTAGTG GGATATCATT TGCCATCAAA GACCGCTTGA AAATCCTAAA AGTTGAAAAT
GATGATTCTA TTGTTGTGAA AATTGTGCCC AGAGCAGGGT GCGAGGATGT AACTATTCAG
AACATCGAGG TTGTGATGGA TCCCGTAGAC AATATTGTAC CTTTAGCGGA GAGCCTGGTT
GTGCAAGATC GGAACAGCGA TGAACTTACT TTGGAGGGCC CGACTGCGCT GGATTCGAAT
TCGGACGACT CTGGCTCGGA GTGATAAATT AAATCGTGTC GTTGTGTATC TTGTATATGT
GTGATCACTT TATAAGTTTA AGTTTGTATT CGAATCGCCA ATGTGGTGAT ATTTGCATTT
TGCCATTATT AATAATAATC CAATGCTCTT GTAGAAGAAA TTGCTCTGAG TGTCTAGAGT
TTTGTCTGAA TGGGTACGTA CGGTTACTTT TTTGTACGTC AATAATGCAC TTATTTGACT
TA
PPO-N protein sequence (SEQ ID NO: 18):
MASFSFYTLP TSTSTIKNPL FSKSSSHVKH SHRFRSSCKA AADSNDKYVE NPDTPKLILP
KSPSLDTQNV DRRNLLLGLG GLYSAANFTS IPSAFGVPIE APDINISKCV TATVRGVSAE
AIRGLTCCPP VFDSSAKPAP YEFPDNQVIR MRPAAQRVSA DYKKDFRKAV EIMKGYNDND
PHSWTQQAKV HCAYCNGAYT QVKSGLEFEK YIIQVHNSWL FFPFHRWYLY FLEKIMGKAL
GDDTFALPYW NWDHPTGMTI PAMYEDKLKN PDGNVDTPEN TRFNSLFDPL RNTSHIAPAL
IDFQYYPQKQ EVYNCADQIE INLSIMYNQM IANALDTKSF FGGELVAGEN PNENKKAGSI
EDGVHTIAHQ WVGNNRLKNG EDMGNFYSAG YDPLFYGHHA NVDRMWKIWK GMNRRHHAPS
STDWLDASYV FYDENRKLVR VYNRDCVDTR TMGYDYERSE IPWIRNRPNP HPKGGKDKGN
ARKPDKATVK DLSFPVRLNQ TLEVRVMRPA KRTTEDKERT EIAIEKLVLQ GVRYDCERFV
KFDVIMNDPD NGVDVTPVDT EFLGYFSRLP HGMVAENRMK EISGISFAIK DRLKILKVEN
DDSIVVKIVP RAGCEDVTIQ NIEVVMDPVD NIVPLAESLV VQDRNSDELT LEGPTALDSN
SDDSGSE
PPO-O gene sequence (SEQ ID NO: 19):
TTGAATTGTT TATTATAAAG ATTTAGCAGA AATGAAAAGC TCTTTGCATT GATCACAAGG
TTCTTTAACT TCTTTTTAGC AAACCCAACA TGGCCGGAAT ACCAACTACA CCTGCCACTT
TTCCGATGAG TCTAGACAAA CCAACGACGG TGATGGTGGC GAGGCCAGCA AAGAAGGAAA
GAGAGAAGGA GGAAGAAGAA GTATTGGTGA TAGAAGGGAT AGAAATCAAT AGAAATGAGT
TTGTGAAGTT TGATGTGTTC ATTAACGATG AGGATGAGGA GACAGCGGCT GGTGGTGGGG
CTGAGAAAGC TGAGTGTGCC GGTAGCTTTG TGAACGTGCC ACATAAGCAC AGGAACGGAC
ATAGTGGTGA TGGTGGGAAG GTGAAAAAGA CACAGTTGAG AATTGGAATA AGTGAGTTAT
TGGAGGATTT GGGTGTCGAA GAAGATGACG AAGATGTGGT GGTGAAATTG GTGCCAAGGT
GTGAAAATGT TCATGTCACA ATTGGTGGTA TTAAGATTGA GAATGAGTGA TTAATTAATT
AATTAATGAG TTCTTATCTT AGTTATTTTC TTTTGTTATC GAGTGGTTTA TGATCCCTTA
TAATAAATGT TATACTTAAT GTTGTCAAGA TC
PPO-O protein sequence (SEQ ID NO: 20):
MAGIPTTPAT FPMSLDKPTT VMVARPAKKE REKEEEEVLV IEGIEINRNE FVKFDVFIND
EDEETAAGGG AEKAECAGSF VNVPHKHRNG HSGDGGKVKK TQLRIGISEL LEDLGVEEDD
EDVVVKLVPR CENVHVTIGG IKIENE
PPO-P gene sequence (SEQ ID NO: 21):
TTTCCCACAA TCCGCGTCTA TATAATGGAG CTCAGCAGAA CGGATTTAAC ACCTACGACA
ATGATGGCTT CATATATCTT TTCCACCGTT CCCTCAGCCA CCGAAGTCAC CACCAACAAC
TTCTCCCATT CCTCGATATT TTCCAAAACT TCCACTCACC GATTCAAATA TACTCATGAA
AACCAAACCC ATCGTTTCAA AGTCTCATGC AACAAAACCT CAGACGACAA ATATGATACA
CTGGAAACAT CACTTGACAA GAAGAATGTA GACCGAAGAA ACTTGCTTCT GGGGCTCGGT
GGAACTCTCT ACGGTGCCGC CAACTTGACC TTCCTCCCGT CCGCCTTCTC GGTGCCCATT
GCTGCTCCCA ATGTTTCAGA CTGTGCTATT GCCAGTAAAG GTATACACAA CATCAAAGAT
GCTGTAAGGG GGGTAGCTTG TTGCCCACCA GTACTGACAC TAAATTCCCC AAAAAATTAC
GTCTTCCCGA AGGAGACCGC AGTTCGTATC CGTCCGGCAG CACAAAGAGC CTCTGACGAT
TACATTGACA AGTATAAAGC AGCAATTAAG GCCATGAGGG ATCTCCCAGA TGACCACCCA
CACAGTTTCA AGCAACAAGC CAAGATCCAT TGCGCTTATT GCAATGGCTC TTACACTCAA
AAAGAGAGTG GCAAGGAATA TGAACACCTC ACACTCCAGA TTCATAACTC GTGGCTCTTC
TTTCCTTTCC ACCGGTGGTA CCTCTATTTC TACGAAAGGA TATTGGGAAA GTTGATTGAC
GACCCAACTT TCGCGATACC ATACTGGAAT TGGGACAACC CCACCGGAAT GATAATCCCT
GACTTGTTTG AAAAACCCAT CCAAGTAAGG GAACGCAAAG AAAACCCCGT CTTTGACGCT
TACAGGGATG CCAGACACCT CCCACCAGCT CTTGTTGATA TCGATTATAA CGGTGAAGAC
CGTGGCGTTT CATGTATAGA CCAAATAACT ATTAATTTGT CTGCAATGTA TAAGCAGATG
ATCAGTAATG CTAGTGACCC AACAAGCTTC TTCGGTGGCA GATACGTCGC TGGGATGGAC
CACGATGACA AAAATAGTCA TGGAAATCCA TCGGTTGGGT CCATAGAAGC TGGTTGTCAC
ACAGCGGTGC ACCGATGGGT GGCTGATCCT CGGATGCCGA ACAATGAAGA CATGGGAAAC
TTCTACTCCG CAGGGTATGA CCCTATCTTT TATGCCCACC ACGCCAATGT TGACCGGATG
TGGAAAATCT GGAAAGAGTT GGGTATCAGG GGACACCGTG AACCAACCGA CAAGGACTGG
CTAGATGCAT CATACGTGTT TTACGATGAG AATGAAGAAC TTGTACGTGT CTATAACCGA
GACTGTGTGG ACTTGAATAA GCTTAACTAC GACTATGAAA CATCCCGTAT CCCGTGGGCC
AGGAATCGGC CGATCCCACG TGCTAAGAAT CCTCAAATGG CAGCGAGGTC AGCCCGAATG
GGGAGGAGTT TTCATGACGT GCAATTTCCG GTGAAGTTAG ACGGGATAGT AAAGGTGCTA
GTGAAGAGAC CTTATGTAAA CAGGACTAAG GAGGAGAAGG AGAAAGCAAA TGAGATATTG
ATGTTGAATG GGATTTGTTT TGATAGCGAG AAGTTTGTAA AGTTTGATGT GTATGTGGAT
GACAAGGACG ATGAACCAGA AACCACTGCG GCTGATAGCG AGTTTGCAGG TAGCTTTGCG
CAGTTGCCTC ACCATCAATC AGGCGAGAAG ATGTTCATGA CAAGTGCCGC GAGATTCGGG
TTAACAGAGT TGTTGGAGGA CATTGAAGCT GAAGATGATG AATCTATTAT GGTGACTTTG
GTTCCTAGGA CAGGGTCCGA TGATATCACA ATTTCAGACG TCAAGATTGA GCTGGTCCCC
ATCGTTTGAA CTTCAATAAT GTAATCGCAT TTTCATGTCC ATGTAATATG TTCGTTTTCT
GTGTTGTGTT TAATTAGGAG AATTTGCTGA GTTCTCTTAA CCTCAAAAAA GGGCAATAAG
TTGGTAGCGT TAATGTGTTA TCACTCGTGC ATCTCTATTT CAATTAAAGT TGATCAACTA
ATAAAAAATT TTTTGTTG
PPO-P protein sequence (SEQ ID NO: 22):
MELSRTDLTP TTMMASYIFS TVPSATEVTT NNFSHSSIFS KTSTHRFKYT HENQTHRFKV
SCNKTSDDKY DTLETSLDKK NVDRRNLLLG LGGTLYGAAN LTFLPSAFSV PIAAPNVSDC
AIASKGIHNI KDAVRGVACC PPVLTLNSPK NYVFPKETAV RIRPAAQRAS DDYIDKYKAA
IKAMRDLPDD HPHSFKQQAK IHCAYCNCSY TQKESGKEYE HLTLQIHNSW LFFPFHRWYL
YFYERILGKL IDDPTFAIPY WNWDNPTGMI IPDLFEKPIQ VRERKENPVF DAYRDARHLP
PALVDIDYNG EDRGVSCIDQ ITINLSAMYK QMISNASDPT SFFGGRYVAG MDHDDKNSHG
NPSVGSIEAG CHTAVHRWVA DPRMPNNEDM GNFYSAGYDP IFYAHHANVD RMWKIWKELG
IRGHREPTDK DWLDASYVFY DENEELVRVY NRDCVDLNKL NYDYETSRIP WARNRPIPRA
KNPQMAARSA RMGRSFHDVQ FPVKLDGIVK VLVKRPYVNR TREEKEKANE ILMLNGICFD
SEKFVKFDVY VDDKDDEPET TAADSEFAGS FAQLPHHQSG EKMFMTSAAR FGLTELLEDI
EAEDDESIMV TLVPRTGSDD ITISEIKIEL VPIV
PPO-Q gene sequence (SEQ ID NO: 23):
TTTTTTGGGA ATATAAATAG TGAACGCTAG TTCTCGTAAT TCGGGTAGGT GTATGATGGC
TTCTTTTAAC TTGTACACCG TTCCCGCCGC CACTACGGCC ACCACCAACA TAATCAAAAC
TAACTACTAC CAACTTAAGA CTCAAGCAAA GCAAACCCAT CGCTTGAAAG CGTCATGCAA
CGCAATCCCG GATAAAAACA ATGATAAAGC ACTTGAAACT TCACTTCAAA TGATAAACAT
TGACCGGAGA AACATAATAC TCCGCCTCGG TGGTCTCTTT GTGGCCAGCA ACATGACCTC
GGTTCCTTTG GCCTACGCTA ATGCAATTGC AGCTCGGTCT AATCACTCGG TTTGTGCTGC
TTCGCCTTTA GGCATACAGA ATCTTGGAAC CCCCGTAAAG GAACCCATGG AGAATAGGGA
AGATGTAGAC ACGAATAAGC TCGGATACGA ATACAAATGG TCCGAAATCC CATGGGGAAG
GAGTCAACCG ACTGAATATG GCAAGGATTC AAAATTTGTA GATAAGTCTA TAGGAATAGA
GAAGAAGGTG GGGGAGGTGG AATTTCCAGT GAAGTTAAAC AAGACAGTTA AGGTACTCGT
GAAGAGGCCG GCTGTTAATA GGACCAAGGA GGACAAACAG AAAGCGAATG AGATTTTGTT
GGTAAATGGA GTGAGATTCG ATGGTGAGAA GTACGTCAAG TTCGATGTCT TTGTCAACGA
CATAGACAAT GGAACCGAGA CCACCCCGGC TGACAGTGAG TTTGCTGGTA GTTTCGCACA
GCTTCCGCAT GGCAAAACCG ACAGGATGAT GATGATGAGC GGAGTTAGGT TTGGGTTAAC
GGAGCTTTTG GAGGACATAA AAGCTGAAGA TGATGAATAT GTTTTGGTGA AATTGGTGCC
TAGGACTGGG TGTGATGACG TTACAGTTTC CGAGATCAAG ATTGAACTGG AGGGAATAAT
GTCAATGGAC AAGAAGTCAA AGTTTGTTGA ATCTGAATAT GAGAATGTGA TTGATTACTT
GTACATATGT TTATAG
PPO-Q protein sequence (SEQ ID NO: 24):
MMASFNLYTV PAATTATTNI IKTNYYQLKT QAKQTHRLKA SCNAIPDKNN DKALETSLQM
INIDRRNIIL RLGGLFVASN MTSVPLAYAN AIAARSNHSV CAASPLGIQN LGTPVKEPME
NREDVDTNKL GYEYKWSEIP WGRSQPTEYG KDSKFVDKSI GIEKKVGEVE FPVKLNKTVK
VLVKRPAVNR TKEDKQKANE ILLVNGVRFD GEKYVKFDVF VNDIDNGTET TPADSEFAGS
FAQLPHGKTD RMMMMSGVRF GLTELLEDIK AEDDEYVLVK LVPRTGCDDV TVSEIKIELE
GIMSMDKKSK FVESEYENVI DYLYICL
PPO-R gene sequence (SEQ ID NO: 25):
GTTCGAAGAA GTGAGTGGAG TAACCCAAAA CAAAATAACA ATGGCTTCTT TCCAACTTGT
TAACCCCTTC GCCAGCACCA CAAGAAAACT GCCAGATTCC ACCTCCAGCC GTCGTCTCAA
GACTCACCCT CAAAAAAACC ACCGCTTCAA AGTCTCATGC AACGTCGCAC AAGATGGCAA
TGAGAAGCTA CTCTTAGTCC CAGATAGCAA AAACCTTATA CTACCTAAAC CATCACTCGA
CACGCTTAAT GTAGATCGGA GGAACTTGCT ACTGGGACTC GGTGGCCTTT ACAGCACCGT
CAACTTCACC TCTCTTCCGG CGGCCATTGC CGCCCCTATC ACCACGCCTG ACATCTCCAC
ATGCATCCCG TCAGAGCAAG GCTTCAACGT GCAGGACTCC GTAAGAAGCA ACCAATGTTG
TCCGCCGATG ATGACCACAA CCCCGAAAGA CTTTGTGTTC CCAAAAGACA AAACAATTCG
GGTTAGACCA GCGGCACATA GAGCCACCCC CGAGTACATA GCAAAGTACA AAGCAGCAAT
CCAAGCGATG AAAGATCTCC CAGATGACCA CCCACATAGT TTCGTTCAAC AAGCTAAAAT
TCATTGCGCT TACTGCAACG GTGGTTATAC TCAAGTCGCA AGTGGTTATG CTGATAAGCA
ACTCCAGATT CACAACTCAT GGCTCTTCTT TCCTTTCCAT CGCTGGTACT TGTATTTCTA
CGAGAGAATC CTCGGGAAGT TAATTGATGA TCCCACTTTC GCTTTACCTT ACTGGAACTG
GGACAATCCC GCCGGAATGT CATTTCCAGC TTTTTTTGAA ACCGACGGCA GAGAAAACCC
TGTCTTTGAC GCGTTCCGCA ACGTCAACCA TGTATCACCA GAAACAGTTG TCGATCTCGA
CTACAATGGC TCAGATAGTG GCGCTCCTTG TCTTCAACAG ATAAGCACCA ATCTTGCTGC
AATGTATAAG CAGATGATCA GCAACGCTAC TGACCCGTTA AGTTTCTTTG GTGGCGAGTT
TCGGGCTGGA GATGACCCTT TTGGAAATAG TGACCCATCT GTCGGATCAA TAGAGGCTGG
TTGTCACACT GCGATGCACA GATGGACGGG AAATCCGAGA ATGCCAAACA ACGAGGACAT
GGGGAATTTC TACTCTGCGG GGTACGACCC TGCGTTCTAC GTCCACCATG CGAATGTTGA
CCGTATGTGG AAAGTATGGA AGGATTTAGG TATCAAAGGA CACACTGAAC CTACGGACCC
TGATTGGCTT AATGCATCAT ATGTGTTTTA TGATGAAAAC GAAGAGCTTG TACGTGTTTA
CAACAAAGAT TGTGTCCAAA CTGAAAACTT AAAATATGAT TTCGAATTAT CCCCACTCCC
TTGGCTCAAG AACCGACCGG TTGCACATAC CAAACCAGAG ACCACCACGA AACCTGTTGA
AAAGGTTAAA GTGCCGGACG TGAAGTTCCC CATTAAGCTA GACAAGATAC AGAAGGTCCT
TGTGAAGCGG CCAGCGAAAA ACCGAAGCCA ATCAGAAAAA GAAAAAGCGA CTGAGCAGTT
GTTGATCAAA GGAATCAAGT TTAATGTCTC CAAGTTCGTC AAGTTTGATG TGTTTGTTAA
TGACCAAGAT GATGTTCCCA CAAGCTCTGC ATCCGAGAGC GAGTTTGCAG GTAGTTTCGC
ACAGTTGCCT CATCACCATG GTGGCCACAA AAAGTTAATG ACAAGTGCAG CAAGGTTCGG
TTTAACAGAG CTTTTGGAGG ACATCGGAGC CGAGGATGAT GAGTATATTT TGGTGACATT
GGTGCCAAAG GTAGGGGCCG AAGATCTCAC CGTTGATGAA ATCAAAGTGG AGTTGGTTCC
TATTGTTTAA ACAACCATGT AATCTTATCT ACACAATAAG GTATATATTT AAATAATTCG
TAGGCCTTGC ATTCCATTGC ATGGTTGCGA CTTTTATGTA CGAATATTAA TAACTTCATT
GTGTCCTTTT TCTACGAGTA AATGGAGTGA ACTCAACATC TTTTGCTTGT TC
PPO-R protein sequence (SEQ ID NO: 26):
MASFQLVNPF ASTTRKLPDS TSSRRLKTHP QKNHRFKVSC NVAQDGNEKL LLVPDSKNLI
LPKPSLDTLN VDRRNLLLGL GGLYSTVNFT SLPAAIAAPI TTPDISTCIP SEQGFNVQDS
VRSNQCCPPM MTTTPKDFVF PKDKTIRVRP AAHRATPEYI AKYKAAIQAM KDLPDDHPHS
FVQQAKIHCA YCNGGYTQVA SGYADKQLQI HNSWLFFPFH RWYLYFYERI LGKLIDDPTF
ALPYWNWDNP AGMSFPAFFE TDGKRNPVFD AFRNVNHVSP ETVVDLDYNG SDSGAPCLQQ
ISTNLAAMYK QMISNATDPL SFFGGEFRAG DDPFGNSDPS VGSIEAGCHT AMHRWTGNPR
MPNNEDMGNF YSAGYDPAFY VHHANVDRMW KVWKDLGIKG HTEPTDPDWL NASYVFYDEN
EELVRVYNKD CVQTENLKYD FELSPLPWLK NRPVAHTKPE TTTKPVEKVK VPDVKFPIKL
DKIQKVLVKR PAKNRSQSEK EKATEQLLIK GIKFNVSKFV KFDVFVNDQF DVPTSSASES
EFAGSFAQLP HHHGGHKKLM TSAARFGLTE LLEDIGAEDD EYILVTLVPK VGAEDLTVDE
IKVELVPIV
PPO-S gene sequence (SEQ ID NO: 27):
AAAGGAGTAT GAAGCACACC AAACCAACAT GGCTTCTTTG AGCTTTACTT TAGCCACTCC
CACCACCTCT TCCTCGCCGT TCTTTTCACA AACAACCACC AAACAACGAC GGTTGATGAA
GACACATGGG AAGCAAACCC ATCGCTTCCA AGTCTCATGC AACGTCTCAT CAAATAACCA
TGAAAAACCA CTCCCCAAAA ACCCTCAACC ACAAAAACTT ATACTACCAC AAACATCACT
CGACTTGCAG AACGTCGACA GAAGGAATTT GCTCCTGGGT CTCGGCGGAG TCTACAGCAC
CGCCACCTTG TCCGGTCTGC CACCGGCCTT TGCAGAAGCT ATCAAGGCTC CGTTCAATCA
GCCTGATCGA CCATGCAAAG ATGCCGTATC CGGCTTCGAC ATTAATAAAA AGCTACTTAG
ACCTATTGAC TGTTGCCCTC TGTCCAAAAA TGGCCCGGAG AGTCATTTCA AGTTCCCTGA
TAAATCAAGC AAAACTCGCA TCAGATATCC ACTACACAAA CTTCCAGTCG GGTATCTCGA
TAAATATATG GATGCGATTC AGAAAATGAA GGATCTCCCA GATAGCGACC CACGCAGTTT
CAATAACCAA GCTAAAGTTC ATTGCGCTTA CTGCAATGGC AGTTACACTC AAAACGGTCA
AGAACTCCAG ATTCACAACT CCTGGCTCTT CTTTCCCTTC CATCGGTGGT ACCTTTATTT
CTACGAGAGG ATACTGGGAG ATCTCATTGG TGATTCGACA TTCGGGTTAC CCTACTGGAA
CTGGGACAAC CCCGAAGGAA TGACAATTCC ACACTTCTTC GTAGAGAAAC AATGTAACAA
CTATAAGTTC GAAAACGGAG AAAACCCTCT ATATGATAAG TATCGGGACG AAAGTCACCT
TCGGTATGAA TTGGTCGATC TTGACTACTC AGGGAGAAAC CGCGACCTGT GTTACGATCA
GAAAGAAATC AATCTGGCTA CTATGAATAG GCAGATGATG CGCAACGCCT TTGATGCAAC
AAGCTTCTTC GGTGGCAAAT ATGTAGCCGG TGATGAACCG ATTCCCCGAG GAGATAATGT
AGTTGGATCC GTGGAGGCTG GTTGTCATAC GGCTGTTCAC AGATGGGTTG GGAACCCTGA
TCCAAAAGGG AATAAAGAGG ACATGGGCAA CTTCTACTCT GCGGGATATG ATCCTTTGTT
CTACGTCCAC CATTCTAATG TGGACCGAAT GTGGACTCTT TGGAAGCAAA TGGGAGGCAA
AGAACCGACA GATACTGACT GGGAAAACGC GTCATACGTG TTTTACGATG AGAAACAAAA
TCCCGTACGT GTCTACAACA AACAATCGGT GGATTTGAGC AACCTTAAAT ACGAATACCA
CAGCTCAGCC ACTCCATGGA CGGATAGACC ACCAAGATCA CGCTGCAACA GACCCGGTTA
CCCGAAGAGG AACAACACAA AGGACTTCCC AAATCAGAAG GACCCGCCAG AAGCTTTGAC
ATTAACCGAT AGTACTGTGA GGCTTCGAGT AAAGAGGCCT CCTGCTTCTA AAAACAGGAA
CGCTGAGCAA AAGAAAAGTG AAAAAGAGAT CTTGTGCTTG ATTGGAATCA GTTTCGATTG
TACCGAAGCT GCAAAATTTG ACGTGTTTGT GAATGATTGT GACGAAGAAC AGATCACCCC
GTGTGATAGT GAGAATGTGG GTTCTTTCGC GGCTGTTCCA CATGCTAAAG GCATGGCAAT
GGGTTGCAAG TCTGGGATGA GGTTTTCGTT AACAGAGTTG TTGGAGGAAA CAAAAGCTGA
GGGGGATGAG TCTATTCGGG TGACAATTGT GCCGAGGACG ACACCCGGCA AGAAAGTCAA
AGTCACCATT GATGCTATCG AGATCCGGTT GATTCCGGTT CTTGAAAAAT AATCAAACTG
ATCAAATGCG TTGATGATGG ATTCCACAAT CACGAAATGA ATAAGTTGGA GTCTGTGTAG
TTCGATAAAC GTACGTTGTG ATTTCTAATG CATGTTTTAT TTGAATAGAA ATTTGTTTTA
AACAAAAGTC TCTTCATTTC
PPO-S protein sequence (SEQ ID NO: 28):
MASLSFTLAT PTTSSSPFFS QTTTKQRRLM KTHGKQTHRF QVSCNVSSNN HEKPLPKNPQ
PQKLILPQTS LDLQNVDRRN LLLGLGGVYS TATLSGLPPA FAEAIKAPFN QPDRPCKDAV
SGFDINKKLL RPIDCCPLSK NGPESHFKFP DKSSKTRIRY PLHKLPVGYL DKYMDAIQKM
KDLPDSDPRS FNNQAKVHCA YCNGSYTQNG QELQIHNSWL FFPFHRWYLY FYERILDGLI
GDSTFGLPYW NWDNPEGMTI PHFFVEKQCN NYKFENGENP LYDKYRDESH LRYELVDLDY
SGRNRDLCYD QKEINLATMN RQMMRNAFDA TSFFGGKYVA DGEPIPRGDN VVGSVEAGCH
TAVHRWVGNP DPKGNKEDMG NFYSAGYDPL FYVHHSNVDR MWTLWKQMGG KEPTDTDWEN
ASYVFYDEKQ NPVRVYNKQS VDLSNLKYEY HSSATPWTDR PPRSRCNRPG YPKRNNTKDF
PNQKDPPEAL TLTDSTVRLR VKRPPASKNR NAEQKKSEKE ILCLIGISFD CTEAAKFDVF
VNDCDEEQIT PCDSENVGSF AAVPHAKGMA MGCKSGMRFS LTELLEETKA EGDESIRVTI
VPRTTPGKKV KVTIDAIEIR LIPVLEK

PPO genes may have different sequences among different varieties of lettuce. In some embodiments, a PPO gene has at least 99%, 98%, 97%, 96%, 95%, 90%, 85%, 80%, 75%, or 70% identify to SEQ ID NOs:1, 3, 5, 7, 9, 11, 13, 15, 17, 19, 21, 23, 25, or 27. In some embodiments, the PPO protein sequences have at least 99%, 98%, 97%, 96%, 95%, 90%, 85%, 80%, 75%, or 70% identity to SEQ ID NOs:2, 4, 6, 8, 10, 12, 14, 16, 18, 20, 22, 24, 26, or 28.

In one embodiment of the lettuce plant of the present application, the mutation in a PPO gene occurs in both alleles of the PPO gene. In other words, the mutation is a homozygous mutation.

In another embodiment of the lettuce plant of the present application, the mutation in a PPO gene occurs in only one allele of the PPO gene. In other words, the mutation is a heterozygous mutation.

Lettuce plants may contain only homozygous PPO gene mutations, only heterozygous PPO gene mutations, or a combination of homozygous and heterozygous PPO gene mutations.

In another embodiment of the lettuce plant of the present application, different mutations in a PPO gene occur in each allele of the PPO gene such that both alleles comprise mutations in the PPO gene.

Mutations of PPO genes may be made throughout the gene sequence. Mutations may also be made by targeting nucleotides in the tyrosinase domain of the PPO genes. The mutation may be an insertion, a deletion, a missense mutation, a splice junction mutation, or any combination of mutation thereof. In some embodiments, mutations substantially reduce or inactivate the function of the PPO protein. For example, a mutation may mutate an amino acid required for enzymatic function of the PPO enzyme. The mutation may create a frameshift resulting in a premature stop codon, such that a functional PPO protein is not made. Non-limiting examples of such mutations with respect to the sequences provided herein to exemplify PPO genes include those shown in Table 1.

TABLE 1
Exemplary Mutations in PPO Genes of Lettuce

| PPO | | | | Reference |
|---|---|---|---|---|
| gene | Allele | Mutation type | Location | Sequence |
| PPO-A | PPO-A1 | Deletion of 26 | between bp 971-996 | SEQ ID NO: 1 |
| | | or 27 bp | or 997 | |
| PPO-A | PPO-A2 | Insertion of T | between bp 976-977 | SEQ ID NO: 1 |
| | | or A | | |
| PPO-B | PPO-B1 | Insertion of T | between bp 1007- | SEQ ID NO: 3 |
| | | or A | 1008 | |
| PPO-B | PPO-B2 | Insertion of TA | between bp 1007- | SEQ ID NO: 3 |
| | | | 1008 | |
| PPO-B | PPO-B3 | Deletion of T | bp 1008 | SEQ ID NO: 3 |
| PPO-D | PPO-D1 | Insertion of T | between bp 1131- | SEQ ID NO: 7 |
| | | | 1132 | |
| PPO-E | PPO-E1 | Insertion of T | between bp 1112- | SEQ ID NO: 9 |
| | | or A | 1113 | |
| PPO-G | PPO-G1 | Deletion of G | bp 987 | SEQ ID NO: 11 |
| PPO-G | PPO-G2 | Insertion of T | between bp 988-989 | SEQ ID NO: 11 |
| PPO-G | PPO-G3 | Deletion of CG | bp 986-987 | SEQ ID NO: 11 |
| PPO-R | PPO-R1 | Insertion of CC | between bp 1024- | SEQ ID NO: 25 |
| | | | 1025 | |
| PPO-R | PPO-R2 | Insertion of A | between bp 1024- | SEQ ID NO: 25 |
| | | | 1025 | |
| PPO-S | PPO-S1 | Insertion of T | between bp 1044- | SEQ ID NO: 27 |
| | | | 1045 | |
| PPO-S | PPO-S2 | Deletion of 3 | bp 1045-1047 | SEQ ID NO: 27 |
| | | bp | | |
| PPO-S | PPO-S3 | Deletion of 7 | bp 1045-1051 | SEQ ID NO: 27 |
| | | bp | | |

In the lettuce plants of the present application, mutations in the PPO genes reduce the amount and/or activity of PPO protein(s) to confer improved plant traits in the lettuce plant. In one embodiment, mutations in the PPO genes reduce the amount of PPO protein(s) in the mutated lettuce plant compared to a wild type lettuce plant (i.e., a lettuce plant without any mutations in its PPO genes). In another embodiment, mutations in the PPO genes reduce the activity of PPO protein(s) in the mutated lettuce plant compared to a wild type lettuce plant. In yet another embodiment, mutations in the PPO genes reduce the amount and activity of PPO protein in the mutated lettuce plant compared to a wild type lettuce plant.

In the lettuce plants of the present application, mutation in the PPO genes reduce the expression of PPO gene(s) to confer improved plant traits in the lettuce plant. In one embodiment, mutations in the PPO genes reduce the amount of PPO gene expression in the mutated lettuce plant compared to a wild type lettuce plant.

Alteration of PPO genes, PPO gene expression, PPO protein amounts, or PPO enzymatic activity may be determined using standard procedures in the art, for example, as described by Sambrook et al., Molecular Cloning: A Laboratory Manual, Second Edition, Cold Spring Harbor, NY, Cold Spring Harbor Press (1989), and Ausubel et al., Current Protocols in Molecular Biology, New York, NY, John Wiley & Sons (1989), which are hereby incorporated by reference in their entirety.

Mutation of PPO genes may be determined using any method that identifies nucleotide differences between wild type and mutant sequences. These methods may include, for example and without limitation, PCR, sequencing, denaturing high pressure liquid chromatography (dHPLC), constant denaturant capillary electrophoresis (CDCE), temperature gradient capillary electrophoresis (TGCE), or by fragmentation using enzymatic cleavage, such as used in the high throughput method described by Colbert et al., “High-Throughput Screening for Induced Mutations,” Plant Physiology 126:480-484 (2001), which is hereby incorporated by reference in its entirety. Multiple plants and multiple PPO gene mutations may be assessed simultaneously by Next Generation Sequencing (“NGS”) approaches.

The “expression” of a PPO gene refers to the transcription of a PPO gene. PPO gene expression levels may be measured by any means known in the art such as, without limitation, qRTPCR (quantitative real time PCR), semi-quantitative PCR, RNA-seq, and Northern blot analysis.

In some embodiments, the expression of a PPO gene is 90% or less, 80% or less, 70% or less, 60% or less, 50% or less, 40% or less, 30% or less, 20% or less, 10% or less, or 5% or less of the expression of a PPO gene in wild type lettuce plant. In some embodiments, the expression of a PPO gene is undetectable.

The “amount” of a protein refers to the level of a particular protein, for example PPO-D, which may be measured by any means known in the art such as, without limitation, Western blot analysis, ELISA, other forms of immunological detection, or mass spectrometry.

In some embodiments, the amount of a PPO protein is 90% or less, 80% or less, 70% or less, 60% or less, 50% or less, 40% or less, 30% or less, 20% or less, 10% or less, or 5% or less of the amount of a PPO protein in wild type lettuce plant. In some embodiments, the amount of a PPO protein is undetectable.

PPO protein “activity” or “PPO activity” refers to the enzymatic activity of the PPO protein(s). PPO protein activity may be measured biochemically by methods known in the art including, but not limited to, the detection of products formed by the enzyme in the presence of any number of heterologous substrates, for example, catechol. PPO protein activity may also be measured functionally, for example, by assessing its effects on phenotypic traits of a lettuce plant, such as leaf browning or other traits described herein.

In one embodiment, the lettuce plant of the present application has a reduced activity of PPO that is 99% or less of the activity of PPO in wild type lettuce. In some embodiments, the lettuce plant has a reduced activity of PPO that is 90% or less of the activity of PPO in wild type lettuce. In some embodiments, the lettuce plant has a reduced activity of PPO that is 80% or less of the activity of PPO in wild type lettuce. In some embodiments, the lettuce plant has a reduced activity of PPO that is 70% or less of the activity of PPO in wild type lettuce. In some embodiments, the lettuce plant has a reduced activity of PPO that is 60% or less of the activity of PPO in wild type lettuce. In some embodiments, the lettuce plant has a reduced activity of PPO that is 50% or less of the activity of PPO in wild type lettuce. In some embodiments, the lettuce plant has a reduced activity of PPO that is 40% or less of the activity of PPO in wild type lettuce. In some embodiments, the lettuce plant has a reduced activity of PPO that is 30% or less of the activity of PPO in wild type lettuce. In some embodiments, the lettuce plant has a reduced activity of PPO that is 20% or less of the activity of PPO in wild type lettuce. In some embodiments, the lettuce plant has a reduced activity of PPO that is 10% or less of the activity of PPO in wild type lettuce. In some embodiments, the lettuce plant has a reduced activity of PPO that is 5% or less, 4% or less, 3% or less, 2% or less or 1% or less or 0% of the activity of PPO in wild type lettuce. In some embodiments, the lettuce plant has undetectable PPO activity. The reduction in PPO activity may vary depending on a number of factors including, but not limited to, the source of the PPO, the developmental stage of a plant or plant material, the method of cultivation, the harvesting conditions, the experimental conditions, and combinations and variations thereof.

As discussed supra, lettuce plants of the present application comprise a mutation in each of at least two different PPO genes. For example, a lettuce plant may comprise a mutation in any two of the following PPO genes: PPO-A, PPO-B, PPO-C, PPO-D, PPO-E, PPO-G, PPO-J, PPO-M, PPO-N, PPO-O, PPO-P, PPO-Q, PPO-R, or PPO-S.

In one embodiment, the lettuce plant comprises a mutation in at least its PPO-B gene and one or both of its PPO-S or PPO-G genes. In some embodiments, the lettuce plant comprising a mutation in at least its PPO-B gene and one or both of its PPO-S or PPO-G genes further comprises a mutation in one or both of its PPO-D and PPO-E genes. In further embodiments, the lettuce plant comprising a mutation in at least its PPO-B gene and one or both of its PPO-S or PPO-G genes further comprises a mutation in one or more of PPO-A, PPO-C, PPO-O, and PPO-R. In some embodiments, the lettuce plant comprising a mutation in at least its PPO-B gene and one or both of its PPO-S or PPO-G genes further comprises a mutation in PPO-S. In some embodiments, the lettuce plant comprising a mutation in at least its PPO-B gene and one or both of its PPO-S or PPO-G genes further comprises a mutation in PPO-G.

The present application also encompasses lettuce plants with any of the preceding embodiments and lettuce plants that further comprise a mutation in one or more of PPO-A, PPO-C, PPO-O, PPO-P, or PPO-R.

In some embodiments of the lettuce plant of the present application, the mutation is a frameshift mutation. In some embodiments of the lettuce plant of the present application, the frameshift mutation is a knockout mutation causing a premature stop codon.

In some embodiments, the lettuce plant of the present application comprises a PPO-D knockout, which is caused by a nucleotide insertion in the PPO-D gene sequence between nucleotides 1131 and 1132 of SEQ ID NO:7, or an equivalent thereof. As used herein, the term “or an equivalent thereof” means an equivalent PPO gene in a different lettuce plant, which may or may not be identical in sequence, but is sufficiently similar to identify the sequence as the same PPO gene.

In some embodiments, the lettuce plant of the present application comprises a PPO-E knockout, which is caused by a nucleotide insertion between nucleotides 1112 and 1113 of SEQ ID NO:9, or an equivalent thereof.

In some embodiments, the lettuce plant of the present application comprises a PPO-G knockout, which is caused by a nucleotide insertion between nucleotides 988 and 989 of SEQ ID NO:11, or an equivalent thereof, or a deletion (e.g., of two nucleotides) between nucleotides 986-987 of SEQ ID NO:11, or an equivalent thereof, or a deletion (e.g., of one nucleotide) at nucleotide 987 of SEQ ID NO:11, or an equivalent thereof.

In some embodiments, the lettuce plant of the present application comprises a PPO-S knockout, which is caused by a nucleotide insertion between nucleotides 1044-1045 of SEQ ID NO:27, or an equivalent thereof, or by a deletion (e.g., of three nucleotides) between nucleotides 1045-1047 of SEQ ID NO:27, or an equivalent thereof, or by a deletion (e.g., of 7 nucleotides) between nucleotides 1045-1051 of SEQ ID NO:27, or an equivalent thereof.

In some embodiments, the lettuce plant of the present application comprises a PPO-A knockout, which is caused by an insertion between nucleotides 976 and 977 of SEQ ID NO:1, or an equivalent thereof, or by a deletion of 26 or 27 nucleotides between nucleotides 971-996 or 971-997 of SEQ ID NO:1, or an equivalent thereof.

In some embodiments, the lettuce plant of the present application comprises a PPO-B knockout, which is caused by an insertion of one nucleotide between nucleotides 1007 and 1008 or an insertion of two nucleotides between nucleotides 1007 and 1008 of SEQ ID NO:3, or an equivalent thereof, or by a deletion (e.g., of one nucleotide) of nucleotide 1008 of SEQ ID NO:3, or an equivalent thereof.

In some embodiments, the lettuce plant of the present application comprises a PPO-R knockout, which is caused by an insertion of one or two nucleotides between nucleotides 1024 and 1025 of SEQ ID NO:25, or an equivalent thereof.

### Lettuce Plants with Combinations of Mutant PPO Genes

PPO mutant plants of the present application may have combinations of knockouts among their PPO genes. Such combinations may create plants with the best shelf-life (or other trait) performance.

For example, best performing plants in terms of some or all phenotypes such as reduced browning, reduced tip-burn, reduced yellowing of the midvein, increased levels of polyphenolics, increased shelf life, increased vitamins, increased vitamin retention, reduced fermentation, and increased carbohydrate retention may be those plants with knockouts of at least their PPO-B and PPO-S genes. Therefore, the present application is directed to plants comprising knockout mutations of at least the PPO-B and PPO-S genes. Examples of plants with improved non-browning and reduced tip burn traits, with mutations in PPO-B and PPO-S genes are 15.1.17.8, 15.1.7.22, 15.1.9.1, 14.2.24.21, 14.2.24.5, and 14.2.97.6, described below.

In some embodiments, the plants further comprise a knockout mutation of the PPO-D and PPO-E genes (i.e., PPO-B, PPO-D, PPO-E, and PPO-S knockouts). Examples of plants with improved traits with mutations in PPO-B, PPO-D, PPO-E, and PPO-S are 14.2.24.21, 14.2.24.5, 14.2.73.14, 14.2.96.13, 14.2.96.9, 14.2.97.17, and 14.2.97.6, described below. In further embodiments, the plants also comprise a knockout mutation of a PPO-A gene (i.e., PPO-A, PPO-B, PPO-D, PPO-E, and PPO-S knockouts). Examples of such plants are 14-2-73-14, 14-2-96-17, 14.2.96.13, and 14.2.96.9, described below. In further embodiments, the plants also include a knockout mutation of a PPO-G gene (i.e., PPO-A, PPO-B, PPO-D, PPO-E, PPO-G, and PPO-S knockouts). Examples of such plants are 14.2.96.13 and 14.2.96.9, described below. In some embodiments, the plants comprise a knockout mutation of the PPO-G gene, but not the PPO-A gene (i.e., PPO-B, PPO-D, PPO-E, PPO-G, and PPO-S knockouts). Example plants include 14.2.24.21 and 14.2.24.5, described below.

In some embodiments, the plants comprise PPO-B and PPO-S gene knockouts and further comprise a knockout mutation of the PPO-R gene (i.e., PPO-B, PPO-R, and PPO-S knockout). Examples include 15-1-7-22 and 15-1-9-1, described below. In some embodiments, the PPO-B, PPO-R, and PPO-S gene knockouts further comprise a homozygous knockout of the PPO-G gene (i.e., PPO-B, PPO-G, PPO-R, and PPO-S knockouts). An example plant is 15-1-9-1, described below.

In some embodiments, a lettuce plant of the present application comprises knockouts of the PPO-B, PPO-G, and PPO-R genes (i.e., PPO-B, PPO-G, and PPO-R knockouts). An example plant is 15.1.9.11, described below.

In some embodiments, a lettuce plant of the present application comprises both homozygous and heterozygous knockouts of PPO genes. In some embodiments, the plant comprises a modification of at least a heterozygous mutation of its PPO-E gene and at least a heterozygous mutation of its PPO-D or PPO-G genes; where when the mutation of both PPO-E and PPO-D or PPO-G are heterozygous mutations, there is a homozygous mutation of both PPO-O and PPO-R. In some embodiments, the PPO-E mutation is a homozygous mutation. In some embodiments, the PPO-D mutation is a homozygous mutation. In some embodiments, both the PPO-D and PPO-E mutations are homozygous mutation and the plant further comprises a heterozygous or homozygous mutation of the PPO-S gene. In some embodiments, each of the mutations of PPO-D, PPO-E, and PPO-S gene are homozygous mutations. In some of these, the plants further comprise at least a heterozygous mutation of the PPO-G gene.

The mutations may also be homozygous mutations of PPO-D and PPO-E genes where the plant further comprises at least a heterozygous or homozygous mutation of its PPO-G gene. The mutations may also be homozygous mutations of PPO-E and PPO-S genes and the plant further comprises at least a heterozygous mutation of the PPO-G gene.

In the foregoing described plants, the plants may further comprise at least a heterozygous mutation of at least one PPO gene of PPO-A, PPO-B, PPO-O, PPO-R, or PPO-P. As such, in some embodiments the plant comprises:
(a) homozygous mutations of PPO-A, PPO-D, PPO-E, and PPO-S; and a heterozygous mutation of PPO-B;
(b) homozygous mutations of PPO-B, PPO-D, PPO-E, PPO-G, and PPO-S;
(c) homozygous mutations of PPO-B, PPO-D, PPO-E, PPO-G, and PPO-S; and a heterozygous mutation of PPO-A;
(d) homozygous mutations of PPO-A, PPO-B, and PPO-S; and heterozygous mutations of PPO-G and PPO-E;
(e) homozygous mutations of PPO-O and PPO-R; and heterozygous mutations of PPO-B, PPO-G, PPO-E, PPO-S, PPO-P, and PPO-D;
(f) homozygous mutations of PPO-D, PPO-E, PPO-O, and PPO-S; and heterozygous mutations of PPO-B and PPO-R;
(g) homozygous mutations of PPO-G, PPO-O, and PPO-R; and heterozygous mutations of PPO-E, PPO-S, PPO-P, and PPO-D; or
(h) homozygous mutations of PPO-B, PPO-D, PPO-E, PPO-O, PPO-R, and PPO-S; and a heterozygous mutation of PPO-G.

In some embodiments, the mutation is a knockout of the gene.

### Phenotypic, Organoleptic, and Nutritional Aspects of PPO Mutant Lines

The PPO mutations described herein confer multiple beneficial phenotypes on the lettuce plant at harvest and during multiple days after harvest. These phenotypes include, without limitation, reduced browning, reduced tip-burn, reduced yellowing of the midvein, increased levels of polyphenolics, increased shelf life, increased vitamins, increased vitamin retention, reduced fermentation, and increased carbohydrate retention.

For example, the present application is directed to a lettuce plant that exhibits reduced-browning. As used herein, “reduced-browning” (or similar terminology) means that when lettuce leaves are harvested, cut, sliced, or processed in a manner where cell wall destruction takes place, browning will be detectably less than in a control (wild type) lettuce variety. Any reduction in browning (such as a reduction in browning visible to the naked eye relative to a control) may be advantageous.

In one embodiment, the rate of browning of lettuce leaves produced from a PPO mutant plant is reduced relative to leaves a control plant. In another embodiment, the total quantity or degree of browning of lettuce leaves produced from a PPO mutant plant is reduced relative to leaves from a control plant.

Any detectable level of reduced browning that is detectable to the naked eye may constitute a reduction in browning. Beyond this, reduced browning may be detected by a device, such as a chromameter, even if not visible to the human eye. Browning may be determined by known methods including, but not limited to, spectroscopy (e.g., light absorption, laser-induced fluorescence spectroscopy, time-delayed integration spectroscopy, large aperture spectrometer); colorimetry (e.g., tristimulus, “spekol” spectrocolorimeter); and visual inspection/scoring.

The PPO mutant plant may be considered reduced-browning if the PPO mutant sample visual score is at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 60%, at least 70%, at least 80% or at least 90% less than the control.

The skilled person would appreciate that browning may vary depending on a number of factors including, but not limited to, the manner and ambient conditions in which plant material is bruised. For example, lettuce leaves stored at 4° C. may show different browning characteristics from lettuce leaves stored at 24° C., as detected by the eye or by an instrument, such as a chromameter, or like devices.

The present application is also directed to a lettuce plant that exhibits reduced tip burn. As used herein, “tip burn” means a browning of the edges or tips of lettuce leaves that is a physiological response to a lack of calcium in growing tissues.

In some embodiments, the lettuce plant of the present application exhibits less tip burn as compared to a wild type variety under the same conditions. In some embodiments, the lettuce of the present application exhibits no tip burn at harvest. In some embodiments, the lettuce plant exhibits at least 10% less, at least 20% less, at least 30% less, at least 40% less, at least 50% less, at least 60% less, at least 70% less, at least 80% less, or at least 90% less tip burn at harvest compared to a control plant.

In some embodiments, the lettuce plant of the present application exhibits less yellowing of the midvein compared to a wild type variety under the same conditions as assessed by midvein scoring. In other embodiments, the lettuce plant exhibits less than 5% yellowing through day 7 post-harvest as assessed by midvein scoring. In other embodiments, the lettuce plant exhibits less than 5% yellowing through day 14 post-harvest as assessed by midvein scoring. In still other embodiments, the lettuce plant exhibits less than 20% yellowing through day 22 post-harvest as assessed by midvein scoring.

In some embodiments, the lettuce plant of the present application exhibits longer shelf life compared to a wild type variety under the same conditions. Shelf life can be assessed by a number of factors by organoleptic scoring, for example and without limitation, on a qualitative basis across several categories, including: off odor, typical aroma, moisture, texture, leaf color, decay/mold, cut edge discoloration, and taste. A total score combining values from each category provides an overall assessment of a plant. In some embodiments, shelf life is scored after processing the leaf plant by cutting the leaves into pieces. In some embodiments, shelf life is scored after processing the leaf plant by cutting the leaves into pieces and packaging the cut leaves. In some embodiments, the shelf life is scored after storage under optimal conditions of light and temperature. In some embodiments, the shelf life is scored after storage under suboptimal conditions of light and temperature.

In some embodiments, the shelf life of the lettuce plant of the present application exhibits more than 1 day, more than 2 days, more than 3 days, more than 4 days, more than 5 days, more than 6 days, more than 7 days, more than 8 days, more than 9 days, more than 10 days, more than 11 days, more than 12 days, more than 13 days, more than 14 days, more than 15 days, more than 16 days, more than 17 days, more than 18 days, more than 19 days, or more than 21 days of commercially-suitable shelf life compared to a wild type variety under the same conditions.

In some embodiments, the shelf life of the lettuce plant of the present application exhibits reduced failure rate at days after harvest compared to a wild type variety under the same conditions as assessed by organoleptic scoring. As used herein, the “failure rate” means the percentage of replicates at a particular time point of a shelf life study that, assessed by organoleptic scoring, is unsuitable for marketability. In some embodiments, the shelf life of the lettuce plant of the present application exhibits a reduced failure rate of 13 days after harvest as assessed by organoleptic scoring. In some embodiments, the lettuce plant exhibits 0% failure rate 13 days post-harvest as assessed by organoleptic scoring. In some embodiments, the lettuce plant exhibits less than 5% failure rate 13 days post-harvest as assessed by organoleptic scoring. In some embodiments, the lettuce plant exhibits less than 10% failure rate 13 days post-harvest as assessed by organoleptic scoring. In some embodiments, the shelf life of the lettuce plant of the present application exhibits reduced failure rate of 21 days after harvest as assessed by organoleptic scoring. In some embodiments, the lettuce plant exhibits 0% failure rate 21 days post-harvest as assessed by organoleptic scoring. In some embodiments, the lettuce plant exhibits less than 5%, less than 10%, less than 20%, less than 30%, less than 40%, or less than 50% failure rate 21 days post-harvest as assessed by organoleptic scoring.

A recognized problem that is associated with harvested vegetables or harvested vegetable parts is that the levels of plant phytochemicals, such as plant secondary metabolites, start to decrease almost immediately post-harvest. For example, as harvested vegetables are processed for freezing and/or canning or are simply placed in refrigerators, they lose much of their nutritional content in terms of the levels of phytochemicals found therein. Such phytochemicals include vitamins, e.g., vitamins A, C, E, K, and/or folate, carotenoids such as beta-carotene, lycopene, the xanthophyll carotenoids such as lutein and zeaxanthin, phenolics comprising the flavonoids such as the flavonols (e.g., quercetin, rutin, caffeic acids), sugars, and other food products such as anthocyanins, among many others.

The lettuce plants of the present application also exhibit higher levels of polyphenolics in comparison to a wild type variety. In one embodiment, the level of polyphenolics is 5%, 10%, 15%, 20% or more than 20% higher than a wild type variety. The lettuce plants of the present application also retain higher levels of polyphenolics after harvest in comparison to a wild type variety. In one embodiment, the level of polyphenolics is 5%, 10%, 15%, 20%, or greater than 20% higher than a wild type variety at 7, 14, or 21 days after harvest.

The lettuce plants of the present application also exhibit higher levels of vitamin A or beta-carotene in comparison to a wild type variety. In one embodiment, the level of vitamin A is higher than a wild type variety. In some embodiments, the level of vitamin A is at least 5%, 10%, 15%, 20%, 25%, 30%, 35%, 40%, 45%, 50%, 60%, 70%, 80%, 90%, 100%, or more than 100% higher than the level of vitamin A in a wild type variety. The lettuce plants of the present application also retain higher levels of beta carotene at 21 days after harvest in comparison to a wild type variety under the same conditions. In one embodiment, the level of beta carotene is at least 5%, 10%, 15%, 20%, 25%, 30%, 35%, 40%, 45%, 50%, 60%, 70%, 80%, 90%, 100%, or more than 100% higher than the level of beta carotene in a wild type variety at 21 days after harvest in comparison to a wild type variety under the same conditions.

The lettuce plants of the present application also exhibit higher levels of vitamin C in comparison to a wild type variety. In one embodiment, the level of vitamin C is higher than a wild type variety. In some embodiments, the level of vitamin C is at least 5%, 10%, 15%, 20%, 25%, 30%, 35%, 40%, 45%, 50%, 60%, 70%, 80%, 90%, 100%, or more than 100% higher than the level of vitamin C in a wild type variety. The lettuce plants of the present application also retain higher levels of vitamin C at 21 days after harvest in comparison to a wild type variety under the same conditions. In one embodiment, the level of vitamin C is at least 10-fold, 15-fold, 20-fold, 25-fold, 30-fold or 35-fold higher than the level of vitamin C in a wild type variety at 21 days after harvest in comparison to a wild type variety under the same conditions.

The lettuce plants of the present application also exhibit higher levels of vitamin K in comparison to a wild type variety. In one embodiment, the level of vitamin K is higher than a wild type variety. In some embodiments, the level of vitamin K is at least 5%, 10%, 15%, 20%, 25%, 30%, 35%, 40%, 45%, 50%, 60%, 70%, 80%, 90%, 100%, or more than 100% higher than the level of vitamin K in a wild type variety.

In another embodiment, the lettuce plants of the present application also exhibit reduced fermentation levels over time after harvest in comparison to a wild type variety under the same conditions. In some embodiments, the lettuce plants of the present application produce less CO₂ over time after harvest in comparison to a wild type variety under the same conditions. In some embodiments, the level of CO₂ produced is at least 5%, 10%, 15%, or 20% less than the level of level of CO₂ produced by a wild type variety at 6, 10, 14, 17, or 21 days after harvest.

In some embodiments, the lettuce plants of the present application also retain higher carbohydrate levels over time after harvest in comparison to a wild type variety under the same conditions. In some embodiments, the carbohydrate levels are at least 10%, 20%, 30%, 40%, 50%, 60%, 70%, 80%, 90%, 100%, or more than 100% higher than the level of carbohydrates of a wild type variety at 30 days after harvest.

### Constructs for Mutating PPO Genes

Vectors may be used to modify PPO genes in lettuce plants of the present application. Such vectors comprise at least one polynucleotide encoding a gRNA that targets a PPO gene of interest operably linked to a promoter that is active in plants. In some embodiments, the vector comprises a plurality of sequences encoding gRNAs targeting one or more PPO genes of interest. In some embodiments, the gRNA encoding sequences are arranged in a polycistronic arrangement.

In some embodiments, the gRNAs target at least one of the PPO-A, PPO-B, PPO-D, PPO-E, PPO-G, PPO-O, PPO-P, PPO-R, PPO-C, PPO-J, PPO-M, PPO-N, PPO-Q, and PPO-S genes in a plant. In some embodiments, the gRNAs target at least two of the PPO-A, PPO-B, PPO-D, PPO-E, PPO-G, PPO-O, PPO-P, PPO-R, PPO-C, PPO-J, PPO-M, PPO-N, PPO-Q, and PPO-S genes in a plant.

To achieve combinations of PPO gene mutations disclosed herein, one of skill in the art may design gRNAs to target the combinations of PPO genes desired. For example, a vector may comprise sequences encoding gRNAs that target PPO-B and PPO-S genes. In other embodiments, the vector comprises sequences encoding gRNAs that target PPO-B, PPO-R, and PPO-S genes. In other embodiments, the vector comprises sequences encoding gRNAs that target PPO-B, PPO-G, PPO-R, and PPO-S genes. In other embodiments, the vector comprises sequences encoding gRNAs that target PPO-B, PPO-D, PPO-E, and PPO-S genes. In still other embodiments, the vector comprises sequences encoding gRNAs that target PPO-B, PPO-G, and PPO-R genes. In other embodiments, the vector comprises sequences encoding gRNAs that target PPO-B, PPO-D, PPO-E, PPO-G, and PPO-S genes. In further embodiments, the vector comprises sequences encoding gRNAs that target PPO-A, PPO-B, PPO-D, PPO-E, and PPO-S genes. In still other embodiments, the vector comprises sequences encoding gRNAs that target PPO-A, PPO-B, PPO-D, PPO-E, PPO-G, and PPO-S genes.

In some embodiments, a gRNA may target multiple PPO genes simultaneously. In some embodiments, a gRNA may target PPO-A, PPO-B and/or PPO-C. In some embodiments, a gRNA may target PPO-D, PPO-E, PPO-N and/or PPO-P.

gRNAs targeting PPO genes for knockout may be predicted using a number of available programs to design gRNA sequences based on the nucleic acid sequences of the PPO genes. Without limiting thereto, the PPO genes targeted in the present application have the nucleic acid sequences of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27. Without limitation, gRNA sequences of the present application include SEQ ID NOs:35-40 and SEQ ID NOs:119-124 (Tables 2 and 6).

Vectors may comprise a polynucleotide encoding a gene editing nuclease operably linked to a promoter that is operable in plants. The nuclease may be any nuclease that acts with gRNAs to make double stranded cuts in target sites in the chromosome. Examples of gene editing nucleases that may be used include, but are not limited to, Cas9, MAD7, Cpf1, and chimeric synthetic nucleases such as SynNuc1 (SEQ ID NO:30). SynNuc1 may be encoded by the polynucleotide sequence of SEQ ID NO:29. Non-limiting examples of gene and protein sequences of synthetic nucleases are provided as follows:

SynNuc1 gene sequence (SEQ ID NO: 29):
ATGGGTTTGG ACTCTACTGC GCCTAAGAAA AAGAGAAAAG TCGGGATTCA CGGCGTGCCA
GCGGCCATGA CTCAGTTCGA GGGTTTCACT AACCTGTACC AAGTGTCCAA GACCCTGAGG
TTCGAGCTGA TCCCGCAAGG AAAGACCCTG AAGCACATCC AGGAACAGGG CTTCATCGAA
GAGGATAAGG CCCGCAACGA CCACTACAAG GAACTGAAGC CCATTATTGA TCGGATCTAC
AAGACCTACG CCGACCAGTG CTTGCAGCTG GTGCAGCTGG ACTGGGAAAA TCTGTCCGCC
GCGATTGATT CCTACCGGAA GGAGAAAACC GAAGAGACTC GCAACGCTCT CATTGAGGAA
CAGGCCACCT ACCGGAACGC CATTCACGAC TACTTTATTG GCCGCACTGA CAACCTCACC
GATGCAATCA ACAAGCGCCA CGCCGAGATC TACAAGGGCC TGTTCAAGGC GGAACTGTTT
AACGGGAAGG TCCTGAAGCA ACTGGGAACT GTGACCACCA CCGAGCATGA GAACGCCCTG
CTCCGCTCCT TCGACAAGTT CACCACCTAC TTCTCGGGAT TCTACGAGAA TCGCAAAAAC
GTGTTCAGCG CGGAAGATAT CTCAACCGCC ATCCCCCACC GGATTGTGCA GGACAACTTC
CCTAAGTTCA AGGAAAACTG CCATATCTTC ACGCGCCTGA TCACTGCTGT GCCGAGTCTG
AGAGAGCACT TCGAGAACGT GAAGAAGGCT ATCGGCATCT TCGTGTCCAC CTCGATTGAG
GAAGTGTTCT CCTTCCCGTT CTACAATCAG CTCCTGACTC AAACCCAGAT TGACCTGTAC
AACCAGCTTC TGGGGGGGAT TTCCCGGGAA GCGGGAACTG AGAAGATCAA GGGACTCAAC
GAAGTGCTGA ACCTGGCAAT CCAGAAGAAC GACGAAACCG CGCACATCAT CGCAAGCCTC
CCTCACCGCT TCATTCCTCT GTTCAAGCAA ATTCTTTCCG ACCGCAACAC CCTGTCGTTC
ATCCTGGAAG AATTCAAGAG CGACGAAGAA GTCATTCAGA GCTTCTGCAA GTACAAGACT
CTGCTGAGGA ACGAAAACGT GCTGGAAACC GCCGAGGCCC TGTTCAACGA ACTGAACTCA
ATCGACCTGA CGCACATTTT CATTTCCCAT AAGAAGCTGG AAACTATCTC CTCCGCCCTC
TGTGACCACT GGGACACCCT GAGAAATGCG TTGTATGAGC GCCGGATCTC CGAGTTGACT
GGGAAGATTA CTAAGTCCGC GAAGGAAAAA GTGCAGCGCT CCCTGAAACA CGAAGATATC
AACCTTCAGG AGATCATCTC AGCCGCCGGA AAGGAACTGT CAGAGGCCTT CAAGCAAAAG
ACTTCAGAGA TCCTGTCGCA CGCCCACGCC GCTTTGGACC AGCCCCTGCC CACCACCCTG
AAGAAGCAGG AAGAAAAGGA AATCCTGAAG TCTCAGCTCG ACTCACTGCT TGGGCTGTAC
CATCTCCTCG ATTGGTTCGC CGTCGACGAG TCCAACGAAG TCGACCCGGA ATTCTCGGCC
CGGCTGACCG GTATCAAGCT TGAGATGGAG CCAAGCCTCT CCTTTTACAA CAAGGCCCGG
AACTACGCCA CCAAAAAGCC TTACTCAGTG GAAAAGTTCA AGCTTAACTT TCAAATGCCG
ACCCTGGCCA GCGGCTGGGA CGTGAACAAG GAGAAGAACA ACGGCGCCAT CCTGTTTGTG
AAGAACGGAC TGTATTACCT TGGAATTATG CCCAAACAGA AGGGTCGCTA CAAGGCACTG
TCCTTCGAGC CGACCGAAAA GACTTCGGAA GGTTTTGACA AGATGTACTA CGATTACTTC
CCGGACGCGG CTAAGATGAT CCCCAAGTGC AGCACTCAGC TGAAGGCCGT GACCGCACAC
TTTCAAACCC ATACCACCCC GATTCTTCTG AGCAACAACT TTATCGAGCC ACTGGAGATT
ACCAAGGAAA TCTACGACCT GAACAACCCC GAAAAGGAAC CTAAAAAGTT TCAGACCGCC
TACGCCAAGA AAACTGGCGA CCAGAAGGGA TACAGAGAAG CCCTCTGCAA GTGGATTGAC
TTCACCCGGG ATTTCCTGTC CAAGTACACT AAGACCACTT CCATTGACCT CTCGTCGCTG
CGGCCGTCCT CGCAATACAA GGACCTGGGG GAGTACTACG CCGAGCTCAA CCCGCTGCTC
TACCACATAA GCTTCCAGCG GATTGCCGAG AAAGAAATCA TGGACGCCGT CGAAACCGGA
AAGCTGTACC TCTTCCAAAT CTATAACAAG GACTTCGCGA AGGGTCACCA TGGAAAGCCA
AACCTCCACA CCCTCTATTG GACCGGACTC TTCTCGCCGG AAAACCTGGC CAAGACATCC
ATCAAGTTGA ATGGGCAGGC CGAACTCTTC TACCGCCCCA AGTCTCGGAT GAAGCGAATG
GCCCACCGGC TGGGAGAAAA GATGCTCAAC AAGAAGCTAA AGGACCAAAA GACCCCAATC
CCTGACACCC TGTACCAGGA ACTGTACGAT TACGTGAACC ACAGGCTTAG CCACGACTTA
TCCGACGAAG CCCGGGCGCT GCTGCCGAAC GTCATCACCA AGGAAGTGTC GCACGAGATC
ATCAAGGACC GCCGGTTTAC CTCCGACAAA TTCTTCTTCC ACGTGCCCAT TACCCTGAAC
TACCAGGCCG CCAACTCACC CGGATTCATC AACGACCGGA TCCTGCAGTA TATCGCGAAG
GAGAAGGATC TTCACGTGAT CGGAATTGAC CGGGGGGAGA GGAACCTGAT CTACGTGTCC
GTGATTGACA CTTGTGGGAA CATCGTAGAG CAGAAGTCCT TCAACATCGT GAACGGCTAC
GACTACCAGA TCAAGCTCAA GCAACAGGAG GGCGCACGGC AGATCGCAAG AAAGGAATGG
AAGGAAATCG GAAAAATCAA GGAAATCAAA GAGGGATACC TGAGCCTCGT CATCCACGAG
ATCAGCAAGA TGGTCATTAA GTACAATGCG ATCATCGCCA TGGAGGACTT GTCCTACGGA
TTCAAGAAAG GACGGTTCAA AGTGGAGAGA CAAGTGTATC AGAAGTTCGA GACTATGCTC
ATCAACAAGC TGAACTACCT GGTGTTCAAG GATATCAGCA TTACGGAAAA CGGCGGACTC
CTCAAGGGAT ACCAGCTGAC TTACATTCCC GATAAGCTGA AGAATGTCGG TCATCAGTGC
GGCTGTATTT TCTACGTGCC GGCAGCCTAC ACCTCCAAGA TCGACCCTAC TACCGGTTTC
GTGAACATTT TTAAGTTCAA AGATCTCACC GTGGACGCAA AGCGCGAATT CATTAAGAAG
TTCGACTCAA TCCGCTACGA CAGCGAGAAG AACCTGTTCT GCTTCACTTT CGACTACAAC
AACTTCATTA CCCAAAACAC CGTCATGTCC AAGTCCAGCT GGAGCGTGTA CACCTATGGA
GTGCGGATCA AGCGGCGGTT TGTGAACGGC CGGTTCTCGA ATGAGTCCGA CACAATTGAT
ATCACCAAAG ATATGGAAAA GACACTGGAG ATGACTGATA TCAACTGGAG GGATGGCCAC
GATTTGAGAC AGGACATTAT TGACTACGAG ATAGTCCAGC ATATCTTTGA GATTTTCAGA
CTGACCGTGC AGATGCGCAA TTCCCTGTCG GAACTGGAAG ATCGGGACTA CGATAGACTG
ATTAGCCCCG TGCTGAACGA AAACAACATC TTCTACGATT CCGCCAAAGC TGGAGATGCG
CTGCCAAAAG ACGCTGACGC TAACGGCGCC TACTGCATCG CGCTGAAGGG CCTCTACGAA
ATCAAGCAAA TCACCGAGAA CTGGAAGGAG GACGGAAAGT TCTCCCGCGA CAAGCTGAAG
ATCTCAAACA AGGACTGGTT TGACTTCATC CAGAACAAGC GGTACCTGAA GCGCCCTGCT
GCTACCAAAA AGGCCGGCCA GGCCAAGAAG AAAAAGGGCT CGTACCCCTA CGATGTGCCG
GATTAGGCCT ACCCTTACGA TGTCCCCGAC TACGCTTACC CGTACGACGT GCCTGACTAC
GCCTAA
SynNuc1 protein sequence (SEQ ID NO: 30):
MGLDSTAPKK KRKVGIHGVP AAMTQFEGFT NLYQVSKTLR FELIPQGKTL KHIQEQGFIE
EDKARNDHYK ELKPIIDRIY KTYADQCLQL VQLDWENLSA AIDSYRKEKT EETRNALIEE
QATYRNAIHD YFIGRTDNLT DAINKRHAEI YKGLFKAELF NGKVLKQLGT VTTTEHENAL
LRSFDKFTTY FSGFYENRKN VFSAEDISTA IPHRIVQDNF PKFKENCHIF TRLITAVPSL
REHFENVKKA IGIFVSTSIE EVFSFPFYNQ LLTQTQIDLY NQLLGGISRE AGTEKIKGLN
EVLNLAIQKN DETAHIIASL PHRFIPLFKQ ILSDRNTLSF ILEEFKSDEE VIQSFCKYKT
LLRNENVLET AEALFNELNS IDLTHIFISH KKLETISSAL CDHWDTLRNA LYERRISELT
GKITKSAKEK VQRSLKHEDI NLQEIISAAG KELSEAFKQK TSEILSHAHA ALDQPLPTTL
KKQEEKEILK SQLDSLLGLY HLLDWFAVDE SNEVDPEFSA RLTGIKLEME PSLSFYNKAR
NYATKKPYSV EKFKLNFQMP TLASGWDVNK EKNNGAILFV KNGLYYLGIM PKQKGRYKAL
SFEPTEKTSE GFDKMYYDYF PDAAKMIPKC STQLKAVTAH FQTHTTPILL SNNFIEPLEI
TKEIYDLNNP EKEPKKFQTA YAKKTGDQKG YREALCKWID FTRDFLSKYT KTTSIDLSSL
RPSSQYKDLG EYYAELNPLL YHISFQRIAE KEIMDAVETG KLYLFQIYNK DFAKGHHGKP
NLHTLYWTGL FSPENLAKTS IKLNGQAELF YRPKSRMKRM AHRLGEKMLN KKLKDQKTPI
PDTLYQELYD YVNHRLSHDL SDEARALLPN VITKEVSHEI IKDRRFTSDK FFFHVPITLN
YQAANSPGFI NDRILQYIAK EKDLHVIGID RGERNLIYVS VIDTCGNIVE QKSFNIVNGY
DYQIKLKQQE GARQIARKEW KEIGKIKEIK EGYLSLVIHE ISKMVIKYNA IIAMEDLSYG
FKKGRFKVER QVYQKFETML INKLNYLVFK DISITENGGL LKGYQLTYIP DKLKNVGHQC
GCIFYVPAAY TSKIDPTTGF VNIFKFKDLT VDAKREFIKK FDSIRYDSEK NLFCFTFDYN
NFITQNTVMS KSSWSVYTYG VRIKRRFVNG RFSNESDTID ITKDMEKTLE MTDINWRDGH
DLRQDIIDYE IVQHIFEIFR LTVQMRNSLS ELEDRDYDRL ISPVLNENNI FYDSAKAGDA
LPKDADANGA YCIALKGLYE IKQITENWKE DGKFSRDKLK ISNKDWFDFI QNKRYLKRPA
ATKKAGQAKK KKGSYPYDVP DYAYPYDVPD YAYPYDVPDY A

Useful promoters for driving expression of the polynucleotide encoding gRNAs encoding PPO genes of interest are promoters operable in plants including, but not limited, to an Arabidopsis thaliana U6 promoter, a 35S promoter, and a CsVMV promoter. The promoter that drives expression of the gRNA(s) may be the same or different than the promoter that drives expression of the gene editing nuclease. Exemplary promoter sequences are given in SEQ ID NOs:31-34.

tU6 promoter (SEQ ID NO: 31):
AAAAGCTTCG TTGAACAACG GAAACTCGAC TTGCCTTCCG
CACAATACAT CATTTCTTCT TAGCTTTTTT TCTTCTTCTT
CGTTCATACA GTTTTTTTTT GTTTATCAGC TTACATTTTC
TTGAACCGTA GCTTTCGTTT TCTTCTTTTT AACTTTCCAT
TCGGAGTTTT TGTATCTTGT TTCATAGTTT GTCCCAGGAT
TAGAATGATT AGGCATCGAA CCTTCAAGAA TTTGATTGAA
TAAAACATCT TCATTCTTAA GATATGAAGA TAATCTTCAA
AAGGCCCCTG GGAATCTGAA AGAAGAGAAG CAGGCCCATT
TATATGGGAA AGAACAATAG TATTTCTTAT ATAGGCCCAT
TTAAGTTGAA AACAATCTTC AAAAGTCCCA CATCGCTTAG
ATAAGAAAAC GAAGCTGAGT TTATATACAG CTAGAGTCGA
AGTAGTGATT
CaMV 35S promoter (SEQ ID NO: 32):
GGTCCGATGT GAGACTTTTC AACAAAGGGT AATATCCGGA
AACCTCCTCG GATTCCATTG CCCAGCTATC TGTCACTTTA
TTGTGAAGAT AGTGGAAAAG GAAGGTGGCT CCTACAAATG
CCATCATTGC GATAAAGGAA AGGCCATCGT TGAAGATGCC
TCTGCCGACA GTGGTCCCAA AGATGGACCC CCACCCACGA
GGAGCATCGT GGAAAAAGAA GACGTTCCAA CCACGTCTTC
AAAGCAAGTG GATTGATGTG ATGGTCCGAT GTGAGACTTT
TCAACAAAGG GTAATATCCG GAAACCTCCT CGGATTCCAT
TGCCCAGCTA TCTGTCACTT TATTGTGAAG ATAGTGGAAA
AGGAAGGTGG CTCCTACAAA TGCCATCATT GCGATAAAGG
AAAGGCCATC GTTGAAGATG CCTCTGCCGA CAGTGGTCCC
AAAGATGGAC CCCCACCCAC GAGGAGCATC GTGGAAAAAG
AAGACGTTCC AACCACGTCT TCAAAGCAAG TGGATTGATG
TGATATCTCC ACTGACGTAA GGGATGACGC ACAATCCCAC
TATCCTTCGC AAGACCCTTC CTCTATATAA GGAAGTTCAT
TTCATTTGGA GAGGACACGC GACAAGCTGA CTCTAGCAGA
TCCTCCAGA
CsVMV promoter (SEQ ID NO: 33):
CAGAAGGTAA TTATCCAAGA TGTAGCATCA AGAATCCAAT
GTTTACGGGA AAAACTATGG AAGTATTATG TGAACTCAGC
AAGAAGCAGA TCAATATGCG GCACATATTC AACCTATGTT
CAAAAATGAA GAATGTACAG ATACAAGATC CTATACTGCC
AGAATACGAA GAAGAATACA TAGAAATTGA AAAAGAAGAA
CCAGGCGAAG AAAAGAATCT TGAAGACGTA AGCACTGACG
ACAACAATGA AAAGAAGAAG ATAAGGTCGG TGATTGTGAA
AGAGACATAG AGGACACATG TAAGGTGGAA AATGTAAGGG
CGGAAAGTAA CCTTATCACA AAGGAATCTT ATCCCCCACT
ACTTATCCTT TTATATTTTT CCGTGTCATT TTTGCCCTTG
AGTTTTCCTA TATAAGGAAC CAAGTTCGGC ATTTGTGAAA
ACAAGAAAAA ATTTGGTGTA AGCTATTTTC TTTGAAGTAC
TGAGGATACA ACTTCAGAGA AATTTGTAAG TTTG
AtUBQ 10 promoter (SEQ ID NO: 34):
GAACTTATTC AAAGAATGTT TTGTGTATCA TTCTTGTTAC
ATTGTTATTA ATGAAAAAAT ATTATTGGTC ATTGGACTGA
ACACGAGTGT TAAATATGGA CCAGGCCCCA AATAAGATCC
ATTGATATAT GAATTAAATA ACAAGAATAA ATCGAGTCAC
CAAACCACTT GCCTTTTTTA ACGAGACTTG TTCACCAACT
TGATACAAAA GTCATTATCC TATGCAAATC AATAATCATA
CAAAAATATC CAATAACACT AAAAAATTAA AAGAAATGGA
TAATTTCACA ATATGTTATA CGATAAAGAA GTTACTTTTC
CAAGAAATTC ACTGATTTTA TAAGCCCACT TGCATTAGAT
AAATGGCAAA AAAAAACAAA AAGGAAAAGA AATAAAGCAC
GAAGAATTCT AGAAAATACG AAATACGCTT CAATGCAGTG
GGACCCACGG TTCAATTATT GCCAATTTTC AGCTCCACCG
TATATTTAAA AAATAAAACG ATAATGCTAA AAAAATATAA
ATCGTAACGA TCGTTAAATC TCAACGGCTG GATCTTATGA
CGACCGTTAG AAATTGTGGT TGTCGACGAG TCAGTAATAA
ACGGCGTCAA AGTGGTTGCA GCCGGCACAC ACGAGTCGTG
TTTATCAACT CAAAGCACAA ATACTTTTCC TCAACCTAAA
AATAAGGCAA TTAGCCAAAA ACAACTTTGC GTGTAAACAA
CGCTCAATAC ACGTGTCATT TTATTATTAG CTATTGCTTC
ACCGCCTTAG CTTTCTCGTG ACCTAGTCGT CCTCGTCTTT
TCTTCTTCTT CTTCTATAAA ACAATACCCA AAGAGCTCTT
CTTCTTCACA ATTCAGATTT CAATTTCTCA AAATCTTAAA
AACTTTCTCT CAATTCTCTC TACCGTGATC AAGGTAAATT
TCTGTGTTCC TTATTCTCTC AAAATCTTCG ATTTTGTTTT
CGTTCGATCC CAATTTCGTA TATGTTCTTT GGTTTAGATT
CTGTTAATCT TAGATCGAAG ACGATTTTCT GGGTTTGATC
GTTAGATATC ATCTTAATTC TCGATTAGGG TTTCATAGAT
ATCATCCGAT TTGTTCAAAT AATTTGAGTT TTGTCGAATA
ATTACTCTTC GATTTGTGAT TTCTATCTAG ATCTGGTGTT
AGTTTCTAGT TTGTGCGATC GAATTTGTCG ATTAATCTGA
GTTTTTCTGA TTAACAG

### Methods of Modifying PPO Genes in Plants

Another aspect of the present application is directed to a method of reducing the PPO activity of a lettuce plant. This method involves introducing a mutation into each of at least two different PPO genes of a lettuce plant, where said mutations reduce the amount and/or activity of PPO protein compared to a wild type lettuce plant.

Mutating or otherwise modifying PPO genes in a plant such as lettuce may be done by any method known in the art such that the combinations of PPO gene mutations disclosed herein are achieved. PPO genes of the present application include PPO-A, PPO-B, PPO-C, PPO-D, PPO-E, PPO-G, PPO-J, PPO-M, PPO-N, PPO-O, PPO-P, PPO-Q, PPO-R, and PPO-S. Any method known in the art to make lettuce with any of the following PPO gene mutations is embraced by the present application: mutations in at least two different PPO genes; PPO-B and PPO-S mutations; PPO-B and PPO-R mutations; PPO-B, PPO-R, and PPO-S mutations; PPO-B, PPO-G, PPO-R, and PPO-S mutations; PPO-B, PPO-G, and PPO-R mutations; PPO-B, PPO-D, PPO-E, PPO-G, and PPO-S mutations; PPO-A, PPO-B, PPO-D, PPO-E, and PPO-S mutations; and PPO-A, PPO-B, PPO-D, PPO-E, PPO-G, and PPO-S mutations. Any combination of PPO gene mutation that imparts improved plant traits, including those described herein, may be made according to this aspect of the present application.

Examples of how mutations may be made include, but are not limited to, homologous recombination, insertional mutagenesis to mutate a gene by inserting a sequence into the coding sequence of the gene, the use of gene editing nucleases (e.g., TALENs, zinc finger nucleases, meganucleases, and CRISPR/Cas9 type editing), serine recombinases, chemical mutagenesis, radiation, and recombinagenic oligonucleotides.

Gene editing is a type of genetic engineering in which DNA is inserted, replaced, or removed from a genome using artificially engineered nucleases, or “molecular scissors.” The nucleases create specific double-stranded breaks (“DSBs”) at desired locations in the genome, and harness the cell's endogenous mechanisms to repair the induced break by natural processes of homologous recombination (“HR”) and nonhomologous end-joining (“NHEJ”). There are currently four main families of engineered nucleases being used: Zinc finger nucleases (“ZFNs”), Transcription Activator-Like Effector Nucleases (“TALENs”), the CRISPR/Cas system, and engineered meganuclease with a re-engineered homing endonucleases. Any method of genome engineering may be used in the embodiments of the present application.

ZFNs are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms. ZFNs consist of an engineered zinc finger DNA-binding domain fused to the cleavage domain of the FokI restriction endonuclease. ZFNs can be used to induce double-stranded breaks (DSBs) in specific DNA sequences and thereby promote site-specific homologous recombination with an exogenous template. The exogenous template contains the sequence that is to be introduced into the genome.

TALEN is a sequence-specific endonuclease that includes a transcription activator-like effector (“TALE”) and a FokI endonuclease. The transcription activator-like effector is a DNA binding protein that has a highly conserved central region with tandem repeat units of 34 amino acids. The base preference for each repeat unit is determined by two amino acid residues called the repeat-variable di-residue, which recognizes one specific nucleotide in the target DNA. Arrays of DNA-binding repeat units can be customized for targeting specific DNA sequences. As with ZFNs, dimerization of two TALENs on targeted specific sequences in a genome results in FokI-dependent introduction of double stranded breaks, stimulating homology directed repair (“HDR”) and Non-homologous end joining (NHEJ) repair mechanisms.

Meganucleases with re-engineered homing nucleases can also be used to effect genome modification in lettuce in the methods described herein. Meganucleases are endodeoxyribonucleases characterized by a large recognition site (double-stranded DNA sequences of 12 to 40 base pairs). This site generally occurs only once in any given genome. For example, the 18-base pair sequence recognized by the I-Scel meganuclease would on average require a genome twenty times the size of the human genome to be found once by chance. Meganucleases are considered to be the most specific naturally occurring restriction enzymes. Among meganucleases, the LAGLIDADG family of homing endonucleases has become a valuable tool for the study of genomes and genome engineering over the past fifteen years. By modifying their recognition sequence through protein engineering, the targeted sequence can be changed.

CRISPR/Cas type RNA-guided endonucleases provide an efficient system for inducing genetic modifications in genomes of many organisms and can be used in the methods described herein to introduce one or more genetic modifications in a lettuce plant genome that result in suppression or altered activity of a native lettuce PPO gene. In some embodiments the endonuclease is SynNuc1, but can also be an endonuclease from one of many related CRISPR systems that have been described. Non-limiting examples of gene editing nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, Mad7, SynNuc1, homologs thereof, or modified versions, and endonuclease inactive versions thereof. CRISPR/Cas systems can be a type I, a type II, or a type III system.

Use of such systems for gene editing has been widely described. For example, the use of CRISPR guide RNA in conjunction with CRISPR-Cas9 technology to target RNA is described in Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339:819-23 (2013); and Gaj et al., “ZFN, TALEN, and CRISPR/Cas-based Methods for Genome Engineering,” Cell 31:397-405 (2013), which are hereby incorporated by reference in their entirety. Other nucleases that may be used in gene editing include, but are not limited to, Cpf1, MAD7, and synthetic nucleases such as SynNuc1 (SEQ ID NO:30). In some embodiments, the nuclease is SynNuc1.

There are two distinct components to a CRISPR/Cas system, a guide RNA and an endonuclease, such as Cas9. The guide RNA is a combination of the endogenous bacterial CRISPR RNA (“crRNA”) and trans-activating crRNA (“tracrRNA”) into a single chimeric guide RNA (“gRNA”) transcript. The gRNA combines the targeting specificity of the crRNA with the scaffolding properties of the tracrRNA into a single transcript. When the gRNA and Cas9 are expressed in the cell, the genomic target sequence can be modified or permanently disrupted. The gRNA/Cas9 complex is recruited to the target sequence by the base-pairing between the gRNA sequence which has a region of the complementarity to the target sequence in the genomic DNA. For successful binding of Cas9, the genomic target sequence must also contain the correct Protospacer Adjacent Motif (“PAM”) sequence immediately following the target sequence. The binding of the gRNA/Cas9 complex localizes the Cas9 to the genomic target sequence so that the wild type Cas9 can cut both strands of DNA causing a DSB. Cas9 generates DSBs through the combined activity of two nuclease domains, RuvC and HNH. Cas9 will cut 3-4 nucleotides upstream of the PAM sequence. A DSB can be repaired through one of two general repair pathways: (1) NHEJ DNA repair pathway or (2) the HDR pathway. The NHEJ repair pathway often results in insertions/deletions (InDels) at the DSB site that can lead to frameshifts and/or premature stop codons, effectively disrupting the open reading frame (ORF) of the targeted gene. The HDR pathway requires the presence of a repair template, which is used to fix the DSB. HDR faithfully copies the sequence of the repair template to the cut target sequence. Specific nucleotide changes can be introduced into a targeted gene by the use of HDR with a repair template. CRISPR specificity can be controlled by level of homology and binding strength of the specific gRNA for a given gene target, or by modification of the Cas endonuclease itself. For example, a D10A mutant of the RuvC domain, retains only the HNH domain and generates a DNA nick rather than a DSB.

When the guide RNA and the gene editing endonuclease are expressed in the cell, the genomic target sequence can be modified or permanently disrupted. The guide RNA/gene editing endonuclease complex is recruited to the target sequence by the base-pairing between the guide RNA sequence and the complementary sequence of the target sequence in the genomic DNA. In some embodiments, CRISPR gene editing is used to generate a lettuce PPO gene mutation by causing nucleotide insertions or deletions (indels) at the DSB site. In some embodiments, a point mutation, insertions, deletions, or any combination thereof, can be generated in a PPO gene in the lettuce genome. In yet other embodiments, multiple PPO gene targets can be modified using CRISPR gene editing in a single experiment using single or multiple guide RNAs having specificity for the different gene targets. In some embodiments, two PPO gene targets are mutated. In other embodiments, more than two PPO genes are mutated. Thus, a wide range of genetic modifications in the lettuce genome using the described methods and CRISPR gene editing can be attained.

Although insertion of recombinant DNA into the plant genome may be used to generate genome modifications using CRISPR gene editing, such genome modifications can be achieved without inserting recombinant DNA into the lettuce genome. In some embodiments, a ribonucleotide particle or ribonucleoprotein (“RNP”) is preassembled and delivered to a target lettuce explant. Furthermore, where recombinant DNA is inserted into the lettuce plant genome to effect genome modification, plants having edited genomes, but lacking recombinant DNA, may be obtained by segregation using standard breeding techniques such as crossing and backcrossing. Alternatively, transient expression of gene editing components, including guide RNA and a native or modified endonuclease through recombinant DNA in lettuce cells can also result in genome modification, since gene editing components are no longer required once the desired modification is generated. In such embodiments, mutations are effected in the plant cell genome as a result of transient expression of gene editing constructs, and such mutations are inherited in future generations without the need to segregate away transgenes used to express the gene editing components required for genome modification.

In other embodiments, genetic modification results in reduced gene expression of PPO genes. Reduction of gene expression can be achieved by a variety of techniques including antisense gene suppression, co-suppression, ribozymes, microRNA or genome editing. In some embodiments, recombinant DNA antisense molecules may include sequences that correspond to one or more PPO genes or sequences that effect control over the gene expression or over a splicing event. Thus, the antisense sequence may correspond to a gene coding region, 5′-untranslated region (UTR), the 3′-UTR, intron or any combination of these. In other embodiments, catalytic polynucleotides referred to as ribozymes or deoxyribozymes may be expressed and targeted to a specific mRNA of interest, such one or more PPO genes. The expressed catalytic nucleic acid contains an antisense sequence for specific recognition of a target nucleic acid, and a nucleic acid cleaving enzymatic activity. The ribozyme hybridizes to and cleaves the target nucleic acid molecule resulting in suppression of gene activity. Alternatively, RNA interference (RNAi) may be used specifically inhibiting the production of one or more PPO genes. PPO genes can be silenced by small interfering RNA (siRNA) molecules that cause endonucleatic cleavage of the target mRNA molecules or by microRNA (miRNA) molecules that suppress translation of the mRNA molecule. The design and production of suitable dsRNA molecules for PPO genes may be down-regulated by co-suppression. The mechanism of co-suppression is thought to involve post-transcriptional gene silencing (PTGS) and may be similar to antisense suppression. It involves introducing an extra copy of a gene or a fragment thereof into a plant in the sense orientation with respect to a promoter for its expression.

In one embodiment, the present application relates to a lettuce plant with reduced expression of at least two PPO genes and/or reduced amount/or activity of the PPO proteins, where reduced expression of the at least PPO gene and/or reduced activity of the at least two PPO proteins is achieved by genomic editing. In one embodiment, the present application relates to a lettuce plant with at least two genome edited PPO genes, where the lettuce plant exhibits characteristics selected from the group consisting of reduced tip burn, reduced browning, reduced yellowing of the midvein, increased polyphenolics, increased vitamins, increased vitamin retention, lower levels of CO2 production, and higher levels of carbohydrates as compared to a wild type plant. In one embodiment, the method of introducing a human-induced mutation into the at least two PPO genes is carried out by gene editing.

In other embodiments, a polynucleotide structure for modification of a lettuce plant genome comprises one or more RNPs. In such embodiments, polynucleotide structures are employed outside of a plant cell to produce the guide RNA and endonuclease components, which are then pre-assembled and delivered to a target plant tissue. In such embodiments, multiple guide RNAs targeting multiple lettuce PPO genes can be designed and assembled with a single type of RNA guided endonuclease, for example SynNuc1. A lettuce plant part having regenerable cells is targeted for delivery of RNP structures.

Chemical and/or radiation mutagenesis can be used to develop the mutations of the present application. These mutagens can create point mutations, deletions, insertions, transversions, and/or transitions, or combinations thereof. Radiation mutagenesis includes, without limitation, ultraviolet light, x-rays, gamma rays, and fast neutrons. Chemical mutagens include, but are not limited to, ethyl methanesulfonate (EMS), methylmethane sulfonate (MMS), N-ethyl-N-nitrosourea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (DEB), and the like), 2-methoxy-6-chloro-9[3-(ethyl-2-chloro-ethyl)aminopropylamino]acridine dihydrochloride (ICR-170), sodium azide, formaldehyde, or combinations thereof.

One of skill in the art will understand that a variety of lettuce plant materials including, but not limited to, seeds, leaves, stems, roots, vegetative buds, floral buds, meristems, embryos, cotyledons, endosperm, sepals, petals, pistils, carpels, stamens, anthers, microspores, pollen, pollen tubes, ovules, ovaries, other plant tissue or plant cells including protoplasts, may be edited and/or mutagenized in order to create the PPO-mutated lettuce plants disclosed herein.

Introduction of polynucleotide constructs or RNPs into plants may be performed by introducing the constructs or RNPs into protoplasts. Protoplasts may be made by any means known in the art such as, but not limited to that found in Engler & Grogan, “Isolation, Culture and Regeneration of Lettuce Leaf Mesophyll Protoplasts,” Plant Sci. Lett. 28:223-229 (1983); Nishio, “Simple and Efficient Protoplast Culture Procedure of Lettuce, Lactuca sativa L.,” Jap. J. Breeding 38(2):165-171 (1988), which are hereby incorporated by reference in their entirety.

Delivery of DNA constructs for modification of a plant genome can be accomplished by plant transformation, including, for example, infection with a microbe, such as Rhizobia or Agrobacterium infection. The Ti (or Ri) plasmid of Agrobacterium enables the highly successful transfer of a foreign nucleotide molecule into plant cells. A variation of Agrobacterium transformation uses vacuum infiltration in which whole plants are used (Senior, “Uses of Plant Gene Silencing,” Biotechnology and Genetic Engineering Reviews 15:79-119 (1998), which is hereby incorporated by reference in its entirety). In some embodiments, transformation involves fusion of protoplasts with other entities, either minicells, cells, lysosomes, or other fusible lipid-surfaced bodies (Fraley et al., “Liposome-Mediated Delivery of Tobacco Mosaic Virus RNA into Tobacco Protoplasts: A Sensitive Assay for Monitoring Liposome-Protoplast Interactions,” Proc. Natl. Acad. Sci. USA 79:1859-63 (1982), which is hereby incorporated by reference in its entirety).

Wounding of a target plant tissue prior to or during DNA delivery, for example using Agrobacterium or a Rhizobia species, such as Ensifer adhaerens, may also be employed to cause transformation. Various methods of wounding are employed in plant transformation methods, including for example, microprojectile bombardment; treatment with glass beads; cutting, scratching or slicing; sonication; or silicon carbide fibers or whiskers.

Transformation of protoplasts may be performed using any method known in the art including, but not limited to polyethylene glycol treatment (Lelivelt et al., “Plastid Transformation in Lettuce (Lactuca sativa L.) by Polyethylene Glycol Treatment of Protoplasts,” Meth. Mol. Biol. 1132:317-330 (2014); Lelivelt et al., “Stable Plastid Transformation in Lettuce (Lactuca sativa L.),” Plant Mol. Biol. 58:763-774 (2005), which are hereby incorporated by reference in their entirety); using Sheen's protocol (Sheen, J. (2002) at URL genetics.mgh.harvard.edu/sheenweb/); Yoo & Sheen, “Arabidopsis Mesophyll Protoplasts: A Versatile Cell System for Transient Gene Expression Analysis,” Nat. Protocol. 2(7):1565-1572 (2007), which are hereby incorporated by reference in their entirety); gene gun delivery (biolistics); electroporation, nanoparticle-based gene delivery, such as RNPs, gold nanoparticles, starch nanoparticles, silica nanoparticles and the like, and Agrobacterium-mediated delivery (for a review of these methods, see Demirer & Landry, “Delivering Genes to Plants SBE Supplement,” (2017) SBE Supplement: Plant Synth. Biol. 40-45, which is hereby incorporated by reference in its entirety) and a serine recombinase-mediated delivery.

The gene edited protoplasts may be grown into plants with the mutated PPO genes of choice. Methods of cultivating protoplasts into plants may be done by any means known in the art. See, for example, Enomoto and Ohyama, “Regeneration of Plants from Protoplasts of Lettuce and its Wild Species,” In: Bajaj Y.P.S. (eds) Plant Protoplasts and Genetic Engineering I. Biotechnology in Agriculture and Forestry, vol 8. Springer, Berlin, Heidelberg (1989), which is hereby incorporated by reference in its entirety.

Any method of transformation that results in efficient transformation of the host cell of choice is appropriate for practicing the present application.

A further aspect of the present application is directed to a method of editing PPO genes of lettuce plant. This method involves introducing into a lettuce plant a polynucleotide construct comprising a first nucleic acid sequence encoding a gene editing nuclease, a promoter that is functional in plants operably linked to said first nucleic acid sequence, a second nucleic acid sequence encoding a plurality of gRNAs in a polycistronic arrangement targeting at least two PPO genes of choice to edit, and a second promoter that is functional in plants, operably linked to said second nucleic acid sequence.

Another aspect of the present application is directed to a polynucleotide construct for editing polyphenol oxidase genes of a plant comprising a nucleic acid sequence encoding a plurality of gRNAs targeting at least two PPO genes operably linked to a promoter.

In some embodiments, the lettuce plant of the present application is free of any plant pest sequences. In some embodiments, the lettuce of the present application is free of any selectable marker. In some embodiments, the lettuce of the present application is free of any heterologous nucleotides. In some embodiments, breeding of lettuce plants of the present application is used to select lines comprising the PPO mutations and where the heterologous nucleotides have been segregated away, such that the lettuce genome contains no heterologous nucleotides. In some embodiments, a serine recombinase-mediated method may be used to ensure the lettuce is free of any heterologous nucleotides.

In the serine recombinase-mediated method, the vector comprises at least one att site for integrating into the plant chromosome. The plant contains a complementary pseudo att site and contacting a pair of recombination attachment sites, attB and attP engineered in the vector and the plant's own pseudo att sites, with a corresponding recombinase mediates recombination between the recombination attachment sites. Thus, one can obtain integration of a plasmid that contains one recombination site into a plant cell chromosome that includes the corresponding recombination site. In some embodiments, the att site on the vector is an attP site. In some embodiments, the att site on the vector is an attB site. In some embodiments, the serine recombinase is provided as a polynucleotide encoding the serine recombinase of choice operably linked to a promoter that is operable in the plant. The polynucleotide may be part of the vector that also expresses the gRNAs and editing nuclease or it may be on a separate vector. In other embodiments, a serine recombinase protein is introduced into the same cell as the polynucleotide construct described herein. Useful serine recombinases include, but are not limited to BXB1, SF370.1, SPβc2, A188, φC31, TP901-1, Tn3, and gamma delta.

In some embodiments, a serine recombinase-mediated insertion of the polynucleotide construct of the present application is used and the gRNA-mediated editing is allowed to occur and then the polynucleotide vector is excised from the chromosome using a cognate Recombination Directionality Factor (RDF) (Smith & Thorpe, “Diversity in the Serine Recombinases,” Mol. Microbiol. 44:299-307 (2002), which is hereby incorporated by reference in its entirety) of the serine recombinase used to integrate the polynucleotide construct. In some embodiments the serine recombinase used is an Spβc2 serine recombinase, and the excising RDF is the cognate RDF for Spβc2. With the polynucleotide construct excised from the chromosome, the plant is free of plant pest sequences including any selectable markers.

### Methods of Breeding PPO Mutant Genes into Multiple Types of Lettuce Cultivars

The PPO mutations of the present application can be transferred to other varieties of lettuce through breeding to develop new, unique lettuce cultivars and hybrids. Backcross breeding has been used to transfer genes for a simply inherited, highly heritable trait into a desirable homozygous cultivar or inbred line which is the recurrent parent. The source of the trait to be transferred is called the donor parent, such as the PPO mutant lettuce lines described in the present application. After the initial cross of a plant of the present application to another plant, individuals possessing the PPO mutations of the donor parent are selected and repeatedly crossed (backcrossed) to the recurrent parent. The resulting plants can be selfed to produce plants with homozygous PPO mutations. The resulting plant is expected to have the attributes of the recurrent parent (e.g., cultivar) and the desirable trait, such as the PPO mutations of the present application, transferred from the donor parent. Backcrossing methods can also be used with the lettuce plants of the present application to improve or introduce one or more characteristic into the lettuce cultivar of the present application.

In some embodiments, a plant for breeding is a lettuce such as, but not limited to, a variety of leaf lettuce, romaine lettuce, Frisée lettuce, butter lettuce, Bibb lettuce, Boston lettuce, iceberg lettuce, kale, spinach, radicchio, or endive. In certain embodiments, the lettuce is a romaine lettuce, such as, but not limited to, Green Forest, Paris Island, Avalanche, Rubicon, Musena, Costal Star, Ideal Cos, Topenga, Ridgeline, Green Towers, Helvius, Jerico, Fresh Heart, Claremont, Show Stopper, Spretnak, Caesar, Salvius, Marilyn, Defender, Concept, King Henry, Pipeline, Rome 59, Valley Heart, Wildcat, Bali, or Mondo.

### Molecular Breeding Evaluation Techniques

In some embodiments, the combination of PPO mutations or the breeding of PPO mutations into new lettuce plants, or cultivars are performed using molecular markers to track the mutations. The term “marker” refers to a nucleotide sequence or a fragment of such sequence, e.g., a single nucleotide deletion, used as a point of reference at an identifiable physical location on a chromosome (e.g., restriction enzyme cutting site, gene) whose inheritance can be tracked. Markers can be derived from genomic nucleotide sequences or from expressed nucleotide sequences (e.g., from a spliced RNA, cDNA, etc.). The term can also refer to nucleic acid sequences complementary to or flanking a marker. The term can also refer to nucleic acid sequences used as a molecular markers probe, primer, primer pair, or a molecule that can be used to identify the presence of a marker locus, e.g., a nucleic acid probe that is complementary to a marker locus sequence, and is capable of amplifying sequence fragments using PCR and modified PCR reaction methods.

A marker may be tracked using a marker assay. The term “marker assay” refers generally to a molecular markers assay, such as PCR, KASP, PACE or SSR, for example, used to identify whether a certain DNA sequence or SNP, for example, is present in a sample of DNA. For example, a marker assay can include a molecular markers assay, e.g., KASP assay, which can be used to test whether PPO mutation is present. Markers corresponding to genetic polymorphisms between members of a population can be detected by methods commonly used in the art including, PCR-based sequence specific amplification methods, detection of restriction fragment length polymorphisms (RFLPs), detection of amplified variable sequences of the plant genome, detection of simple sequence repeats (SSRs), detection of single nucleotide polymorphisms (SNPs), or detection of amplified fragment length polymorphisms (AFLPs), and detection of randomly amplified polymorphic DNA (RAPD). In other embodiments, nucleic acids may be detected with other high throughput hybridization technologies including microarrays, gene chips, LNA probes, nanoStrings, and fluorescence polarization detection among others. Other forms of nucleic acid detection can include next generation sequencing methods such as DNA SEQ or RNA SEQ using any known sequencing platform including, but not limited to: Roche 454, Solexa Genome Analyzer, AB SOLiD, Illumina GA/HiSeq, Ion PGM, MiSeq, among others.

Detection of markers can be achieved at an early stage of plant growth by harvesting a small tissue sample (e.g., branch, or leaf disk). This approach is preferable when working with large populations as it allows breeders to weed out undesirable progeny at an early stage and conserve growth space and resources for progeny which show more promise. In some embodiments the detection of markers is automated, such that the detection and storage of marker data is handled by a machine. Recent advances in robotics have also led to full service analysis tools capable of handling nucleic acid/protein marker extractions, detection, storage and analysis.

Although certain embodiments have been depicted and described in detail herein, it will be apparent to those skilled in the relevant art that various modifications, additions, substitutions, and the like can be made without departing from the spirit of the present application and these are therefore considered to be within the scope of the present application

The following examples are intended to illustrate but not limit the invention.

### EXAMPLES

### Example 1—Chimeric Nuclease and PPO Gene Editing in Lettuce Protoplasts Construction of SynNuc1 Vector Targeting Eight Lettuce Polyphenol Oxidase (PPO) Genes through a Polycistronic gRNA

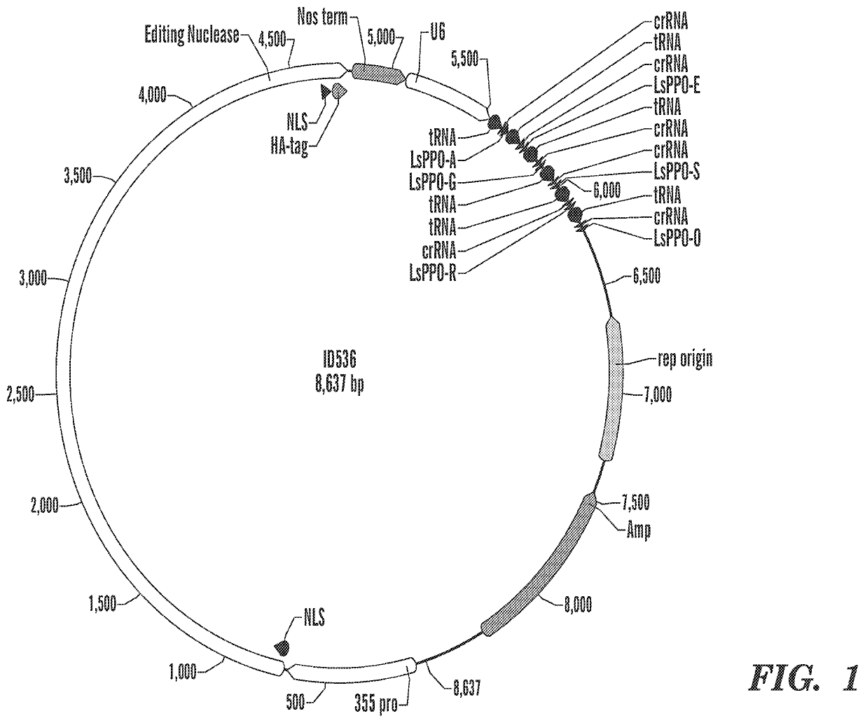

A vector was synthesized containing polycistronic gRNAs containing six independent gRNAs (A, E, G, O, R, and S, SEQ ID NOs:41-46, Table 2). These guide RNAs target eight different lettuce PPO genes (PPO-A, PPO-B, PPO-C, PPO-E, PPO-G, PPO-O, PPO-R, and PPO-S, corresponding to SEQ ID NOs:1, 3, 5, 9, 11, 19, 25, and 27). Vector pID536 also contained a novel chimeric nuclease (SynNuc1, SEQ ID NO:29). A schematic illustration of vector pID536 is shown in FIG. 1. The gRNA for PPO-A (SEQ ID NO:35) targeted three PPO genes (PPO-A, PPO-B, and PPO-C). The gRNAs for PPO-E, PPO-G, PPO-O, PPO-R, and PPO-S (SEQ ID NOs:36-40) targeted respective PPO genes singly.

TABLE 2
Guide RNAs for PPO Gene Editing
Using Cas12a-Type Nuclease

| | | | SEQ |
|---|---|---|---|
| | Targets | | ID |
| Name | Gene(s) | Sequence (5′ to 3′) | NO: |
| gRNA-A | PPO-A, | ATGGGTAGTCCTTATCGTGCAGG | 35 |
| | PPO-B, | | |
| | and | | |
| | PPO-C | | |
| gRNA-E | PPO-E | CGTCCACCATGCGAATGTCGACA | 36 |
| gRNA-G | PPO-G | TTCGGTGGTGAGTATCGTGCCGG | 37 |
| gRNA-O | PPO-O | CGATGAGTCTAGACAAACCAACG | 38 |
| gRNA-R | PPO-R | TTTGGTGGCGAGTTTCGGGCTGG | 39 |
| gRNA-S | PPO-S | ATGCAACAAGCTTCTTCGGTGGC | 40 |

Protoplast cell transfections were performed with a vector ID536 in containing SynNuc1 driven by a CaMV 35S promoter along with a polycistronic gRNAs targeting eight different lettuce PPO genes driven by the Arabidopsis U6 promoter. Protoplasts were isolated from six week old wild type Romaine lettuce plants (about 1 g of leaf tissue) and transfected following Sheen's protocol (Sheen, “A Transient Expression Assay Using Arabidopsis Mesophyll Protoplasts,” available on line at URL genetics.mgh.harvard.edu/sheenweb/(2002); Yoo et al., “Arabidopsis Mesophyll Protoplasts: A Versatile Cell System for Transient Gene Expression Analysis,” Nature Protocols 2:1565-1575 (2007), which are hereby incorporated by reference in their entirety). Transfected protoplasts were incubated at 25° C. in the dark for about 60 hours.

Sixty hours post-transfection, protoplasts from three independent transfection reactions were pooled together and genomic DNA (gDNA) was extracted with 400 μl urea buffer (6.9 M Urea, 350 mM NaCl, 50 mM Tris-Cl pH 8.0, 20 mM EDTA pH 8.0, 1% Sarkosyl) followed by a phenol:chloroform:isoamyl alcohol and a chloroform:isoamyl alcohol steps. DNA precipitation was done at −80° C. for 20 minutes in an equal volume of isopropanol. Finally, DNA was washed once with 70% ethanol and resuspended in 20 μl of distilled (DI) water. gDNA concentration was estimated using a nanodrop 8000 (Thermo Fisher Scientific, Waltham, MA) then diluted at 30 ng/μl for further analysis.

### PCR Amplification of Targeted Region for Gene Editing

Lettuce PDS and/or PPO targeted region were PCR amplified using Phusion Hot Start II (Thermo Fisher Scientific, Waltham, MA) and specific set of primers for each target gene (Table 3, SEQ ID NOs:19-36) using 60 ng/2 μl gDNA following the manufacturer instructions. PCR reactions were run using an Eppendorf MasterCycler EP gradient instrument (Eppendorf, Enfield, CT).

TABLE 3
PCR Primers for Amplification of Gene
Editing Targeted Genes

| | | | | | Am- |
|---|---|---|---|---|---|
| | | Pri- | | SEQ | plicon |
| Target | Primer | mer | Primer | ID | size |
| Gene | Name | ID | Sequence | NO: | (bp) |
| PDS | LsPDS-G2_ | AJ40 | GTTATGTAACTAA | 41 | 200 |
| | DS_AT_F | | CTTTTTCACATTA | | |
| | | | TGC | | |
| | LsPDS-G2_ | AJ41 | ATGTGGGAAGAAA | 42 | |
| | DS_AT_R | | TATCAAATATG | | |
| PPO-A | PPO-A_ZL_ | X35 | ATAGGGTCTCTGG | 43 | 233 |
| | G1A | | CTGCAGA | | |
| | PPO-A_ZL_ | X82 | CGATCGAAACAAA | 44 | |
| | G1S1 | | CCTCCACAAA | | |
| PPO-B | PPO-B_ZL_ | X25 | GGACCATCAACCA | 45 | 217 |
| | G1S | | CCGTCTT | | |
| | PPO-B_ZL_ | X88 | AACTGGGTTATGT | 46 | |
| | G1A1 | | GGTGTGTTCT | | |
| PPO-C | PPO-C_ZL_ | X26 | GCGAAACATCAAC | 47 | 231 |
| | G1S | | CACCGAC | | |
| | PPO-C_ZL_ | X89 | GGTCCAGATATGA | 48 | |
| | G1A1 | | ACCGGGTTAT | | |
| PPO-E | PPO-E_ZL_ | X84 | CTGGGTGGGTAAT | 49 | 227 |
| | G1S1 | | TCTAGGATGG | | |
| | PPO-E_ZL_ | X91 | CTCGGTTGTAGAC | 50 | |
| | G1A1 | | ACGTACAAGA | | |
| PPO-G | PPO-G_ZL_ | X29 | AGGTCACTTGCAA | 51 | 246 |
| | G1S | | GGAGTCG | | |
| | PPO-G_ZL_ | X94 | TTAAGTTCTCTTG | 52 | |
| | G1A1 | | GGTCACCGAC | | |
| PPO-O | PPOO_ZL_ | X86 | ATACCAACTACAC | 53 | 265 |
| | G1S1 | | CTGCCACTTT | | |
| | PPOO_ZL_ | X95 | TATGTCCGTTCCT | 54 | |
| | G1A1 | | GTGCTTATGT | | |
| PPO-R | PPO-R_ZL_ | X33 | TCCGCAACGTCAA | 55 | 222 |
| | G1S | | CCATGTA | | |
| | PPO-R_ZL_ | X96 | CCTCTATTGATCC | 56 | |
| | G1A1 | | GACAGATGGG | | |
| PPO-S | PPO-S_ZL_ | X87 | ACGAAAGTCACCT | 57 | 237 |
| | G1S1 | | TCGGTATGAA | | |
| | PPO-S_ZL_ | Y01 | ATCTGTGAACAGC | 58 | |
| | G1A1 | | CGTATGACAA | | |

### Deep Sequencing of PCR Product to Assess Gene Editing Frequency

Next Generation Sequencing (“NGS”) was used to deeply sequence the specific PCR products for each target gene and assess gene editing frequency by comparing the number of pair reads showing a mutation (indels) at the target site to the number of pair reads depicting a wild type pattern. NGS was performed by the Center for Computational and Integrative Biology DNA Core Facility at Massachusetts General Hospital, Boston, USA using standard protocols.

### Results: SynNuc1 Is Successful in Generating CRISPR-Guided DNA Double-Stranded Breaks in Multiple Genes at Once

All PPO targeted regions were PCR amplified from genomic DNA extracted of these transfected protoplasts then submitted to NGS. Table 4 shows the mutation frequencies observed in all eight lettuce PPO genes at their targeted sites. For example, 0.2% of protoplasts had mutations in PPO-A after transfection with pID536.

TABLE 4
Gene Editing Frequencies Observed in Lettuce
PPO Genes using SynNuc1Gene

| PPO Gene | Mutation Frequency (%) of SynNuc1 |
|---|---|
| PPO-A | 0.2 |
| PPO-B | 0.1 |
| PPO-C | 0.06 |
| PPO-E | 0 |
| PPO-G | 0.07 |
| PPO-O | 0 |
| PPO-R | 0 |
| PPO-S | 0 |

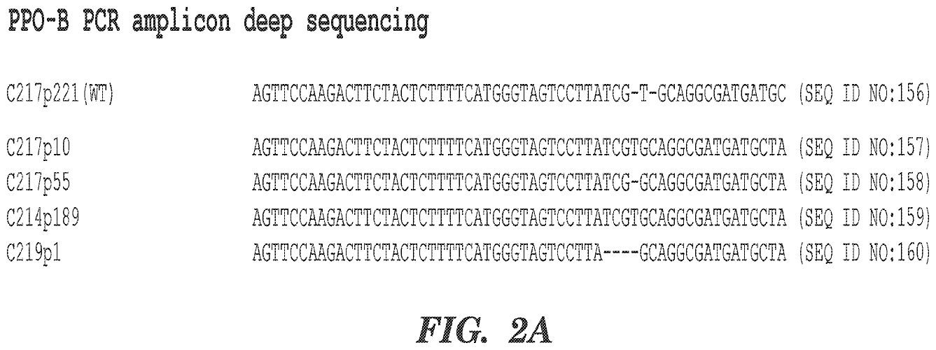

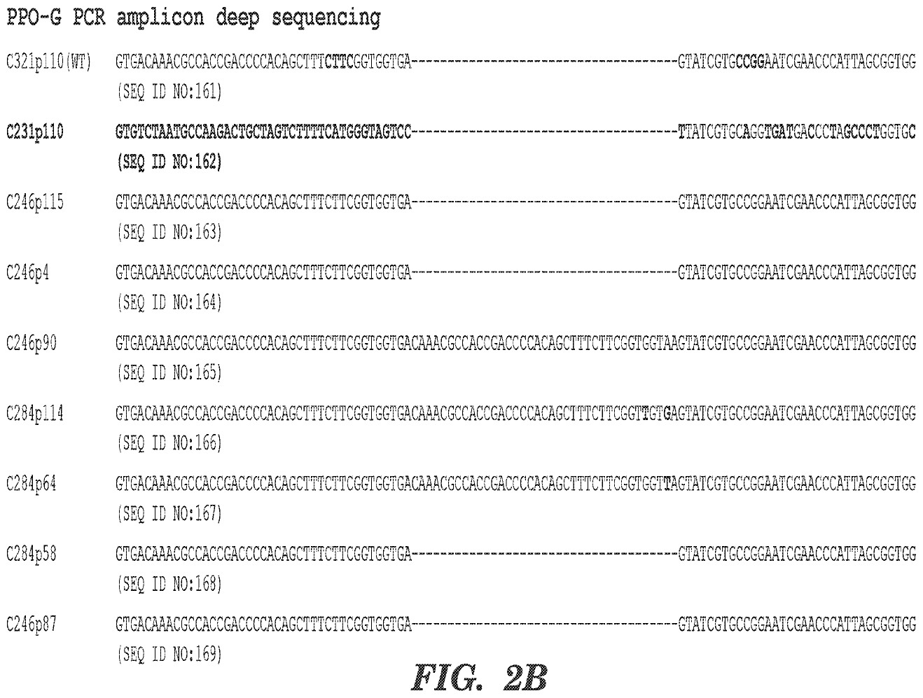

PPO gene mutations were observed in half of the targeted PPO genes. PPO-A, PPO-B, and PPO-C were targeted by the same gRNA while PPO-G was targeted by a different gRNA as were the other four PPO genes (PPO-E, PPO-O, PPO-R, and PPO-S). Mutations generated after NHEJ repair of the breaks created by SynNucl in four PPO genes ranged from one base pair to 5 base pair deletions as well as insertion of one to 37 base pairs. FIGS. 2A-B show examples of mutations generated after repair of the breaks created by SynNuc1 for PPO-B (FIG. 2A) and PPO-G (FIG. 2B). Indels were observed at the targeted cutting site located either 22 base pairs upstream from the protospacer adjacent motif (PAM), TTTN for SynNuc1.

### Example 2—Expression of PPO Genes in Lettuce

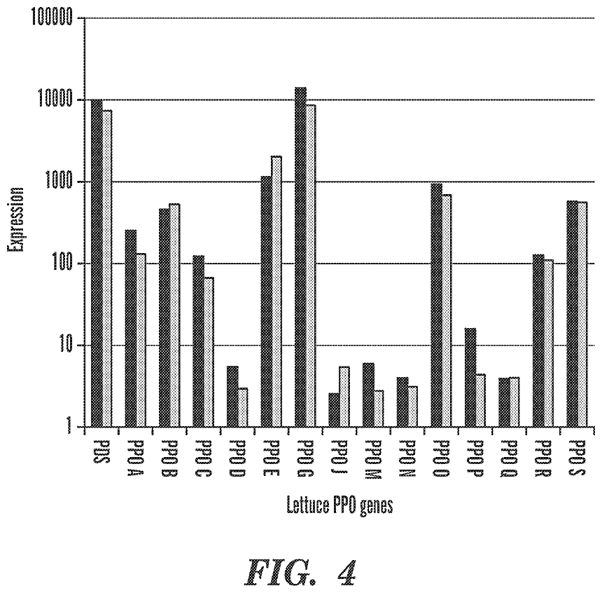

The lettuce genome contains at least 19 putative PPO genes. The expression levels of PPO genes in lettuce vary significantly. FIG. 4 shows the relative expression profile of PPO genes in the leaves of grocery store lettuce. Two sets of primers (shown by black and grey bars) for each PPO gene were used to monitor expression levels in leaves (Table 5, SEQ ID NOs:59-114). Expression of the PDS gene was evaluated using primers SEQ ID NOs:115-118 and used as an expression level control. Eleven PPO genes (excluding PPO-J and PPO-M which had the lowest expression) were selected as targets for gene editing. Romaine lettuce bought at the grocery store was chopped in pieces and three samples were collected: 1) fresh, 2) stored at 4° C. for 4 h, and 3) stored at 4° C. for 3 d before being frozen. RNA was extracted using QIAgen RNeasy Plant Mini Kit following manufacturer instruction. RNA from all three samples were pool together before cDNA synthesis. cDNA was synthesized using Quanta qScript cDNA Synthesis Kit then 100 ng of cDNA was used to perform qRT-PCR.

TABLE 5
PPO Gene Expression Analysis Primers

| | | | | Ampli- |
|---|---|---|---|---|
| | | | SEQ | con |
| Target | Primer | | ID | size |
| Gene | Name | Primer Sequence | NO: | (bp) |
| PPO-A | PPOA 3F | TCTCAAGTTCCCATAGCACGA | 59 | 183 |
| grey | PPOA 3R | TGCATACGCCAATGAGTTTGA | 60 | |
| PPO-A | PPOA 41F | AGCACGAATCACGAACCATC | 61 | 194 |
| black | PPOA 41R | TGAGGTCCGGTGCCATAATC | 62 | |
| PPO-B | PPOB 19F | TTCATATCTGGGCCGGTGAG | 63 | 189 |
| grey | PPOB 19R | ACAAGAACGAAGAATCAAGCCA | 64 | |
| PPO-B | PPOB 21F | CCCTCTGAACAAACGTCCAA | 65 | 158 |
| black | PPOB 21R | TGAACTGGGTTATGTGGTGTG | 66 | |
| PPO-C | PPOC 13F | GGCCTTGGAGGTCTTTATGGT | 67 | 102 |
| grey | PPOC 13R | AGCCGGACCACATTTACTGA | 68 | |
| PPO-C | PPOC 29F | CCATCACAAGCCACCTCTTAC | 69 | 194 |
| black | PPOC 29R | ACGTTTCTCCTATCAAGTTTGCC | 70 | |
| PPO-D | PPOD 1IF | AAATCCCGTGGCTCCATAGTC | 71 | 184 |
| grey | PPOD 11R | TTCGCTTCCTGCTTGTTCTTC | 72 | |
| PPO-D | PPOD 25F | TTTACGTCCACCATGCCAAC | 73 | 186 |
| black | PPOD 25R | AGTCGTATCCCATTGCAGTCA | 74 | |
| PPO-E | PPOE 13F | TGTACGTGTCTACAACCGAGA | 75 | 140 |
| grey | PPOE 13R | CGATTCCAGCAGACTTAGCAG | 76 | |
| PPO-E | PPOE 15F | GCAAATGAGCTGTTGTTCGTG | 77 | 148 |
| black | PPOE 15R | TATGTGGCAACTGTGCGAAAC | 78 | |
| PPO-G | PPOG 8F | CAGTTGCCACACAAACACAAG | 79 | 134 |
| grey | PPOG 8R | GATCCAGACCTTGGGACAATC | 80 | |
| PPO-G | PPOG 22F | ATTAGCGGTGGTGGATCAGTC | 81 | 187 |
| black | PPOG 22R | GTTTGCCTCCCTGCATCTTC | 82 | |
| PPO-J | PPOJ 9F | TATCACGACGCCAAACTTCTC | 83 | 174 |
| grey | PPOJ 9R | CTATGTGCCGCTGGTCTAATC | 84 | |
| PPO-J | PPOJ 10F | ATAAGGCCGTCAGAAGCAGAG | 85 | 200 |
| black | PPOJ 10R | AACGAAACTGTGTGGGTGTTC | 86 | |
| PPO-M | PPOM 8F | GGCCTGAAACTGCTAGAACAA | 87 | 168 |
| grey | PPOM 8R | AACCCGCATACTCGCTATCA | 88 | |
| PPO-M | PPOM 9F | GCGAAGATACGACTGACGACT | 89 | 115 |
| black | PPOM 9R | CCAGCGACATACTTACCACCA | 90 | |
| PPO-N | PPON 19F | ACTTCTACTCCGCAGGCTATG | 91 | 102 |
| grey | PPON 19R | GTGCATGGTGTCTCCTGTTC | 92 | |
| PPO-N | PPON 2IF | TAACGACAATGACCCACACAG | 93 | 148 |
| black | PPON 21R | GTGGAATGGAAAGAAGAGCCA | 94 | |
| PPO-O | PPOO 2F | ACATAAGCACAGGAACGGACA | 95 | 124 |
| grey | PPOO 2R | CACCACCACATCTTCGTCATC | 96 | |
| PPO-O | PPOO 6F | ATGGCCGGAATACCAACTACA | 97 | 199 |
| black | PPOO 6R | CCGCTGTCTCCTCATCCTC | 98 | |
| PPO-P | PPOP 11F | TGTATGTGGATGACAAGGACGA | 99 | 101 |
| grey | PPOP 11R | CTTCTCGCCTGATTGATGGTG | 100 | |
| PPO-P | PPOP 2IF | GGATGCCGAACAATGAAGACA | 101 | 119 |
| black | PPOP 21R | CCTGATACCCAACTCTTTCCAG | 102 | |
| PPO-Q | PPOQ 4F | AGGACCAAGGAGGACAAACAG | 103 | 159 |
| grey | PPOQ 4R | TGCGAAACTACCAGCAAACTC | 104 | |
| PPO-Q | PPOQ 15F | GACAATGGAACCGAGACCAC | 105 | 110 |
| black | PPOQ 15R | CCAAACCTAACTCCGCTCATC | 106 | |
| PPO-R | PPOR 15F | ACGTCAACCATGTATCACCAGA | 107 | 142 |
| grey | PPOR 15R | CTTAACGGGTCAGTAGCGTTG | 108 | |
| PPO-R | PPOR 20F | GCAACTCCAGATTCACAACTCA | 109 | 195 |
| black | PPOR 20R | GCGTCAAAGACAGGGTTTCTC | 110 | |
| PPO-S | PPOS 11F | CCTATTGACTGTTGCCCTCTG | 111 | 120 |
| grey | PPOS 11R | ATCGAGATACCCGACTGGAAG | 112 | |
| PPO-S | PPOS 22F | GGAATTTGCTCCTGGGTCTC | 113 | 126 |
| black | PPOS 22R | ATACGGCATCTTTGCATGGTC | 114 | |
| PDS | PDS 14F | AAATGCTGACGTGGCCTGA | 115 | 119 |
| grey | PDS 14R | CTTTCTCATCCAGTCCTGAACAC | 116 | |
| PDS | PDS 36F | CAGCTGAGGAATGGATTTCAAGA | 117 | 185 |
| black | PDS 36R | GGGTCGACATGGTTCACAATC | 118 | |

### Example 3—Editing of PPO Genes by Agrobacterium-Mediated Transformation and Screening

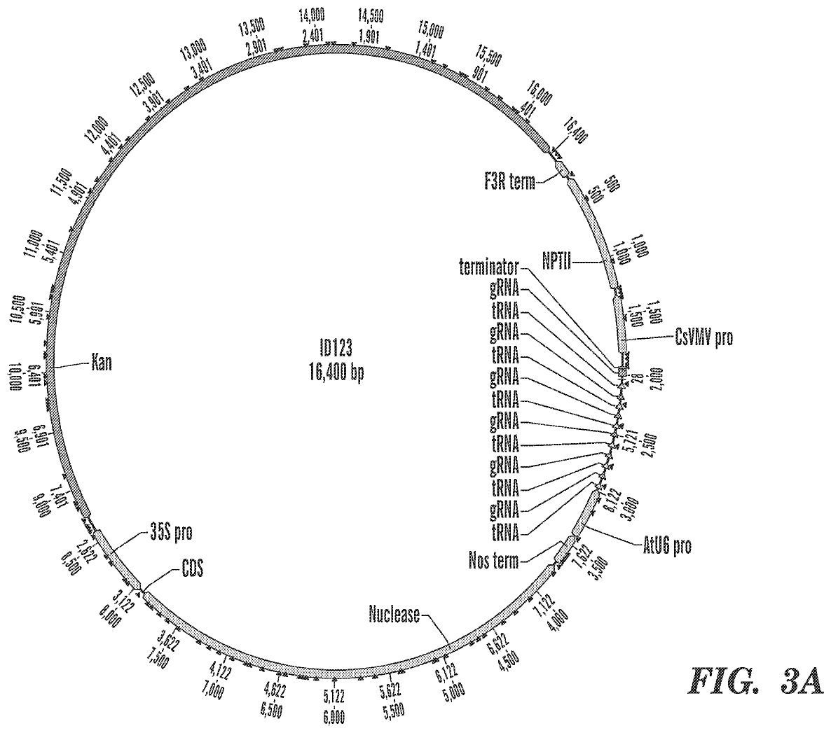

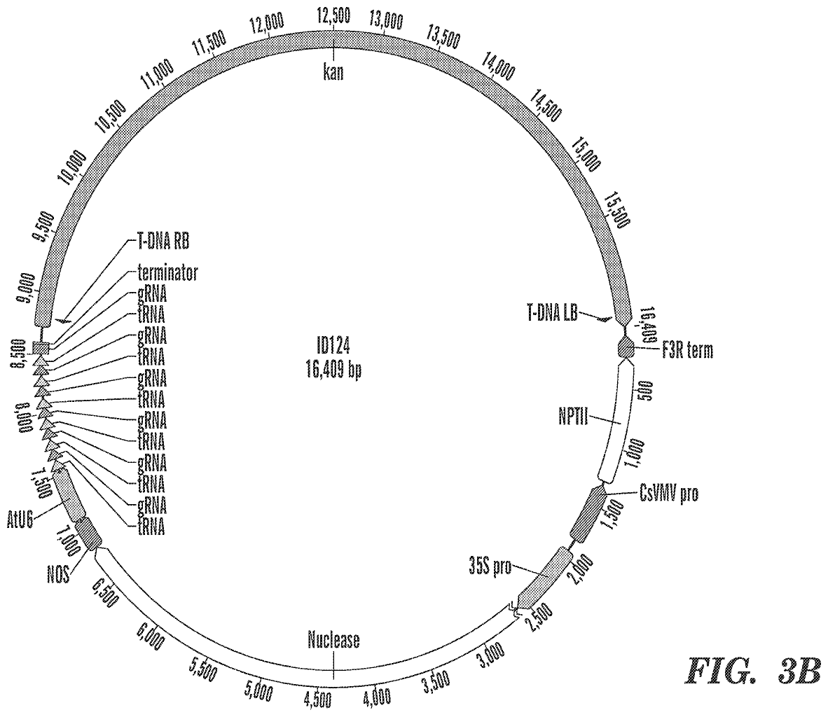

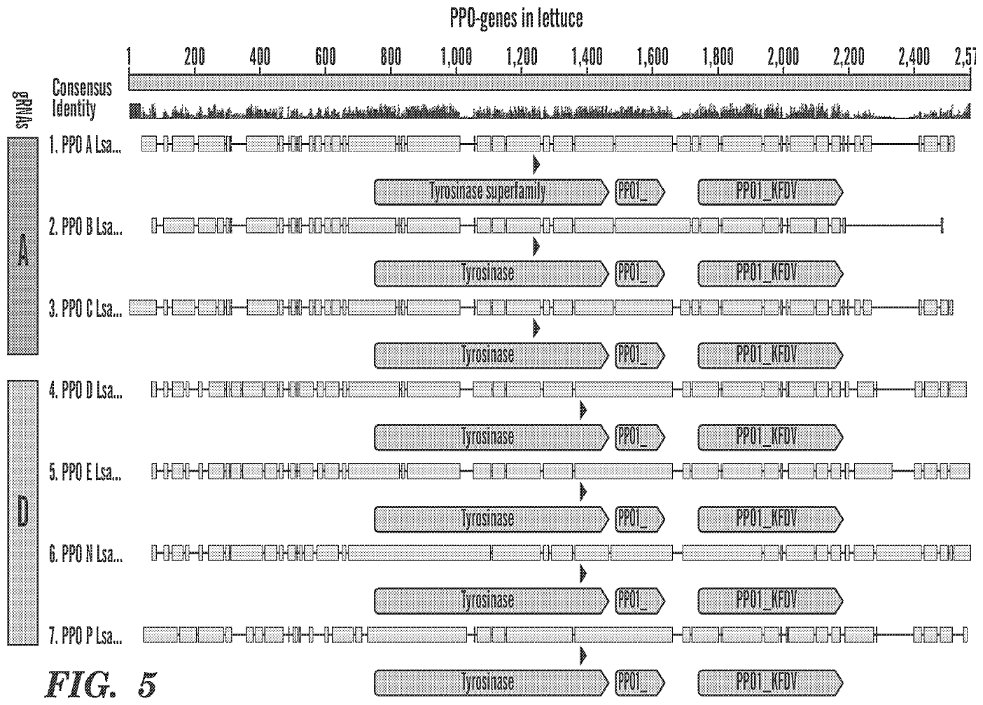

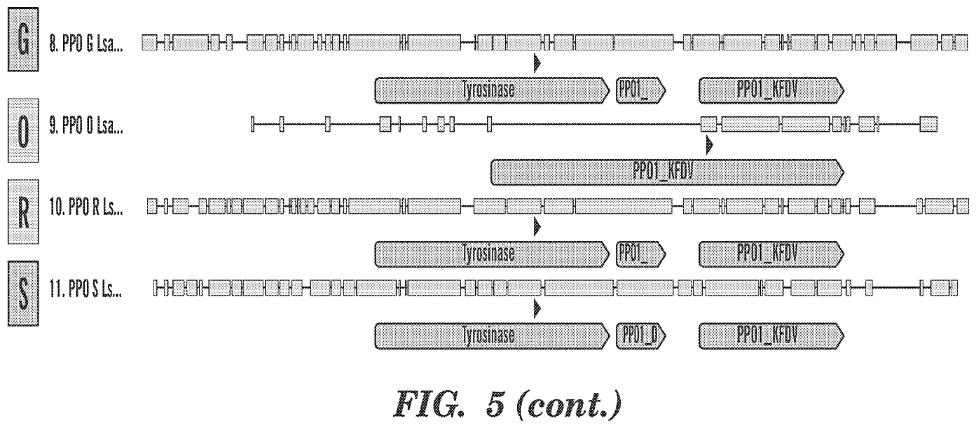

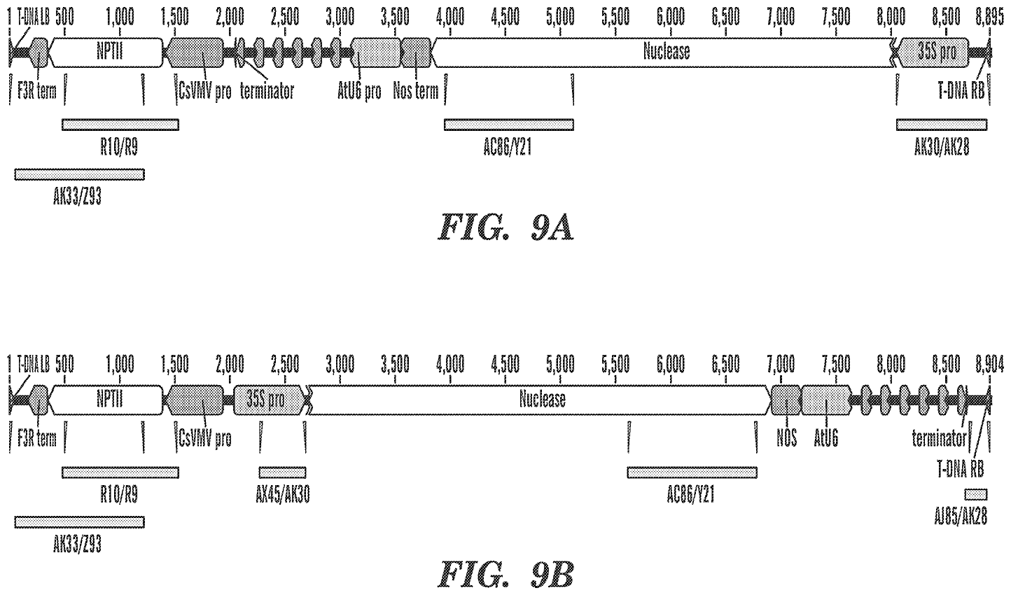

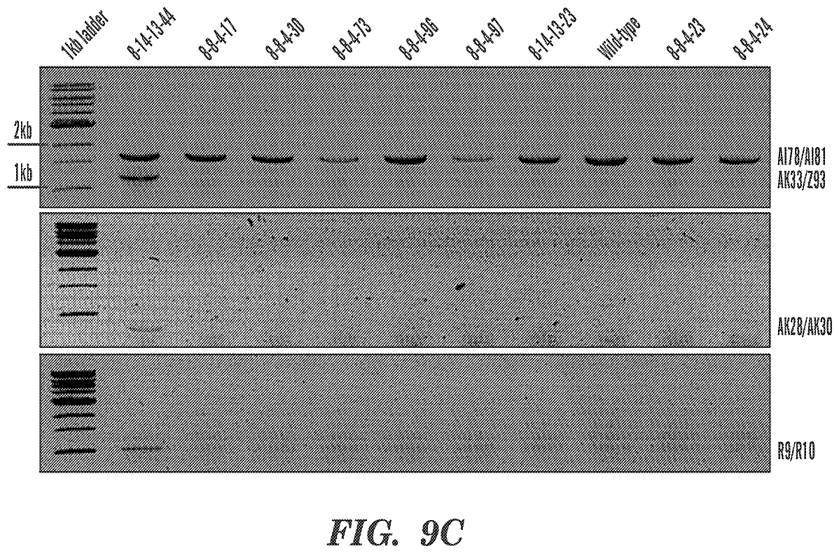

Six gRNAs (gRNA-AA, gRNA-DD, gRNA-GG, gRNA-OO, gRNA-RR, and gRNA-SS, corresponding to SEQ ID NOs:119-124, Table 6) were arranged in a polycistronic fashion on a vector for targeting PPO genes in a romaine lettuce variety. gRNA-AA targets PPO-A, PPO-B, and PPO-C, while gRNA-DD targets PPO-D, PPO-E, PPO-N, and PPO-P. In this way, 11 PPO genes were targeted by the polycistronic construct (FIG. 5). The construct also contained a coding sequence for a Cas9-type gene editing nuclease. Two vectors were made with the construct in opposite orientations, ID123 and ID124 (FIGS. 3A-B and FIGS. 9A-B).

TABLE 6
Guide RNAs for PPO Gene Editing
using Cas9-Type Nuclease

| | Targets | Sequence | SEQ ID |
|---|---|---|---|
| Name | Gene(s) | (5′ to 3′) | NO: |
| gRNA-AA | PPO-A, | TGGGTAGTCCTTATCGTGC | 119 |
| | PPO-B | | |
| | and, | | |
| | PPO-C | | |
| gRNA-DD | PPO-D, | TGGGGAACTTCTACTCCGC | 120 |
| | PPO-E, | | |
| | PPO-N | | |
| | and, | | |
| | PPO-P | | |
| gRNA-GG | PPO-G | TCGGTGGTGAGTATCGTGC | 121 |
| gRNA-OO | PPO-O | AAACCAACGACGGTGATGG | 122 |
| gRNA-RR | PPO-R | TTGGTGGCGAGTTTCGGGC | 123 |
| gRNA-SS | PPO-S | TCGGTGGCAAATATGTAGC | 124 |

Plants were regenerated following Agrobacterium-mediated transformation with ID123 and ID124 using published methods (Curtis, “Lettuce (Lactuca sativa L.),” in Agrobacterium Protocols, Second Edition, Volume 1, eds. Kan Wang, pp449-458 (2006), which is hereby incorporated by reference in its entirety).

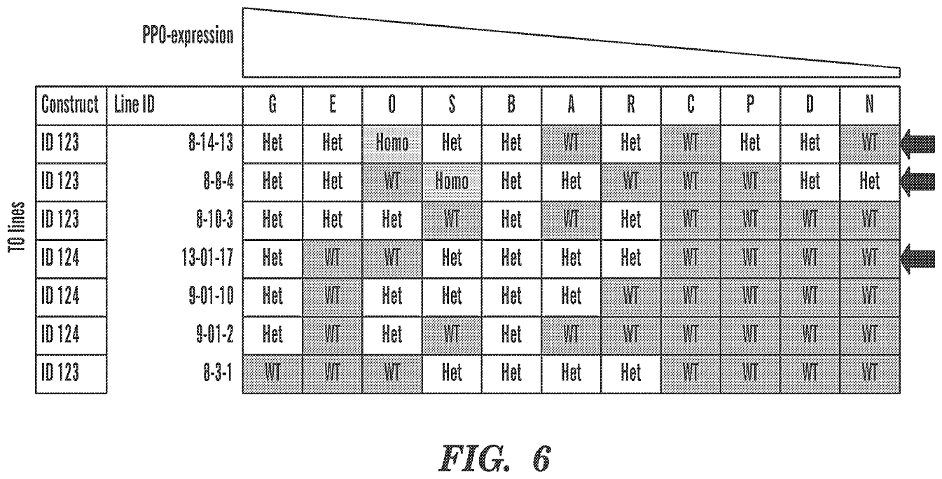

Events were selected on kanamycin and kanamycin positive events were analyzed for the presence of guide RNA and gene editing nuclease coding sequences cassette intactness and transgene copy number. A total 145 T₀ lines from ID123 and 72 T₀ lines from ID124 were obtained. Over 90% of lines from both constructs were low or single copy events. Approximately 50 single or low copy T₀ events from both ID123 and ID124 constructs were analyzed for mutations in PPO genes using Surveyor™ Mutation Detection kit (Integrated DNA Technologies, Newark, NJ), and mutations in targeted PPO genes were further confirmed by PCR-amplicon sequencing. Listed in FIG. 6 are seven key events that had indels in different targeted PPO genes. T₀ lines 8-8-4 and 8-14-13 from ID123 had 7 and 8 PPO genes mutated, respectively. To line 13-01-17 from construct ID124 was also advanced for further analysis. The 13-01-17 T₀ line had 5 PPO genes mutated. Mutations in PPO genes are listed in Table 1.

### Analysis of T1 Segregated Events, Mutations in PPO Genes, and PPO Enzymatic Activity

Thirty-two T₁ siblings from T₀ line 8-8-4 and 20 siblings from line 8-14-13 were screened for presence or absence of the expression cassette and mutations in targeted PPO genes. All transgene free T₁ siblings were also analyzed for PPO enzymatic activity. Seven millimeter leaf disks harvested from the outermost healthy leaf free of the mid-rib were flash frozen then grind in a Tissuelyzer for 1 minute@25 Hz. Samples were re-frozen in liquid nitrogen and homogenized for an additional minute before adding 250 μl of protein extraction buffer containing 100 mM sodium phosphate pH 6.8, 1% PVPP and, 1% NP −40 and shake by hand for 30 seconds. After microcentrifugation for 30 seconds at maximum speed, 125 μl of the supernatant was transferred to a filter plate. The flow through collected after microcentrifugation for one minute at maximum speed was used as crude protein extract in the PPO assay. PPO assay was performed in a flat bottom 96-well assay plate containing 10 mM pyrocatechol in a solution containing 100 mM sodium phosphate pH 6.8 and 0.015% SDS. Thirty microliter of crude protein extract was quickly added to the pyrocatechol substrate using a multichannel pipet and measurement of PPO activity at 410 nm was rapidly initiated since activity will level off within a few minutes. Table 7 below, shows the transgene-free T₁ siblings, their PPO genotypes, and PPO enzymatic activity in leaves.

TABLE 7
Mutations Observed in T₁ Plants

| ID | | | PPO-activity |
|---|---|---|---|
| Control | Homo-PPO | Het-PPO | High |
| 8-8-4-5 | ADES | B | Undetectable |
| 8-8-4-23 | BDES | — | Low |
| 8-8-4-24 | BDEGS | — | Undetectable |
| 8-8-4-30 | DES | BA | Low |
| 8-8-4-73 | BES | GA | Low |
| 8-8-4-88 | BDES | GA | Low |
| 8-8-4-96 | BDEGS | A | Undetectable |
| 8-8-4-97 | ABES | G | Low |
| 8-8-4-101 | ABS | GE | Undetectable |
| 8-14-13-4 | OR | GESBPD | Undetectable |
| 8-14-13-5 | BOPRS | — | High |
| 8-14-13-7 | DEOS | BR | Undetectable |
| 8-14-13-10 | GOR | ESPD | Undetectable |
| 8-14-13-11 | O | GESBRPD | High |
| 8-14-13-16 | OPS | GBR | Low |
| 8-14-13-38 | BDEORS | G | Undetectable |
| 8-14-13-44 | OPS | GBR | High |
| 13-1-17-1 | ARS | GB | High |
| 13-1-17-4 | A | GSR | High |
| 13-1-17-7 | BS | GR | High |
| 13-1-17-9 | B | SR | High |
| 13-1-17-17 | S | GBA | High |

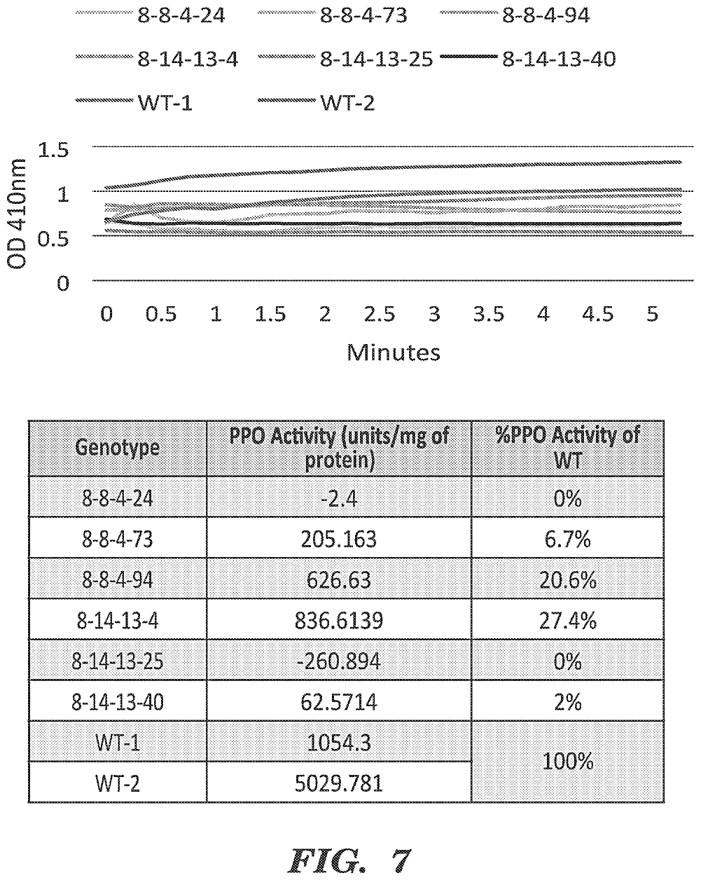

As shown in Table 7, PPO genotypes had variable levels of PPO enzyme activity in leaves. There was no clear single PPO gene found as a sole contributor to the PPO level in lettuce leaves. PPO activity of T1-siblings 8-8-4-24, 8-8-4-73, 8-8-4-94, 8-14-13-4, 8-14-13-25, 8-14-13-40 and two wild type genotypes are shown in FIG. 7.

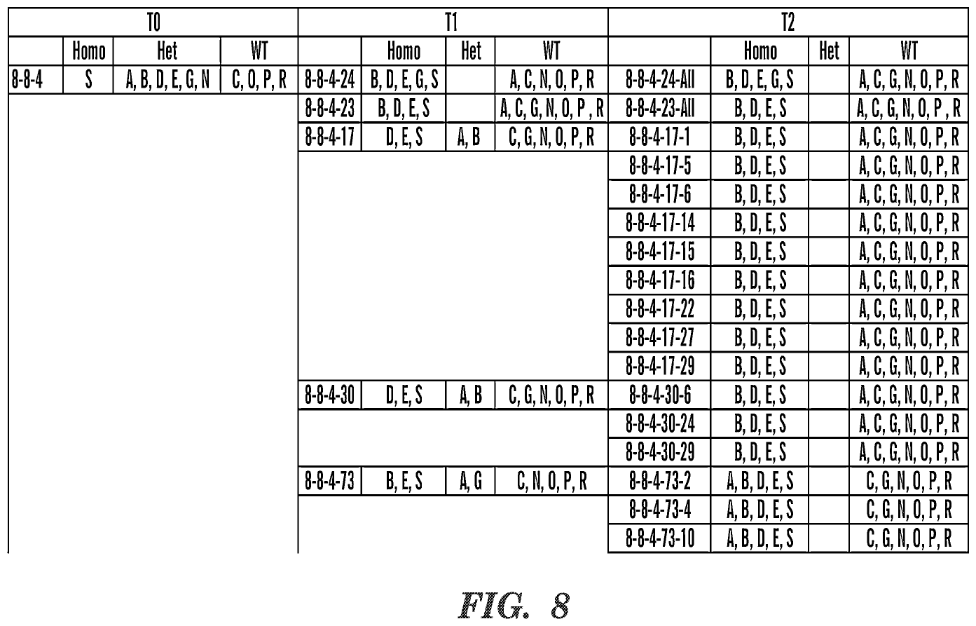

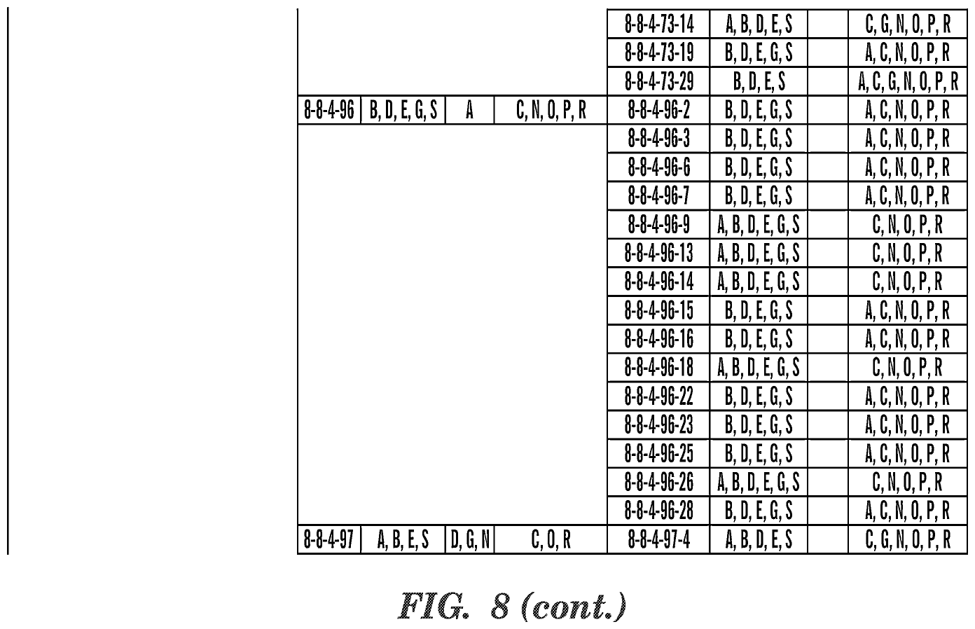

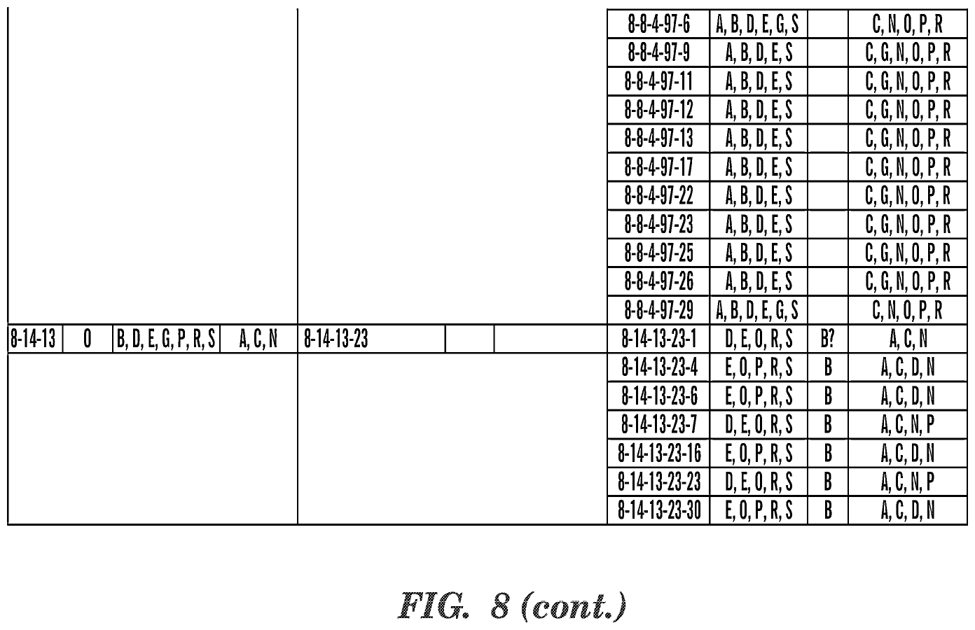

A representative number from each of the T₁ events shown in Table 7 were advanced to T₂ in an effort to fix the heterozygous PPO edits as homozygous. FIG. 8 shows the advanced T₂ siblings with their respective homozygous PPO genes alongside the T₁ and T₀ parents showing the progression to homozygosity.

PPO activity was also measured in the T3 generation plants that were harvested from indoor aeroponic or outdoor field growth environments using the PPO enzyme activity assay as described above. Under aeroponic conditions, the control variety had a PPO specific activity of about 2900 (units/mg protein), whereas GVR-107 (14-2-24-21) had an average PPO specific activity of about 300 (10% of control) and GVR-108 (14-2-24-5) had a PPO specific activity of about 850 (29% of control). Under field conditions, the control lettuce variety had a specific activity of 2240, whereas GVR-102 (14-2-73-14) had an average PPO specific activity of about 680 (30% of control), GVR-105 (15-1-9-11) had an average PPO specific activity of about 280 (12.5% of control), and GVR-110 (14-2-96-13) had an average PPO specific activity of about 130 (6% of control).

### Example 4—Plant Pest Element Screening

As PPO mutant lines were advanced to the T₂ generation, multiple PPO gene edits were fixed, i.e., homozygous at both alleles, and only a small percentage remained heterozygous. At this stage the T₁ parents were screened by PCR to ensure that none had plant pest sequences remaining in the genome. Initial scoring was based on failure to amplify a portion of the NptII gene using NptII primers (SEQ ID NOs:125-126). Absence of the plant pest DNA sequences was confirmed by three additional sets of PCR primers designed to amplify different parts of the construct CsVMV: (R9/R10, SEQ ID NOs:125-126), Border: (AK33/Z93, SEQ ID NOs:129-130), and Border/CaMV35S: (AK28/AK30 SEQ ID NOs:131-132). An additional primer set: (A178/A181 SEQ ID NOs:135-136) which amplifies the endogenous PDS gene was included in all PCR reactions as a positive control for DNA quality. Primers used to screen for plant pest elements are shown in Table 8. FIG. 9A and FIG. 9B are diagrammatic representations of the ID123 and ID124 constructs showing the location of the genetic elements and each primer set.

TABLE 8
Primers Used to Screen for Plant Pest Elements

| | | | | | | Ampli- |
|---|---|---|---|---|---|---|
| | | | | | SEQ | con |
| Target | Plasmid | Primer | Primer | Primer | ID | size |
| Gene | ID | Name | ID | Sequence | NO: | (bp) |
| CsVMV & | ID123 & | CsVMV_AT_ | R9 | TAAGGAAC | 125 | 1018 |
| | | F9138 | | CAAGTTCG | | |
| | | | | GC | | |
| NPTII | ID124 | NPTII_AT_ | R10 | ACACCAAG | 126 | |
| | | R10141 | | CCTTCCAC | | |
| | | | | AG | | |
| CaMV | ID124 | 35S_AT_F | AK30 | GTCTGGAG | 127 | 417 |
| 35S | | | | GATCTGCT | | |
| | | | | AGAGTC | | |
| | | p35S-int_ | AX45 | CGTCTTCA | 128 | |
| | | AT_F | | AAGCAAGT | | |
| | | | | AGG | | |
| LB & | ID123 & | LBATF | AK33 | TGGCAGGA | 129 | 1219 |
| | | | | TATATTGT | | |
| | | | | GGTG | | |
| NPTII | ID124 | Kan_ZL_ | Z93 | GTACTCTT | 130 | |
| | | F1 | | GCCGACTA | | |
| | | | | CAACATC | | |
| RB & | ID123 | 35S_AT_ | AK30 | GTCTGGAG | 131 | 843 |
| | | F | | GATCTGCT | | |
| | | | | AGAGTC | | |
| CaMV | | RB_AT_R | AK28 | GTTTACCC | 132 | |
| 35S | | | | GCCAATAT | | |
| | | | | ATCCT | | |
| RB | ID124 | ID173_ | AJ85 | AGGGCGAA | 133 | 185 |
| | | gRNA_RB_ | | TTCGACCC | | |
| | | GSP1_GW_ | | AGCTTTCT | | |
| | | AT_F | | TG | | |
| | | RB_AT_R | AK28 | GTTTACCC | 134 | |
| | | | | GCCAATAT | | |
| | | | | ATCCT | | |
| PDS | N/A | LsPDS_ | A178 | ATGTCTCT | 135 | 1758 |
| | | ATG_AT_F | | GTTTGGAA | | |
| | | | | ATGTTTC | | |
| | | LsPDS_ | A181 | TCCCATCA | 136 | |
| | | 1.6kb_ | | CTCAATAG | | |
| | | AT_R | | AAAGTTC | | |
| Cas9 | ID123 & | Cas9_ZL_ | Y21 | AATGACAA | 137 | 1169 |
| | ID124 | F5 | | ACTCATCA | | |
| | | | | GGGAAGTG | | |

Plant events were screened with all four primer pairs with the results confirming that no plant pest DNA remained. Results of this PCR screen using the primers shown in FIG. 9C. Primers A178/A181 amplify the endogenous PDS gene and are a PCR positive control. RB primers did not amplify any 8-8-4 lines including the control and all other primer combos indicate absence of pest sequences. T1 PPO mutant lines appear indistinguishable from wild type indicating the absence of plant pest sequences.

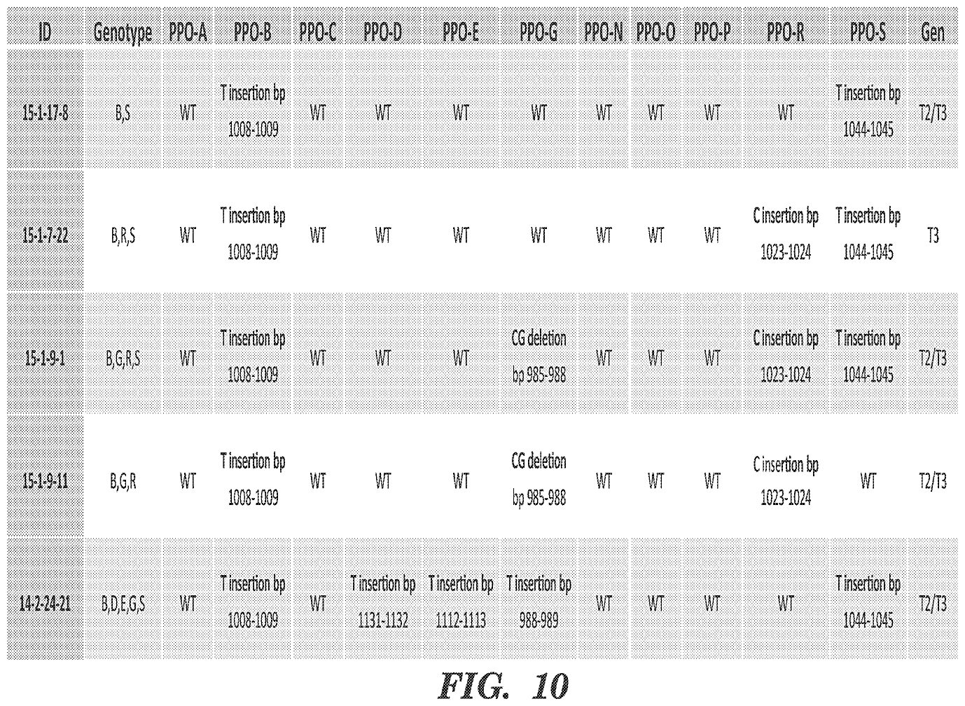

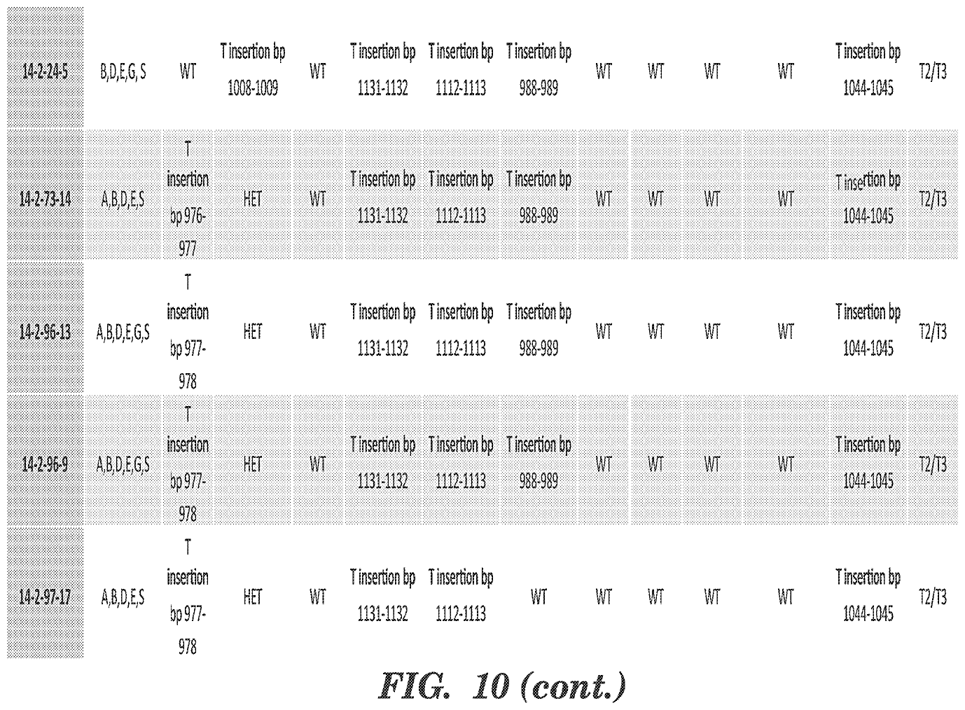

As shown in FIG. 10, the PPO mutant lines were sequenced to confirm the PPO mutations, to be sure of homozygosity, and to check that seed lot purity had been maintained. T₂ plants were sampled and DNA was extracted. PPO genes were amplified by PCR across the region targeted by the respective gRNAs. At the T₂ generation, all the mutations confirmed the earlier sequencing and showed the progression to homozygosity except with respect to PPO-B in 14-2-73-14, 14-2-96-13, 14-2-96-9, and 14-2-97-17 in which the mutation in PPO-B was heterozygous (FIG. 10). Sequencing was repeated twice on T₃ generation plants.

The PPO mutant lines of FIG. 10 were grown to assess the phenotype of non-browning and any other observable trait.

### Example 5—Chromosomal Locations of PPO Genes in the Lettuce Genome

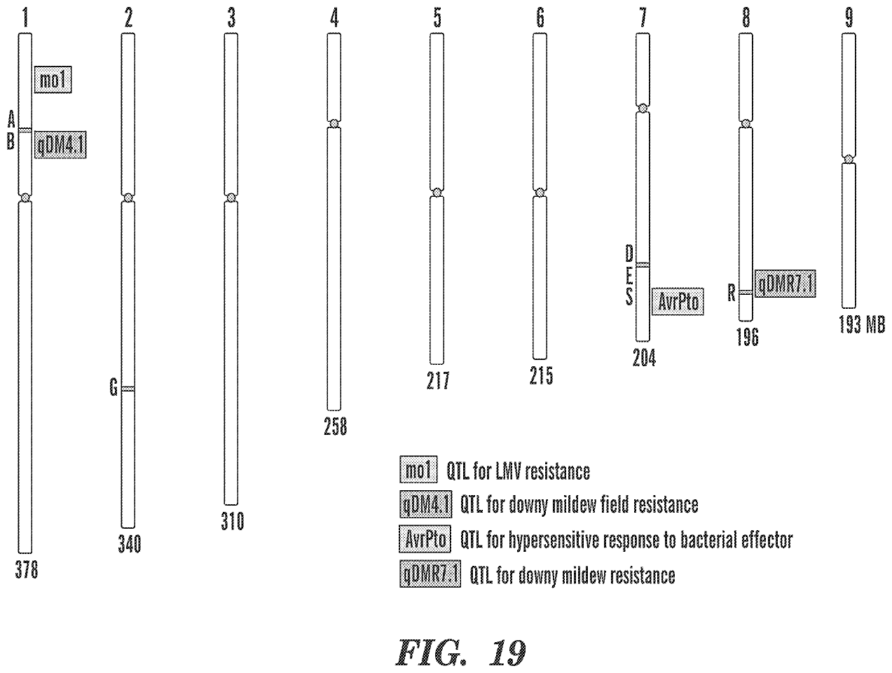

An analysis was completed to pinpoint the locations of the PPO genes across the lettuce genome. This is useful in two ways. From this information, it can be determined if any linkage drag would be associated with breeding the PPO mutations into other lettuce varieties. It can also be determined whether a breeding strategy is feasible. Based on the linkage of the 7 KO PPO genes to only 3 physical locations on 3 chromosomes, it is possible to breed the mutations of the best performing modified lettuce varieties to another variety and carry over the PPO edits. FIG. 19 is a representation of the nine lettuce chromosomes showing the location of 7 PPO genes and their relationship to known QTLs for disease resistance.

### Example 6—Phenotypic Analysis of PPO Mutant Lines—Leaf Tip Burn

Evaluation of T2 lines were grown in aeroponic conditions by AeroFarms (“AF”). Plants were evaluated against unmodified wild type romaine lettuce of the same variety, Isogenic (“control”). It was noted that some of the PPO mutant lines showed greatly enhanced resistance to tip burn. Tip burn is a major issue for lettuce quality and is influenced by factors including lack of calcium in young, rapidly developing leaves. Therefore, the PPO mutant lines shown in FIG. 10 were evaluated for both non-browning and tip burn phenotypes at harvest (day 0), and at days 3, 7, 9, 10, 14, 16, 19, 22, and 26 post-harvest under conditions of light and cold that are considered optimal for this lettuce or at 70° F. in the dark (poor conditions).

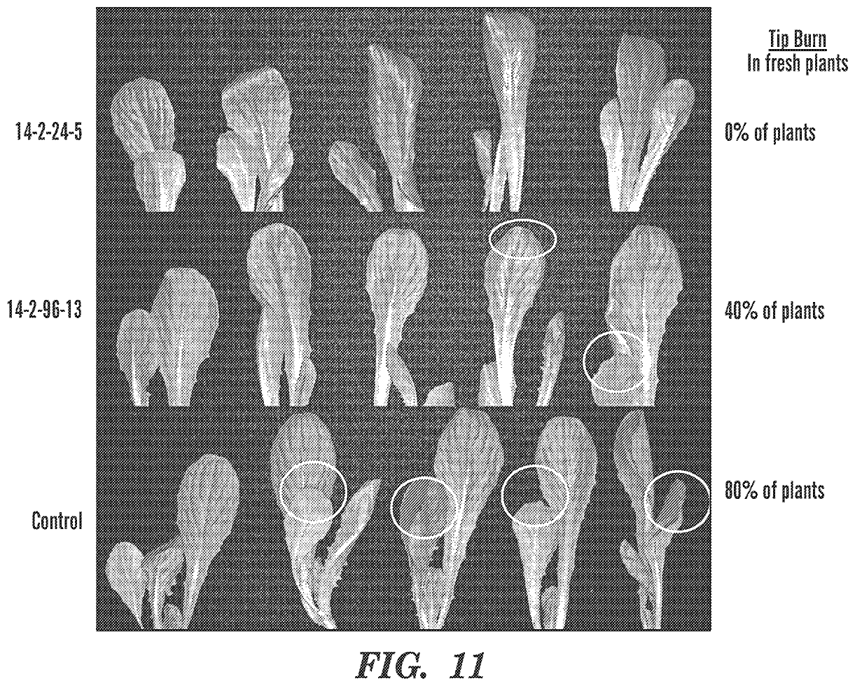

The plants were examined at harvest and it was noted that 80% of the control wild type plants (which are known to exhibit resistance to tip burn) nevertheless exhibited tip burn. In sharp contrast, only 40% of the 15-2-96-13 plants showed tip burn and none of the 14-2-24-5 plants showed tip burn at harvest (FIG. 11). This was a remarkable difference in the new PPO mutant variety.

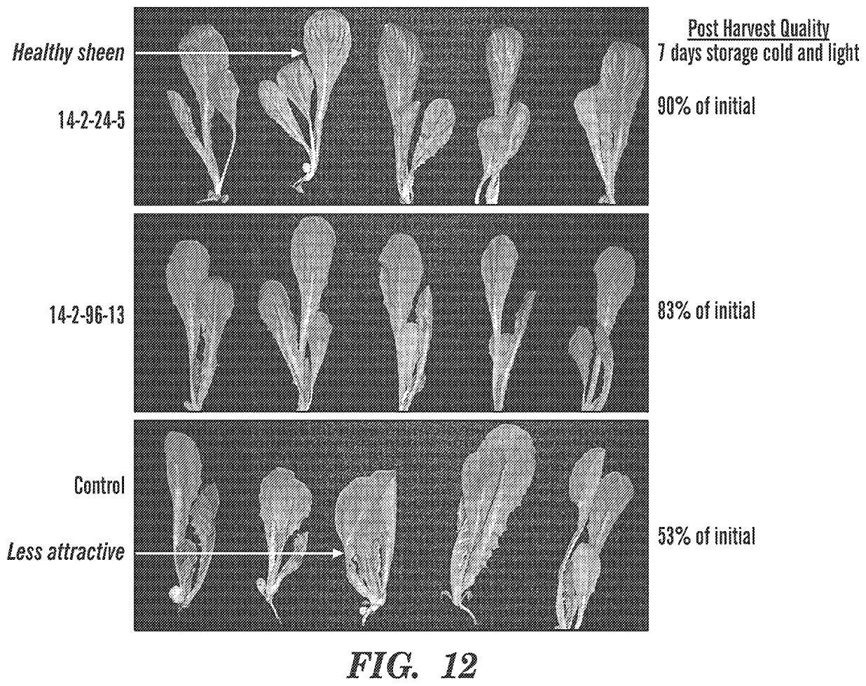

Plants were assessed 7 days post-harvest after storage under optimal conditions. While only 53% of the control plants maintained a healthy sheen and appearance from day 0, 83% of 14-2-96-13 plants and 90% of 14-2-24-5 plants maintained their healthy sheen and appearance (FIG. 12).

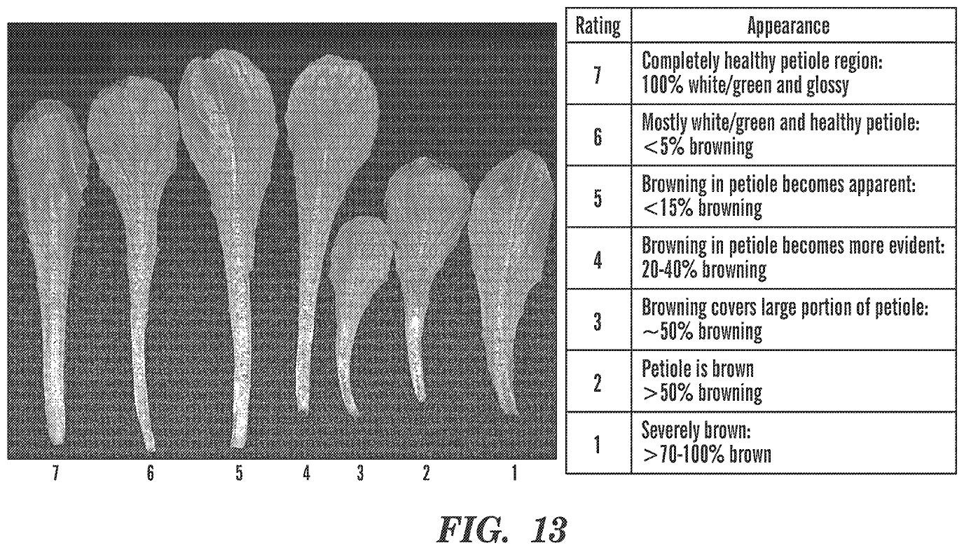

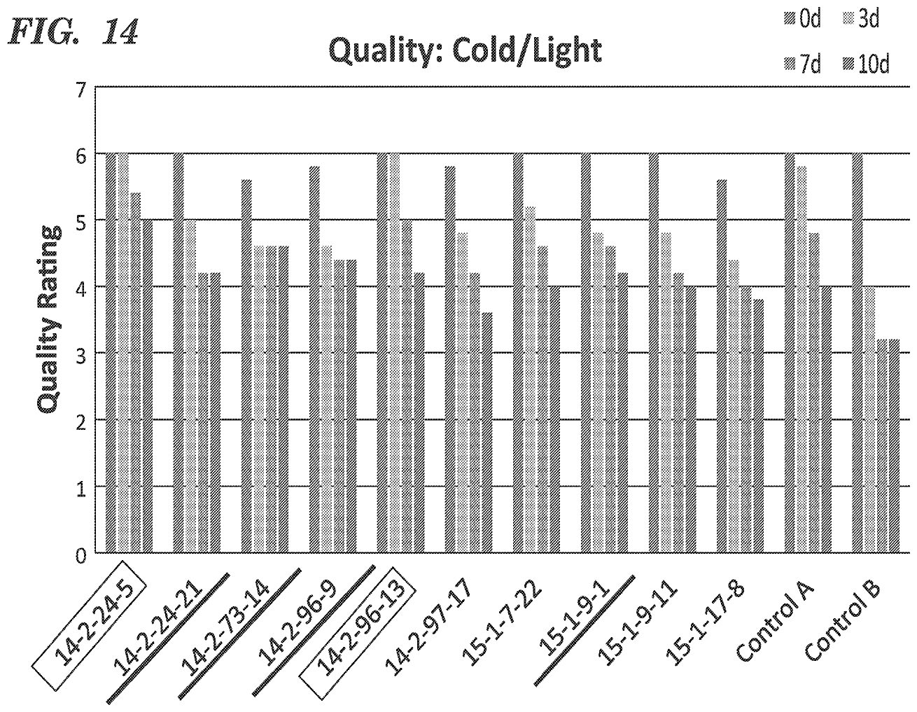

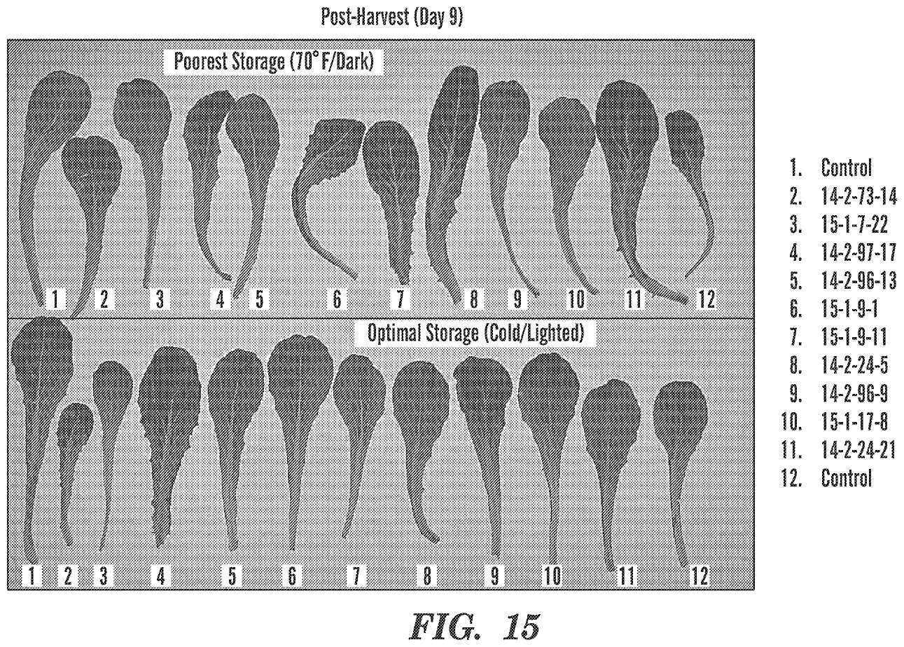

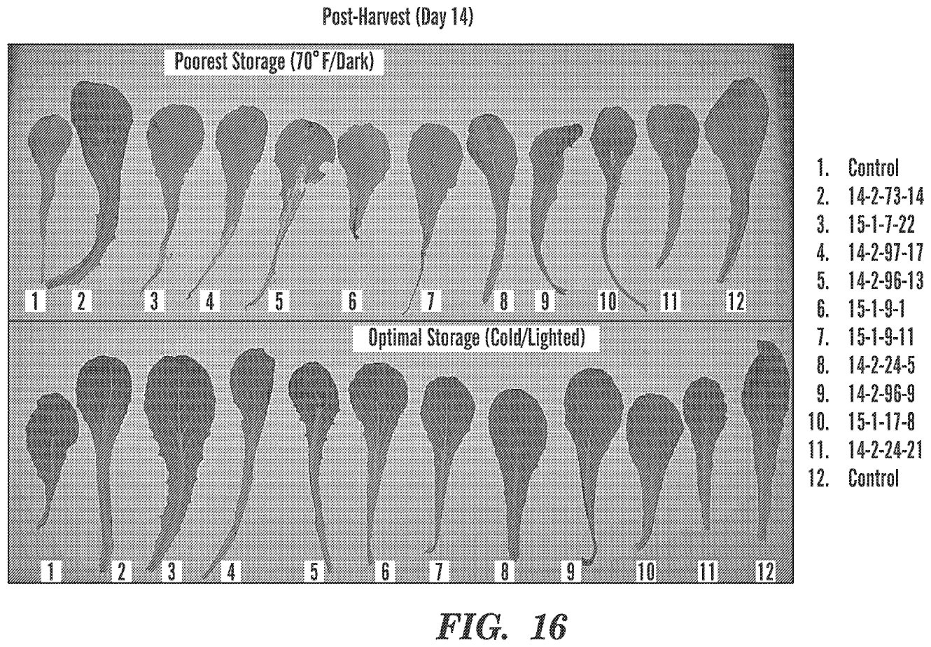

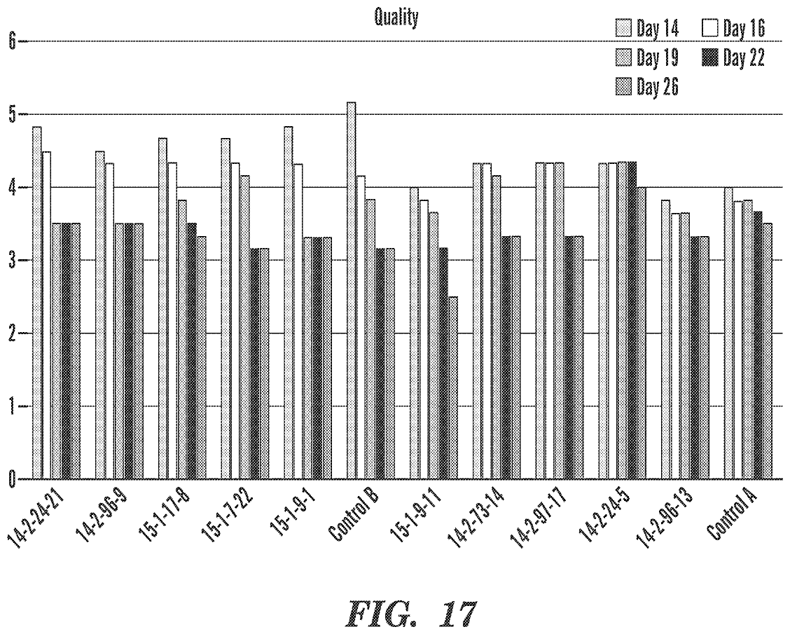

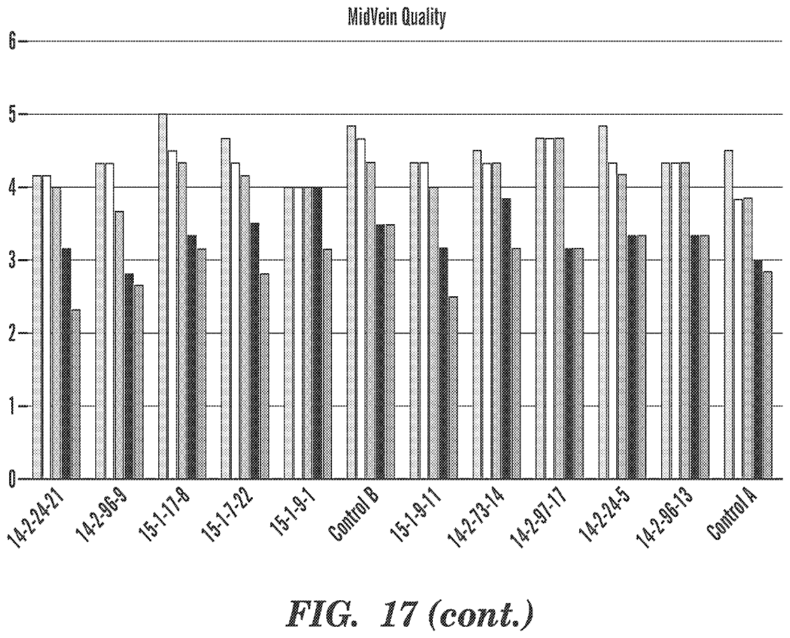

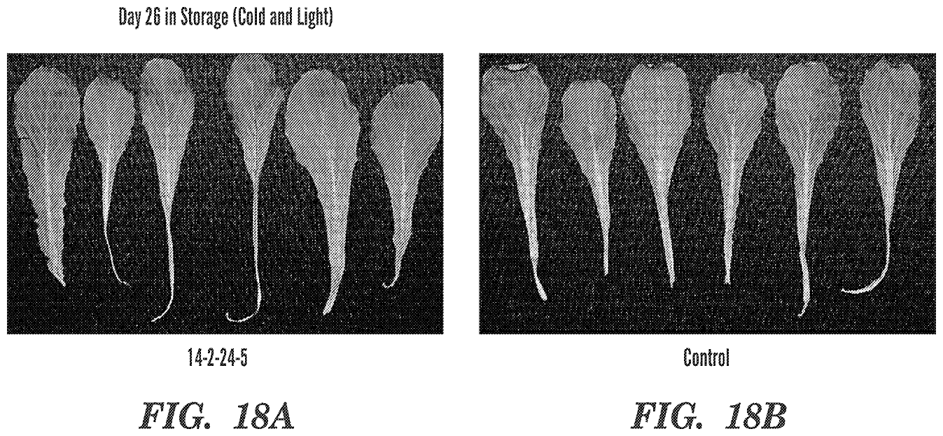

To assess the appearance of the plants, a rating system shown in FIG. 13 (right) was used in which the plants were rated on a 1-7 scale. Examples of leaves exemplary of each rating are shown in FIG. 13 (left). The quality of each of the modified lines and the control lines were assessed at harvest (day 0) and at days 7 and 10 post-harvest after storage under optimal conditions using the 1-7 rating scale. The results are shown in FIG. 14. The PPO mutant lines that are boxed showed the best overall appearance, and the underlined PPO mutant lines also showed good appearance as compared to the control plants through day 10 post-harvest. A comparison of examples of leaves from the PPO mutant lines and the control is shown in FIG. 15 under poorest conditions (top panel) and optimal conditions (bottom panel) at day 9 post-harvest. FIG. 16 shows examples of leaves of the PPO mutant lines and control plant under poorest conditions (top panel) and optimal conditions (bottom panel) at day 14 post-harvest. FIG. 17 shows the assessment of overall quality (left) and midvein quality (right) of control and PPO mutant plants as days 14, 16, 19, 22, and 26 post-harvest. The best PPO mutant lines were 14-2-24-5 and 14-2-95-13. Examples of the leaves of 14-2-24-5 plants and control plants at day 26 post-harvest under optimal conditions is shown in FIG. 18A and FIG. 18B, respectively.

### Example 7—Shelf Life Assessment

Six lines of lettuce that had been edited to knockout PPO genes were selected along with one unmodified parental control to determine the effect of PPO knockout mutations on shelf life of the modified lettuce. The selected varieties were GVR-110 (14-2-96-13) with PPO-A, PPO-B, PPO-D, PPO-E, PPO-G, and PPO-S knockout mutations; GVR-108 (14-2-24-5) with PPO-B, PPO-D, PPO-E, PPO-G, and PPO-S knockout mutations; GVR-105 (15-1-9-11) with PPO-B, PPO-G, and PPO-R knockout mutations; GVR-107 (14-2-24-21) with PPO-B, PPO-D, PPO-E, PPO-G, and PPO-S knockout mutations; GVR-101 (15-1-9-1) with PPO-B, PPO-G, PPO-R, and PPO-S knockout mutations; and GVR-102 (14-2-73-14) with PPO-A, PPO-B, PPO-D, PPO-E, and PPO-S knockout mutations. Clamshell packages containing either a selected line or a control lettuce were stored in a cool room with light. Triplicate clam shells were opened on 3 days (12, 20, and 28 days post-harvest) and scored.

Lettuce with each clamshells were scored on a qualitative basis across several categories, including: off odor, typical aroma, moisture, texture, leaf color, decay/mold, cut edge discoloration, and taste. A total score is an overall rating from the 9 individual scores: 0-5 is product is of excellent quality; 6-8 is product is of very good quality; 9-12 is product is of acceptable quality; 13+ product is of unacceptable quality.

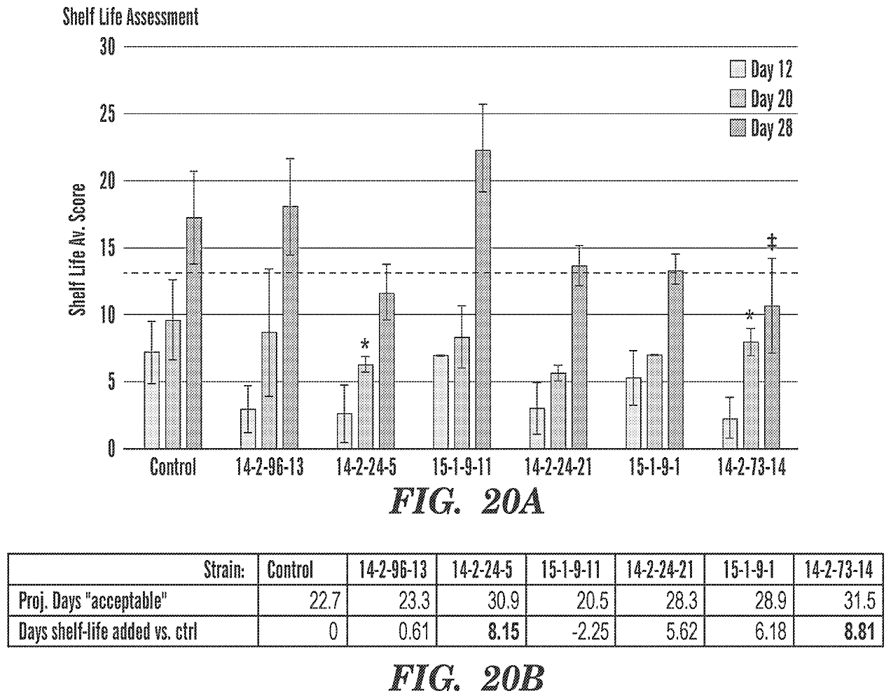

As shown in FIG. 20A, at Day 12 post-harvest, the control and all PPO mutant lines had acceptable shelf life (a shelf life score of less than 13 as shown by the dashed line). At Day 20, the control and all PPO mutant lines had acceptable shelf life scores with lines 14-2-24-5 and 14-2-73-14 having statistically significantly better shelf life scores than the control (*). On Day 28, only PPO mutant line 14-2-73-14 had a statistically significantly better score than the control (‡). FIG. 20B shows the projected number of days that each PPO mutant line would have a shelf life score below 13 (i.e., an “acceptable” score), and the number of days of shelf life improvement over control. PPO mutant lines 14-2-24-5, 14-2-24-21, 15-1-9-1, and 14-2-73-14 each had more than 1 day projected to be added to the shelf life over control, and lines 14-2-24-5 and 14-2-73-14 each had more than 8 days projected to be added to shelf life.

### Example 8—Browning Assay

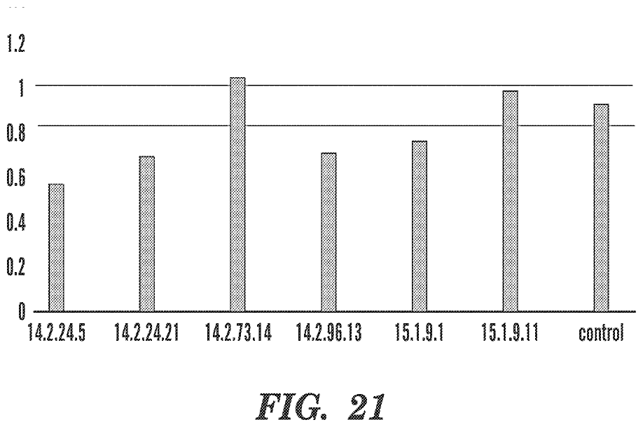

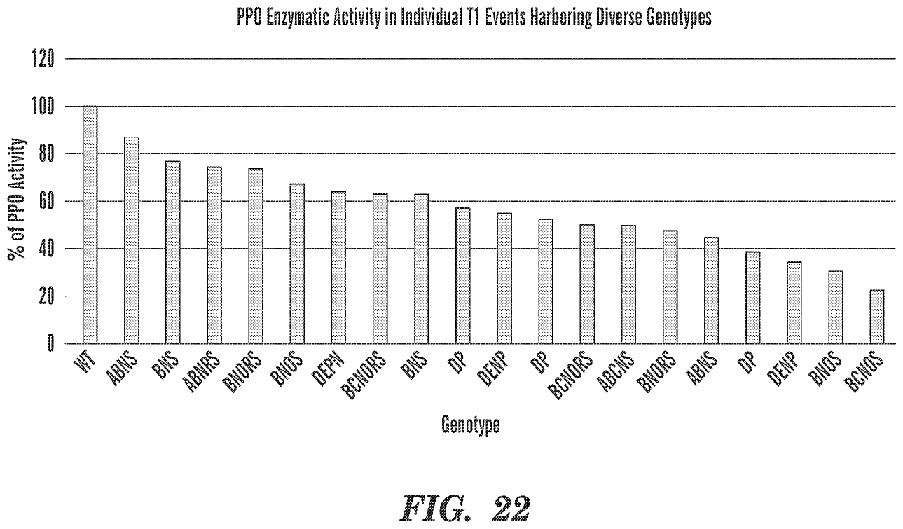

An assay was performed on punched out portions of leaves from each of the 6 PPO mutant lines in Example 7 by examining both area and color intensity of browning across the leaf disk under conditions of cold and light. Four replicates were made for each PPO mutant line and control. The plot shown in FIG. 21 shows the browning rate per day of each PPO mutant line as compared to the control. The grey lines on represent the standard deviation of the control. The graph shows that four of the PPO mutant lines (14-2-24-5, 14-2-24-21, 14-2-96-13, and 15-1-9-1) showed less browning than the control. Leaf discs of 5/16 inch in size were harvested, incubated under cold/light, and browning rate measured using the WinDIAS machine vision system.

### Example 9—High Vitamin Levels and Vitamin Retention in PPO Mutant Lines

PPO mutant lines retained more vitamins after harvest than conventional varieties. PPO mutant lines were grown under both indoor aeroponic (Trial 5) and outdoor field conditions (Trials 1-4, and 6) and tested for vitamin content (Table 9). Trial 1 was field grown lettuce from spring in Salinas, CA, measured at full maturity harvest. Trial 2 was aeroponic grown lettuce in Davis, CA, measured at 21 days after planting. Trial 3 was aeroponic grown lettuce in Davis, CA, measured at 21 days after planting. Trial 4 was outdoor grown lettuce from winter in Yuma, AZ, measured at full maturity harvest. Trial 5 was aeroponic grown lettuce by a partner, measured at 21 days after planting. Trial 6 is outdoor grown lettuce from spring in Salinas, CA, measured at full maturity harvest. In each trial, PPO mutant lines selected from GVR-102 (14-2-73-14), GVR-105 (15-1-9-11), GVR-107 (14-2-24-21), GVR-108 (14-2-24-5), and/or GVR-110 (14-2-96-13) were grown with control lines having no PPO mutations and, in some trials, additional commercial lines (e.g., Red Romaine and/or Isogenic commercial (“GF com”), Trials 4 and 5). Vitamin A was measured in each trial and vitamins C, E, K and folate were measured in a subset of trials. Measurements were taken at two time-points in Trial 5, at harvest and after 21 days of a shelf life study described in Example 13, infra.

TABLE 9
Methods Used for Proximate Analysis

| Analysis | Method |
|---|---|
| Proximate | |
| Protein | AOAC 981.10 |
| Fat | AOAC 960.39 |
| Moisture | AOAC 925.10 |
| Ash | AOAC 923.03 |
| Carbohydrates | Calculated |
| Calories | Calculated |
| Minerals | EPA 7000B |
| K | |
| Vitamin A | AOAC 2001.13 |
| Vitamin K | AOAC 999.15 (HPLC with PCR/FD method) |
| Folate | internal method N248 (based on USP29) |
| Dietary Fiber | AOAC 991.43 |

Most PPO mutant lines, especially GVR-108 and GVR-110, had higher levels of vitamin A compared to control lines in every trial (Table 10). In many trials, PPO mutant lines had double the amount of vitamin A compared to the control line. In trial 5, all of the PPO mutant lines retained double the amount of vitamin A compared to the control at 21 days post-harvest as shown by the second value. The values labeled with an asterisk (*) are significantly higher than the Isogenic control.

Vitamin C was higher in PPO mutant line GVR-110, especially, compared to the Isogenic controls in multiple trials. Vitamin C was retained in some PPO mutant lines after storage (Trial 4). Vitamin E and vitamin K was higher in most PPO mutant lines than controls as well. Folate was higher in the field grown PPO mutant lines compared to the controls in 2 of 3 trials, and GVR-110 had higher folate in all three trials. PPO mutant lines had higher levels of vitamins than control lines and retained more vitamins after storage.

TABLE 10
Vitamin Levels in PPO Mutant Lines and Controls (WT)

| | | Vitamin A | Vitamin C | Vitamin E | Vitamin K | Folate |
|---|---|---|---|---|---|---|
| Sample | | (IU/100 g) | (mg/100 g) | (IU/100 g) | (mg/100 g) | (mg/100 g) |
| Trial 1 | GVR-102 (73.14) | 1177 | | | .126 | .400 |
| (K) | GVR-105 (9.11) | 1353 | | | .202 | .434 |
| | GVR-110 (96.13) | 2200 | | | .130 | .558 |
| | WT | 695 | | | 0 | .266 |
| Trial 2 | GVR-102 (73.14) | 3873 | 1.1 | 0.475 | | |
| (A1) | GVR-105 (9.11) | 3370 | 1.3 | 0.475 | | |
| | GVR-110 (96.13) | 3878 | 3.1 | 0.225 | | |
| | WT | 3355 | 1 | 0.275 | | |
| Trial 3 | GVR-107 (24.21) | 2808 | | | | |
| (A2) | GVR-108 (24.5) | 2940 | | | | |
| | WT | 2863 | | | | |
| Trial 4 | GF Com | 503 | 0.1 | | 0.3 | 0.025 |
| (N) | GVR-102 (73.14) | 947 | 0.2 | | 0.9 | 0.024 |
| | GVR-105 (9.11) | 567 | 0.0 | | 0.4 | 0.030 |
| | GVR-107 (24.21) | 1410 | 0.1 | | 1.2 | 0.039 |
| | GVR-108 (24.5) | 1293 | 0.2 | | 2.1 | 0.041 |
| | GF | 1037 | 0.1 | | 1.0 | 0.043 |
| | GVR-110 (96.13) | 1183 | 0.4 | | 2.5 | 0.049 |
| Trial 5 | Red Romaine | 3934/390 | 470/>10 | 141/157 | | |
| (AF) | GVR-102 (73.14) | 5556/535* | 342/>10 | 112/112 | | |
| | GVR-107 (24.21) | 5676/405* | 378/381* | 111/102 | | |
| | GVR-108 (24.5) | 5345/429* | 431/395* | 115/113 | | |
| | GVR-110 (96.13) | 6757/369 | 445/360* | 109/55 | | |
| | GF | 5946/156 | 398/>10 | 100/104 | | |
| Trial 6 | GVR-102 (73.14) | 980 | | | | 0.0131 |
| (RP) | GVR-108 (24.5) | 1200 | | | | 0.0219 |
| | GVR-110 (96.13) | 1850 | | | | 0.0258 |
| | WT | 230 | | | | 0.0111 |

### Example 10—Lettuce Processing Evaluation for Visual Quality

Lettuce was grown in an open field in Salinas, CA during spring of 2020, harvested, and tested for shelf life. Two to three heads of lettuce from PPO mutant line (GVR-110) and control variety (Isogenic) were removed from the cold room immediately prior to processing. Lettuce was trimmed (outer leaves or broken leaves and tops and butts removed), halved, interior heart tissue removed, and remaining head quartered. Immediately after the head was halved, a photo was taken for maturity assessment. Lettuce was cut with a sharp knife into salad size pieces (approximately 2×2 cm) and then rinsed in 5° C. water containing 50 ppm sodium hypochlorite pH 7.0 for 20 seconds (˜4 volumes water per weight lettuce), manually spun with a salad spinner with 40-50 revolutions, packaged in plastic bags (100-150 g per bag; 6×12×2 inches), and placed at 2.5° C. until all lettuce for that day was cut and packaged. These bags were completely randomized among the evaluation days and placed within 6 hours at 5° C. The above procedure was repeated for Blocks 2 and 3 on two subsequent days. There were three separate bags (replicates) for each evaluation time point.

Three bags per entry were evaluated the day of processing (day 0) and again after 7, 10, 14, 20, and 27 days (depending on quality). Lettuce pieces were evaluated for overall visual quality, discoloration on cut edges, decay, and any other defects or observations, with one score for the entire replicate bag. Photos were taken of the produce in each bag on each evaluation period.

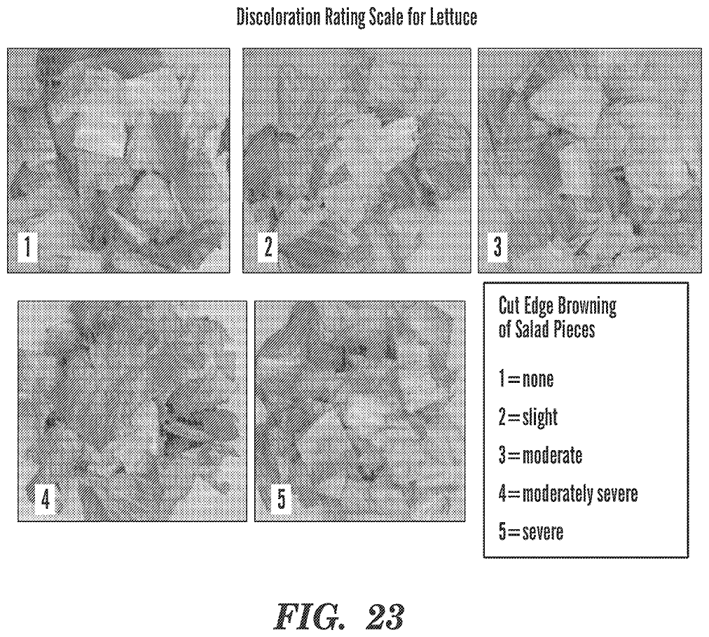

Discoloration defects (leaf surface or stem and ethylene-induced) were scored on a 1 to 5 scale, where 1=none, 2=slight, 3=moderate, 4=moderately severe, and 5=maximum or severe (FIG. 23). This scale was referenced to digital color photographs. A single score was given per sample.

Overall visual quality was scored on a 9 to 1 scale, where 9=excellent, fresh appearance, 7=good, 5=fair, 3=fair (useable but not saleable), 1=unusable. Intermediate numbers were assigned where appropriate. One visual quality score was given to an entire sample. A score of 6 is considered the limit of marketability.

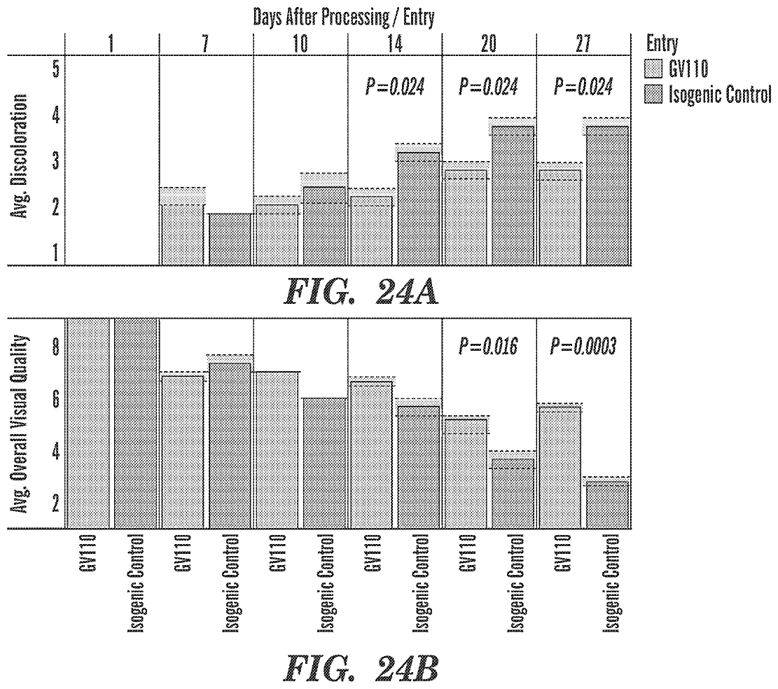

As shown in FIGS. 24A-B, PPO mutant line, GVR-110, outperformed the isogenic control line. The average discoloration of GVR-110 at 14, 20, and 27 days after processing was significantly lower than the control (P<0.024 at each time-point, FIG. 24A). The overall visual quality of PPO mutant line, GVR-110 remained high through 27 days after processing, retaining a score of almost 6 compared to Green Forest score of about 3 at the same stage (FIG. 24B). The improved visual quality of GVR-100 was significantly higher than the isogenic control at 20 days after processing (P<0.016) and 27 days after processing (P<0.0003).

### Example 11—Retention of Polyphenolic Levels in PPO Mutant Lines

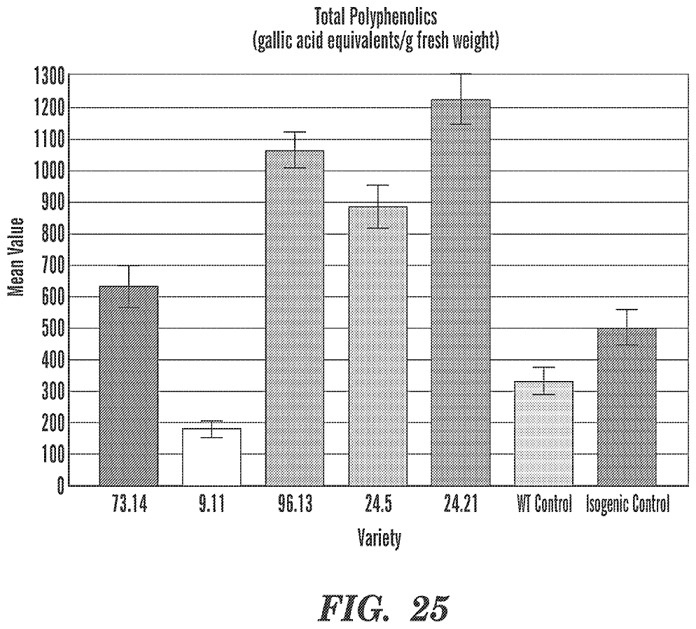

Polyphenolic levels were measured using the Folin-Ciocalteu (F-C) method. In summary, seven millimeter leaf disks harvested in strip-tubes were flash frozen before being homogenized in 2 ml ice-cold 95% methanol in a Tissuelyzer for 5 minutes at 30 Hz. Samples were then incubated at room temperature for 48 hrs in the dark before microcentrifugation for 5 minutes at 13,000 g. One hundred microliter supernatant was collected in fresh microcentrifuge tubes to which 200 μl of 10% F-C reagent was added. After thorough vortexing, 800 μl of 700 mM of sodium carbonate was added and tubes were incubated for 2 hrs at room temperature before measuring the absorbance at 765 nm. Phenolic content was extrapolated using a gallic acid standard curve. Two days after harvest, PPO mutant lines had higher levels of polyphenolics compared to control lettuce lines (FIG. 25). Polyphenolic levels ranged from around 1210 gallic acid equivalents (“GAE”)/g fresh weight (“gFW”) in GVR-107 (14-2-24-21), to 1060 GAE/gFW in GVR-110 (14-2-96-13) and 890 GAE/gFW in GVR-108 (14-2-24-5) compared to only 310-500 GAE/gFW in controls.

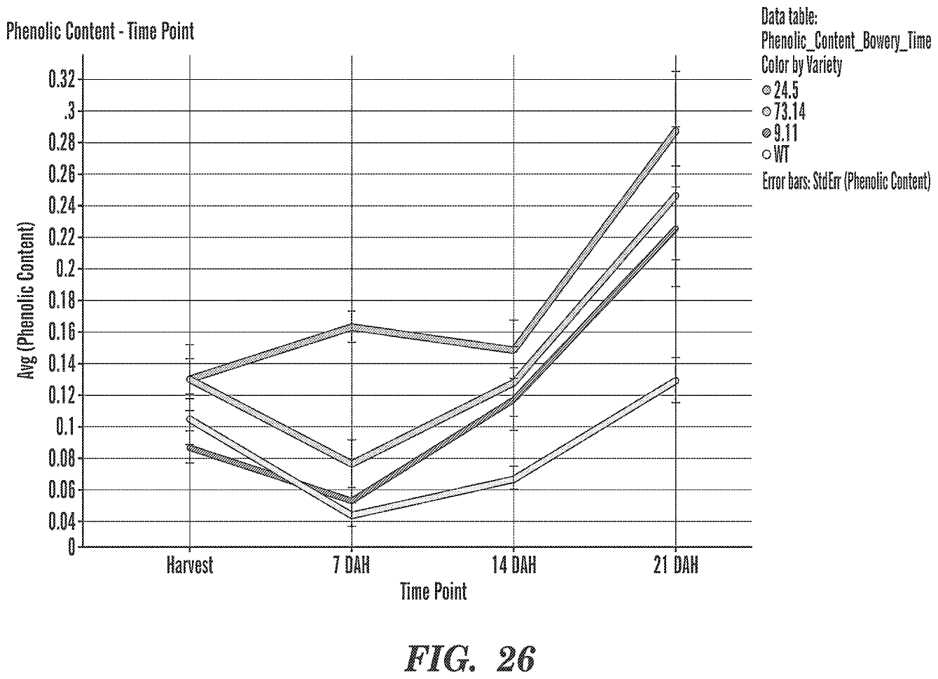

An analysis of phenolic levels at harvest, and at 7, 14, and 21 days after harvest showed that PPO mutant lines retained high levels of phenolics over time (FIG. 26). PPO mutant line GVR-108 (14-2-24-5) had an average phenolic content of 0.13 at harvest, which increased to 0.16 at 7 and 14 days after harvest (“DAH”), and increased further to 0.29 at 21 DAH. In contrast, the control variety started at around 0.11 average phenolic content at harvest, decreased to less than 0.05 at 7 DAH, and then increased to 0.07 at 14 DAH and only reached 0.13 at 21 DAH.

### Example 12—Fermentation, Organoleptic Evaluation, and Nutritional Analysis of Field Grown PPO Mutant Lines

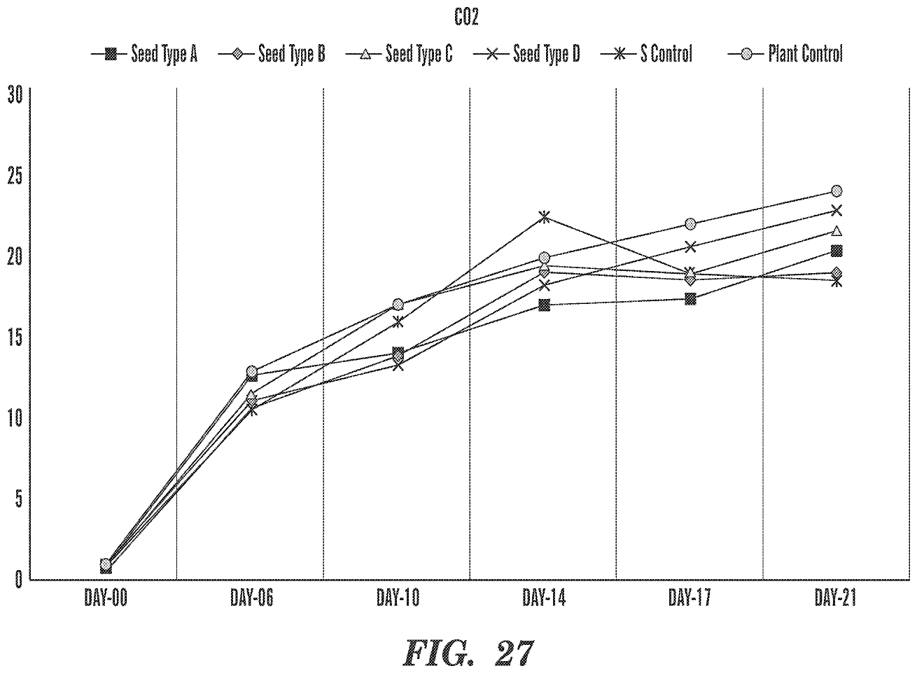

Fermentation in PPO mutant lines compared to controls in chopped lettuce leaves after harvest was assessed by CO₂ production (FIG. 27). Lettuce heads from the field were harvested, trimmed, and cooled prior to transport to a commercial facility of a third party partner. The lettuce leaves were washed, dried, and packaged in 10 ounce Caesar romaine film with a gas flush using the partner's proprietary protocol. Samples were stored in a shelf life cage at 34° F. until day 6, and then transferred into a cooler at 40° F. and stored until expiration. Head space was analyzed for CO₂ at harvest (day 0) and at day 6, 10, 14, 17, and 21 days after harvest. PPO mutant lines GVR-102 (“Seed Type A”), GVR-107 (“Seed Type B”), GVR-108 (“Seed Type C”), and GVR-110 (“Seed Type D”) were evaluated for CO₂ production along with isogenic control (“Plant Control”), and a control supplied by the seed supplier (“S Control”) (FIG. 27). The control accumulated more CO₂ at each time point compared to the majority of the PPO mutant lines. The control had CO₂ levels of 24% by day 21 after harvest compared to only 19% for PPO mutant line GVR-107 (FIG. 27). Plant control samples at days 17-21 after processing were considered strongly fermented. There was only minor fermentation observed in PPO mutant lines GVR-102, 107, 108, and 110.

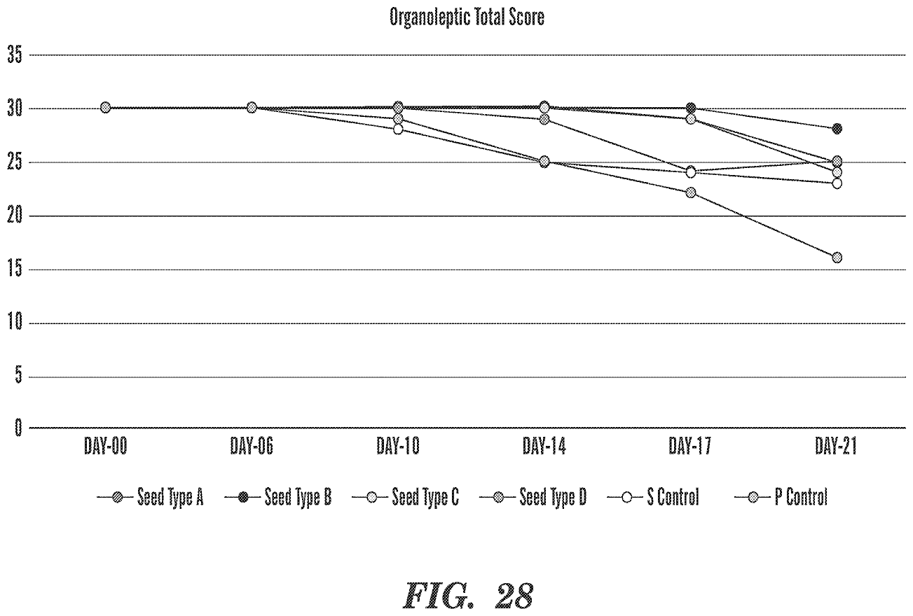

The appearance, color, aroma, flavor, texture and moisture of the PPO mutant lines was tested and compared to the controls at day 0, 6, 10, 14, 17, and 21 days after processing. Each parameter was given a score of 1-5, with 5 being the best score and 1 the worst score. Six bags of lettuce were tested at each time point. The combined scores for each line tested at each time point over time is shown in FIG. 28. Plant control samples at days 17-21 after processing were strongly fermented, bruised and wet and looked poor overall (FIG. 28). All of the PPO mutant lines were considered edible and acceptable with no significant pinking or oxidation except minor fermentation at 21 days after processing.

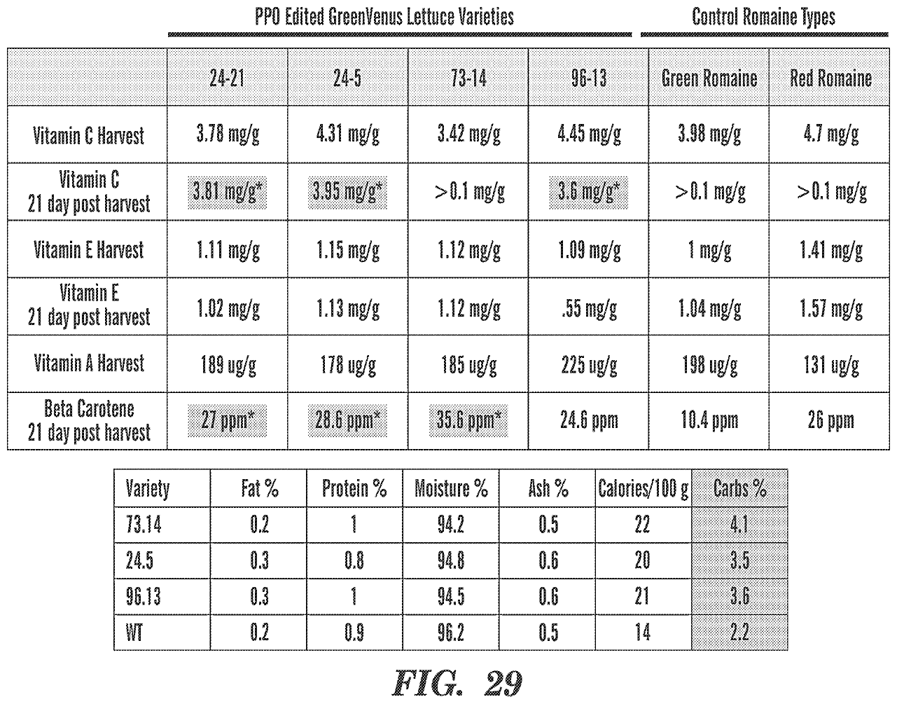

A nutritional evaluation of GVR-102, GVR-108, and GVR-110 and a control was performed at day 30 after processing, and is shown in FIG. 29. As described in Example 9, the PPO mutant lines retained more vitamin A at 30 days after processing. PPO mutant lines also retained more carbohydrates in agreement with the reduction in fermentation seen in PPO mutant lines. Overall, carbohydrates were about 1.6 to 2-fold higher in PPO mutant lines compared to the control.

### Example 13—Shelf Life Study of Aeroponic Grown PPO Mutant Lines

PPO mutant lines GVR-102 (14-2-73-14), GVR-107 (14-2-24-21), GVR-108 (14-2-24-5), and/or GVR-110 (14-2-96-13) were grown with an isogenic control line having no PPO mutations and also with a Red Romaine lettuce variety in flats aeroponically for 14 days using standard light intensity and spectrum. Each line was measured for shelf-life at 0, 7, 13, 21, and 27 days post-harvest. The lines were tested for organoleptic characteristics at 3, 7, 11, 17, and 21 days post-harvest. Vitamins were tested at harvest and at 21 days post-harvest as described in Example 9, supra (Trial 5 (AF)). Multispectral imaging using a Phenospex indicated no significant difference in green color for PPO mutant lines compared to the isogenic control (each was about 0.5 average greenness), and a significant difference compared to Red Romaine (0.2 average greenness).

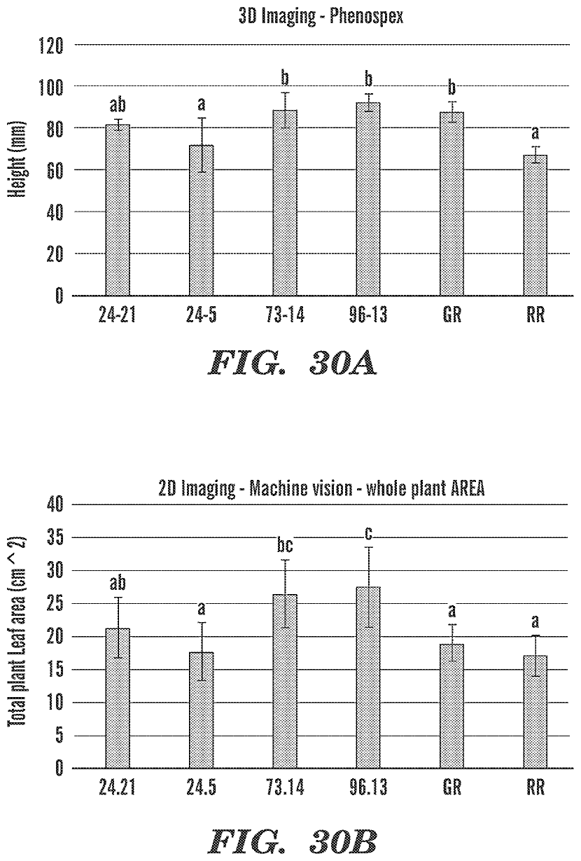

As shown in FIG. 30A, PPO mutant line GVR-108 (“24-5”) was shorter than the green romaine control (“GR”), and was more similar to the Red Romaine variety (“RR”). GVR-102 (“73-14”), GVR-107 (“24-21”), and GVR-110 (“96-13”) were not significantly different in height compared to the control. Total plant leaf area, as shown in FIG. 30B, was significantly higher for PPO mutant lines, GVR-102 (“73-14”), GVR-107 (“24-21”), and GVR-110 (“96-13”) compared to the green romaine control (“GR”). Overall yields, were similar for all PPO mutant lines compared to the isogenic control, except for GVR-108 (“24-5”), which may have been impacted by a pump mutation during the trial.

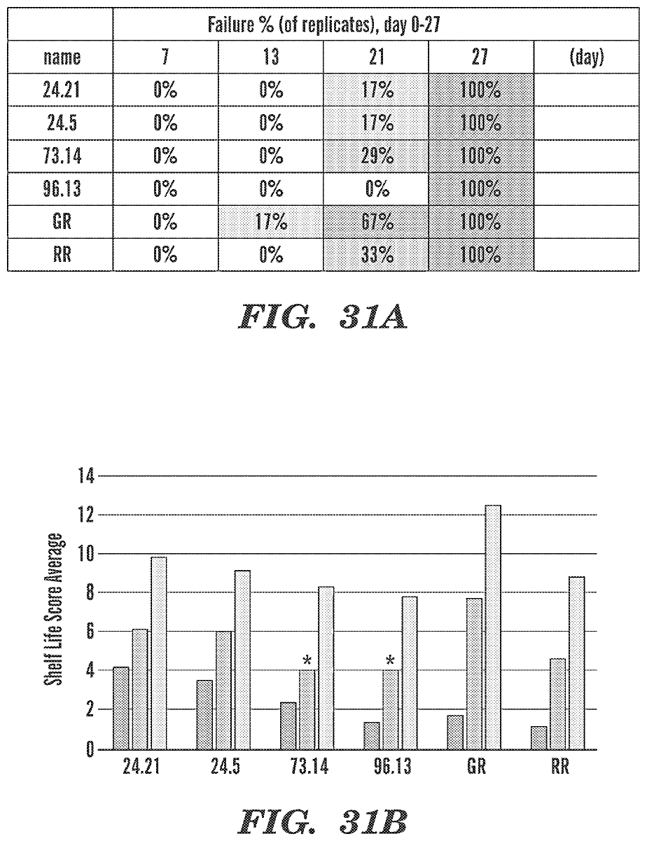

The shelf life evaluation of the PPO mutant lines showed that the failure rate of the PPO mutant lines was lower compared to the controls (FIG. 31A). At day 21, 67% of replicates failed for the isogenic control, whereas 0% failed for GVR-110 (“96-13”), and only 17% failed for GVR-107 (“24-21”) and GVR-108 (“24-5”). Shelf-life scoring averages at 7d, 13d, and 21d after harvest are shown in FIG. 31B. The shelf-life scoring of GVR-102 (“73-14”) and GVR-110 (“96-13”) were significantly better compared to the isogenic control (“GR”) at 13 days post-harvest (FIG. 31B).

Organoleptic evaluation of the lettuce lines in the trial included scoring appearance, taste, texture, aroma and overall sensory on a scale of 1-5 with 1 being poor and 5 meaning the best score. All varieties scored similarly for taste.

### Example 14—Sequencing of Multiple Lettuce Varieties for PPO Genes

A 7-mm leaf disk was collected from 7-10 days old seedlings of three new lettuce varieties, Romaine background PI658678 and SM13-R2 in addition to Coastline, a multi-leaf lettuce. Genomic DNA was extracted from each leaf disk using a CTAB protocol. Genomic DNA was amplified using primer combination listed in Table 11 to amplify the tyrosinase domain of each PPO target gene, and then PCR products were clean-up prior Sanger sequencing. Sequencing data were analyzed using Geneious by alignment to the reference isogenic gene sequences.

TABLE 11
Primers Used To Amplify PPO Genes
In Multiple Varieties

| PPO | Pri- | Primer | | SEQ |
|---|---|---|---|---|
| Tar- | mer | Orien- | | ID |
| get | Name | tation | Primer Sequence | NO: |
| PPO-A | AV67 | F | GGCAGCTCATTTGGCTAATA | 138 |
| | AV70 | R | CGAAGAATCAAGCGAGTCTT | 139 |
| PPO-B | AR93 | F | GCAGCTCATTTGGCTAATG | 140 |
| | AM43 | R | CGAAGAATCAAGCCAATCTT | 141 |
| PPO-D | AM95 | F | GATTCAAGCCATGAAGAATC | 142 |
| | | | TC | |
| | X90 | R | TGTGTCCCTTGATTCCCAAA | 143 |
| | | | TCT | |
| PPO-E | AN06 | F | AGCCACTGCCGATTACAT | 144 |
| | AT29 | R | CGTATGATGCATTTAGCCAGT | 145 |
| PPO-G | AN13 | F | CTAAGAATGAGGCGTTCAGG | 146 |
| | PPOG- | R | GTTTGCCTCCCTGCATCTTC | 147 |
| | 22R | | | |
| PPO-S | PPOS- | F | ATTGCGCTTACTGCAATG | 148 |
| | 11F | | | |
| | X45 | R | TCTGTCGGTTCTTTGCCTCC | 149 |

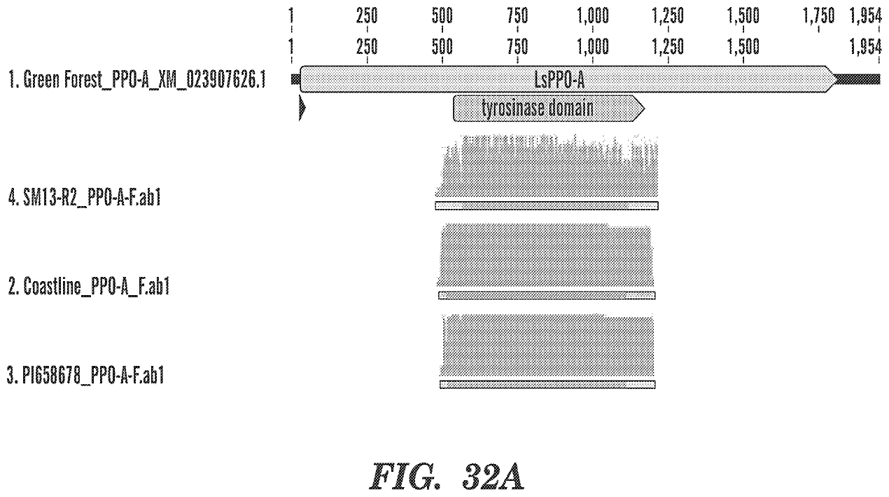

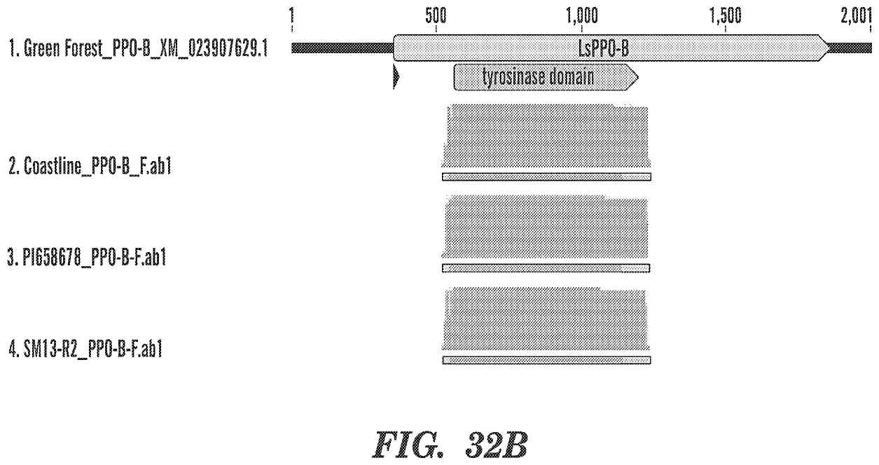

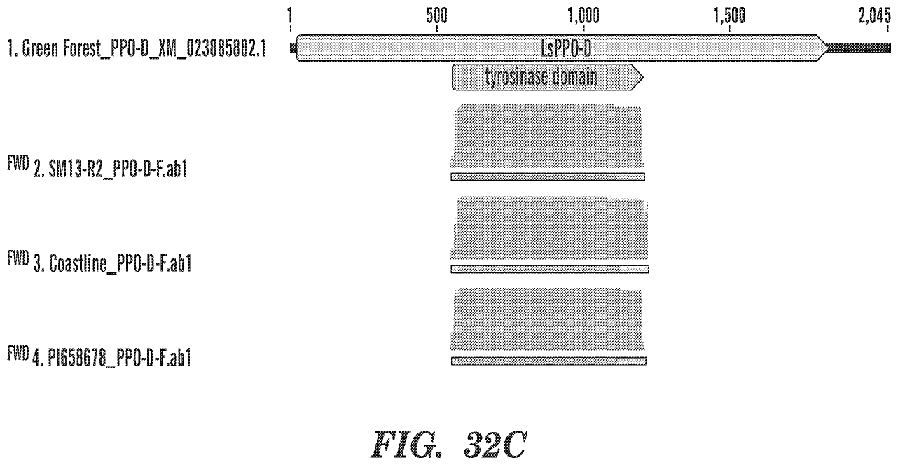

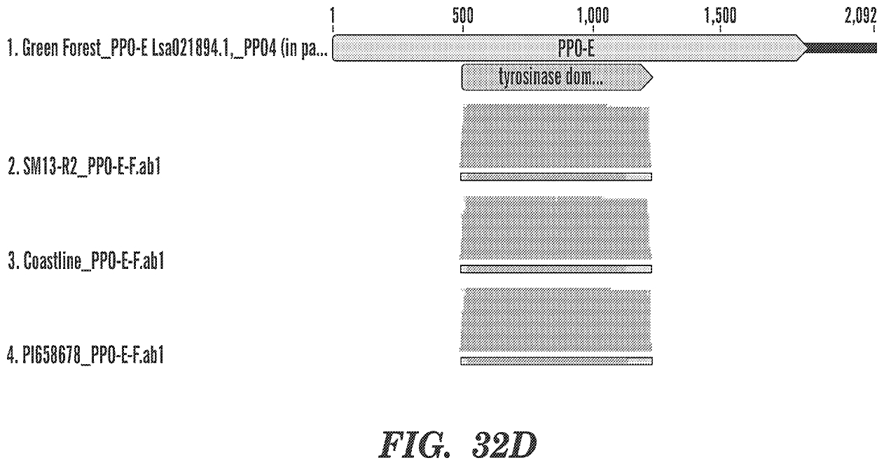

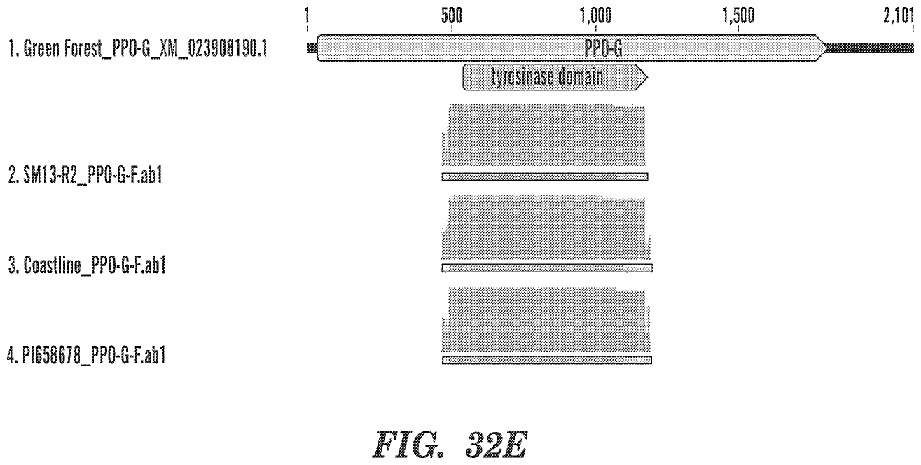

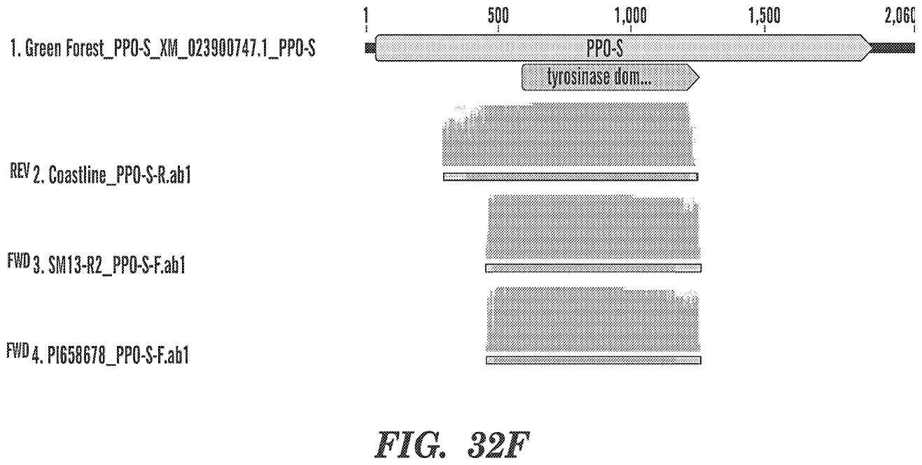

FIGS. 32A-F shown an alignment of the tyrosinase domain of selected PPO target gene from three new lettuce varieties against the isogenic control lettuce reference genes. Alignments in FIGS. 32A-F correspond to PPO-A (FIG. 32A), PPO-B (FIG. 32B), PPO-D (FIG. 32C), D) PPO-E (FIG. 32D), PPO-G (FIG. 32E), and PPO-S (FIG. 32F). The tyrosinase domain of each PPO target gene for the three new lettuce varieties proved to be 100% identical to the isogenic control reference target genes. The consensus tyrosinase domain sequences for each PPO target gene of these multiple lettuce varieties are given in Table 12.

TABLE 12
Consensus Tyrosinase Domains of Multiple Lettuce Variety PPO Genes

| | Tyrosinase Domain Nucleotide | SEQ |
|---|---|---|
| PPO Target | Sequences (5′ to 3′) | ID NO: |
| PPO-A | CCTCGTAGTTTCAAGCAACAAGCTGCTGTTCATTGTGCGTATT | 150 |
| Tyrosinase | GCGATGGGGCATACGATCAAGTCGGTTTCCCTGATCTCGAGCT | |
| Domain | TCAAGTCCATGGCTCATGGTTGTTCTTACCTTTCCACCGCTAT | |
| Consensus | TACTTATACTTCTTCGAGAAAATTTGTGGCAAATTAATCGATG | |
| | ATCCAAATTTCGCAATCCCTTTTTGGAACTGGGATGCACCTGA | |
| | TGGCATGAAGATCCCTGATATTTACACGAATAAGAAATCTCCG | |
| | TTGTACGATGCTCTTCGTGATGCGAAGCATCAACCACCGTCTC | |
| | TGATTGATCTTGACTACAATGGTGACGATGAAAATCTTAGCCG | |
| | ATCGAAACAAACCTCCACAAATCTCACAATTATGTACAGACAA | |
| | ATGGTGTCTAGTTCCAAGACTGCTAGTCTTTTCATGGGTAGTC | |
| | CTTATCGTGCAGGTGATGAGGCTAGCCCTGGCTCTGGCTCGCT | |
| | CGAGAGCATACCACATGGCCCGGTTCATATCTGGACCGGAGAT | |
| | AGGAACCAGCAAAATGGTGAAGACATGGGTAACTTTTATTCTG | |
| | CAGCCAGAGACCCTATTTTCTATGCACATCATGCGAATATCGA | |
| | CAGAATGTGGTCAGTTTGGAAAACTCTA | |
| PPO-B | CCTCGTAGCTTTAAGCAGCAAGCAAATGTTCATTGTGCCTATT | 151 |
| Tyrosinase | GCGATGGCGCGTATGACCAAGTCGGTTTTCCAGATCTGGAGCT | |
| Domain | TCAAGTACATAACTCATGGCTGTTCTTCCCTTTCCATCGCTAT | |
| Consensus | TACATGTACTTCTTCGAGAAAATTTGTGGCAAGTTAATTGATG | |
| | ACCCAAATTTCGCAATTCCATTTTGGAACTGGGATGCACCAGA | |
| | TGGGATGAAAATCCCTGATATTTACACAAATAAGAAATCTTCG | |
| | TTGTATGATCCTCTTCGCGATGTGGACCATCAACCACCGTCTT | |
| | TGATTGATCTTGACTTCAATGGTGTCGACGAAAATCTTAGCCC | |
| | CTCTGAACAAACGTCCAAAAATCTCACAGTTATGTATAGACAA | |
| | ATGGTGTCTAGTTCCAAGACTTCTACTCTTTTCATGGGTAGTC | |
| | CTTATCGTGCAGGCGATGATGCTAGCCCTGGTAGTGGTTCGAT | |
| | CGAGAACACACCACATAACCCAGTTCATATCTGGGCCGGTGAG | |
| | TGGAAGCATAATAATGGCAAAAACATGGGCAAACTTTATTCTG | |
| | CAGCCAGGGACCCTCTTTTCTATGCACATCATGGGAATATTGA | |
| | TAGAATGTGGTCAGTTTGGAAAACACTA | |
| PPO-D | TCCACACAGTTGGAAGCAACAAGCTAAGATCCACTGCGCTTAT | 152 |
| Tyrosinase | TGCAACGGTGGTTACAATCAAGAACAGAGTGGTTTCCCGGACA | |
| Domain | TACAACTCCAGATTCACAACACATGGCTCTTCTTTCCTTTCCA | |
| Consensus | CCGATGGTACCTCTACTTCTACGAGAGGATTTTGGGGAAGTTG | |
| | ATTAATGATCCAACTTTCGCTTTACCATACTGGAACTGGGATA | |
| | ACCCTACCGGAATGGTGCTCCCTGCCATGTTCGAAACCGACGG | |
| | CAAAAGGAACCCTATCTTTGACCCTTACAGGAATGCCACACAC | |
| | CTCCCACCAGCTATCTTTGAAGTGGGATATAATGGGACAGACA | |
| | GTGGCGCCACTTGTATAGACCAGATAAGCGCTAATCTGTCTTT | |
| | GATGTACAAGCAAATGATCACCAACGCTCCTGATACAACAACG | |
| | TTCTTCGGTGGAGAATTTGTTGCTGGGGATGACCCTCTTAACA | |
| | AAGAGTTTAACGTTGCTGGGTCCATAGAGGCTGGGGTTCACAC | |
| | TGCGGCGCATAGATGGGTGGGTGATCCTAGGATGGCCAACAGC | |
| | GAGGACATGGGGAACTTCTACTCCGCAGGGTATGATCCTCTCT | |
| | TTTACGTCCACCATGCCAACGTCGACCGGATGTGGAAAATCTG | |
| | GAAAGATTTG | |
| PPO-E | CCCACATAGCTGGAAGCAACAAGGCAAGATTCACTGTGCTTAT | 153 |
| Tyrosinase | TGCAACGGTGGTTACAATCAAGAACAAAGTGGTTACCCGAATT | |
| Domain | TACAACTTCAGATTCACAACTCATGGCTCTTCTTTCCTTTCCA | |
| Consensus | CCGGTGGTACCTCTATTTCTACGAGAAGATATTGGGGAAGTTG | |
| | ATTAATGATCCAACTTTCGCTCTACCTTACTGGAACTGGGATA | |
| | ACCCTACTGGAATGGTTATTCCTGCCATGTTCGAACAGAACAG | |
| | CAAAACTAACTCTCTGTTTGACCCTTTAAGGGATGCGAAACAC | |
| | CTCCCACCTTCTATCTTTGATGTTGAATATGCTGGTGCAGACA | |
| | CTGGTGCCACTTGTATAGACCAGATAGCCATTAATCTGTCTTC | |
| | AATGTACAGACAGATGGTCACCAACTCCACTGATACAAAACGA | |
| | TTCTTCGGTGGCGAATTTGTAGCTGGAAATGACCCTCTTGCGA | |
| | GCGAGTTCAACGTAGCTGGGACCGTAGAAGCTGGGGTTCACAC | |
| | TGCGGCTCACCGCTGGGTGGGTAATTCTAGGATGGCCAACAGC | |
| | GAAGACATGGGGAACTTCTACTCCGCAGGATATGATCCTCTCT | |
| | TTTACGTCCACCATGCGAATGTCGACAGGATGTGGCAAATCTG | |
| | GAAAGATATT | |
| PPO-G | TCCTCGAAACTTTCTGCAACAAGCACACATTCACTGTGCTTAC | 154 |
| Tyrosinase | TGCAATGGCGCTTACACTCAATCTTCAAGTGGATTTCCCGATA | |
| Domain | TTGAAATCCAGATTCATAACTCATGGCTGTTCTTCCCCTTCCA | |
| Consensus | CCGTTGGTATCTCTACTTTTACGAGAGAATCCTGGGGAGCTTG | |
| | ATCGATGATCCCACTTTCGCTTTGCCATTCTGGAACTGGGACA | |
| | CCCCTGCCGGAATGACAATTCCGAAATACTTTAACGATCCCAA | |
| | AAACGCAGTTTTTGATCCCAAAAGAAACCAAGGTCACTTGCAA | |
| | GGAGTCGTCGATCTGGGTTACAATGGGAAAGATTCAGACACTA | |
| | CTGATATCGAAAAGGTGAAGAACAATCTCGCGATAATGTATCG | |
| | TCAAATGGTGACAAACGCCACCGACCCCACAGCTTTCTTCGGT | |
| | GGTGAGTATCGTGCCGGAATCGAACCCATTAGCGGTGGTGGAT | |
| | CAGTCGAACAAAGCCCACACACACCTGTTCACCGGTGGGTCGG | |
| | TGACCCAAGAGAACTTAACGGTGAAAACCTCGGTAACTTCTAC | |
| | TCCGCCGGTCGTGACACGCTCTTTTACTGTCACCATTCCAACG | |
| | TCGATCGAATGTGGTCGTTGTGGAAGATGCAG | |
| PPO-S | CCCACGCAGTTTCAATAACCAAGCTAAAGTTCATTGCGCTTAC | 155 |
| Tyrosinase | TGCAATGGCAGTTACACTCAAAACGGTCAAGAACTCCAGATTC | |
| Domain | ACAACTCCTGGCTCTTCTTTCCCTTCCATCGGTGGTACCTTTA | |
| Consensus | TTTCTACGAGAGGATACTGGGAGATCTCATTGGTGATTCGACA | |
| | TTCGGGTTACCCTACTGGAACTGGGACAACCCCGAAGGAATGA | |
| | CAATTCCACACTTCTTCGTAGAGAAACAATGTAACAACTATAA | |
| | GTTCGAAAACGGAGAAAACCCTCTATATGATAAGTATCGGGAC | |
| | GAAAGTCACCTTCGGTATGAATTGGTCGATCTTGACTACTCAG | |
| | GGAGAAACCGCGACCTGTGTTACGATCAGAAAGAAATCAATCT | |
| | GGCTACTATGAATAGGCAGATGATGCGCAACGCCTTTGATGCA | |
| | ACAAGCTTCTTCGGTGGCAAATATGTAGCCGGTGATGAACCGA | |
| | TTCCCCGAGGAGATAATGTAGTTGGATCCGTGGAGGCTGGTTG | |
| | TCATACGGCTGTTCACAGATGGGTTGGGAACCCTGATCCAAAA | |
| | GGGAATAAAGAGGACATGGGCAACTTCTACTCTGCGGGATATG | |
| | ATCCTTTGTTCTACGTCCACCATTCTAATGTGGACCGAATGTG | |
| | GACTCTTTGGAAGCAAATG | |

### Example 15—Fusarium Testing of PPO Mutant Lines

Twenty-four 2″ pots of each material were sown in the greenhouse. Plants were grown for 21 days (between 23 and 27°° C.) in fertilized peat potting medium. Plants were inoculated with 5 ml of a spore suspension of Fusarium oxysporum f. sp. lactucae race 1 (grown in Czapek-Dox broth, adjusted to a concentration of 1 million spores/ml). Two replicates of up to 8 plants per line/variety were randomly ordered. Roots were evaluated at 21 days post inoculation following a 1-9 scale (1, susceptible and 9, resistant).

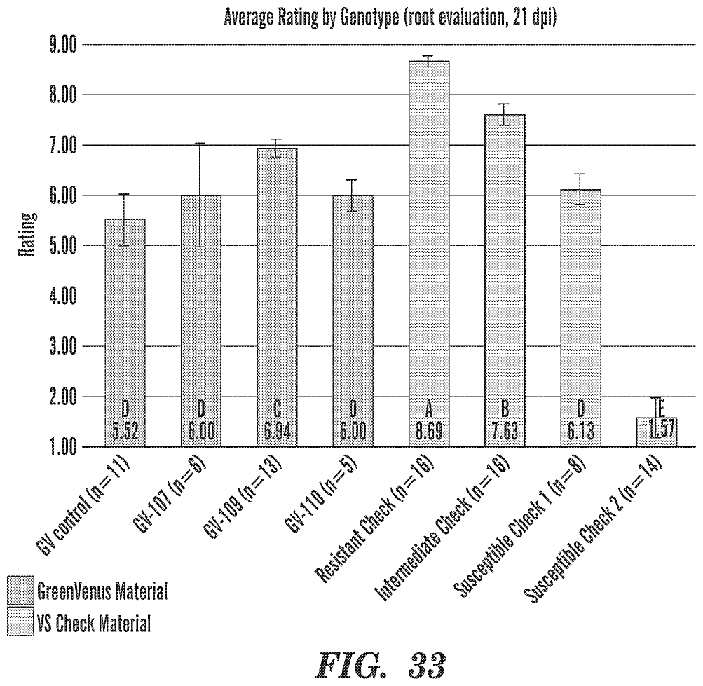

As shown in FIG. 33, although some reports in the literature show that some PPOs may be important for disease resistance, unexpectedly, no significant differences were observed between PPO edited lines compared to their isogenic controls.

### Example 16—Field Yield of PPO Mutant Lines

Lettuce varieties of the present application along with the respective market standard Romaine were planted in full bed trials in Salinas, CA (planted in spring and harvested mid-summer). Romaine heads were quantified for yield and head characteristics as in weight, height, heart length, and core length.

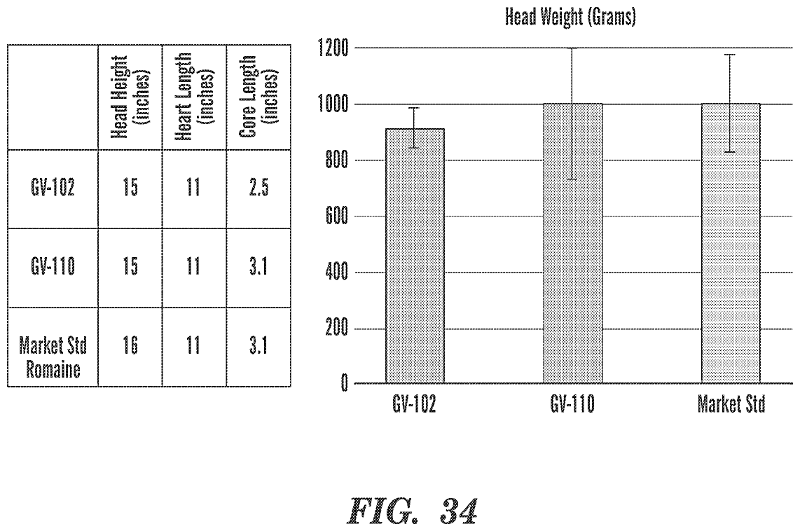

Data as shown in FIG. 34 indicate that the lettuce varieties with multiple PPO edits performed equivalent to market standard.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A lettuce plant comprising a mutation in each of at least two different PPO genes, wherein said mutations reduce the activity of PPO protein compared to a wild type lettuce plant.

2. The lettuce plant of claim 1, wherein at least one of said mutations occurs in both alleles of the PPO gene.

3. The lettuce plant of claim 1, wherein at least one of said mutations occurs in only one allele of the PPO genes.

4. The lettuce plant according any one of the preceding claims, wherein the at least two different PPO genes are selected from the group consisting of PPO-A, PPO-B, PPO-C, PPO-D, PPO-E, PPO-G, PPO-O, PPO-P, PPO-R, PPO-N, PPO-M, PPO-J, PPO-Q, and PPO-S.

5. The lettuce plant according to any one of the preceding claims, wherein the at least two different PPO genes comprise PPO-B and at least one of PPO-S and PPO-G.

6. The lettuce plant according to claim 5 further comprising:
a mutation in one or more of PPO-A, PPO-C, PPO-O, PPO-P, and PPO-R.

7. The lettuce plant according to claim 5 or claim 6, wherein the plant comprises a mutation in PPO-S.

8. The lettuce plant according to any one of claims 5-7, wherein the plant comprises a mutation in PPO-G.

9. The lettuce plant according to any one of claims 5-8 further comprising:
a mutation in one or more of PPO-D and PPO-E.

10. The lettuce plant of any one of the preceding claims, wherein said lettuce is leaf lettuce, romaine lettuce, Frisée lettuce, butter lettuce, Bibb lettuce, Boston lettuce, iceberg lettuce, kale, spinach, radicchio, or endive.

11. The lettuce plant of claim 10, wherein said lettuce is romaine lettuce.

12. The lettuce plant of any one of the preceding claims, wherein said mutations reduce the amount of active PPO protein in the lettuce plant.

13. The lettuce plant of any one of the preceding claims, wherein said mutations reduce the activity of PPO protein in the lettuce plant.

14. The lettuce plant of any one of the preceding claims, wherein the activity of PPO protein is reduced by at least 10%.

15. The lettuce plant of any one of the preceding claims, wherein the activity of PPO protein is reduced by at least 30%.

16. The lettuce plant of any one of the preceding claims, wherein the activity of PPO protein is reduced by at least 50%.

17. The lettuce plant of any one of the preceding claims, wherein the lettuce plant at harvest comprises a characteristic selected from the group consisting of: reduced tip burn compared to a wild type variety under the same conditions; higher levels of polyphenolics compared to a wild type variety under the same conditions; higher levels of vitamin A or beta-carotene compared to a wild type variety under the same conditions; higher levels of vitamin C compared to a wild type variety under the same conditions; and higher levels of vitamin K compared to a wild type variety under the same conditions.

18. The lettuce plant of any one of the preceding claims, wherein the lettuce plant post-harvest comprises a characteristic selected from the group consisting of: reduced browning; reduced tip burn; increased shelf life; higher levels of vitamin A; higher levels of vitamin C; lower levels of CO₂ production; higher levels of carbohydrates; less yellowing as assessed by midvein scoring; and higher levels of polyphenolics compared to a wild type variety under the same conditions.

19. The lettuce plant of claim 18, wherein said post-harvest is 7, 13, 14, 21, 27, or 30 days after harvest.

20. The lettuce plant of any one of the preceding claims, wherein the mutations encode a knockout mutation.

21. The lettuce plant of any one of the preceding claims, wherein the mutation is an insertion between nucleotides 1112 and 1113 of SEQ ID NO:9.

22. The lettuce plant of any one of the preceding claims, wherein the mutation is an insertion between nucleotides 988 and 989 of SEQ ID NO:11, a deletion at nucleotide 987 of SEQ ID NO:11, or a deletion between nucleotides 986 and 987 of SEQ ID NO:11.

23. The lettuce plant of any one of the preceding claims, wherein the mutation is an insertion between nucleotides 1131 and 1132 of SEQ ID NO:7.

24. The lettuce plant of any one of the preceding claims, wherein the mutation is an insertion between nucleotides 1044 and 1045 of SEQ ID NO:27, a deletion of three nucleotides between nucleotides 1045-1047 of SEQ ID NO:27, or a deletion of seven nucleotides between nucleotides 1045-1051 of SEQ ID NO:27.

25. The lettuce plant of any one of the preceding claims, wherein the mutation is an insertion between nucleotides 976 and 977 of SEQ ID NO:1, a deletion of 26 nucleotides between nucleotides 971-996 of SEQ ID NO:1, or a deletion of 27 nucleotides between nucleotides 971-997 of SEQ ID NO:1.

26. The lettuce plant of any one of the preceding claims, wherein the mutation is an insertion between nucleotides 1007 and 1008 of SEQ ID NO:3 or a deletion of nucleotide 1008 of SEQ ID NO:3.

27. The lettuce plant of any one of the preceding claims, wherein the mutation is an insertion between nucleotides 1023 and 1024 of SEQ ID NO:25.

28. The lettuce plant of any one of the preceding claims, wherein the lettuce plant is free of any plant pest sequences.

29. The lettuce plant of any one of the preceding claims, wherein the lettuce plant is free of any selectable marker.

30. A method of making a lettuce plant with reduced PPO activity, said method comprising:
introducing a mutation into each of at least two different PPO genes of a lettuce plant, wherein said mutations reduce the amount and/or activity of PPO protein compared to a wild type lettuce plant.

31. The method of claim 30, wherein the mutations encode a knockout or a knock-down mutation.

32. The method of claim 30 or claim 31, wherein the mutation is an insertion between nucleotides 1112 and 1113 of SEQ ID NO:9.

33. The method of any one of claims 30-32, wherein the mutation is an insertion between nucleotides 988 and 989 of SEQ ID NO:11, a deletion at nucleotide 987 of SEQ ID NO:11, or a deletion between nucleotides 986 and 987 of SEQ ID NO:11.

34. The method of any one of claims 30-33, wherein the mutation is an insertion between nucleotides 1131 and 1132 of SEQ ID NO: 7.

35. The method of any one of claims 30-34, wherein the mutation is an insertion between nucleotides 1044 and 1045 of SEQ ID NO:27, a deletion of three nucleotides between nucleotides 1045-1047 of SEQ ID NO:27, or a deletion of seven nucleotides between nucleotides 1045-1051 of SEQ ID NO:27.

36. The method of any one of claims 30-35, wherein the mutation is an insertion between nucleotides 976 and 977 of SEQ ID NO:1, a deletion of 26 nucleotides between nucleotides 971-996 of SEQ ID NO:1, or a deletion of 27 nucleotides between nucleotides 971-997 of SEQ ID NO:1.

37. The method of any one of claims 30-36, wherein the mutation is an insertion between nucleotides 1007 and 1008 of SEQ ID NO:3 or a deletion of nucleotide 1008 of SEQ ID NO:3.

38. The method of any one of claims 30-37, wherein the mutation is an insertion between nucleotides 1023 and 1024 of SEQ ID NO:25.

39. The method of any one of claims 30-38, wherein said lettuce is leaf lettuce, romaine lettuce, Frisée lettuce, butter lettuce, Bibb lettuce, Boston lettuce, iceberg lettuce, kale, spinach, radicchio, or endive.

40. The method of claim 39, wherein said lettuce is romaine lettuce.

41. The method of any one of claims 30-40, wherein said mutations reduce the amount of active PPO protein in the lettuce plant.

42. The method of any one of claims 30-41, wherein said mutations reduce the activity of PPO protein in the lettuce plant.

43. The method of any one of claims 30-42, wherein active PPO protein is reduced by at least 10%.

44. The method of any one of claims 30-43, wherein active PPO protein is reduced by at least 30%.

45. The method of any one of claims 30-44, wherein active PPO protein is reduced by at least 50%.

46. The lettuce plant of any one of the preceding claims, wherein the lettuce plant at harvest comprises a characteristic selected from the group consisting of: reduced tip burn compared to a wild type variety under the same conditions; higher levels of polyphenolics compared to a wild type variety under the same conditions; higher levels of vitamin A or beta-carotene compared to a wild type variety under the same conditions; higher levels of vitamin C compared to a wild type variety under the same conditions; and higher levels of vitamin K compared to a wild type variety under the same conditions.

47. The lettuce plant of any one of the preceding claims, wherein the lettuce plant post-harvest comprises a characteristic selected from the group consisting of reduced browning; reduced tip burn; increased shelf life; higher levels of vitamin A; higher levels of vitamin C; lower levels of CO₂ production; less rigorous modified atmosphere packaging; higher levels of carbohydrates; less yellowing as assessed by midvein scoring; and higher levels of polyphenolics compared to a wild type variety under the same conditions.

48. The lettuce plant of claim 47, wherein said post-harvest is 7, 13, 14, 21, 27, or 30 days after harvest.

49. A method of editing PPO genes of lettuce plant, said method comprising:
introducing into a lettuce plant a polynucleotide construct comprising:
a first nucleic acid sequence encoding a gene editing nuclease;
a promoter that is functional in plants operably linked to said first nucleic acid sequence;
a second nucleic acid sequence encoding one or more gRNA in a polycistronic arrangement targeting at least two PPO genes of choice to edit; and
a second promoter that is functional in plants, operably linked to said second nucleic acid sequence.

50. The method of claim 49, wherein said gene editing nuclease is a Cas9 nuclease, a MAD7 nuclease, a Cpf1 nuclease, or a chimeric nuclease.

51. The method of claim 50, wherein said chimeric nuclease comprises the amino acid sequence of SEQ ID NO:30.

52. The method of any one of claims 49 to 51, wherein said first promoter and said second promoter are each selected from AtU6 promoter, 35S promoter, and CsVMV promoter.

53. The method of any one of claims 49 to 52 further comprising:
a third nucleic acid sequence encoding a selectable marker operably linked to a third promoter that is functional in plants.

54. The method of claim 53, wherein said selectable marker is a drug resistance gene, a fluorescent protein, or an enzyme.

55. The method of claim 54, wherein said selectable marker is a kanamycin resistance gene or a neomycin resistance gene.

56. The method of any one of claims 49 to 55 further comprising:
regenerating said protoplast into a plant.

57. A polynucleotide construct for editing polyphenol oxidase genes of a lettuce plant comprising:
a nucleic acid sequence encoding a plurality of gRNAs targeting at least two PPO genes operably linked to a promoter.

58. The polynucleotide construct of claim 57, wherein the PPO genes are selected from the group consisting of PPO-A, PPO-B, PPO-C, PPO-D, PPO-E, PPO-G, PPO-O, PPO-P, PPO-R, PPO-N, PPO-M, PPO-J, PPO-Q, and PPO-S.

59. The polynucleotide of claim 57 or claim 58, wherein said polynucleotide comprises a second nucleic acid sequence encoding a gene editing enzyme.

60. The polynucleotide of claim 58, wherein said gene editing enzyme is Cas9, MAD7, Cpf1, or SynNuc1.
